(19) United States
(12) Patent Application Publication
(10) Pub. No.: US 2011/0177029 A1
(43) Pub. Date: Jul. 21, 2011

(54) O-LINKED GLYCOSYLATION USING N-ACETYLGLUCOSAMINYL TRANSFERASES

(75) Inventor: Shawn DeFrees, North Wales, PA (US)

(73) Assignee: NOVO NORDISK A/S, Bagsvaerd (DK)

(21) Appl. No.: 12/663,056

(22) PCT Filed: Jun. 4, 2008
(86) PCT No.: PCT/US08/65825

§ 371 Date: Feb. 9, 2010

Related U.S. Application Data
(60) Provisional application No. 60/941,926, filed on Jun. 4, 2007.

(51) Int. Cl. A61K 38/21; C07H 19/10; C12P 21/00; C07K 14/51; C07K 14/535; C07K 14/56; C07K 14/61; C07K 19/00; A61K 38/18; A61K 38/19; A61K 38/27; A61K 39/395; C07H 21/00; C12N 15/63; C12N 1/00; C12N 5/10

(52) U.S. Cl. 424/85.7; 536/26.23; 435/68.1; 530/399; 530/351; 530/387.3; 514/8.8; 424/85.1; 514/11.4; 424/134.1; 536/23.4; 536/23.52; 536/23.51; 536/23.5; 435/320.1; 435/243; 435/325

(57) ABSTRACT

The present invention provides covalent conjugates between a polypeptide and a modifying group, such as a water-soluble polymer (e.g., PEG). The amino acid sequence of the polypeptide includes one or more O-linked glycosylation sequence, each being a substrate for a GIcNAc transferase. The modifying group is covalently linked to the polypeptide via a glycosyl-linking group interposed between and covalently linked to both the polypeptide and the modifying group. In one embodiment, a glucosamine linking group is directly attached to an amino acid residue of the O-linked glycosylation sequence. The invention further provides methods of making polypeptide conjugates. The present invention also provides non-naturally occurring polypeptides that include at least one O-linked linked glycosylation sequence of the invention, wherein each glycosylation sequence is a substrate for a GIcNAc transferase. The invention further provides pharmaceutical compositions that include a polypeptide conjugate of the invention.

### CROSS-REFERENCES TO RELATED APPLICATIONS

This application claims priority under 35 U.S.C. §119(e) to U.S. Provisional Patent Application No. 60/941,926 filed on Jun. 4, 2007, which is incorporated herein by reference in its entirety for all purposes.

### FIELD OF THE INVENTION

The invention pertains to the field of peptide modification by glycosylation. In particular, the invention relates to peptide conjugates including a polymeric modifying group and methods of preparing glycosylated peptides using glycosylation sequences, which are recognized as a substrate by a GlcNAc transferase.

### BACKGROUND OF THE INVENTION

The administration of glycosylated and non-glycosylated polypeptides for engendering a particular physiological response is well known in the medicinal arts. For example, both purified and recombinant hGH are used for treating conditions and diseases associated with hGH deficiency, e.g., dwarfism in children. Other examples involve interferon, which has known antiviral activity as well as granulocyte colony stimulating factor, which stimulates the production of white blood cells.

The lack of expression systems that can be used to manufacture polypeptides with wild-type glycosylation patterns has limited the use of such polypeptides as therapeutic agents. It is known in the art that improperly or incompletely glycosylated peptides can be immunogenic, leading to neutralization of the peptide and/or the development of an allergic response. Other deficiencies of recombinantly produced glycopeptides include suboptimal potency and rapid clearance from the bloodstream.

One approach to solving the problems inherent in the production of glycosylated peptide therapeutics has been to modify the peptides in vitro after their expression. Post-expression in vitro modification of polypeptides has been used for both the modification of existing glycan structures and the attachment of glycosyl moieties to non-glycosylated amino acid residues. A comprehensive selection of recombinant eukaryotic glycosyltransferases has become available, making in vitro enzymatic synthesis of mammalian glycoconjugates with custom designed glycosylation patterns and glycosyl structures possible. See, for example, U.S. Pat. Nos. 5,876,980; 6,030,815; 5,728,554; 5,922,577; as well as WO/9831826; US2003180835; and WO 03/031464.

In addition, polypeptides have been derivatized with one or more non-saccharide modifying groups, such as water soluble polymers. An exemplary polymer that has been conjugated to peptides is poly(ethylene glycol) (“PEG”). PEG-conjugation, which increases the molecular size of the polypeptide, has been used to reduce immunogenicity and to prolong the time that the PEG-conjugated polypeptides stays in circulation. For example, U.S. Pat. No. 4,179,337 to Davis et al. discloses non-immunogenic polypeptides such as enzymes and peptide-hormones coupled to polyethylene glycol (PEG) or polypropylene glycol (PPG).

The principal method for the attachment of PEG and its derivatives to polypeptides involves non-specific bonding through an amino acid residue (see e.g., U.S. Pat. No. 4,088,538 U.S. Pat. No. 4,496,689, U.S. Pat. No. 4,414,147, U.S. Pat. No. 4,055,635, and PCT WO 87/00056). Another method of PEG-conjugation involves the non-specific oxidation of glycosyl residues of a glycopeptide (see e.g., WO 94/05332).

In these non-specific methods, PEG is added in a random, non-specific manner to reactive residues on a polypeptide backbone. This approach has significant drawbacks, including a lack of homogeneity of the final product, and the possibility of reduced biological or enzymatic activity of the modified polypeptide. Therefore, a derivatization method for therapeutic peptides that results in the formation of a specifically labeled, readily characterizable and essentially homogeneous product is highly desirable.

Specifically modified, homogeneous peptide therapeutics can be produced in vitro through the use of enzymes. Unlike non-specific methods for attaching a modifying group (e.g., a synthetic polymer) to a peptide, enzyme-based syntheses have the advantages of regioselectivity and stereoselectivity. Two principal classes of enzymes for use in the synthesis of labeled peptides are glycosyltransferases (e.g., sialyltransferases, oligosaccharyltransferases, N-acetylglucosaminyltransferases), and glycosidases. These enzymes can be used for the specific attachment of sugars which can subsequently be altered to comprise a modifying group. Alternatively, glycosyltransferases and modified glycosidases can be used to directly transfer modified sugars to a peptide backbone (see e.g., U.S. Pat. No. 6,399,336, and U.S. Patent Application Publications 20030040037, 20040132640, 20040137557, 20040126838, and 20040142856, each of which are incorporated by reference herein). Methods combining both chemical and enzymatic approaches are also known (see e.g., Yamamoto et al., Carbohydr. Res. 305: 415-422 (1998) and U.S. Patent Application Publication 20040137557, which is incorporated herein by reference).

Carbohydrates are attached to glycopeptides in several ways of which N-linked to asparagine and O-linked to serine and threonine are the most relevant for recombinant glycoprotein therapeuctics. O-linked glycosylation is found on secreted and cell surface associated glycoproteins of all eukaryotic cells. There is great diversity in the structures created by O-linked glycosylation. Such glycans are produced by the catalytic activity of hundreds of enzymes (glycosyltransferases) that are resident in the Golgi complex. Diversity exists at the level of the glycan structure and in positions of attachment of O-glycans to the protein backbones. Despite the high degree of potential diversity, it is clear that O-linked glycosylation is a highly regulated process that shows a high degree of conservation among multicellular organisms.

Unfortunately, not all polypeptides comprise an O-linked glycosylation sequence as part of their amino acid sequence. In addition, existing glycosylation sequences may not be suitable for the attachment of a modifying group to a polypeptide. As an example, such modification may cause an undesirable decrease in biological activity of the modified polypeptide. Thus, there is a need in the art for methods that permit both the precise creation of glycosylation sequences and the ability to precisely direct the modification to those sites. The current invention addresses these and other needs.

### SUMMARY OF THE INVENTION

The present invention relates to glycosylation and modification of polypeptides, preferably polypeptides of therapeutic value, that include O-linked glycosylation sequences, which are a substrate for a glucosamine transferase (e.g., GlcNAc-transferase). In one embodiment, the polypeptide is a non-naturally occurring polypeptide including an O-linked glycosylation sequence, which is not present or not present at the same position in the corresponding parent polypeptide.

The present invention describes the discovery that enzymatic glycoconjugation and glycoPEGylation reactions can be specifically targeted to certain O-linked glycosylation sequences within a polypeptide. In particular, glucosamine-moieties, which are optionally derivatized with a polymeric modifying group, are enzymatically transferred to an amino acid residue of a polypeptide. This amino acid residue is part of an O-linked glycosylation sequence, which is recognized as a substrate by an enzyme, such as an O-GlcNAc transferase (OGT), also referred to herein as a GlcNAc transferase.

One advantage of the current invention is that the modified sugar, which is preferably a modified glucosamine moiety, can be covalently attached directly to an amino acid side chain of a polypeptide. Unexpectedly, the inventor has discovered that certain glycosyltransferases used in this process can not only add glycosyl residues directly to the polypeptide backbone but most importantly, exhibit significant tolerance with respect to the glycosyl donor molecule, which these enzymes use as a substrate. For example, certain GlcNAc transferases are capable of adding a glucosamine moiety, which is modified with a polymeric modifying group, directly to an amino acid residue of the polypeptide. As a result, glycosylation of the polypeptide prior to glycoconjugation with a modified sugar residue is not necessary, however possible.

Another advantage of the present invention is that the glycosyltransferase that catalyzes the glycoconjugation reaction (e.g., glycoPEGylation) can be produced utilizing a bacterial expression system. In a particularly preferred embodiment, the glycosyltransferase (e.g., GlcNAc transferase) is expressed in E. coli. Due to these and other advantages, the invention provides time- and cost-efficient production routes to polypeptide conjugates that include modifying groups, such as water-soluble polymers.

### Polypeptides Including an O-Linked Glycosylation Sequence

In one embodiment, the O-glycosylation sequence of the invention is present in the parent polypeptide (e.g., a wild-type polypeptide). In another embodiment, the O-linked glycosylation sequence is introduced into the parent polypeptide by mutation. Accordingly, the present invention provides a non-naturally occurring polypeptide corresponding to a parent polypeptide and having an amino acid sequence containing at least one O-linked glycosylation sequence of the invention that is not present, or not present at the same position, in the corresponding parent polypetide. In one example, each O-linked glycosylation sequence is a substrate for a GlcNAc-transferase. In another example, the O-linked glycosylation sequence includes an amino acid sequence, which is a member selected from Formulae (I) to (VI):

(B¹)ₐP(B²)_{b}US(B³)_{c} (I)

(B¹)ₐP(B²)_{b}UT(B³)_{c} (II)

(B⁴)_{d}PSZ(B⁵)ₑ (III)

(B⁴)_{d}PTZ(B⁵)ₑ (IV)

(B⁶)_{f}S(B⁷)_{g}P(B⁸)ₕ (V)

(B⁶)_{f}T(B⁷)_{g}P(B⁸)ₕ (VI)

In Formulae (I) to (VI), b and g are integers selected from 0 to 2 and a, c, d, e, f and h are integers selected from 0 to 5. T is threonine, S is serine, P is proline, U is an amino acid selected from V, S, T, E, Q and uncharged amino acids, and Z is an amino acid selected from P, E, Q, S, T and uncharged amino acids. Each B¹, B², B³, B⁴, B⁵, B⁶, B⁷ and B⁸ is a member independently selected from an amino acid.

In addition, the present invention provides an isolated nucleic acid that encodes the non-naturally occurring polypeptide of the invention. The invention further provides an expression vector, as well as a cell that includes the above nucleic acid. The invention further provides a library of non-naturally occurring polypeptides, wherein each member of the library includes at least one O-linked glycosylation sequence of the invention. Also provided are methods of making and using such libraries.

### Polypeptide Conjugates

The invention further provides a covalent conjugate between a non-naturally occurring polypeptide and a polymeric modifying group, wherein the non-naturally occurring polypeptide corresponds to a parent-polypeptide and has an amino acid sequence including an exogenous O-linked glycosylation sequence that is not present, or not present at the same position, in the corresponding parent polypeptide. In one example, the O-linked glycosylation sequence is a substrate for a GlcNAc-transferase and includes at least one amino acid residue having a hydroxyl group. The polymeric modifying group is covalently attached to the polypeptide at the hydroxyl group of the O-linked glycosylation sequence via a glycosyl linking group. The parent polypeptide is preferably a therapeutic polypeptide.

In an exemplary embodiment, the polypeptide conjugate of the invention includes a moitey according to Formula (VII), wherein q can be 0 or 1:

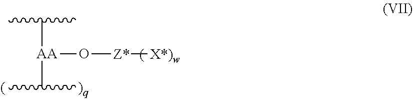

In Formula (VII) w is an integer selected from 0 and 4. In one example, w is selected from 0 and 1. AA-O is a moiety derived from an amino acid residue having a side chain, which is substituted with a hydroxyl group (e.g., serine or threonine), wherein the amino acid is located within an O-linked glycosylation sequence of the invention. When q is 1, then the amino acid is an internal amino acid of the polypeptide, and when q is 0, then the amino acid is an N-terminal or C-terminal amino acid. Z* is a member selected from a glucosamine-moiety, a glucosamine-mimetic moiety, an oligosaccharide comprising a glucosamine-moiety and an oligosaccharide comprising a glucosamine-mimetic moiety. X* is a member selected from a polymeric modifying group and a glycosyl linking group including a polymeric modifying group. In one example, Z* is a glucosamine-moiety (e.g., GlcNAc or GlcNH) and X* is a polymeric modifying group.

The invention also provides pharmaceutical compositions including a covalent conjugate of the invention and a pharmaceutically acceptable carrier.

### Modified Sugar Nucleotides

The invention further provides a compound having a structure according to Formula (XI):

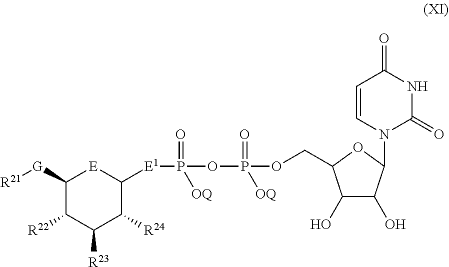

wherein each Q is a member independently selected from H, a negative charge and a salt counter-ion (i.e., cation). E is a member selected from NH, O, S, and CH₂. E¹ is a member selected from O and S. G is a member selected from —CH₂— and C=A, wherein A is a member selected from O, S and NR²⁷, wherein R²⁷ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. R²¹, R²², R²³ and R²⁴ are members independently selected from H, OR²⁵, SR²⁵, NR²⁵R²⁶, NR²⁵S(O)₂R²⁶, S(O)₂NR²⁵R²⁶, NR²⁵C(O)R²⁶, C(O)NR²⁵R²⁶, C(O)OR²⁵, acyl, substituted or unsubstituted alkyl, substituted or unsubstituted heteroalkyl, substituted or unsubstituted aryl, substituted or unsubstituted heteroaryl and substituted or unsubstituted heterocycloalkyl, wherein R²⁵ and R²⁶ are members independently selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl. In an exemplary embodiment, at least one of R²¹, R²², R²³, R²⁴ and R²⁷ includes a polymeric modifying group.

### Methods of Forming Polypeptide Conjugates

The invention further provides a method of forming a covalent conjugate between a polypeptide and a polymeric modifying group, wherein the polypeptide includes an O-linked glycosylation sequence (e.g., an exogenous O-linked glycosylation sequence) that includes an amino acid residue with a side chain having a hydroxyl group. The O-linked glycosylation sequence is a substrate for a GlcNAc-transferase. The polymeric modifying group is covalently linked to the polypeptide via a glucosamine-linking group interposed between and covalently linked to both the polypeptide and the modifying group. The method includes the step of: (i) contacting the polypeptide with a glucosamine-donor that includes a glucosamine-moiety covalently linked to a polymeric modifying group, in the presence of a GlcNAc-transferase under conditions sufficient for the GlcNAc-transferase to transfer the glucosamine-moiety from the glucosamine-donor onto the hydroxyl group of the O-linked glycosylation sequence. Exemplary glucosamine moieties include GlcNAc and GlcNH.

Additional aspects, advantages and objects of the present invention will be apparent from the detailed description that follows.

### BRIEF DESCRIPTION OF THE DRAWINGS

FIG. 1 is an exemplary amino acid sequence for human OGT with accession number O15294 (SEQ ID NO: 1).

FIG. 2 is an exemplary amino acid sequence for recombinant human OGT Δ176 (SEQ ID NO: 2).

FIG. 3 is an exemplary amino acid sequence for recombinant human OGT Δ182 (SEQ ID NO: 3).

FIG. 4 is an exemplary amino acid sequence for recombinant human OGT Δ182-His₈ (SEQ ID NO: 4).

FIG. 5 is an exemplary amino acid sequence for recombinant human OGT Δ382 (SEQ ID NO: 5).

FIG. 6 is an exemplary amino acid sequence for recombinant human OGT Δ382-His₈ (SEQ ID NO: 6).

FIG. 7 is an exemplary amino acid sequence for recombinant His₇-human OGT Δ382 (SEQ ID NO: 7).

FIG. 8 is an exemplary amino acid sequence for recombinant MBP-tagged human OGT Δ182 (SEQ ID NO: 8).

FIG. 9 is an exemplary amino acid sequence for recombinant MBP-tagged human OGT Δ382 (SEQ ID NO: 9).

FIG. 10 is an exemplary amino acid sequence for Factor VIII (SEQ ID NO: 10).

FIG. 11 is an exemplary amino acid sequence for Factor VIII (SEQ ID NO: 11).

FIG. 12 is an exemplary Factor VIII amino acid sequence, wherein the B-domain (amino acid residues 741-1648) is removed (SEQ ID NO: 12). Exemplary polypeptides of the invention include those in which the deleted B-domain is replaced with at least one amino acid residue (B-domain replacement sequence). In one embodiment, the B-domain replacement sequence between Arg⁷⁴⁰ and Glu¹⁶⁴⁹ includes at least one O-linked or N-linked glycosylation sequence.

FIG. 13 is an exemplary amino acid sequence for B-domain deleted Factor VIII (SEQ ID NO: 13).

FIG. 14 is an exemplary amino acid sequence for B-domain deleted Factor VIII (SEQ ID NO: 14).

FIG. 15 is an exemplary amino acid sequence for B-domain deleted Factor VIII (SEQ ID NO: 15).

FIG. 16 demonstrates the bacterial expression of human OGT constructs. Total cell lysates were analyzed by SDS-PAGE. Recombinant OGT is boxed. The first lane represents a molecular weight marker, respectively and the second lane was left empty. FIG. 16A: Untagged human OGT Δ176 (SEQ ID NO: 2) was expressed in W3110 and trxB gor supp mutant E. coli (FIG. 16A, lanes 3 and 4, respectively). FIG. 16B: C-terminally His₈ tagged OGT Δ382 (SEQ ID NO: 6) (FIG. 16B, lanes 3 and 4), His₈ tagged OGT Δ182 (SEQ ID NO: 4, FIG. 16B, lane 7), and N-terminally His₇ tagged OGT Δ382 (SEQ ID NO: 7, FIG. 16B, lanes 5 and 6) were expressed in W3110 and trxB gor supp mutant E. coli.

### DETAILED DESCRIPTION OF THE INVENTION

### I. Abbreviations

PEG, poly(ethyleneglycol); m-PEG, methoxy-poly(ethylene glycol); PPG, poly(propyleneglycol); m-PPG, methoxy-poly(propylene glycol); Fuc, fucose or fucosyl; Gal, galactose or galactosyl; GalNAc, N-acetylgalactosamine or N-acetylgalactosaminyl; Glc, glucose or glucosyl; GlcNAc, N-acetylglucosamine or N-acetylglucosaminyl; GlcNH, glucosamine or glucosaminyl; Man, mannose or mannosyl; ManAc, mannosamine acetate or mannosaminyl acetate; Sia, sialic acid or sialyl; and NeuAc, N-acetylneuramine or N-acetylneuraminyl.

### II. Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures of analytical and synthetic organic chemistry described below are those well known and commonly employed in the art. Standard techniques, or modifications thereof, are used for chemical syntheses and chemical analyses.

All oligosaccharides described herein are described with the name or abbreviation for the non-reducing saccharide (i.e., Gal), followed by the configuration of the glycosidic bond (α or β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature see, for example, Essentials of Glycobiology Varki et al. eds. CSHL Press (1999).

Oligosaccharides are considered to have a reducing end and a non-reducing end, whether or not the saccharide at the reducing end is in fact a reducing sugar.

The term “glycosyl moiety” means any radical derived from a sugar residue. “Glycosyl moiety” includes mono- and oligosaccharides and encompasses “glycosyl-mimetic moiety.”

The term “glycosyl-mimetic moiety,” as used herein refers to a moiety, which structurally resembles a glycosyl moiety (e.g., a hexose or a pentose). Examples of “glycosyl-mimetic moiety” include those moieties, wherein the glycosidic oxygen or the ring oxygen of a glycosyl moiety, or both, has been replaced with a bond or another atom (e.g., sufur), or another moiety, such as a carbon—(e.g., CH₂), or nitrogen-containing group (e.g., NH). Examples include substituted or unsubstituted cyclohexyl derivatives, cyclic thioethers, cyclic amines as well as moieties including a thioglycosidic bond, and the like. Other examples of “glycosyl-mimetic moiety” include ring structures with double bonds as well as ring structures, wherein one of the ring carbon atoms carries a carbonyl group or another double-bonded substituent, such as a hydrazone moiety. In one example, the “glycosyl-mimetic moiety” is transferred in an enzymatically catalyzed reaction onto an amino acid residue of a polypeptide or a glycosyl moiety of a glycopeptide. This can, for instance, be accomplished by activating the “glycosyl-mimetic moiety” with a leaving group, such as a halogen. In a preferred embodiment, the sugar moiety of a sugar nucleotide constitutes a glycosyl-mimetic moiety and this glycosyl-mimetic moiety, which is optionally derivatized with a modifying group, is enzymatically transferred from a sugar nucleotide (e.g., modified sugar nucleotide) onto an amino acid residue of a polypeptide using a glycosyltransferase (e.g., GlcNAc-transferase). The word “glycosyl” in the term “glycosyl-mimetic moiety” may be replaced with a word describing a specific sugar moiety and the resulting term refers to a moiety, which structurally resembles the specific sugar moiety. For example, “GlcNAc-mimetic moiety” refers to a “glycosyl-mimetic moiety” resembling an N-acetylglucosamine moiety.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

The term “uncharged amino acid” refers to amino acids, that do not include an acidic (e.g., —COOH) or basic (e.g., —NH₂) functional group. Basic amino acids include lysine (K) and arginine (R). Acidic amino acids include aspartic acid (D) and glutamic acid (E). “Uncharged amino acids include, e.g., glycine (G), alanine (A), valine (V), leucine (L), phenylalanine (F), but also those amino acids that include —OH or —SH groups (e.g., threonine (T), serine (S), tyrosine (Y) and cysteine (C)).

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Peptide” refers to a polymer in which the monomers are amino acids and are joined together through amide bonds. Peptides of the present invention can vary in size, e.g., from two amino acids to hundreds or thousands of amino acids. A larger peptide is alternatively referred to as a “polypeptide” or “protein”. Additionally, unnatural amino acids, for example, β-alanine, phenylglycine, homoarginine and homophenylalanine are also included. Amino acids that are not gene-encoded may also be used in the present invention. Furthermore, amino acids that have been modified to include reactive groups, glycosylation sequences, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are petides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

In the present application, amino acid residues are numbered (typically in the superscript) according to their relative positions from the N-terminal amino acid (e.g., N-terminal methionine) of the polypeptide, which is numbered “1”. The N-terminal amino acid may be a methionine (M), numbered “1”. The numbers associated with each amino acid residue can be readily adjusted to reflect the absence of N-terminal methionine if the N-terminus of the polypeptide starts without a methionine. It is understood that the N-terminus of an exemplary polypeptide can start with or without a methionine.

The term “wild-type polypeptide” refers to a naturally occurring polypeptide, which optionally and naturally includes an O-linked glycosylation sequence of the invention.

The term “parent polypeptide” refers to any polypeptide, which has an amino acid sequence, which does not include an “exogenous” O-linked glycosylation sequence of the invention. However, a “parent polypeptide” may include one or more naturally occurring (endogenous) O-linked glycosylation sequence. For example, a wild-type polypeptide may include the O-linked glycosylation sequence PVS. The term “parent polypeptide” refers to any polypeptide including wild-type polypeptides, fusion polypeptides, synthetic polypeptides, recombinant polypeptides (e.g., therapeutic polypeptides) as well as any variants thereof (e.g., previously modified through one or more replacement of amino acids, insertions of amino acids, deletions of amino acids and the like) as long as such modification does not amount to forming an O-linked glycosylation sequence of the invention. In one embodiment, the amino acid sequence of the parent polypeptide, or the nucleic acid sequence encoding the parent polypeptide, is defined and accessible to the public. For example, the parent polypeptide is a wild-type polypeptide and the amino acid sequence or nucleotide sequence of the wild-type polypeptide is part of a publicly accessible protein database (e.g., EMBL Nucleotide Sequence Database, NCBI Entrez, ExPasy, Protein Data Bank and the like). In another example, the parent polypeptide is not a wild-type polypeptide but is used as a therapeutic polypeptide (i.e., authorized drug) and the sequence of such polypeptide is publicly available in a scientific publication or patent. In yet another example, the amino acid sequence of the parent polypeptide or the nucleic acid sequence encoding the parent polypeptide was accessible to the public at the time of the invention. In one embodiment, the parent polypeptide is part of a larger structure. For example, the parent polypeptide corresponds to the constant region (F_{c}) region or C_{H}2 domain of an antibody, wherein these domains may be part of an entire antibody. In one embodiment, the parent polypeptide is not an antibody of unknown sequence.

The term “mutant polypeptide” or “polypeptide variant” refers to a form of a polypeptide, wherein the amino acid sequence of the polypeptide differs from the amino acid sequence of its corresponding wild-type form, naturally existing form or any other parent form. A mutant polypeptide can contain one or more mutations, e.g., replacement, insertion, deletion, etc. which result in the mutant polypeptide.

The term “non-naturally occurring polypeptide” or “sequon polypeptide” refers to a polypeptide variant that includes in its amino acid sequence at least one “exogenous O-linked glycosylation sequence” of the invention (O-linked glycosylation sequence that is not present or not present at the same position in the corresponding wild-type form or any other parent form) but may also include one or more endogenous (e.g., naturally occurring) O-linked glycosylation sequence. A “non-naturally occurring polypeptide” can contain one or more O-linked glycosylation sequence of the invention and in addition may include other mutations, e.g., replacements, insertions, deletions, truncations etc.

The term “exogenous O-linked glycosylation sequence” refers to an O-linked glycosylation sequence of the invention that is introduced into the amino acid sequence of a parent polypeptide (e.g., wild-type polypeptide), wherein the parent polypeptide does either not include an O-linked glycosylation sequence or includes an O-linked glycosylation sequence at a different position. In one example, an O-linked glycosylation sequence is introduced into a wild-type polypeptide that does not have an O-linked glycosylation sequence. In another example, a wild-type polypeptide naturally includes a first O-linked glycosylation sequence at a first position. A second O-linked glycosylation is introduced into this wild-type polypeptide at a second position. This modification results in a polypeptide having an “exogenous O-linked glycosylation sequence” at the second position. The exogenous O-linked glycosylation sequence may be introduced into the parent polypeptide by mutation. Alternatively, a polypeptide with an exogenous O-linked glycosylation sequence can be made by chemical synthesis.

The term “corresponding to a parent polypeptide” (or grammatical variations of this term) is used to describe a sequon polypeptide of the invention, wherein the amino acid sequence of the sequon polypeptide differs from the amino acid sequence of the corresponding parent polypeptide only by the presence of at least one exogenous O-linked glycosylation sequence of the invention. Typically, the amino acid sequences of the sequon polypeptide and the parent polypeptide exhibit a high percentage of identity. In one example, “corresponding to a parent polypetide” means that the amino acid sequence of the sequon polypeptide has at least about 50% identity, at least about 60%, at least about 70%, at least about 80%, at least about 90%, at least about 95% or at least about 98% identity to the amino acid sequence of the parent polypeptide. In another example, the nucleic acid sequence that encodes the sequon polypeptide has at least about 50% identity, at least about 60%, at least about 70%, at least about 80%, at least about 90%, at least about 95% or at least about 98% identity to the nucleic acid sequence encoding the parent polypeptide.

The term “introducing (or adding etc.) a glycosylation sequence (e.g., an O-linked glycosylation sequence) into a parent polypeptide” (or grammatical variations thereof), or “modifying a parent polypeptide” to include a glycosylation sequence (or grammatical variations thereof) do not necessarily mean that the parent polypeptide is a physical starting material for such conversion, but rather that the parent polypeptide provides the guiding amino acid sequence for the making of another polypeptide. In one example, “introducing a glycosylation sequence into a parent polypeptide” means that the gene for the parent polypeptide is modified through appropriate mutations to create a nucleotide sequence that encodes a sequon polypeptide. In another example, “introducing a glycosylation sequence into a parent polypeptide” means that the resulting polypeptide is theoretically designed using the parent polypeptide sequence as a guide. The designed polypeptide may then be generated by chemical or other means.

The term “lead polypeptide” refers to a non-naturally occurring polypeptide including at least one O-linked glycosylation sequence of the invention, that can be effectively glycosylated or glycoPEGylated. For a polypeptide of the invention to qualify as a lead polypeptide, such polypeptide, when subjected to suitable reaction conditions, is glycosylated or glycoPEGylated with a reaction yield of at least about 50%, preferably at least about 60%, more preferably at least about 70% and even more preferably about 80%, about 85%, about 90% or about 95%. Most preferred are those lead polypeptides of the invention, which can be glycosylated or glycoPEGylated with a reaction yield of greater than 95%. In one preferred embodiment, the lead polypeptide is glycosylated or glycoPEGylated in such a fashion that only one amino acid residue of each O-linked glycosylation sequence is glycosylated or glycoPEGylated (mono-glycosylation).

The term “library” refers to a collection of different polypeptides each corresponding to a common parent polypeptide. Each polypeptide species in the library is referred to as a member of the library. Preferably, the library of the present invention represents a collection of polypeptides of sufficient number and diversity to afford a population from which to identify a lead polypeptide. A library includes at least two different polypeptides. In one embodiment, the library includes from about 2 to about 10 members. In another embodiment, the library includes from about 10 to about 20 members. In yet another embodiment, the library includes from about 20 to about 30 members. In a further embodiment, the library includes from about 30 to about 50 members. In another embodiment, the library includes from about 50 to about 100 members. In yet another embodiment, the library includes more than 100 members. The members of the library may be part of a mixture or may be isolated from each other. In one example, the members of the library are part of a mixture that optionally includes other components. For example, at least two sequon polypeptides are present in a volume of cell-culture broth. In another example, the members of the library are each expressed separately and are optionally isolated. The isolated sequon polypeptides may optionally be contained in a multi-well container, in which each well contains a different type of sequon polypeptide.

The term “C_{H}2” domain of the present invention is meant to describe an immunoglobulin heavy chain constant C_{H}2 domain. In defining an immunoglobulin C_{H}2 domain reference is made to immunoglobulins in general and in particular to the domain structure of immunoglobulins as applied to human IgG1 by Kabat E. A. (1978) Adv. Protein Chem. 32:1-75.

The term “polypeptide comprising a C_{H}2 domain” or “polypeptide comprising at least one C_{H}2 domain” is intended to include whole antibody molecules, antibody fragments (e.g., Fc domain), or fusion proteins that include a region equivalent to the C_{H}2 region of an immunoglobulin.

The term “polypeptide conjugate,” refers to species of the invention in which a polypeptide is glycoconjugated with a sugar moiety (e.g., modified sugar) as set forth herein. In a representative example, the polypeptide is a non-naturally occurring polypeptide having an O-linked glycosylation sequence not present in the corresponding wild-type or parent polypeptide.

“Proximate a proline residue” or “in proximity to a proline residue” as used herein refers to an amino acid that is less than about 10 amino acids removed from a proline residue, preferably, less than about 9, 8, 7, 6 or 5 amino acids removed from a proline residue, more preferably, less than 4, 3, 2 or 1 residues removed from a proline residue. The amino acid “proximate a proline residue” may be on the C- or N-terminal side of the proline residue.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

The term “glucosamine” or “glucosamine moiety” refers to any glycosyl or glycosyl-mimetic moiety, in which the relative stereochemistry for the ring-substituents is the same as in glucose or N-acetyl-glucosamine. Exemplary “glucosamine moieties” are represented by Figure (VIIIa):

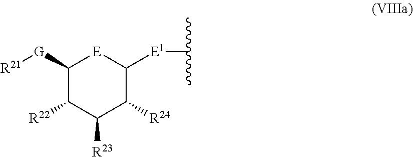

wherein G, E, E¹, R²¹, R²², R²³ and R²⁴ are defined as for Figure (VIII), below. Formula (VIIIa) includes modified and non-modified glucosamine analogs. In Formula (VIIIa), R²¹, R²², R²³, R²⁴ and R²⁷ optionally include a modifying group (e.g., a polymeric modifying group). One or more of the ring substituents R²², R²³ and R²⁴ can be hydrogen. Preferred glucosamine moieties include GlcNAc and GlcNH, optionally modified with a polymeric modifying group.

As used herein, the term “modified sugar,” refers to a naturally- or non-naturally-occurring carbohydrate. In one embodiment, the “modified sugar” is enzymatically added onto an amino acid or a glycosyl residue of a polypeptide using a method of the invention. The modified sugar is selected from a number of enzyme substrates including, but not limited to sugar nucleotides (mono-, di-, and tri-phosphates), activated sugars (e.g., glycosyl halides, glycosyl mesylates) and sugars that are neither activated nor nucleotides. The “modified sugar” is covalently functionalized with a “modifying group.” Useful modifying groups include, but are not limited to, polymeric modifying groups (e.g., water-soluble polymers), therapeutic moieties, diagnostic moieties, biomolecules and the like. In one embodiment, the modifying group is not a naturally occurring glycosyl moiety (e.g., naturally occurring polysaccharide). The modifying group is preferably non-naturally occurring. In one example, the “non-naturally occurring modifying group” is a polymeric modifying group, in which at least one polymeric moiety is non-naturally occurring. In another example, the non-naturally occurring modifying group is a modified carbohydrate. The locus of functionalization with the modifying group is selected such that it does not prevent the “modified sugar” from being added enzymatically to a polypeptide. “Modified sugar” also refers to any glycosyl mimetic moiety that is functionalized with a modifying group and which is a substrate for a natural or modified enzyme, such as a glycosyltransferase.

As used herein, the term “polymeric modifying group” is a modifying group that includes at least one polymeric moiety (polymer). The polymeric modifying group added to a polypeptide can alter a property of such polypeptide, for example, its bioavailability, biological activity or its half-life in the body. Exemplary polymers include water soluble and water insoluble polymers. A polymeric modifying group can be linear or branched and can include one or more independently selected polymeric moieties, such as poly(alkylene glycol) and derivatives thereof. In one example, the polymer is non-naturally occurring. In an exemplary embodiment, the polymeric modifying group includes a water-soluble polymer, e.g., poly(ethylene glycol) and derivatived thereof (PEG, m-PEG), poly(propylene glycol) and derivatives thereof (PPG, m-PPG) and the like. In a preferred embodiment, the poly(ethylene glycol) or poly(propylene glycol) has a molecular weight that is essentially homodisperse. In one embodiment the polymeric modifying group is not a naturally occurring polysaccharide.

The term “water-soluble” refers to moieties that have some detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine). An exemplary polysaccharide is poly(sialic acid). An exemplary poly(ether) is poly(ethylene glycol), e.g., m-PEG. Poly(ethylene imine) is an exemplary polyamine, and poly(acrylic) acid is a representative poly(carboxylic acid).

The polymer backbone of the water-soluble polymer can be poly(ethylene glycol) (i.e. PEG). However, it should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including alkoxy PEG, difunctional PEG, multiarmed PEG, forked PEG, branched PEG, pendent PEG (i.e. PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(—PEG-OH)ₘ in which R represents the core moiety, such as glycerol or pentaerythritol, and m represents the number of arms. Multi-armed PEG molecules, such as those described in U.S. Pat. No. 5,932,462, which is incorporated by reference herein in its entirety, can also be used as the polymer backbone.

Many other polymers are also suitable for the invention. Polymer backbones that are non-peptidic and water-soluble, with from 2 to about 300 termini, are particularly useful in the invention. Examples of suitable polymers include, but are not limited to, other poly(alkylene glycols), such as polypropylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), such as described in U.S. Pat. No. 5,629,384, which is incorporated by reference herein in its entirety, as well as copolymers, terpolymers, and mixtures thereof. Although the molecular weight of each chain of the polymer backbone can vary, it is typically in the range of from about 100 Da to about 100,000 Da, often from about 5,000 Da to about 80,000 Da.

The term “homodisperse” refers to a polymer, in which a substantial proportion of the polymer molecules in a sample of the polymer are of approximately the same molecular weight.

The term “glycoconjugation,” as used herein, refers to the enzymatically mediated conjugation of a modified sugar species to an amino acid or glycosyl residue of a polypeptide, e.g., a mutant human growth hormone of the present invention. In one example, the modified sugar is covalently attached to one or more modifying groups. A subgenus of “glycoconjugation” is “glycol-PEGylation” or “glyco-PEGylation”, in which the modifying group of the modified sugar is poly(ethylene glycol) or a derivative thereof, such as an alkyl derivative (e.g., m-PEG) or a derivative with a reactive functional group (e.g., H₂N-PEG, HOOC-PEG).

The terms “large-scale” and “industrial-scale” are used interchangeably and refer to a reaction cycle that produces at least about 250 mg, preferably at least about 500 mg, and more preferably at least about 1 gram of glycoconjugate at the completion of a single reaction cycle.

The term “O-linked glycosylation sequence” or “sequon” refers to any amino acid sequence (e.g., containing from about 3 to about 10 amino acids, preferably about 3 to about 9 amino acids) that includes an amino acid residue having a hydroxyl group (e.g., serine or threonine). In one embodiment, the O-linked glycosylation sequence is a substrate for an enzyme, such as a glycosyltransferase, preferably when part of an amino acid sequence of a polypeptide. In a typical embodiment, the enzyme transfers a glycosyl moiety onto the O-linked glycosylation sequence by modifying the above described hydroxyl group, which is referred to as the “site of glycosylation”. The invention distinguishes between an O-linked glycosylation sequence that is naturally occurring in a wild-type polypeptide or any other parent form thereof (endogenous O-linked glycosylation sequence) and an “exogenous O-linked glycosylation sequence”. A polypeptide that includes an exogenous O-linked glycosylation sequence can also be termed “sequon polypeptide”. The amino acid sequence of a parent polypeptide may be modified to include an exogenous O-linked glycosylation sequence through recombinat technology, chemical syntheses or other means.

The term, “glycosyl linking group,” as used herein refers to a glycosyl residue to which a modifying group (e.g., PEG moiety, therapeutic moiety, biomolecule) is covalently attached; the glycosyl linking group joins the modifying group to the remainder of the conjugate. In the methods of the invention, the “glycosyl linking group” becomes covalently attached to a glycosylated or unglycosylated polypeptide, thereby linking the modifying group to an amino acid and/or glycosyl residue of the polypeptide. A “glycosyl linking group” is generally derived from a “modified sugar” by the enzymatic attachment of the “modified sugar” to an amino acid and/or glycosyl residue of the polypeptide. The glycosyl linking group can be a saccharide-derived structure that is degraded during formation of modifying group-modified sugar cassette (e.g., oxidation→Schiff base formation→reduction), or the glycosyl linking group may be intact. An “intact glycosyl linking group” refers to a linking group that is derived from a glycosyl moiety in which the saccharide monomer that links the modifying group and to the remainder of the conjugate is not degraded, e.g., oxidized, e.g., by sodium metaperiodate. “Intact glycosyl-linking groups” of the invention may be derived from a naturally occurring oligosaccharide by addition of glycosyl unit(s) or removal of one or more glycosyl unit from a parent saccharide structure. A “glycosyl linking group” may include a glycosyl-mimetic moiety. For example, the glycosyl transferase (e.g., GlcNAc transferase) used to add the modified sugar to a glycosylated or non-glycosylated polypeptide, exhibits tolerance for a glycosyl-mimetic substrate (e.g., a modified sugar in which the sugar moiety is a glycosyl-mimetic moiety, e.g., a GlcNAc-mimetic moiety). The transfer of the modified glycosyl-mimetic sugar results in a conjugate having a glycosyl linking group that is a glycosyl-mimetic moiety.

The term “targeting moiety,” as used herein, refers to species that will selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments, transferrin, HS-glycoprotein, coagulation factors, serum proteins, β-glycoprotein, G-CSF, GM-CSF, M-CSF, EPO and the like.

As used herein, “therapeutic moiety” means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is bound to a carrier, e.g, multivalent agents. Therapeutic moiety also includes proteins and constructs that include proteins. Exemplary proteins include, but are not limited to, Erythropoietin (EPO), Granulocyte Colony Stimulating Factor (GCSF), Granulocyte Macrophage Colony Stimulating Factor (GMCSF), Interferon (e.g., Interferon-α, -β, -γ), Interleukin (e.g., Interleukin II), serum proteins (e.g., Factors VII, VIIa, VIII, IX, and X), Human Chorionic Gonadotropin (HCG), Follicle Stimulating Hormone (FSH) and Lutenizing Hormone (LH) and antibody fusion proteins (e.g. Tumor Necrosis Factor Receptor ((TNFR)/Fc domain fusion protein)).

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Also encompassed within the scope of the term “anti-tumor drug,” are conjugates of peptides with anti-tumor activity, e.g. TNF-α. Conjugates include, but are not limited to those formed between a therapeutic protein and a glycoprotein of the invention. A representative conjugate is that formed between PSGL-1 and TNF-α.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diptheria toxin, and snake venom (e.g., cobra venom).

As used herein, “a radioactive agent” includes any radioisotope that is effective in diagnosing or destroying a tumor. Examples include, but are not limited to, indium-111, cobalt-60. Additionally, naturally occurring radioactive elements such as uranium, radium, and thorium, which typically represent mixtures of radioisotopes, are suitable examples of a radioactive agent. The metal ions are typically chelated with an organic chelating moiety.

Many useful chelating groups, crown ethers, cryptands and the like are known in the art and can be incorporated into the compounds of the invention (e.g., EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D.C., 1982, pp. 370-387; Kasina et al., Bioconjugate Chem., 9: 108-117 (1998); Song et al., Bioconjugate Chem., 8: 249-255 (1997).

As used herein, “pharmaceutically acceptable carrier” includes any material, which when combined with the conjugate retains the conjugates' activity and is preferably non-reactive with the subject's immune systems. “Pharmaceutically acceptable carrier” includes solids and liquids, such as vehicles, diluents and solvents. Examples include, but are not limited to, any of the standard pharmaceutical carriers such as a phosphate buffered saline solution, water, emulsions such as oil/water emulsion, and various types of wetting agents. Other carriers may include sterile solutions and tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “ameliorating” or “ameliorate” refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

The term “therapy” refers to “treating” or “treatment” of a disease or condition including preventing the disease or condition from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “effective amount” or “an amount effective to” or a “therapeutically effective amount” or any gramatically equivalent term means the amount that, when administered to an animal or human for treating a disease, is sufficient to effect treatment for that disease.

The term “isolated” refers to a material that is substantially or essentially free from components, which are used to produce the material. For peptide conjugates of the invention, the term “isolated” refers to material that is substantially or essentially free from components, which normally accompany the material in the mixture used to prepare the peptide conjugate.

“Isolated” and “pure” are used interchangeably. Typically, isolated peptide conjugates of the invention have a level of purity preferably expressed as a range. The lower end of the range of purity for the peptide conjugates is about 60%, about 70% or about 80% and the upper end of the range of purity is about 70%, about 80%, about 90% or more than about 90%.

When the peptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or similar means).

“Essentially each member of the population,” as used herein, describes a characteristic of a population of peptide conjugates of the invention in which a selected percentage of the modified sugars added to a peptide are added to multiple, identical acceptor sites on the peptide. “Essentially each member of the population” speaks to the “homogeneity” of the sites on the peptide conjugated to a modified sugar and refers to conjugates of the invention, which are at least about 80%, preferably at least about 90% and more preferably at least about 95% homogenous.

“Homogeneity,” refers to the structural consistency across a population of acceptor moieties to which the modified sugars are conjugated. Thus, in a peptide conjugate of the invention in which each modified sugar moiety is conjugated to an acceptor site having the same structure as the acceptor site to which every other modified sugar is conjugated, the peptide conjugate is said to be about 100% homogeneous. Homogeneity is typically expressed as a range. The lower end of the range of homogeneity for the peptide conjugates is about 50%, about 60%, about 70% or about 80% and the upper end of the range of purity is about 70%, about 80%, about 90% or more than about 90%.

When the peptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The purity of the peptide conjugates is typically determined by one or more methods known to those of skill in the art, e.g., liquid chromatography-mass spectrometry (LC-MS), matrix assisted laser desorption mass time of flight spectrometry (MALDITOF), capillary electrophoresis, and the like.

“Substantially uniform glycoform” or a “substantially uniform glycosylation pattern,” when referring to a glycopeptide species, refers to the percentage of acceptor moieties that are glycosylated by the glycosyltransferase of interest (e.g., fucosyltransferase). For example, in the case of a α1,2 fucosyltransferase, a substantially uniform fucosylation pattern exists if substantially all (as defined below) of the Galβ1,4-GlcNAc-R and sialylated analogues thereof are fucosylated in a peptide conjugate of the invention. It will be understood by one of skill in the art, that the starting material may contain glycosylated acceptor moieties (e.g., fucosylated Galβ1,4-GlcNAc-R moieties). Thus, the calculated percent glycosylation will include acceptor moieties that are glycosylated by the methods of the invention, as well as those acceptor moieties already glycosylated in the starting material.

The term “substantially” in the above definitions of “substantially uniform” generally means at least about 40%, at least about 70%, at least about 80%, or more preferably at least about 90%, and still more preferably at least about 95% of the acceptor moieties for a particular glycosyltransferase are glycosylated.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “alkyl” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted aryl, substituted or unsubstituted heteroaryl, substituted or unsubstituted heterocycloalkyl, —OR′, ═O, ═NR′, ═N—OR′, —NR′R″, SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R″′ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted heteroalkyl, substituted or unsubstituted aryl, substituted or unsubstituted heteroaryl, substituted or unsubstituted heterocycloalkyl, —OR′, ═O, ═NR′, ═N—OR′, —NR′R″, —SR′, -halogen, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R″′)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R″′ and R″″ groups when more than one of these groups is present.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B-, wherein A and B are independently —CRR′—, —O—, —NR—, —S—, —S(O)—, —S(O)₂—, S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R′, R″, R′″ and R″″ are preferably independently selected from hydrogen or substituted or unsubstituted (C₁-C₆)alkyl.

As used herein, the term “acyl” describes a substituent containing a carbonyl residue, C(O)R. Exemplary species for R include H, halogen, alkoxy, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems may include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si), boron (B) and phosphorus (P).

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, and substituted or unsubstituted heterocycloalkyl groups.

The term “pharmaceutically acceptable salts” includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

The graphic representations of racemic, ambiscalemic and scalemic or enantiomerically pure compounds used herein are taken from Maehr, J. Chem. Ed., 62: 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but not implying any absolute stereochemistry; and wedge outlines and dotted or broken lines denote enantiomerically pure compounds of indeterminate absolute configuration.

The terms “enantiomeric excess” and diastereomeric excess” are used interchangeably herein. Compounds with a single stereocenter are referred to as being present in “enantiomeric excess,” those with at least two stereocenters are referred to as being present in “diastereomeric excess.”

The compounds of the present invention may also contain unnatural proportions of atomic isotopes at one or more of the atoms that constitute such compounds. For example, the compounds may be radiolabeled with radioactive isotopes, such as for example tritium (³H), deuterium (²D), iodine-125 (¹²⁵I) or carbon-14 (¹⁴C). All isotopic variations of the compounds of the present invention, whether radioactive or not, are intended to be encompassed within the scope of the present invention.

“Reactive functional group,” as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application or modification for a particular purpose is within the ability of one of skill in the art (see, for example, Sandler and Karo, eds. ORGANIC FUNCTIONAL GROUP PREPARATIONS, Academic Press, San Diego, 1989).

“Non-covalent protein binding groups” are moieties that interact with an intact or denatured polypeptide in an associative manner. The interaction may be either reversible or irreversible in a biological milieu. The incorporation of a “non-covalent protein binding group” into a chelating agent or complex of the invention provides the agent or complex with the ability to interact with a polypeptide in a non-covalent manner. Exemplary non-covalent interactions include hydrophobic-hydrophobic and electrostatic interactions. Exemplary “non-covalent protein binding groups” include anionic groups, e.g., phosphate, thiophosphate, phosphonate, carboxylate, boronate, sulfate, sulfone, sulfonate, thiosulfate, and thiosulfonate.

A “glycosyltransferase truncation” or a “truncated glycosyltransferase” or grammatical variants, refer to a glycosyltransferase that has fewer amino acid residues than a naturally occurring glycosyltransferase, but that retains certain enzymatic activity. Truncated glycosyltransferases include, e.g., truncated GnT1 enzymes, truncated GalT1 enzymes, truncated ST3GalIII enzymes, truncated GalNAc-T2 enzymes, truncated Core-1-GalT1 enzymes, amino acid residues from about 32 to about 90 (see e.g., the human enzyme); truncated ST3Gall enzymes, truncated ST6GalNAc-1 enzymes, and truncated GalNAc-T2 enzymes. Any number of amino acid residues can be deleted so long as the enzyme retains activity. In some embodiments, domains or portions of domains can be deleted, e.g., a signal-anchor domain can be deleted leaving a truncation comprising a stem region and a catalytic domain; a signal-anchor domain and a portion of a stem region can be deleted leaving a truncation comprising the remaining stem region and a catalytic domain; or a signal-anchor domain and a stem region can be deleted leaving a truncation comprising a catalytic domain. Glycosyltransferase truncations can also occur at the C-terminus of the protein. For example, some GalNAcT enzymes, such as GalNAc-T2, have a C-terminal lectin domain that can be deleted without diminishing enzymatic activity.

“Refolding expression system” refers to a bacteria or other microorganism with an oxidative intracellular environment, which has the ability to refold disulfide-containing protein in their proper/active form when expressed in this microorganism. Exemplars include systems based on E. coli (e.g., Origami™ (modified E. coli trxB-/gor-), Origami 2™ and the like), Pseudomonas (e.g., fluorescens). For exemplary references on Origami™ technology see, e.g., Lobel et al., Endocrine 2001, 14(2): 205-212; and Lobel et al., Protein Express. Purif. 2002, 25(1): 124-133, each incorporated herein by reference.

### III. Introduction

The present invention provides polypeptides that include one or more O-linked linked glycosylation sequence, wherein each glycosylation sequence is a substrate for a glycosyltransferase (e.g., a GlcNAc transferase). The enzyme catalyzes the transfer of a glycosyl moiety (e.g., a glucosamine moiety) from a glycosyl donor molecule (e.g., UDP-GlcNAc) onto an oxygen atom of an amino acid side chain (site of glycosylation), wherein the amino acid (e.g., serine or threonine) is part of the O-linked glycosylation sequence. In an alternative embodiment, the amino acid includes a sufhydryl group (e.g., cysteine) instead of a hydroxyl group.

The invention also provides polypeptide conjugates, in which a modified sugar moiety is attached either directly (e.g., through a glycoPEGylation reaction) or indirectly (e.g., through an intervening glycosyl residue) to an O-linked or S-linked glycosylation sequence located within the polypeptide. Also provided are methods for making the conjugates of the invention.

The glycosylation and glycoPEGylation methods of the invention can be practiced on any polypeptide incorporating an O-linked or S-linked glycosylation sequence. In one embodiment, the glycosylation sequence is introduced into the amino acid sequence of a parent polypeptide by mutation to create a non-naturally occurring polypeptide of the invention. The parent polypeptide can be any polypeptide. Examples include wild-type polypeptides and those polypeptides, which have already been modified from their naturally occurring counterpart (e.g., by mutation). In a preferred embodiment, the parent polypeptide is a therapeutic polypeptide, such as a human growth hormone (hGH), erythropoietin (EPO) or a therapeutic antibody. Accordingly, the present invention provides conjugates of therapeutic polypeptides that include within their amino acid sequence one or more glycosylation sequence, independently selected from S-linked and O-linked glycosylation sequences.

In various examples, the methods of the invention provide polypeptide conjugates with increased therapeutic half-life due to, for example, reduced clearance rate, or reduced rate of uptake by the immune or reticuloendothelial system (RES). Moreover, the methods of the invention provide a means for masking antigenic determinants on peptides, thus reducing or eliminating a host immune response against the peptide. Selective attachment of targeting agents to a peptide using an appropriate modified sugar can also be used to target a peptide to a particular tissue or cell surface receptor that is specific for the particular targeting agent.

In addition, the methods of the invention can be used to modulate the “biological activity profile” of a parent polypeptide. The inventors have recognized that the covalent attachment of a modifying group, such as a water soluble polymer (e.g., mPEG) to a parent polypeptide using the methods of the invention can alter not only bioavailability, pharmacodynamic properties, immunogenicity, metabolic stability, biodistribution and water solubility of the resulting polypeptide species, but can also lead to the reduction of undesired therapeutic activities or to the augmentation of desired therapeutic activities. For example, the former has been observed for the hematopoietic agent erythropoietin (EPO). Certain chemically PEgylated EPO variants showed reduced erythropoietic activity while the tissue-protective activity of the wild-type polypeptide was maintained. Such results are described e.g., in U.S. Pat. No. 6,531,121; WO2004/096148, WO2006/014466, WO2006/014349, WO2005/025606 and WO2002/053580. Exemplary cell-lines, which are useful for the evaluation of differential biological activities of selected polypeptides are summarized in Table 1, below:

TABLE 1
Cell-lines used for biological evaluation of various polypeptides

| Polypeptide | Cell-line | Biological Activity |
|---|---|---|
| EPO | UT7 | erythropoiesis |
| | SY5Y | neuroprotection |
| BMP-7 | MG-63 | osteoinduction |
| | HK-2 | nephrotoxicity |
| NT-3 | Neuro2 | neuroprotection (TrkC binding) |
| | NIH3T3 | neuroprotection (p75 binding) |

In one embodiment, a polypeptide conjugate of the invention shows reduced or enhanced binding affinity to a biological target protein (e.g., a receptor), a natural ligand or a non-natural ligand, such as an inhibitor. For instance, abrogating binding affinity to a class of specific receptors may reduce or eliminate associated cellular signaling and downstream biological events. Hence, the methods of the invention can be used to create polypeptide conjugates, which have identical, similar or different therapeutic profiles than the parent polypeptide from which the conjugates are derived. The methods of the invention can be used to identify glycoPEGylated therapeutics with specific (e.g., improved) biological functions and to “fine-tune” the therapeutic profile of any therapeutic polypeptide or other biologically active polypeptide.

### IV. Compositions

### Polypeptides

The present invention provides a non-naturally occurring polypeptide corresponding to a parent polypeptide and having an amino acid sequence containing at least one exogenous O-linked glycosylation sequence of the invention, wherein the O-linked glycosylation sequence is not present, or not present at the same position, in the corresponding parent polypeptide, from which the non-naturally occurring polypeptide is derived.

In one example, the amino acid sequence of the polypeptide provided by the present invention includes an O-linked glycosylation sequence, which (when part of the polypeptide), is a substrate for one or more wild-type, mutant or truncated glycosyltransferase. Preferred glycosyltransferases include GlcNAc transferases. Exemplary GlcNAc transferases are represented by SEQ ID NOs: 1-9 and 228 to 230.

In an exemplary embodiment, the non-naturally occurring polypeptide of the invention is generated by altering the amino acid sequence of a parent polypeptide (e.g., wild-type polypeptide) by mutation. The resulting polypeptide variant includes at least one “O-linked glycosylation sequence” that is either not present or not present at the same position, in the corresponding parent polypetide. The amino acid sequence of the non-naturally occurring polypeptide may contain a combination of naturally occurring (endogenous) and non-naturally occurring (exogenous) O-linked glycosylation sequences as long as at least one exogenous O-linked glycosylation sequence is present.

The parent polypeptide can be any polypeptide. Exemplary parent polypeptides include wild-type polypeptides and fragments thereof as well as peptides, which are modified from their naturally occurring counterpart (e.g., by previous mutation or truncation). In one embodiment, the polypeptide is a therapeutic polypeptide, such as those used as pharmaceutical agents (i.e., authorized drugs). A non-limiting selection of polypeptides is shown in FIG. 28 of U.S. patent application Ser. No. 10/552,896 filed Jun. 8, 2006, which is incorporated herein by reference. Accordingly, the present invention provides glycoconjugates of therapeutic polypeptides that include within their amino acid sequence one or more O-linked glycosylation sequence of the invention.

Exemplary parent- and wild-type polypeptides include growth factors, such as hepatocyte growth factor (HGF), nerve growth factors (NGF), epidermal growth factors (EGF), fibroblast growth factors (e.g., FGF-1, FGF-2, FGF-3, FGF-4, FGF-5, FGF-6, FGF-7, FGF-8, FGF-9, FGF-10, FGF-11, FGF-12, FGF-13, FGF-14, FGF-15, FGF-16, FGF-17, FGF-18, FGF-19, FGF-20, FGF-21, FGF-22 and FGF-23), blood coagulation factors (e.g., Factor V, Factor VII, Factor VIII, B-domain deleted Factor VIII, partial B-domain deleted Factor VIII, vWF-Factor VIII fusion (e.g., with full-length, B-domain deleted Factor VIII or partial B-domain deleted Factor VIII), Factor IX, Factor X and Factor XIII), hormones, such as human growth hormone (hGH) and follicle stimulating hormone (FSH), as well as cytokines, such as interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18) and interferons (e.g., INF-alpha, INF-beta, INF-gamma).

Other exemplary polypeptides include enzymes, such as glucocerebrosidase, alpha-galactosidase (e.g., Fabrazyme™), acid-alpha-glucosidase (acid maltase), iduronidases, such as alpha-L-iduronidase (e.g., Aldurazyme™), thyroid peroxidase (TPO), beta-glucosidase (see e.g., enzymes described in U.S. patent application Ser. No. 10/411,044), arylsulfatase, asparaginase, alpha-glucoceramidase, sphingomyelinase, butyrylcholinesterase, urokinase and alpha-galactosidase A (see e.g., enzymes described in U.S. Pat. No. 7,125,843).

Other exemplary parent polypeptides include bone morphogenetic proteins (e.g., BMP-1, BMP-2, BMP-3, BMP-4, BMP-5, BMP-6, BMP-7, BMP-8, BMP-9, BMP-10, BMP-11, BMP-12, BMP-13, BMP-14, BMP-15), neurotrophins (e.g., NT-3, NT-4, NT-5), erythropoietins (EPO), growth differentiation factors (e.g., GDF-5), glial cell line-derived neurotrophic factor (GDNF), brain derived neurotrophic factor (BDNF), nerve growth factor (NGF), von Willebrand factor (vWF), vWF-cleaving protease (vWF-protease, vWF-degrading protease), granulocyte colony stimulating factor (G-CSF), granulocyte-macrophage colony stimulating factor (GM-CSF), α₁-antitrypsin (ATT, or α-1 protease inhibitor), tissue-type plasminogen activator (TPA), hirudin, leptin, urokinase, human DNase, insulin, hepatitis B surface protein (HbsAg), human chorionic gonadotropin (hCG), chimeric diphtheria toxin-IL-2, glucagon-like peptides (e.g., GLP-1 and GLP-2), anti-thrombin III (AT-III), prokinetisin, CD4, α-CD20, tumor necrosis factor receptor (TNF-R), P-selectin glycoprotein ligand-1 (PSGL-1), complement, transferrin, glycosylation-dependent cell adhesion molecule (GlyCAM), neural-cell adhesion molecule (N-CAM), TNF receptor-IgG Fc region fusion protein, extendin-4, BDNF, beta-2-microglobulin, ciliary neurotrophic factor (CNTF), fibrinogen, GDF (e.g., GDF-1, GDF-2, GDF-3, GDF-4, GDF-5, GDF-6-15), GDNF and GLP-1. Exemplary amino acid sequences for some of the above listed polypeptides are described in U.S. Pat. No. 7,214,660, all of which are incorporated herein by reference.

Also within the scope of the invention are polypeptides that are antibodies. The term antibody is meant to include antibody fragments (e.g., Fc domains), single chain antibodies, Lama antibodies, nano-bodies and the like. Also included in the term are antibody-fusion proteins, such as Ig chimeras. Preferred antibodies include humanized, monoclonal antibodies or fragments thereof. All known isotypes of such antibodies are within the scope of the invention. Exemplary antibodies include those to growth factors, such as endothelial growth factor (EGF), vascular endothelial growth factors (e.g., monoclonal antibody to VEGF-A, such as ranibizumab (Lucentis™)) and fibroblast growth factors, such as FGF-7, FGF-21 and FGF-23) and antibodies to their respective receptors. Other exemplary antibodies include anti-TNF-alpha monoclonal antibodies (see e.g., U.S. patent application Ser. No. 10/411,043), TNF receptor-IgG Fc region fusion protein (e.g., Enbrel™), anti-HER2 monoclonal antibodies (e.g., Herceptin™), monoclonal antibodies to protein F of respiratory syncytial virus (e.g., Synagis™), monoclonal antibodies to TNF-α (e.g., Remicade™), monoclonal antibodies to glycoproteins, such as IIb/IIIa (e.g., Reopro™), monoclonal antibodies to CD20 (e.g., Rituxan™), CD4 and alpha-CD3, monoclonal antibodies to PSGL-1 and CEA. Any modified (e.g., mutated) version of any of the above listed polypeptides is also within the scope of the invention.

The mutant polypeptides of the invention can be generated using methods known in the art and described herein below.

### O-Linked Glycosylation Sequence

In one embodiment, the O-linked glycosylation sequence of the invention is naturally present in a wild-type polypeptide. In another embodiment, the O-linked glycosylation sequence is not present or not present at the same position, in a parent polpeptide and is introduced into the parent polypeptide by mutation or other means. The O-linked glycosylation sequence of the invention can be any short amino acid sequence (e.g., 1 to 10, preferably about 3 to 9 amino acid residues) encompassing at least one amino acid having a hydroxyl group in its side chain (e.g., serine, threonine). This hydroxyl group marks the site of glycosylation.

Efficiency of glycosylation for each O-linked glycosylation sequence of the invention is dependent on the enzyme as well as on the context of the glycosylation sequence, especially the three-dimensional structure of the polypeptide around the glycosylation site.

### Positioning of O-linked Glycosylation Sequences

In one embodiment, the O-linked or S-linked glycosylation sequence, when part of a polypeptide (e.g., a sequon polypeptide of the invention), is a substrate for a glycosyl transferase. In one example the glycosylation sequence is a substrate for a GlcNAc transferase. In another example, the glycosylation sequence is a substrate for a modified enzyme, such as a truncated GlcNAc transferase. The efficiency, with which each O-linked glycosylation sequence of the invention is glycosylated during an appropriate glycosylation reaction, may depend on the type and nature of the enzyme, and may also depend on the context of the glycosylation sequence, especially the three-dimensional structure of the polypeptide around the glycosylation site.

Generally, an O-linked glycosylation sequence can be introduced at any position within the amino acid sequence of the polypeptide. In one example, the glycosylation sequence is introduced at the N-terminus of the parent polypeptide (i.e., preceding the first amino acid or immediately following the first amino acid) (amino-terminal mutants). In another example, the glycosylation sequence is introduced near the amino-terminus (e.g., within 10 amino acid residues of the N-terminus) of the parent polypeptide. In another example, the glycosylation sequence is located at the C-terminus of the parent polypeptide immediately following the last amino acid of the parent polypeptide (carboxy-terminal mutants). In yet another example, the glycosylation sequence is introduced near the C-terminus (e.g., within 10 amino acid residues of the C-terminus) of the parent polypeptide. In yet another example, the O-linked glycosylation sequence is located anywhere between the N-terminus and the C-terminus of the parent polypeptide (internal mutants). It is generally preferred that the modified polypeptide be biologically active, even if that biological activity is altered from the biological activity of the corresponding parent polypeptide.

An important factor influencing glycosylation efficiencies of sequon polypeptides is the accessibility of the glycosylation site (e.g., a serine or threonine side chain) for the glycosyltransferase (e.g., GlcNAc transferase) and other reaction partners, including solvent molecules. If the glycosylation sequence is positioned within an internal domain of the three-dimensional polypeptide structure, glycosylation will likely be inefficient. Hence, in one embodiment, the glycosylation sequence is introduced at a region of the polypeptide, which corresponds to the polypeptide's solvent exposed surface. An exemplary polypeptide conformation is one, in which the hydroxyl group of the glycosylation sequence is not oriented inwardly, forming hydrogen bonds with other regions of the polypeptide. Another exemplary conformation is one, in which the hydroxyl group is unlikely to form hydrogen bonds.

In one example, the glycosylation sequence is created within a pre-selected, specific region of the parent protein. In nature, glycosylation of the polypeptide backbone usually occurs within loop regions of the polypeptide and typically not within helical or beta-sheet structures. Therefore, in one embodiment, the sequon polypeptide of the invention is generated by introducing an O-linked glycosylation sequence into an area of the parent polypeptide, which corresponds to a loop domain.

For example, the crystal structure of the protein BMP-7 contains two extended loop regions between Ala⁷² and Ala⁸⁶ as well as Ile⁹⁶ and Pro¹⁰³. Generating BMP-7 mutants, in which the O-linked glycosylation sequence is placed within those regions of the polypeptide sequence, may result in polypeptides, wherein the mutation causes little or no disruption of the original tertiary structure of the polypeptide.

However, the inventors have discovered that introduction of an O-linked glycosylation sequence at an amino acid position that falls within a beta-sheet or alpha-helical conformation can also lead to sequon polypeptides, which are efficiently glycosylated at the newly introduced O-linked glycosylation sequence. Introduction of an O-linked glycosylation sequence into a beta-sheet or alpha-helical domain may cause structural changes to the polypeptide, which, in turn, enable efficient glycosylation (see e.g., U.S. patent application Ser. No. 11/781,885 filed Jul. 23, 2007, incorporated herein by reference in its entirety for all purposes.

The crystal structure of a protein can be used to identify those domains of a wild-type or parent polypeptide that are most suitable for introduction of an O-linked glycosylation sequence and may allow for the pre-selection of promising modification sites.

When a crystal structures is not available, the amino acid sequence of the polypeptide can be used to pre-select promising modification sites (e.g., prediction of loop domains versus alpha-helical domains). However, even if the three-dimensional structure of the polypeptide is known, structural dynamics and enzyme/receptor interactions are variable in solution. Hence, the identification of suitable mutation sites as well as the selection of suitable glycosylation sequences, may involve the creation of several sequon polypeptides (e.g., libraries of sequon polypeptides of the invention) and testing those variants for desirable characteristics using appropriate screening protocols, e.g., those described herein.

In one embodiment, the parent polypeptide is an antibody or antibody fragment. In one example, the constant region (e.g., C_{H}2 domain) of an antibody or antibody fragment is modified with an O-linked glycosylation sequence of the invention. In one example, the O-linked glycosylation sequence is introduced in such a way that a naturally occurring N-linked glycosylation sequence is replaced or functionally impaired. In another embodiment sequon scanning is performed through a selected area of the C_{H}2 domain creating a library of antibodies, each including an exogenous O-linked glycosylation sequence of the invention. In yet another embodiment, resulting polypeptide variants are subjected to an enzymatic glycosylation reaction adding a glycosyl moiety to the introduced glycosylation sequence. Those variants that are sufficiently glycosylated can be anlyzed for their ability to bind a suitable receptor (e.g., F_{c} receptor, such as F_{c}γRIIIa). In one embodiment, such glycosylated antibody or antibody fragment exhibits increased binding affinity to the F_{c} receptor when compared with the parent antibody or a naturally glycosylated version thereof. This aspect of the invention is further described in U.S. Provisional Patent Application 60/881,130 filed Jan. 18, 2007, the disclosure of which is incorporated herein in its entirety. The described modification can change the effector function of the antibody. In one embodiment, the glycosylated antibody variant exhibits reduced effector function, e.g., reduced binding affinity to a receptor found on the surface of a natural killer cell or on the surface of a killer T-cell.

In another embodiment, the O-linked or S-linked glycosylation sequence is not introduced within the parent polypeptide sequence, but rather the sequence of the parent polypeptide is extended though addition of a peptide linker fragment to either the N- or C-terminus of the parent polypeptide, wherein the peptide linker fragment includes an O-linked or S-linked glycosylation sequence of the invention, such as “PVS”. The peptide linker fragment can have any number of amino acids. In one embodiment the peptide linker fragment includes at least about 5, at least about 10, at least about 15, at least about 20, at least about 30, at least about 50 or more than 50 amino acid residues. The peptide linker fragment optionally includes an internal or terminal amino acid residue that has a reactive functional group, such as an amino group (e.g., lysine) or a sufhydryl group (e.g., cysteine). Such reactive functional group may be used to link the polypeptide to another moiety, such as another polypeptide, a cytotoxin, a small-molecule drug or another modifying group of the invention. This aspect of the invention is further described in U.S. Provisional Patent Application 60/881,130 filed Jan. 18, 2007, the disclosure of which is incorporated herein in its entirety. In an exemplary embodiment, the peptide linker fragment includes a lysine residue that serves as a branching point for the linker, e.g., the amino group of the lysine serves as an attachment point for an “arm” of the linker. In an exemplary embodiment, the lysine replaces the methionine moiety. In another exemplary embodiment, the linker fragment is dimerized with another linker fragment of identical or different structure through formation of a disulfide bond.

In one embodiment, the parent polypeptide that is modified with a peptide linker fragment of the invention is an antibody or antibody fragment. In one example according to this embodiment, the parent polypeptide is scFv. Methods described herein can be used to prepare scFvs of the present invention in which the scFv or the linker is modified with a glycosyl moiety or a modifying group attached to the peptide through a glycosyl linking group. Exemplary methods of glycosylation and glycoconjugation are set forth in, e.g., PCT/US02/32263 and U.S. patent application Ser. No. 10/411,012, each of which is incorporated by reference herein in its entirety.

The inventors have discovered that glycosylation is most efficient when the O-linked glycosylation sequence includes a proline (P) residue near the site of glycosylation (e.g., serine or threonine residue). In one embodiment, the proline residue precedes (is found toward the N-terminus of) the glycosylation site. Exemplary glycosylation sites of the invention according to this embodiment include PVS, PB²VT, and P(B²)₂VT. Typically, 0 to 5, preferably 0 to 4 and more preferably, 0 to 3 amino acids are found between the proline residue and the glycosylation site. In another embodiment, the proline residue is found toward the C-terminus of the glycosylation site. Exemplary O-linked glycosylation sites of the invention according to this embodiment include SB⁷TP and SB⁷SP.

In one embodiment, certain amino acid residues are included into the O-linked glycosylation sequence to modulate expressability of the mutated polypeptide in a particular organism, such as E. coli, proteolytic stability, structural characteristics and/or other properties of the polypeptide.

In one embodiment, the O-linked glycosylation sequence of the invention includes an amino acid sequence, which is a member selected from Formulae (I) to (VI), shown below:

(B¹)ₐP(B²)_{b}US(B³)_{c} (I)

(B¹)ₐP(B²)_{b}UT(B³)_{c} (II)

(B⁴)_{d}PSZ(B⁵)ₑ (III)

(B⁴)_{d}PTZ(B⁵)ₑ (IV)

(B⁶)_{f}S(B⁷)_{g}P(B⁸)ₕ (V)

(B⁶)_{f}T(B⁷)_{g}P(B⁸)ₕ (VI)

In Formulae (I) to (VI), the integers b and g are independently selected from 0 to 2. the integers a, c, d, e, f and h are independently selected from 0 to 5. T is threonine, S is serine and P is proline. U is a member selected from V (valine), S (serine), T (threonine), E (glutamic acid), Q (glutamine) and uncharged amino acids. Z is a member selected from P, E, Q, S, T and uncharged amino acids, and each B¹, B², B³, B⁴, B⁵, B⁶, B⁷ and B⁸ is a member independently selected from an amino acid.

In an exemplary embodiment, the polypeptide of the invention contains an O-linked glycosylation sequence that is a member selected from the formulae:

| | |
|---|---|
| (B¹)ₐ P V S (B³)_{c}; | (SEQ ID NO: 16) |
| (B¹)ₐ P V T (B³)_{c}; | (SEQ ID NO: 17) |
| (B¹)ₐ P S S (B³)_{c}; | (SEQ ID NO: 18) |
| (B¹)ₐ P S T (B³)_{c}; | (SEQ ID NO: 19) |
| (B¹)ₐ P T S (B³)_{c}; | (SEQ ID NO: 20) |
| (B¹)ₐ P B² V T (B³)_{c}; | (SEQ ID NO: 21) |
| (B¹)ₐ P B² V S (B³)_{c}; | (SEQ ID NO: 22) |
| (B¹)ₐ P K U T (B³)_{c}; | (SEQ ID NO: 23) |
| (B¹)ₐ P K U S (B³)_{c}; | (SEQ ID NO: 24) |
| (B¹)ₐ P Q U T (B³)_{c}; | (SEQ ID NO: 25) |
| (B¹)ₐ P Q U S (B³)_{c}; | (SEQ ID NO: 26) |
| (B¹)ₐ P (B²)₂ V S (B³)_{c}; | (SEQ ID NO: 27) |
| (B¹)ₐ P (B²)₂ V T (B³)_{c}; | (SEQ ID NO: 28) |
| (B¹)ₐ P (B²)₂ T S (B³)_{c}; | (SEQ ID NO: 29) |
| (B¹)ₐ P (B²)₂ T T (B³)_{c}; | (SEQ ID NO: 30) |
| (B⁴)_{d} P T P (B⁵)ₑ; | (SEQ ID NO: 31) |
| (B⁴)_{d} P T E (B⁵)ₑ; | (SEQ ID NO: 32) |
| (B⁴)_{d} P S A (B⁵)ₑ; | (SEQ ID NO: 33) |
| (B⁶)_{f} S B⁷ T P (B⁸)ₕ; | (SEQ ID NO: 34) |
| and | |
| (B⁶)_{f} S B⁷ S P (B⁸)ₕ, | (SEQ ID NO: 35) |

wherein a, b, c, d, e, f, g, h, B¹, B², B³, B⁴, B⁵, B⁶, B⁷ and B⁸ are defined as hereinabove.

In another exemplary embodiment, the O-linked glycosylation sequence of the invention includes an amino acid sequence, which is a member selected from:

| | |
|---|---|
| PVS, | (SEQ ID NO: 36) |
| PVSG, | (SEQ ID NO: 37) |
| PVSGS, | (SEQ ID NO: 38) |
| VPVS, | (SEQ ID NO: 39) |
| VPVSG, | (SEQ ID NO: 40) |
| VPVSGS, | (SEQ ID NO: 41) |
| PVSR, | (SEQ ID NO: 42) |
| PVSRE, | (SEQ ID NO: 43) |
| PVSA, | (SEQ ID NO: 44) |
| PVSAS, | (SEQ ID NO: 45) |
| APVS, | (SEQ ID NO: 46) |
| APVSA, | (SEQ ID NO: 47) |
| APVSAS, | (SEQ ID NO: 48) |
| APVSS, | (SEQ ID NO: 49) |
| APVSSS, | (SEQ ID NO: 50) |
| PVSS, | (SEQ ID NO: 51) |
| PVSSA, | (SEQ ID NO: 52) |
| PVSSAP, | (SEQ ID NO: 53) |
| IPVS, | (SEQ ID NO: 54) |
| PVSR, | (SEQ ID NO: 55) |
| PVSRE, | (SEQ ID NO: 56) |
| IPVSR, | (SEQ ID NO: 57) |
| VPVS, | (SEQ ID NO: 58) |
| VPVSS, | (SEQ ID NO: 59) |
| VPVSSA, | (SEQ ID NO: 60) |
| RPVS, | (SEQ ID NO: 61) |
| RPVSS, | (SEQ ID NO: 62) |
| RPVSSA, | (SEQ ID NO: 63) |
| PVT, | (SEQ ID NO: 64) |
| PSS, | (SEQ ID NO: 65) |
| PSST, | (SEQ ID NO: 66) |
| PSSTA, | (SEQ ID NO: 67) |
| PPSS, | (SEQ ID NO: 68) |
| PPSST, | (SEQ ID NO: 69) |
| PSSG, | (SEQ ID NO: 70) |
| PSSGF, | (SEQ ID NO: 71) |
| SPST, | (SEQ ID NO: 72) |
| SPSTS, | (SEQ ID NO: 73) |
| SPSTSP, | (SEQ ID NO: 74) |
| SPSS, | (SEQ ID NO: 75) |
| SPSSG, | (SEQ ID NO: 76) |
| SPSSGF, | (SEQ ID NO: 77) |
| PST, | (SEQ ID NO: 78) |
| PSTS, | (SEQ ID NO: 79) |
| PSTST, | (SEQ ID NO: 80) |
| PSTV, | (SEQ ID NO: 81) |
| PSTVS, | (SEQ ID NO: 82) |
| PSVT, | (SEQ ID NO: 83) |
| PSVTI, | (SEQ ID NO: 84) |
| PSVS, | (SEQ ID NO: 85) |
| PAVT, | (SEQ ID NO: 86) |
| PAVTA, | (SEQ ID NO: 87) |
| PAVTAA, | (SEQ ID NO: 88) |
| KPAVT, | (SEQ ID NO: 89) |
| KPAVTA, | (SEQ ID NO: 90) |
| PAVS, | (SEQ ID NO: 91) |
| PQQS, | (SEQ ID NO: 92) |
| PQQSA, | (SEQ ID NO: 93) |
| PQQSAS, | (SEQ ID NO: 94) |
| PQQT, | (SEQ ID NO: 95) |
| PKGS, | (SEQ ID NO: 96) |
| PKGSR, | (SEQ ID NO: 97) |
| PKGT, | (SEQ ID NO: 98) |
| PKSS, | (SEQ ID NO: 99) |
| PKSSA, | (SEQ ID NO: 100) |
| PKSSAP, | (SEQ ID NO: 101) |
| PKST, | (SEQ ID NO: 102) |
| PADTS, | (SEQ ID NO: 103) |
| PADTSD, | (SEQ ID NO: 104) |
| PADTT, | (SEQ ID NO: 105) |
| PIKVT, | (SEQ ID NO: 106) |
| PIKVTE, | (SEQ ID NO: 107) |
| PIKVS, | (SEQ ID NO: 108) |
| SPST, | (SEQ ID NO: 109) |
| SPSTS, | (SEQ ID NO: 110) |
| SPTS, | (SEQ ID NO: 111) |
| SPTSP, | (SEQ ID NO: 112) |
| PTSP, | (SEQ ID NO: 113) |
| SPTSP, | (SEQ ID NO: 114) |
| SPSA, | (SEQ ID NO: 115) |
| SPSAK, | (SEQ ID NO: 116) |
| TSPS, | (SEQ ID NO: 117) |
| TSPSA, | (SEQ ID NO: 118) |
| LPTP, | (SEQ ID NO: 119) |
| LPTPP, | (SEQ ID NO: 120) |
| PTPP, | (SEQ ID NO: 121) |
| PTPPL, | (SEQ ID NO: 122) |
| VPTE, | (SEQ ID NO: 123) |
| VPTET, | (SEQ ID NO: 124) |
| PTE, | (SEQ ID NO: 125) |
| PTET, | (SEQ ID NO: 126) |
| TSETP, | (SEQ ID NO: 127) |
| ITSETP, | (SEQ ID NO: 128) |
| ASVSP, | (SEQ ID NO: 129) |
| SASVSP, | (SEQ ID NO: 130) |
| VETP, | (SEQ ID NO: 131) |
| VETPR, | (SEQ ID NO: 132) |
| ETPR, | (SEQ ID NO: 133) |
| ACTQ, | (SEQ ID NO: 134) |
| ACTQG and | (SEQ ID NO: 135) |
| CTQG, | (SEQ ID NO: 136) |

wherein each threonine (T) independently can optionally be replaced with serine (S) and each serine independently can optionally be replaced with threonine.

Other exemplary O-linked glycosylation sequences are disclosed in T. M. Leavy and C. R. Bertozzi, Bioorg. Med. Chem. Lett. 2007, 17: 3851-3854, the disclosure of which is incorporated herein by reference in its entirety for all purposes. In one example, the O-linked glycosylation sequence includes one of the following amino acid sequences: PIPVSRE, RIPVSRE, RIPVSRA, PIPVSRA, RIPVSRP, PIPVSRP, AIPVSRA and AIPVSRP. O-linked glycosylation sequences, which glycosylate with high efficiency and those, which cause the enzyme to add only one glycosyl residue per glycosylation sequence are generally preferred.

### Non-Naturally Occurring Polypeptides

The O-linked glycosylation sequences of the invention can be part of any parent or wild-type polypeptide. In one embodiment, the parent sequence is mutated in such a way that the O-linked-glycosylation sequence is inserted into the parent sequence adding the entire length and respective number of amino acids to the amino acid sequence of the parent polypeptide. In another embodiment, the O-linked glycosylation sequence replaces one or more amino acids of the parent polypeptide. In an exemplary embodiment, the mutation is introduced into the parent peptide using one or more of the pre-existing amino acids to be part of the O-linked glycosylation sequence. For instance, a proline residue in the parent peptide is maintained and those amino acids immediately preceding and/or following the proline are mutated to create an O-linked-glycosylation sequence of the invention. In another exemplary embodiment, the O-linked glycosylation sequence is created employing a combination of amino acid insertion and replacement of existing amino acids.

### Libraries of Mutant Polypeptides

One strategy for the identification of polypeptides, which are glycosylated or glycoPEGylated efficiently (e.g., with a satisfactory yield) when subjected to a glycosylation or glycoPEGylation reaction, is to insert an O-linked glycosylation sequence of the invention at a variety of different positions within the amino acid sequence of a parent polypeptide, including e.g., beta-sheet domains and alpha-helical domains, and then to test a number of the resulting sequon polypeptides for their ability to function as an efficient substrate for a glycosyltransferase, such as human GlcNAc transferase.

Hence, in another aspect, the invention provides a library of sequon polypeptides including a plurality of different members, wherein each member of the library corresponds to a common parent polypeptide and includes at least one independently selected exogenous O-linked or S-linked glycosylation sequence of the invention. In one embodiment, each member of the library includes the same O-linked glycosylation sequence, each at a different amino acid position within the parent polypeptide. In another embodiment, each member of the library includes a different O-linked glycosylation sequence, however at the same amino acid position within the parent polypeptide. O-linked glycosylation sequences, which are useful in conjunction with the libaries of the invention are described herein. In one embodiment, the O-linked glycosylation sequence used in a library of the invention has an amino acid sequence according to Formula (I). In another embodiment, the O-linked glycosylation sequence used in a library of the invention has an amino acid sequence according to Formula (II). In one embodiment, the O-linked glycosylation sequence used in a library of the invention has an amino acid sequence according to Formula (III). In one embodiment, the O-linked glycosylation sequence used in a library of the invention has an amino acid sequence according to Formula (IV). In one embodiment, the O-linked glycosylation sequence used in a library of the invention has an amino acid sequence according to Formula (V). In one embodiment, the O-linked glycosylation sequence used in a library of the invention has an amino acid sequence according to Formula (VI).

In one embodiment, in which each member of the library has a common O-linked glycosylation sequence, the parent polypeptide has an amino acid sequence that includes “m” amino acids. In one example, the library of sequon polypeptides includes (a) a first sequon polypeptide having the O-linked glycosylation sequence at a first amino acid position (AA)ₙ within the parent polypeptide, wherein n is a member selected from 1 to m; and (b) at least one additional sequon polypeptide, wherein in each additional sequon polypeptide the O-linked glycosylation sequence is introduced at an additional amino acid position, each additional amino acid position selected from (AA)ₙ₊ₓ and (AA)ₙ₋ₓ, wherein x is a member selected from 1 to (m−n). For example, a first sequon polypeptide is generated through introduction of a selected O-linked glycosylation sequence at the first amino acid position. Subsequent sequon polypeptides may then be generated by introducing the same O-linked glycosylation sequence at an amino acid position, which is located further towards the N- or C-terminus of the parent polypeptide.

In this context, when n−x is 0 (AA₀) then the glycosylation sequence is introduced immediately preceding the N-terminal amino acid of the parent polypeptide. An exemplary sequon polypeptide may have the partial sequence: “PVSM¹ . . . ”

The first amino acid position (AA)ₙ can be anywhere within the amino acid sequence of the parent polypeptide. In one embodiment, the first amino acid position is selected (e.g., at the beginning of a loop domain).

Each additional amino acid position can be anywhere within the parent polypeptide. In one example, the library of sequon polypeptides includes a second sequon polypeptide having the O-linked glycosylation sequence at an amino acid position selected from (AA)ₙ₊ₚ and (AA)ₙ₋ₚ, wherein p is selected from 1 to about 10, preferably from 1 to about 8, more preferably from 1 to about 6, even more preferably from 1 to about 4 and most preferably from 1 to about 2. In one embodiment, the library of sequon polypeptides includes a first sequon polypeptide having an O-linked glycosylation sequence at amino acid position (AA)ₙ and a second sequon polypeptide having an O-linked glycosylation sequence at amino acid position (AA)ₙ₊₁ or (AA)ₙ₋₁.

In another example, each of the additional amino acid position is immediately adjacent to a previously selected amino acid position. In yet another example, each additional amino acid position is exactly 1, 2, 3, 4, 5, 6, 7, 8, 9 or 10 amino acid(s) removed from a previously selected amino acid position.

Introduction of an O-linked or S-linked glycosylation sequence “at a given amino acid position” of the parent polypeptide means that the mutation is introduced starting immediately next to the given amino acid position (towards the C-terminus). Introduction can occur through full insertion (not replacing any existing amino acids), or by replacing any number of existing amino acids.

In an exemplary embodiment, the library of sequon polypeptides is generated by introducing the O-linked glycosylation sequence at consecutive amino acid positions of the parent polypeptide, each located immediately adjacent to the previously selected amino acid position, thereby “scanning” the glycosylation sequence through the amino acid chain, until a desired, final amino acid position is reached. Immediately adjacent means exactly one amino acid position further towards the N- or C-terminus of the parent polypeptide. For instance, the first mutant is created by introduction of the glycosylation sequence at amino acid position AAₙ. The second member of the library is generated through introduction of the glycosylation site at amino acid position AAₙ₊₁, the third mutant at AAₙ₊₂, and so forth. This procedure has been termed “sequon scanning”. One of skill in the art will appreciate that sequon scanning can involve designing the library so that the first member has the glycosylation sequence at amino acid position (AA)ₙ, the second member at amino acid position (AA)ₙ₊₂, the third at (AA)ₙ₊₄ etc. Likewise, the members of the library may be characterized by other strategic placements of the glycosylation sequence. For example:

A) member 1: (AA)ₙ; member 2: (AA)ₙ₊₃; member 3: (AA)ₙ₊₆; member 4: (AA)ₙ₊₉ etc.
B) member 1: (AA)ₙ; member 2: (AA)ₙ₊₄; member 3: (AA)ₙ₊₈; member 4: (AA)ₙ₊₁₂ etc.
C) member 1: (AA)ₙ; member 2: (AA)ₙ₊₅; member 3: (AA)ₙ₊₁₀; member 4: (AA)ₙ₊₁₅ etc.

In one embodiment, a first library of sequon polypeptides is generated by scanning a selected O-linked or S-linked glycosylation sequence of the invention through a particular region of the parent polypeptide (e.g., from the beginning of a particular loop region to the end of that loop region). A second library is then generated by scanning the same glycosylation sequence through another region of the polypeptide, “skipping” those amino acid positions, which are located between the first region and the second region. The part of the polypeptide chain that is left out may, for instance, correspond to a binding domain important for biological activity or another region of the polypeptide sequence known to be unsuitable for glycosylation. Any number of additional libraries can be generated by performing “sequon scanning” for additional stretches of the polypeptide. In an exemplary embodiment, a library is generated by scanning the O-linked glycosylation sequence through the entire polypeptide introducing the mutation at each amino acid position within the parent polypeptide.

In one embodiment, the members of the library are part of a mixture of polypeptides. For example, a cell culture is infected with a plurality of expression vectors, wherein each vector includes the nucleic acid sequence for a different sequon polypeptide of the invention. Upon expression, the culture broth may contain a plurality of different sequon polypeptides, and thus includes a library of sequon polypeptides. This technique may be usefull to determine, which sequon polypeptide of a library is expressed most efficiently in a given expression system.

In another embodiment, the members of the library exist isolated from each other. For example, at least two of the sequon polypeptides of the above mixture may be isolated. Together, the isolated polypeptides represent a library. Alternatively, each sequon polypeptide of the library is expressed separately and the sequon polypeptides are optionally isolated. In another example, each member of the library is synthesized by chemical means and optionally purified.

The library of mutant polypeptides according to the invention can be generated using any of the O-linked glycosylation sequences described herein. In a preferred embodiment, the library is generated using an O-linked glycosylation sequence, which is a member selected from:

(B¹)ₐ P V S (B³)_{c};
(B¹)ₐ P V T (B³)_{c};
(B¹)ₐ P S S (B³)_{c};
(B¹)ₐ P S T (B³)_{c};
(B¹)ₐ P T S (B³)_{c};
(B¹)ₐ P B² V T (B³)_{c};
(B¹)ₐ P B² V S (B³)_{c};
(B¹)ₐ P K U T (B³)_{c};
(B¹)ₐ P K U S (B³)_{c};
(B¹)ₐ P Q U T (B³)_{c};
(B¹)ₐ P Q U S (B³)_{c};
(B¹)ₐ P (B²)₂ V S (B³)_{c};
(B¹)ₐ P (B²)₂ V T (B³)_{c};
(B¹)ₐ P (B²)₂ T S (B³)_{c};
(B¹)ₐ P (B²)₂ T T (B³)_{c};
(B⁴)_{d} P T P (B⁵)ₑ;
(B⁴)_{d} P T E (B⁵)ₑ;
(B⁴)_{d} P S A (B⁵)ₑ;
(B⁶)_{f} S B⁷ T P (B⁸)ₕ;
and
(B⁶)_{f} S B⁷ S P (B⁸)ₕ.

### Exemplary Non-Naturally Occurring Polypeptides

An exemplary parent polypeptide is recombinant human BMP-7. The selection of BMP-7 as an exemplary parent polypeptide is for illustrative purposes and is not meant to limit the scope of the invention. A person of skill in the art will appreciate that any parent polypeptide (e.g., those set forth herein) are equally suitable for the following exemplary modifications. Any polypeptide variant thus obtained falls within the scope of the invention. Biologically active BMP-7 variants of the present invention include any BMP-7 polypeptide, in part or in whole, that includes at least one modification that does not result in substantial or entire loss of its biological activity as measured by any suitable functional assay known to one skilled in the art. The following sequence (140 amino acids) represents a biologically active portion of the full-lengthh BMP-7 sequence:

(SEQ ID NO: 137)

M¹STGSKQRSQNRSKTPKNQEALRMANVAENSSSDQRQACKKHELYVSF
RDLGWQDWIIAPEGYAAYYCEGECAFPLNSYMNATNHAIVQTLVHFINP
ETVPKPCCAPTQLNAISVLYFDDSSNVILKKYRNMVVRACGCH

Exemplary mutant BMP-7 polypeptides, which are based on the above parent polypeptide sequence, are listed in Tables 2 to 11, below. In a preferred embodiment, mutant polypeptides are generated taking the substrate requirements of the glycosyltransferase into consideration.

In one exemplary embodiment, mutations are introduced into the wild-type BMP-7 amino acid sequence (SEQ ID NO: 137) replacing the corresponding number of amino acids in the parent sequence, resulting in a mutant polypeptide that contains the same number of amino acid residues as the parent polypeptide. For instance, directly substituting three amino acids normally in BMP-7 with the O-linked glycosylation sequence “proline-valine-serine” (PVS) and then sequentially moving the PVS sequence towards the C-terminus of the polypeptide provides 137 BMP-7 analogs including the glycosylation site PVS. Exemplary sequences according to this embodiment are listed in Table 2, below.

TABLE 2

Exemplary library of mutant BMP-7 polypeptides including 140 amino acids
wherein three existing amino acids are replaced with the O-linked
glycosylation sequence “PVS”

Introduction at position 1, replacing 3 existing amino acids:
M¹PVSSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 138)
Introduction at position 2, replacing 3 existing amino acids:
M¹SPVSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 139)
Introduction at position 3, replacing 3 existing amino acids:
M¹STPVSQRSQNRSKTPKNQEALRMANVAENSSSDQRQACKKHELYVSFRDLGWQD
WIIAPEGYAAYYCEGECAFPLNSYMNATNHAIVQTLVHFINPETVPKPCCAPTQLNAI
SVLYFDDSSNVILKKYRNMVVRACGCH (SEQ ID NO: 140)
Additional BMP-7 mutants can be generated by “scanning” the glycosylation sequence
through the entire sequence. All mutant BMP-7 sequences thus obtained are within the scope
of the invention. The final mutant polypeptide so generated has the following sequence:
Introduction at position 137, replacing 3 existing amino acids:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACPVS (SEQ ID NO: 141)

In another exemplary embodiment, mutations are introduced into the wild-type BMP-7 amino acid sequence (SEQ ID NO: 137) by adding one or more amino acids to the parent sequence. For instance, the O-linked glycosylation sequence PVS is added to the parent BMP-7 sequence replacing 2, 1 or none of the amino acids in the parent sequence. In one example, the glycosylation sequence is added to the N- or C-terminus of the parent sequence. Exemplary sequences according to this embodiment are listed in Table 3, below.

TABLE 3
Exemplary BMP-7 mutants including PVS (141 to 143 amino acids)

Introduction at position 1, not replacing any existing amino acids (full insertion):
M¹PVSSTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 142)
Introduction at position 1, replacing 1 existing amino acid (S):
M¹PVSTGSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 143)
Introduction at position 1, replacing 2 existing amino acids (ST):
M¹PVSGSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 144)
Introduction at position 138, replacing 2 existing amino acids (CH), adding 1 amino acid:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGPVS (SEQ ID NO: 145)
Introduction at position 139, replacing 1 existing amino acid (H), adding 2 amino acids:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCPVS (SEQ ID NO: 146)
Introduction at position 140, adding 3 amino acids:
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCHPVS (SEQ ID NO: 147)

In another example, the O-linked glycosylation sequence is introduced into the peptide sequence at any amino acid position by adding one or more amino acids to the parent sequence. In this example, the maximum number of added amino acid residues corresponds to the length of the inserted glycosylation sequence. In an exemplary embodiment, the parent sequence is extended by exactly one amino acid. For example, the O-linked glycosylation sequence PVS is added to the parent BMP-7 peptide replacing 2 amino acids normally present in BMP-7. Exemplary sequences according to this embodiment are listed in Table 4, below.

TABLE 4

Exemplary library of mutant BMP-7 polypeptides including 141 amino acids,
wherein two existing amino acids are replaced with the O-linked glycosylation
sequence “PVS”

Introduction at position 1, adding 1 amino acid, replacing 2 amino acids (ST)
M¹PVSGSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 148)
Introduction at position 2, adding 1 amino acid, replacing 2 amino acids (TG)
M¹SPVSSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 149)
Introduction at position 3, adding 1 amino acid, replacing 2 amino acids (GS)
M¹STPVSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 150)
Introduction at position 4, adding 1 amino acid, replacing 2 amino acids (SK)
M¹STGPVSQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 151)
Introduction at position 5, adding 1 amino acid, replacing 2 amino acids (KQ)
M¹STGSPVSRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 152)
Additional BMP-7 mutants can be generated by “scanning” the glycosylation sequence
through the entire sequence in the above fashion. All mutant BMP-7 sequences thus obtained
are within the scope of the invention.

Another example involves the addition of an O-linked glycosylation sequence (e.g., PVS) to the parent BMP-7 peptide replacing 1 amino acid normally present in BMP-7 (double amino acid insertion). Exemplary sequences according to this embodiment are listed in Table 5, below.

TABLE 5

Exemplary library of BMP-7 mutants including PVS; replacement of one
existing amino acid (142 amino acids)

Introduction at position 1, adding 2 amino acids, replacing 1 amino acid (S)
M¹PVSTGSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 153)
Introduction at position 2, adding 2 amino acids, replacing 1 amino acid (T)
M¹SPVSGSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 154)
Introduction at position 3, adding 2 amino acids, replacing 1 amino acid (G)
M¹STPVSSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 155)
Introduction at position 4, adding 2 amino acids, replacing 1 amino acid (S)
M¹STGPVSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 156)
Introduction at position 5, adding 2 amino acids, replacing 1 amino acid (K)
M¹STGSPVSQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 157)
Additional BMP-7 mutants can be generated by “scanning” the glycosylation sequence
through the entire sequence in the above fashion. All mutant BMP-7 sequences thus obtained
are within the scope of the invention.

Yet another example involves the creation of an O-linked glycosylation sequence within the parent BMP-7 sequence replacing none of the amino acids normally present in BMP-7 and adding the entire lengthh of the glycosylation sequence (e.g., triple amino acid insertion for PVS) to any position within the parent peptide. Exemplary sequences according to this embodiment are listed in Table 6, below.

TABLE 6

Exemplary library of BMP-7 mutants including PVS; addition of
3 amino acids (143 amino acids)

Introduction at position 1, adding 3 amino acids
M¹PVSSTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 142)
Introduction at position 2, adding 3 amino acids
M¹SPVSTGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 158)
Introduction at position 3, adding 3 amino acids
M¹STPVSGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 159)
Introduction at position 4, adding 3 amino acids
M¹STGPVSSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 160)
Additional BMP-7 mutants can be generated by “scanning” the
glycosylation sequence through the entire sequence in
the above fashion. All mutant BMP-7 sequences thus obtained
are within the scope of the invention.

Analogues iterations of BMP-7 mutants can be generated using any of the O-linked glycosylation sequences of the invention. For instance, instead of PVS any of SEQ ID NOs x to x can be used. In one example, instead of PVS the sequences PAVT (SEQ ID NO: 86) or PIKVS (SEQ ID NO: 108) can be used. In an exemplary embodiment PIKVS is introduced into the parent peptide replacing 5 amino acids normally present in BMP-7. Exemplary sequences according to this embodiment are listed in Table 7, below.

TABLE 7

Exemplary library of BMP-7 mutants including PIKVS;
replacement of 5 amino acids (140 amino acids)

M¹PIKVSQRSQNRSKTPKNQEALRMANVAENSSSDQRQACKKHELYVSFRDLGWQD
WIIAPEGYAAYYCEGECAFPLNSYMNATNHAIVQTLVHFINPETVPKPCCAPTQLNAI
SVLYFDDSSNVILKKYRNMVVRACGCH (SEQ ID NO: 161)
M¹SPIKVSRSQNRSKTPKNQEALRMANVAENSSSDQRQACKKHELYVSFRDLGWQD
WIIAPEGYAAYYCEGECAFPLNSYMNATNHAIVQTLVHFINPETVPKPCCAPTQLNAI
SVLYFDDSSNVILKKYRNMVVRACGCH (SEQ ID NO: 162)
M¹STPIKVSSQNRSKTPKNQEALRMANVAENSSSDQRQACKKHELYVSFRDLGWQD
WIIAPEGYAAYYCEGECAFPLNSYMNATNHAIVQTLVHFINPETVPKPCCAPTQLNAI
SVLYFDDSSNVILKKYRNMVVRACGCH (SEQ ID NO: 163)
M¹STGPIKVSQNRSKTPKNQEALRMANVAENSSSDQRQACKKHELYVSFRDLGWQD
WIIAPEGYAAYYCEGECAFPLNSYMNATNHAIVQTLVHFINPETVPKPCCAPTQLNAI
SVLYFDDSSNVILKKYRNMVVRACGCH (SEQ ID NO: 164)
M¹STGSPIKVSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 165)

Additional BMP-7 mutants can be generated by “scanning” the glycosylation sequence through the entire sequence in the above fashion. All mutant BMP-7 sequences thus obtained are within the scope of the invention.

In another example the O-linked glycosylation sequence PIKVS is added to the wild-type BMP-7 sequence at or close to either the N- or C-terminal of the parent sequence, adding 1 to 5 amino acids to the wild-type. Exemplary sequences according to this embodiment are listed in Table 8, below.

TABLE 8

Exemplary libraries of BMP-7 mutants including PIKVS
(141-145 amino acids)

Amino-terminal mutants:
Introduction at position 1, adding 5 amino acids
M¹PIKVSSTGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 166)
Introduction at position 1, adding 4 amino acids, replacing 1 amino acid (S)
M¹PIKVSTGSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 167)
Introduction at position 1, adding 3 amino acids, replacing 2 amino acids (ST)
M¹PIKVSGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 168)
Introduction at position 1, adding 2 amino acids, replacing 3 amino acids (STG)
M¹PIKVSSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 169)
Introduction at position 1, adding 1 amino acids, replacing 4 amino acids (STGS)
M¹PIKVSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 170)
Carboxy-terminal mutants
Introduction at position 140, adding 5 amino acids
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCHPIKVS (SEQ ID NO: 171)
Introduction at position 139, adding 4 amino acids, replacing 1 amino acid (H)
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCPIKVS (SEQ ID NO: 172)
Introduction at position 138, adding 3 amino acids, replacing 2 amino acid (CH)
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGPIKVS (SEQ ID NO: 173)
Introduction at position 137, adding 2 amino acids, replacing 3 amino acid (GCH)
M¹STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACPIKVS (SEQ ID NO: 174)
Introduction at position 136, adding 1 amino acids, replacing 4 amino acid (CGCH)
M¹ STGSKQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRAPIKVS (SEQ ID NO: 175)

Yet another example involves insertion of the O-linked glycosylation sequence TSETP (SEQ ID NO: 127) into the wild-type BMP-7 sequence, adding 1 to 5 amino acids to the parent sequence. Exemplary sequences according to this embodiment are listed in Table 9, below.

TABLE 9
Exemplary library of BMP-7 mutants including TSETP

Insertion of one amino acid
M¹TSETPKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 176)
M¹STSETPQRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 177)
M¹STTSETPRSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 178)
M¹STGTSETPSQNRSKTPKNQEALRMANVAENSSSDQRQACKKHELYVSFRDLGWQ
DWIIAPEGYAAYYCEGECAFPLNSYMNATNHAIVQTLVHFINPETVPKPCCAPTQLN
AISVLYFDDSSNVILKKYRNMVVRACGCH (SEQ ID NO: 179)
Additional BMP-7 mutants can be generated by “scanning” the glycosylation sequence
through the entire sequence in the above fashion. All mutant BMP-7 sequences thus obtained
are within the scope of the invention.
Insertion of two amino acids
M¹TSETPSKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 180)
M¹STSETPKQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 181)
M¹STTSETPQRSQNRSKTPKNQEALRMANVAENSSSDQRQACKKHELYVSFRDLGW
QDWIIAPEGYAAYYCEGECAFPLNSYMNATNHAIVQTLVHFINPETVPKPCCAPTQL
NAISVLYFDDSSNVILKKYRNMVVRACGCH (SEQ ID NO: 182)
Additional BMP-7 mutants can be generated by “scanning” the glycosylation sequence
through the entire sequence in the above fashion. All mutant BMP-7 sequences thus obtained
are within the scope of the invention.
Insertion of three amino acids
M¹TSETPGSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 183)
M¹STSETPSKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 184)
M¹STTSETPKQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 185)
M¹STGTSETPQRSQNRSKTPKNQEALRMANVAENSSSDQRQACKKHELYVSFRDLG
WQDWIIAPEGYAAYYCEGECAFPLNSYMNATNHAIVQTLVHFINPETVPKPCCAPTQ
LNAISVLYFDDSSNVILKKYRNMVVRACGCH (SEQ ID NO: 186)
Additional BMP-7 mutants can be generated by “scanning” the glycosylation sequence
through the entire sequence in the above fashion. All mutant BMP-7 sequences thus obtained
are within the scope of the invention.
Insertion of four amino acids
M¹TSETPTGSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 187)
M¹STSETPGSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 188)
M¹STTSETPSKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 189)
M¹STGTSETPKQRSQNRSKTPKNQEALRMANVAENSSSDQRQACKKHELYVSFRDL
GWQDWIIAPEGYAAYYCEGECAFPLNSYMNATNHAIVQTLVHFINPETVPKPCCAPT
QLNAISVLYFDDSSNVILKKYRNMVVRACGCH (SEQ ID NO: 190)
Additional BMP-7 mutants can be generated by “scanning” the glycosylation sequence
through the entire sequence in the above fashion. All mutant BMP-7 sequences thus obtained
are within the scope of the invention.
Insertion of five amino acids
M¹TSETPSTGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 191)
M¹STSETPTGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 192)
M¹STTSETPGSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 193)
M¹STGTSETPSKQRSQNRSKTPKNQEALRMANVAENSSSDQRQACKKHELYVSFRD
LGWQDWIIAPEGYAAYYCEGECAFPLNSYMNATNHAIVQTLVHFINPETVPKPCCAP
TQLNAISVLYFDDSSNVILKKYRNMVVRACGCH (SEQ ID NO: 194)
Additional BMP-7 mutants can be generated by “scanning” the glycosylation sequence
through the entire sequence in the above fashion. All mutant BMP-7 sequences thus obtained
are within the scope of the invention.

Other examples for mutant polypeptides containing O-linked glycosylation sequences are disclosed in U.S. Provisional Patent Applications 60/710,401 filed Aug. 22, 2005; and 60/720,030, filed Sep. 23, 2005; WO2004/99231 and WO2004/10327, which are incorporated herein by reference for all purposes.

In order to identify optimal positions for the O-linked glycosylation sequence within the parent peptide (e.g., with respect to glycosylation, glycoPEGylation and biological activity), a variety of mutants are created and then screened for desired properties (“Sequon Scan”). In an exemplary embodiment, the mutation site is “moved” along the parent peptide from the N-terminal side of the preselected peptide region towards the C-terminus (e.g., one amino acid at a time).

In one example, the O-linked glycosylation sequence (e.g., PVS) is placed at all possible amino acid positions within selected peptide regions either by substitution of existing amino acids and/or by insertion. Exemplary sequences according to this embodiment are listed in Table 10 and Table 11, below.

TABLE 10
Exemplary library of BMP-7 mutants including
PVS between A⁷³ and A⁸²
Substitution of existing amino acids A⁷³ to A⁸²

(SEQ ID NO: 195)

A⁷³FPLNSYMNA⁸²TNHAIVQTLVHFI⁹⁵NPETVPKP¹⁰³

(SEQ ID NO: 196)

P⁷³VSLNSYMNA⁸²TNHAIVQTLVHFI⁹⁵NPETVPKP¹⁰³

(SEQ ID NO: 197)

A⁷³PVSNSYMNA⁸²TNHAIVQTLVHFI⁹⁵NPETVPKP¹⁰³

(SEQ ID NO: 198)

A⁷³FPVSSYMNA⁸²TNHAIVQTLVHFI⁹⁵NPETVPKP¹⁰³

(SEQ ID NO: 199)

A⁷³FPPVSYMNA⁸²TNHAIVQTLVHFI⁹⁵NPETVPKP¹⁰³

(SEQ ID NO: 200)

A⁷³FPLPVSMNA⁸²TNHAIVQTLVHFI⁹⁵NPETVPKP¹⁰³

(SEQ ID NO: 201)

A⁷³FPLNPVSNA⁸²TNHAIVQTLVHFI⁹⁵NPETVPKP¹⁰³

(SEQ ID NO: 202)

A⁷³FPLNSPVSA⁸²TNHAIVQTLVHFI⁹⁵NPETVPKP¹⁰³

(SEQ ID NO: 203)

A⁷³FPLNSYPVS⁸²TNHAIVQTLVHFI⁹⁵NPETVPKP¹⁰³

TABLE 11

Exemplary library of BMP-7 mutants including PVS
between I⁹⁵ and P¹⁰³

Substitution of existing amino acids I⁹⁵ to P¹⁰³

| | |
|---|---|
| A⁷³FPLNSYMNA⁸²TNHAIVQTLVHFP⁹⁵VSETVPKP¹⁰³ | (SEQ ID NO: 204) |
| A⁷³FPLNSYMNA⁸²TNHAIVQTLVHFI⁹⁵PVSTVPKP¹⁰³ | (SEQ ID NO: 205) |
| A⁷³FPLNSYMNA⁸²TNHAIVQTLVHFI⁹⁵NPVSVPKP¹⁰³ | (SEQ ID NO: 206) |
| A⁷³FPLNSYMNA⁸²TNHAIVQTLVHFI⁹⁵NPPVSPKP¹⁰³ | (SEQ ID NO: 207) |
| A⁷³FPLNSYMNA⁸²TNHAIVQTLVHFI⁹⁵NPEPVSKP¹⁰³ | (SEQ ID NO: 208) |
| A⁷³FPLNSYMNA⁸²TNHAIVQTLVHFI⁹⁵NPETPVSP¹⁰³ | (SEQ ID NO: 209) |
| A⁷³FPLNSYMNA⁸²TNHAIVQTLVHFI⁹⁵NPETVPVS¹⁰³ | (SEQ ID NO: 210) |

Insertion (with one amino acid added) between existing
amino acids A⁷³ to A⁸²

| | |
|---|---|
| P⁷³VSPLNSYMNA⁸³TNHAIVQTLVHFI⁹⁶NPETVPKP¹⁰⁴ | (SEQ ID NO: 211) |
| A⁷³PVSLNSYMNA⁸³TNHAIVQTLVHFI⁹⁶NPETVPKP¹⁰⁴ | (SEQ ID NO: 212) |
| A⁷³FPVSNSYMNA⁸³TNHAIVQTLVHFI⁹⁶NPETVPKP¹⁰⁴ | (SEQ ID NO: 213) |
| A⁷³FPPVSSYMNA⁸³TNHAIVQTLVHFI⁹⁶NPETVPKP¹⁰⁴ | (SEQ ID NO: 214) |
| A⁷³FPLPVSYMNA⁸³TNHAIVQTLVHFI⁹⁶NPETVPKP¹⁰⁴ | (SEQ ID NO: 215) |
| A⁷³FPLNPVSMNA⁸³TNHAIVQTLVHFI⁹⁶NPETVPKP¹⁰⁴ | (SEQ ID NO: 216) |
| A⁷³FPLNSPVSNA⁸³TNHAIVQTLVHFI⁹⁶NPETVPKP¹⁰⁴ | (SEQ ID NO: 217) |
| A⁷³FPLNSYPVSA⁸³TNHAIVQTLVHFI⁹⁶NPETVPKP¹⁰⁴ | (SEQ ID NO: 218) |
| A⁷³FPLNSYMPVS⁸³TNHAIVQTLVHFI⁹⁶NPETVPKP¹⁰⁴ | (SEQ ID NO: 219) |

Insertion (with one amino acid added) between existing
amino acids I⁹⁵ to P¹⁰³

| | |
|---|---|
| A⁷³FPLNSYMNA⁸²TNHAIVQTLVHFP⁹⁵VSPETVPKP¹⁰⁴ | (SEQ ID NO: 220) |
| A⁷³FPLNSYMNA⁸²TNHAIVQTLVHFI⁹⁵PVSETVPKP¹⁰⁴ | (SEQ ID NO: 221) |
| A⁷³FPLNSYMNA⁸²TNHAIVQTLVHFI⁹⁵NPVSTVPKP¹⁰⁴ | (SEQ ID NO: 222) |
| A⁷³FPLNSYMNA⁸²TNHAIVQTLVHFI⁹⁵NPPVSVPKP¹⁰⁴ | (SEQ ID NO: 223) |
| A⁷³FPLNSYMNA⁸²TNHAIVQTLVHFI⁹⁵NPEPVSPKP¹⁰⁴ | (SEQ ID NO: 224) |
| A⁷³FPLNSYMNA⁸²TNHAIVQTLVHFI⁹⁵NPETPVSKP¹⁰⁴ | (SEQ ID NO: 225) |
| A⁷³FPLNSYMNA⁸²TNHAIVQTLVHFI⁹⁵NPETVPVSP¹⁰⁴ | (SEQ ID NO: 226) |
| A⁷³FPLNSYMNA⁸²TNHAIVQTLVHFI⁹⁵NPETVPPVS¹⁰⁴ | (SEQ ID NO: 227) |

The above substitutions and insertions, e.g., of Tables 3-11, can be made using any other O-linked glycosylation sequences of the invention (e.g., SEQ ID NOs: 36-136). All mutant BMP-7 sequences thus obtained are within the scope of the invention.

In another exemplary embodiment, one or more O-glycosylation sequences, such as those set forth above is inserted into a blood coagulation Factor, e.g., Factor VII, Factor VIII or Factor IX polypeptide. As set forth in the context of BMP-7, the O-glycosylation sequence can be inserted in any of the various motifs exemplified with BMP-7. For example, the O-glycosylation sequence can be inserted into the wild type sequence without replacing any amino acid(s) native to the wild type sequence. In an exemplary embodiment, the O-glycosylation sequence is inserted at or near the N- or C-terminus of the polypeptide. In another exemplary embodiment, one or more amino acid residue native to the wild type polypeptide sequence is removed prior to insertion of the O-glycosylation site. In yet another exemplary embodiment, one or more amino acid residue native to the wild type sequence is a component of the O-glycosylation sequence (e.g., a proline) and the O-glycosylation sequence encompasses the wild type amino acid(s). The wild type amino acid(s) can be at either terminus of the O-glycosylation sequence or internal to the O-glycosylation sequence.

Furthermore, any preexisting N-linked glycosylation sequence can be replaced with an O-linked glycosylation sequence of the invention. In addition, an O-linked glycosylation sequence can be inserted adjacent to one or more N-linked glycosylation sequences. In a preferred embodiment, the presence of the O-linked glycosylation sequence prevents the glycosylation of the N-linked glycosylation sequence.

In a particular example, the polypeptide is Factor VIII. Factor VIII and Factor VIII variants are know in the art. For example, U.S. Pat. No. 5,668,108 describes Factor VIII variants, in which the aspartic acid at position 1241 is replaced by a glutamic acid. U.S. Pat. No. 5,149,637 describes Factor VIII variants comprising the C-terminal fraction, either glycosylated or unglycosylated, and U.S. Pat. No. 5,661,008 describes Factor VIII variants comprising amino acids 1-740 linked to amino acids 1649 to 2332 by at least 3 amino acid residues. Therefore, variants, derivatives, modifications and complexes of Factor VIII are well known in the art, and are encompassed in the present invention. Expression systems for the production of Factor VIII are also well known in the art, and include prokaryotic and eukaryotic cells, as exemplified in U.S. Pat. Nos. 5,633,150, 5,804,420, and 5,422,250. Any of the above discussed Factor VIII sequences may be modified to include an exogenous O-linked or S-linked glycosylation sequence of the invention.

When the parent polypeptide is Factor VIII, the O-linked glycosylation sequence can be inserted into the A-, B-, or C-domain according to any of the motifs set forth above. More than one O-linked glycosylation site can be inserted into a single domain or more than one domain; again, according to any of the motifs above. For example, an O-glycosylation site can be inserted into each of the A, B and C domains, the A and C domains, the A and B domains or the B and C domains. Alternatively, an O-linked glycosylation sequence can flank the A and B domain or the B and C domain.

In another exemplary embodiment, the Factor VIII polypeptide is a B-domain deleted (BDD) Factor VIII polypeptide. In this embodiment, the O-linked glycosylation sequence can be inserted into the peptide linker joining the 80 Kd and 90 Kd subunits of the Factor VIII heterodimer. Alternatively, the O-linked glycosylation sequence can flank the A domain and the linker or the C domain and linker. As set forth above in the context of BMP-7, the O-linked glycosylation sequence can be inserted without replacement of existing amino acids, or may be inserted replacing one or more amino acids of the parent polypeptide.

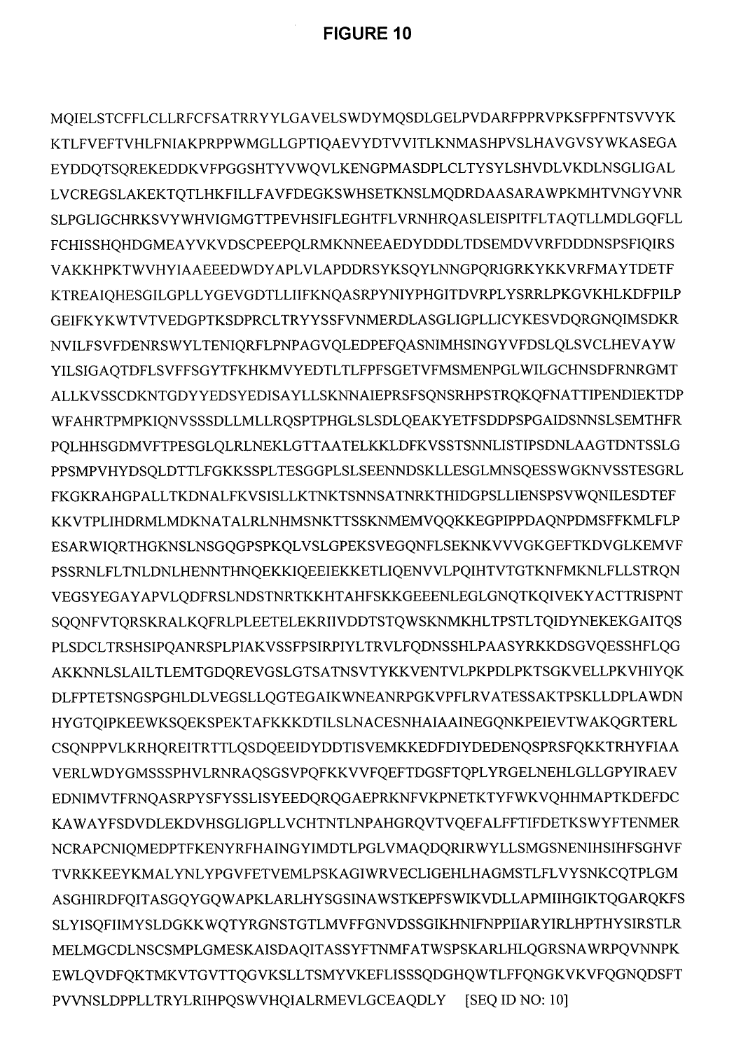

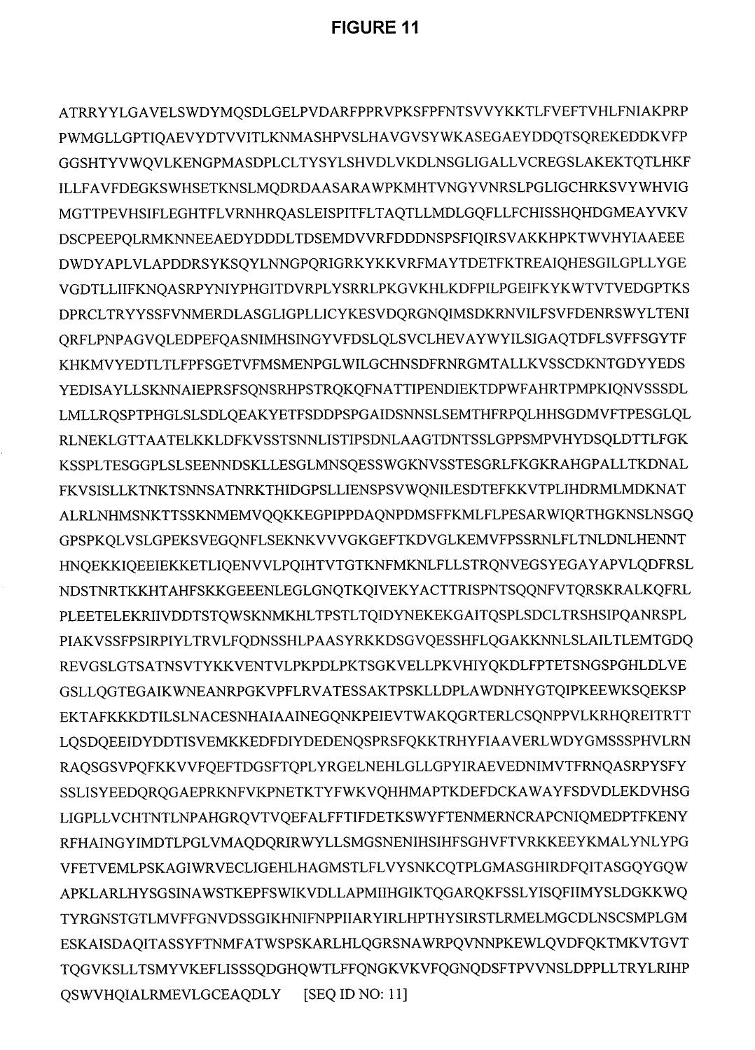

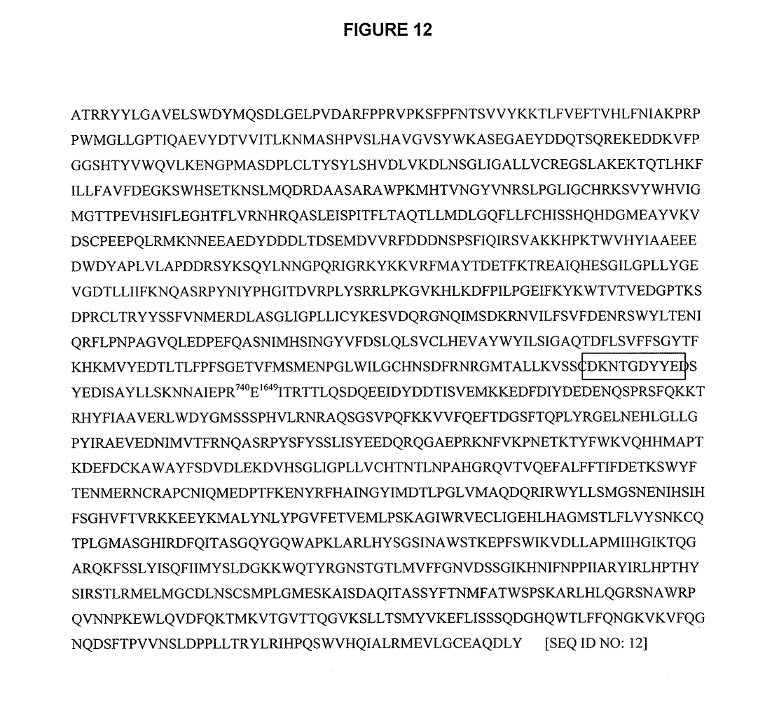

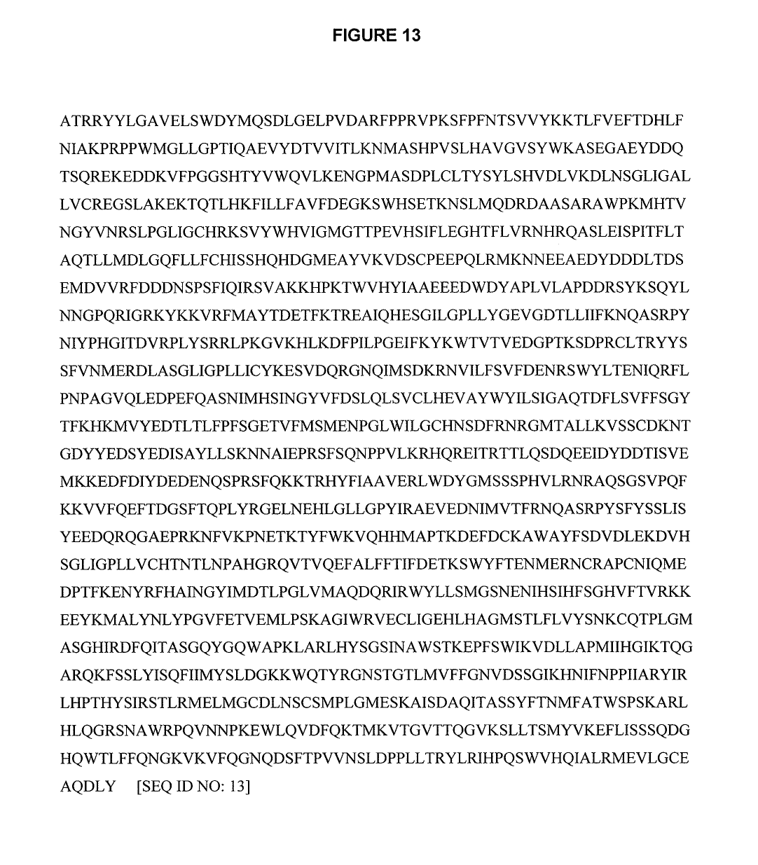

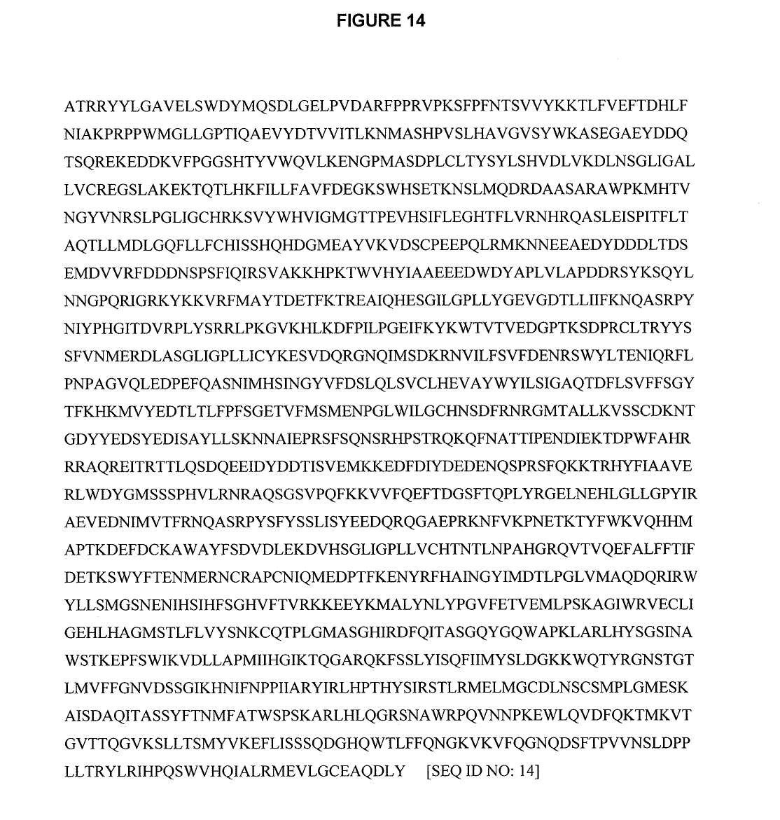

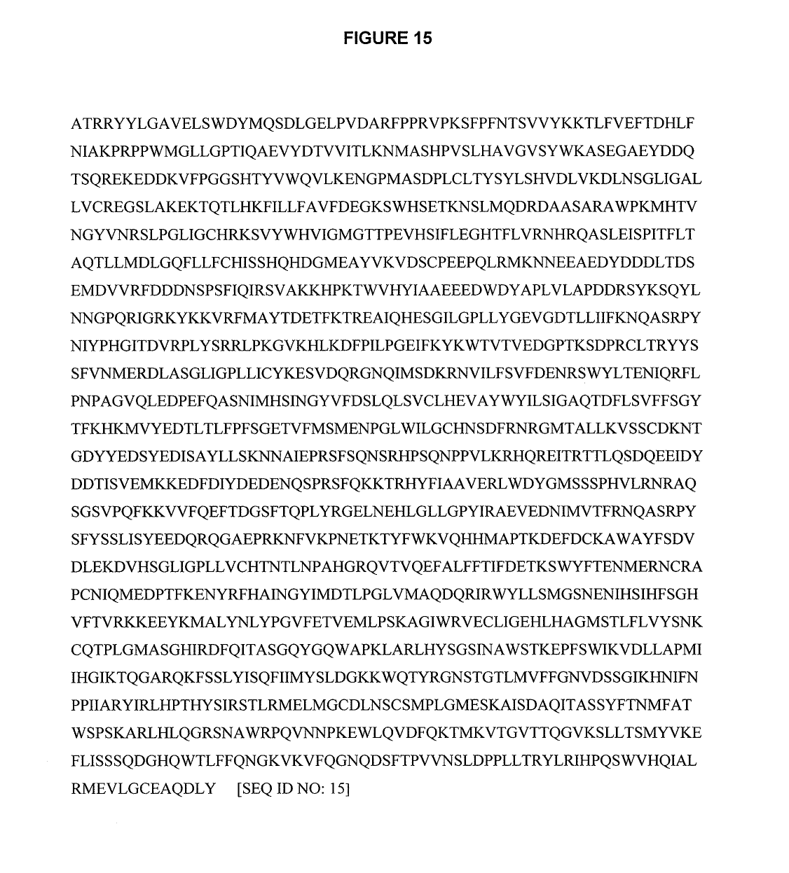

In one example, the Factor VIII is a full-length or wild-type Factor VIII polypeptide. An exemplary amino acid sequence for full-lenth Factor VIII polypeptides are shown in FIGS. 10 (SEQ ID NO: 10) and 11 (SEQ ID NO: 11). In yet another example, the polypeptide is a Factor VIII polypeptide, in which the B-domain includes less amino acid residues than the B-domain of wild-type or full-length Factor VIII. Those Factor VIII polypeptides are referred to as B-domain deleted or partial B-domain deleted Factor VIII. A person of skill in the art will be able to identify the B-domain within a given Factor VIII polypeptide. Exemplary amino acid sequences for B-domain deleted Factor VIII polypeptides include those sequences shown in FIGS. 12-15 (SEQ ID NOs: 12-15). Another exemplary Factor VIII sequence is disclosed in Sandberg et al., Seminars in Hematology 38(2):4-12 (2000), the disclosure of which is incorporated herein by reference.

In a further exemplary embodiment, the parent polypeptide is hGH and the O-glycosylation site is added according to any of the above-recited motifs.

As will be apparent to one of skill in the art, polypeptides including more than one mutant O-linked glycosylation sequence of the invention are also within the scope of the present invention. Additional mutations may be introduced to allow for the modulation of polypeptide properties, such e.g., biological activity, metabolic stability (e.g., reduced proteolysis), pharmacokinetics and the like.

Once a variety of mutants are prepared, they can be evaluated for their ability to function as a substrate for O-linked glycosylation or glycoPEGylation, for instance using a GlcNAc transferase. Succesfull glycosylation and/or glycoPEGylation may be detected and quantified using methods known in the art, such as mass spectroscopy (e.g., MALDI-TOF or Q-TOF), gel electrophoresis (e.g., in combination with densitometry) or chromatographic analyses (e.g., HPLC). Biological assays, such as enzyme inhibition assays, receptor-binding assays and/or cell-based assays can be used to analyze biological activities of a given polypeptide conjugate. Evaluation strategies are described in more detail herein, below (see e.g., “Identification of Lead polypeptides”. It will be within the abilities of a person skilled in the art to select and/or develop an appropriate assay system useful for the chemical and biological evaluation of each mutant polypeptide.

### Polypeptide Conjugates

In another aspect, the present invention provides a conjugate between a polypeptide of the invention (e.g., a mutant polypeptide) and a selected modifying group, in which the modifying group is conjugated to the polypeptide through a glycosyl linking group, e.g., an intact glycosyl linking group. The glycosyl linking group is either directly bound to an amino acid residue within an O-linked glycosylation sequence of the invention, or, alternatively, it is bound to an O-linked glycosylation sequence through one or more additional glycosyl residues. Methods of preparing the conjugates of the invention are set forth herein and in U.S. Pat. Nos. 5,876,980; 6,030,815; 5,728,554; and 5,922,577, as well as WO 98/31826; WO2003/031464; WO2005/070138; WO2004/99231; WO2004/10327; WO2006/074279; and U.S. Patent Application Publication 2003180835, the disclosures of which are incorporated herein by reference for all purposes.

The conjugates of the invention will typically correspond to the general structure:

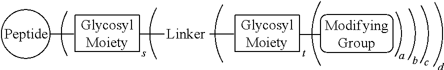

in which the symbols a, b, c, d and s represent a positive, non-zero integer; and t is either 0 or a positive integer. The “modifying group” is a polymeric moiety (e.g., a water-soluble polymer, such as PEG), therapeutic agent, a bioactive agent, a detectable label or the like. The linker can be any of a wide array of linking groups, infra. Alternatively, the linker may be a single bond. The identity of the peptide is without limitation.

Exemplary peptide conjugates include an O-linked glucosamine residue (e.g., GlcNAc or GlcNH). In one embodiment, the glucosamine moiety itself is derivatized with a modifying group and represents the glycosyl linking group. In another embodiment, additional glycosyl residues are attached to the peptide-bound glucosamine moiety. For example, another GlcNAc or GlcNH, a Gal or Sia residue, each of which can act as the glycosyl linking group, is added to the first glucosamine moiety. In representative embodiments, the O-linked saccharyl residue is a member selected from a modified glucosamine-mimetic moiety, GlcNAc-X*, GlcNH-X*, Glc-X*, GlcNAc-GlcNAc-X*, GlcNAc-GlcNH-X*, GlcNH-GlcNAc-X*, GlcNAc-Gal-X*, GlcNH-Gal-X*, GlcNAc-Sia-X*, GlcNH-Sia-X*, GlcNAc-Gal-Sia-X*, GlcNH-Gal-Sia-X*, GlcNAc-GlcNAc-Gal-Sia-X*, GlcNAc-GlcNAc-Man-X*, GlcNAc-GlcNAc-Man(Man)₂ (optionally including one or more modifying group) or GlcNAc-Gal-Gal-Sia-X*, in which X* is a modifying group. In the above examples, each GlcNAc independently can optionally be replaced with GlcNH.

In an exemplary embodiment, the polypeptide is a non-naturally occurring polypeptide that includes an exogenous O-linked glycosylation sequence of the invention. The polypeptide is preferably O-glycosylated within the glycosylation sequence with a glucosamine moiety. Additional sugar residues can be added to the resulting O-linked glucosamine moiety using glycosyltransferases known to add to GlcNAc or GlcNH (e.g., galactosyltransferases, fucosyltransferases, glucosyltransferases, mannosyltransferases and GlcNAc transferases). Together these methods can result in glycosyl structures including two or more sugar residues.

The modifying group is covalently attached to a polypeptide through a glycosyl linking group, which is interposed between the polypeptide and the modifying group. The glycosyl linking group is covalently attached to either an amino acid residue of the polypeptide or to a glycosyl residue of a glycopeptide. As discussed herein, the modifying group is essentially any species that can be attached to a glycosyl or glycosyl-mimetic moiety, resulting in a “modified sugar”. The modified sugar can be incorporated into a glycosyl donor (e.g., modified sugar nucleotide), which is recognized by an appropriate transferase enzyme, which appends the modified sugar onto the polypeptide or glycopeptide.

Exemplary modifying groups are selected from glycosidic (e.g., dextrans, polysialic acids) and non-glycosidic modifying groups and include polymers (e.g., PEG) and polypeptides (e.g., enzymes, antibodies, antigens, etc.). Exemplary non-glycosidic modifying groups are selected from linear and branched and can include one or more independently selected polymeric moieties, such as poly(alkylene glycol) and derivatives thereof. In an exemplary embodiment, the modifying group is a water-soluble polymeric group, e.g., poly(ethylene glycol) and derivatived thereof (PEG, m-PEG) or poly(propylene glycol) and derivatives thereof (PPG, m-PPG) and the like. In a preferred embodiment, the poly(ethylene glycol) or poly(propylene glycol) has a molecular weight that is essentially homodisperse. Additional modifying groups are described herein below. In one embodiment, the glycosyl linking group is covalently linked to at least one polymeric, non-glycosidic modifying group.

In one embodiment, the present invention provides polypeptide conjugates that are highly homogenous in their substitution patterns. Using the methods of the invention, it is possible to form peptide conjugates in which essentially all of the modified sugar moieties across a population of conjugates of the invention are attached to a structurally identical amino acid or glycosyl residue. Thus, in an exemplary embodiment, the invention provides a polypeptide conjugate including one or more water-soluble polymeric moiety covalently bound to an amino acid residue (e.g., threonine) within an O-linked glycosylation sequence of the polypeptide through a glycosyl linking group. In one example, each amino acid residue having a glycosyl linking group attached thereto has the same structure. In another exemplary embodiment, essentially each member of the population of water-soluble polymeric moieties is bound via a glycosyl linking group to a glycosyl residue of the polypeptide, and each glycosyl residue of the peptide to which the glycosyl linking group is attached has the same structure.

Thus the invention provides a covalent conjugate between a non-naturally occurring polypeptide and a polymeric modifying group, wherein the polypeptide corresponds to a parent-polypeptide. The amino acid sequence of the non-naturally occurring polypeptide includes at least one exogenous O-linked glycosylation sequence that is not present, or not present at the same position, in the corresponding parent polypeptide. In a preferred embodiment, the O-linked glycosylation sequence is a substrate for a GlcNAc-transferase. In one example, the O-linked glycosylation sequence includes an amino acid residue having a hydroxyl group (e.g., serine or threonine), and the polymeric modifying group is covalently linked to the polypeptide at the hydroxyl group of the O-linked glycosylation sequence via a glycosyl linking group.

In an exemplary embodiment, the conjugate of the invention has a structure according to Formula (VII), wherein w is an integer selected from 0 and 1 and q is an integer selected from 0 and 1:

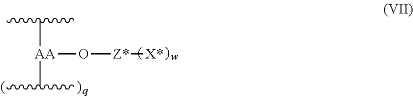

In Formula (VII), AA-0 is a moiety derived from an amino acid residue having a side chain, which is substituted with a hydroxyl group (e.g., serine or threonine), wherein the amino acid is located within an O-linked glycosylation sequence of the invention. When q is 1, then the amino acid is an internal amino acid of the polypeptide, and when q is 0, then the amino acid is an N-terminal or C-terminal amino acid. Z* is a member selected from a glucosamine-moiety, a glucosamine-mimetic moiety, an oligosaccharide comprising a glucosamine-moiety and an oligosaccharide comprising a glucosamine-mimetic moiety. X* is a member selected from a polymeric modifying group and a glycosyl linking group including a polymeric modifying group. In one example, Z* is a glucosamine-moiety and X* is a polymeric modifying group.

In one exemplary embodiment, X* is a polymeric modifying group. In another exemplary embodiment, Z* is a member selected from GlcNAc, GlcNH, Glc, GlcNAc-Fuc, GlcNAc-GlcNAc, GlcNH-GlcNH, GlcNAc-GlcNH, GlcNH-GlcNAc, GlcNAc-Gal, GlcNH-Gal, GlcNAc-Sia, GlcNH-Sia, GlcNAc-Gal-Sia, GlcNH-Gal-Sia, GlcNAc-GlcNAc-Gal-Sia, GlcNH-GlcNH-Gal-Sia, GlcNAc-GlcNH-Gal-Sia, GlcNH-GlcNAc-Gal-Sia, GlcNAc-GlcNAc-Man, GlcNAc-GlcNAc-Man(Man)₂, GlcNAc-Gal-Gal-Sia and other combinations of GlcNAc, GlcNH, Gal, Glc, Man, Fuc and Sia. In one embodiment, X* is a polymeric modifying group and Z* is a member selected from GlcNAc and GlcNH.

### Glycosyl Linking Group

The saccharide component of the modified sugar, when interposed between the polypeptide and a modifying group, becomes a “glycosyl linking group.” In an exemplary embodiment, the glycosyl linking group is formed from a mono- or oligo-saccharide that, after modification with a modifying group, is a substrate for an appropriate glycosyltransferase. In another exemplary embodiment, the glycosyl linking group is formed from a glycosyl-mimetic moiety. The polypeptide conjugates of the invention can include glycosyl linking groups that are mono- or multi-valent (i.e., mono- and multi-antennary structures). Thus, conjugates of the invention include species in which a selected moiety is attached to a peptide via a monovalent glycosyl linking group. Also included within the invention are conjugates in which more than one modifying group is attached to a polypeptide via a multivalent linking group.

In an exemplary embodiment, the covalent conjugate of the invention includes a moiety according to Formula (VIII):

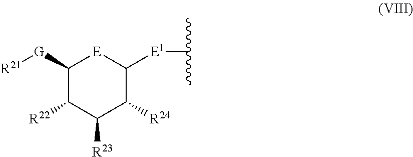

In Formula (VIII), G is a member selected from —CH₂— and C=A, wherein A is a member selected from O, S and NR²⁸, wherein R²⁸ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. E is a member selected from O, S NR²⁷ and CH₂, wherein R²⁷ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. E¹ is a member selected from O and S. R²¹, R²², R²³ and R²⁴ are members independently selected from H, OR²⁵, SR²⁵, NR²⁵R²⁶, NR²⁵S(O)₂NR²⁶, S(O)₂NR²⁵R²⁶, NR²⁵C(O)R²⁶, C(O)NR²⁵R²⁶, C(O)OR²⁵, acyl, substituted or unsubstituted alkyl, substituted or unsubstituted heteroalkyl, substituted or unsubstituted aryl, substituted or unsubstituted heteroaryl and substituted or unsubstituted heterocycloalkyl, wherein R²⁵ and R²⁶ are members independently selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl and a modifying group. Preferably, at least one of R²¹, R²², R²³, R²⁴, R²⁷, and R²⁸ comprises a polymeric modifying group.

In another exemplary embodiment, the covalent conjugate of the invention includes a moiety according to Formula (IX):

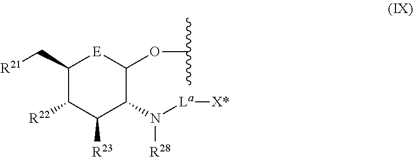

wherein X* is a polymeric modifying group selected from linear and branched; L^{a} is a member selected from a bond and a linker group and R²⁸ is a member selected from H, substituted or unsubstituted alkyl, substituted or unsubstituted heteroalkyl, substituted or unsubstituted aryl, substituted or unsubstituted heteroaryl, substituted or unsubstituted heterocycloalkyl.

In yet another exemplary embodiment, the covalent conjugate of the invention includes a moiety according to Formula (X):

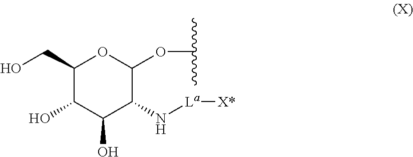

In one example, the modifying group includes a moiety, which is a member selected from:

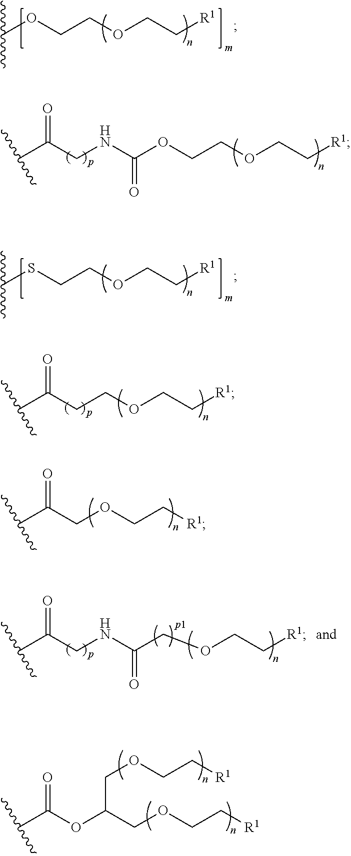

wherein p and p1 are integers independently selected from 1 to 20. Each n is an integer independently selected from 1 to 5000 and m is an integer from 1-5. R¹ is member selected from H, substituted or unsubstituted alkyl, substituted or unsubstituted heteroalkyl, substituted or unsubstituted heterocycloalkyl, substituted or unsubstituted aryl, substituted or unsubstituted heteroaryl, —NR¹²R¹³, —OR¹² and —SiR¹²R¹³, wherein R¹² and R¹³ are members independently selected from substituted or unsubstituted alkyl, substituted or unsubstituted heteroalkyl, substituted or unsubstituted heterocycloalkyl, substituted or unsubstituted aryl, and substituted or unsubstituted heteroaryl. In one example, R¹ is a member selected from OH and OR¹², wherein R¹² is a member selected from C₁, C₂, C₃, C₄, C₅ and C₆ alkyl. In another example, R¹ is a member selected from OH and OMe.

In one example, the modifying group X* is branched and includes at least two polymeric moieties. Exemplary modified sugar moieties are provided below:

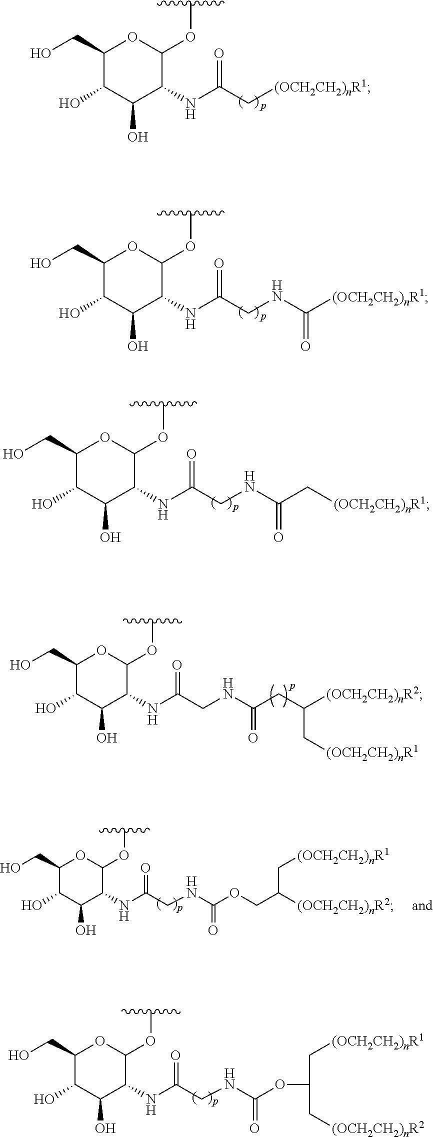

wherein R¹ and R² are members independently selected from OH and OMe, and p is an integer from 1 to 20.

### Modifying Group

The modifying group of the invention can be any chemical moiety. Exemplary modifying groups are discussed below. The modifying groups can be selected for their ability to alter the properties (e.g., biological or physicochemical properties) of a given polypeptide. Exemplary polypeptide properties that may be altered by the use of modifying groups include, but are not limited to, pharmacokinetics, pharmacodynamics, metabolic stability, biodistribution, water solubility, lipophilicity, tissue targeting capabilities and the therapeutic activity profile. Preferred modifying groups are those which improve pharmacodynamics and pharmacokinetics of a modified polypeptide when compared to the corresponding non-modified polypeptide. Other modifying groups may be used to create polypeptides that are useful in diagnostic applications or in vitro biological assay systems.

For example, the in vivo half-life of therapeutic glycopeptides can be enhanced with polyethylene glycol (PEG) moieties. Chemical modification of polypeptides with PEG (PEGylation) increases their molecular size and typically decreases surface- and functional group-accessibility, each of which are dependent on the number and size of the PEG moieties attached to the polypeptide. Frequently, this modification results in an improvement of plasma half-live and in proteolytic-stability, as well as a decrease in immunogenicity and hepatic uptake (Chaffee et al. J. Clin. Invest. 89: 1643-1651 (1992); Pyatak et al. Res. Commun. Chem. Pathol Pharmacol. 29: 113-127 (1980)). For example, PEGylation of interleukin-2 has been reported to increase its antitumor potency in vivo (Katre et al. Proc. Natl. Acad. Sci. USA. 84: 1487-1491 (1987)) and PEGylation of a F(ab′)2 derived from the monoclonal antibody A7 has improved its tumor localization (Kitamura et al. Biochem. Biophys. Res. Commun. 28: 1387-1394 (1990)).

In one embodiment, the in vivo half-life of a peptide derivatized with a PEG moiety by a method of the invention is increased relative to the in vivo half-life of the non-derivatized parent polypeptide. The increase in polypeptide in vivo half-life is best expressed as a range of percent increase relative to the parent polypeptide. The lower end of the range of percent increase is about 40%, about 60%, about 80%, about 100%, about 150% or about 200%. The upper end of the range is about 60%, about 80%, about 100%, about 150%, or more than about 250%.

### Water-Soluble Polymeric Modifying Groups

In one embodiment, the modifying group is a polymeric modifying group selected from linear and branched. In one example, the modifying group includes one or more polymeric moiety, wherein each polymeric moiety is independently selected.

Many water-soluble polymers are known to those of skill in the art and are useful in practicing the present invention. The term water-soluble polymer encompasses species such as saccharides (e.g., dextran, amylose, hyalouronic acid, poly(sialic acid), heparans, heparins, etc.); poly(amino acids), e.g., poly(aspartic acid) and poly(glutamic acid); nucleic acids; synthetic polymers (e.g., poly(acrylic acid), poly(ethers), e.g., poly(ethylene glycol); peptides, proteins, and the like. The present invention may be practiced with any water-soluble polymer with the sole limitation that the polymer must include a point at which the remainder of the conjugate can be attached.

The use of reactive derivatives of the modifying group (e.g., a reactive PEG analog) to attach the modifying group to one or more polypeptide moiety is within the scope of the present invention. The invention is not limited by the identity of the reactive analog.

In a preferred embodiment, the modifying group is PEG or a PEG analog. Many activated derivatives of poly(ethyleneglycol) are available commercially and are described in the literature. It is well within the abilities of one of skill to choose, and synthesize if necessary, an appropriate activated PEG derivative with which to prepare a substrate useful in the present invention. See, Abuchowski et al. Cancer Biochem. Biophys., 7: 175-186 (1984); Abuchowski et al., J. Biol. Chem., 252: 3582-3586 (1977); Jackson et al., Anal. Biochem., 165: 114-127 (1987); Koide et al., Biochem Biophys. Res. Commun., 111: 659-667 (1983)), tresylate (Nilsson et al., Methods Enzymol., 104: 56-69 (1984); Delgado et al., Biotechnol. Appl. Biochem., 12: 119-128 (1990)); N-hydroxysuccinimide derived active esters (Buckmann et al., Makromol. Chem., 182: 1379-1384 (1981); Joppich et al., Makromol. Chem., 180: 1381-1384 (1979); Abuchowski et al., Cancer Biochem. Biophys., 7: 175-186 (1984); Katre et al. Proc. Natl. Acad. Sci. U.S.A., 84: 1487-1491 (1987); Kitamura et al., Cancer Res., 51: 4310-4315 (1991); Boccu et al., Z. Naturforsch., 38C: 94-99 (1983), carbonates (Zalipsky et al., POLY(ETHYLENE GLYCOL) CHEMISTRY: BIOTECHNICAL AND BIOMEDICAL APPLICATIONS, Harris, Ed., Plenum Press, New York, 1992, pp. 347-370; Zalipsky et al., Biotechnol. Appl. Biochem., 15: 100-114 (1992); Veronese et al., Appl. Biochem. Biotech., 11: 141-152 (1985)), imidazolyl formates (Beauchamp et al., Anal. Biochem., 131: 25-33 (1983); Berger et al., Blood, 71: 1641-1647 (1988)), 4-dithiopyridines (Woghiren et al., Bioconjugate Chem., 4: 314-318 (1993)), isocyanates (Byun et al., ASAIO Journal, M649-M-653 (1992)) and epoxides (U.S. Pat. No. 4,806,595, issued to Noishiki et al., (1989). Other linking groups include the urethane linkage between amino groups and activated PEG. See, Veronese, et al., Appl. Biochem. Biotechnol., 11: 141-152 (1985).

Methods for activation of polymers can be found in WO 94/17039, U.S. Pat. No. 5,324,844, WO 94/18247, WO 94/04193, U.S. Pat. No. 5,219,564, U.S. Pat. No. 5,122,614, WO 90/13540, U.S. Pat. No. 5,281,698, and more WO 93/15189, and for conjugation between activated polymers and peptides, e.g. Coagulation Factor VIII (WO 94/15625), hemoglobin (WO 94/09027), oxygen carrying molecule (U.S. Pat. No. 4,412,989), ribonuclease and superoxide dismutase (Veronese at al., App. Biochem. Biotech. 11:141-45 (1985)).

Activated PEG molecules useful in the present invention and methods of making those reagents are known in the art and are described, for example, in WO04/083259.

Activating, or leaving groups, appropriate for activating linear PEGs of use in preparing the compounds set forth herein include, but are not limited to the species:

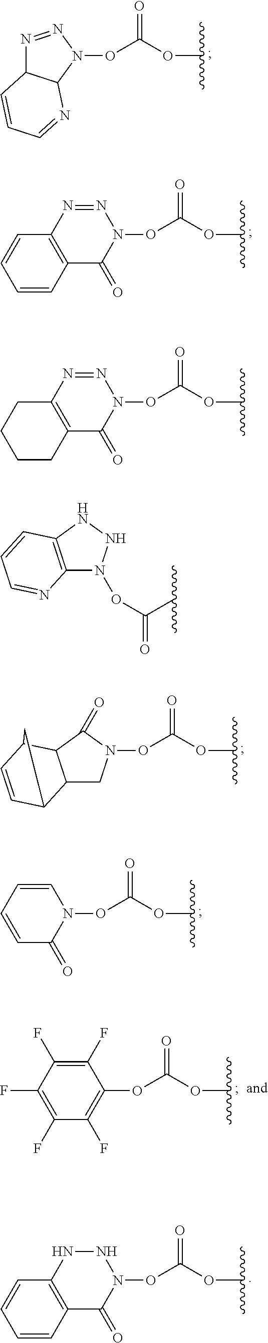

Exemplary water-soluble polymers are those in which a substantial proportion of the polymer molecules in a sample of the polymer are of approximately the same molecular weight; such polymers are “homodisperse.”

The present invention is further illustrated by reference to a poly(ethylene glycol) conjugate. Several reviews and monographs on the functionalization and conjugation of PEG are available. See, for example, Harris, Macronol. Chem. Phys. C25: 325-373 (1985); Scouten, Methods in Enzymology 135: 30-65 (1987); Wong et al., Enzyme Microb. Technol. 14: 866-874 (1992); Delgado et al., Critical Reviews in Therapeutic Drug Carrier Systems 9: 249-304 (1992); Zalipsky, Bioconjugate Chem. 6: 150-165 (1995); and Bhadra, et al., Pharmazie, 57:5-29 (2002). Routes for preparing reactive PEG molecules and forming conjugates using the reactive molecules are known in the art. For example, U.S. Pat. No. 5,672,662 discloses a water soluble and isolatable conjugate of an active ester of a polymer acid selected from linear or branched poly(alkylene oxides), poly(oxyethylated polyols), poly(olefinic alcohols), and poly(acrylomorpholine).

U.S. Pat. No. 6,376,604 sets forth a method for preparing a water-soluble 1-benzotriazolylcarbonate ester of a water-soluble and non-peptidic polymer by reacting a terminal hydroxyl of the polymer with di(1-benzotriazoyl)carbonate in an organic solvent. The active ester is used to form conjugates with a biologically active agent such as a polypeptide.

WO 99/45964 describes a conjugate comprising a biologically active agent and an activated water soluble polymer comprising a polymer backbone having at least one terminus linked to the polymer backbone through a stable linkage, wherein at least one terminus comprises a branching moiety having proximal reactive groups linked to the branching moiety, in which the biologically active agent is linked to at least one of the proximal reactive groups. Other branched poly(ethylene glycols) are described in WO 96/21469, U.S. Pat. No. 5,932,462 describes a conjugate formed with a branched PEG molecule that includes a branched terminus that includes reactive functional groups. The free reactive groups are available to react with a biologically active species, such as a polypeptide, forming conjugates between the poly(ethylene glycol) and the biologically active species. U.S. Pat. No. 5,446,090 describes a bifunctional PEG linker and its use in forming conjugates having a peptide at each of the PEG linker termini.

Conjugates that include degradable PEG linkages are described in WO 99/34833; and WO 99/14259, as well as in U.S. Pat. No. 6,348,558. Such degradable linkages are applicable in the present invention.

The art-recognized methods of polymer activation set forth above are of use in the context of the present invention in the formation of the branched polymers set forth herein and also for the conjugation of these branched polymers to other species, e.g., sugars, sugar nucleotides and the like.

An exemplary water-soluble polymer is poly(ethylene glycol), e.g., methoxy-poly(ethylene glycol). The poly(ethylene glycol) used in the present invention is not restricted to any particular form or molecular weight range. For unbranched poly(ethylene glycol) molecules the molecular weight is preferably between 500 and 100,000. A molecular weight of 2000-60,000 is preferably used and more preferably of from about 5,000 to about 40,000.

Exemplary poly(ethylene glycol) molecules of use in the invention include, but are not limited to, those having the formula:

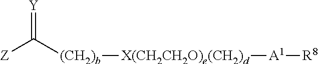

in which R⁸ is H, OH, NH₂, substituted or unsubstituted alkyl, substituted or unsubstituted aryl, substituted or unsubstituted heteroaryl, substituted or unsubstituted heterocycloalkyl, substituted or unsubstituted heteroalkyl, e.g., acetal, OHC—, H₂N—(CH₂)_{q}—, HS—(CH₂)_{q}, or —(CH₂)_{q}C(Y)Z¹. The index “e” represents an integer from 1 to 2500. The indices b, d, and q independently represent integers from 0 to 20. The symbols Z and Z¹ independently represent OH, NH₂, leaving groups, e.g., imidazole, p-nitrophenyl, HOBT, tetrazole, halide, S—R⁹, the alcohol portion of activated esters; —(CH₂)ₚC(Y¹)V, or —(CH₂)ₚU(CH₂)ₛC(Y¹)ᵥ. The symbol Y represents H(2), ═O, ═S, ═N—R¹¹. The symbols X, Y, Y¹, A¹, and U independently represent the moieties O, S, N—R¹¹. The symbol V represents OH, NH₂, halogen, S—R¹², the alcohol component of activated esters, the amine component of activated amides, sugar-nucleotides, and proteins. The indices p, q, s and v are members independently selected from the integers from 0 to 20. The symbols R⁹, R¹⁰, R¹¹ and R¹² independently represent H, substituted or unsubstituted alkyl, substituted or unsubstituted heteroalkyl, substituted or unsubstituted aryl, substituted or unsubstituted heterocycloalkyl and substituted or unsubstituted heteroaryl.

The poly(ethylene glycol) useful in forming the conjugate of the invention is either linear or branched. Branched poly(ethylene glycol) molecules suitable for use in the invention include, but are not limited to, those described by the following formula:

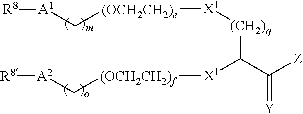

in which R⁸ and R^{8′} are members independently selected from the groups defined for R⁸, above. A¹ and A² are members independently selected from the groups defined for A¹, above. The indices e, f, o, and q are as described above. Z and Y are as described above. X¹ and X^{1′} are members independently selected from S, SC(O)NH, HNC(O)S, SC(O)O, O, NH, NHC(O), (O)CNH and NHC(O)O, OC(O)NH.

In other exemplary embodiments, the branched PEG is based upon a cysteine, serine or di-lysine core. In another exemplary embodiments, the poly(ethylene glycol) molecule is selected from the following structures:

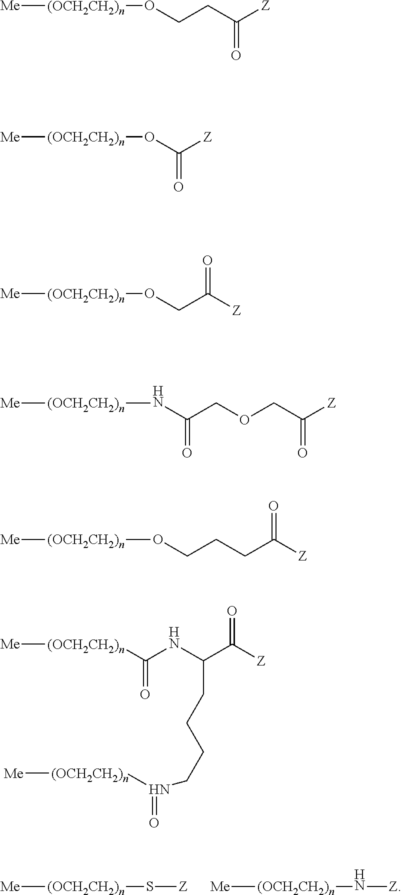

In a further embodiment the poly(ethylene glycol) is a branched PEG having more than one PEG moiety attached. Examples of branched PEGs are described in U.S. Pat. No. 5,932,462; U.S. Pat. No. 5,342,940; U.S. Pat. No. 5,643,575; U.S. Pat. No. 5,919,455; U.S. Pat. No. 6,113,906; U.S. Pat. No. 5,183,660; WO 02/09766; Kodera Y., Bioconjugate Chemistry 5: 283-288 (1994); and Yamasaki et al., Agric. Biol. Chem., 52: 2125-2127, 1998. In a preferred embodiment the molecular weight of each poly(ethylene glycol) of the branched PEG is less than or equal to 40,000 daltons.

Representative polymeric modifying moieties include structures that are based on side chain-containing amino acids, e.g., serine, cysteine, lysine, and small peptides, e.g., lys-lys. Exemplary structures include:

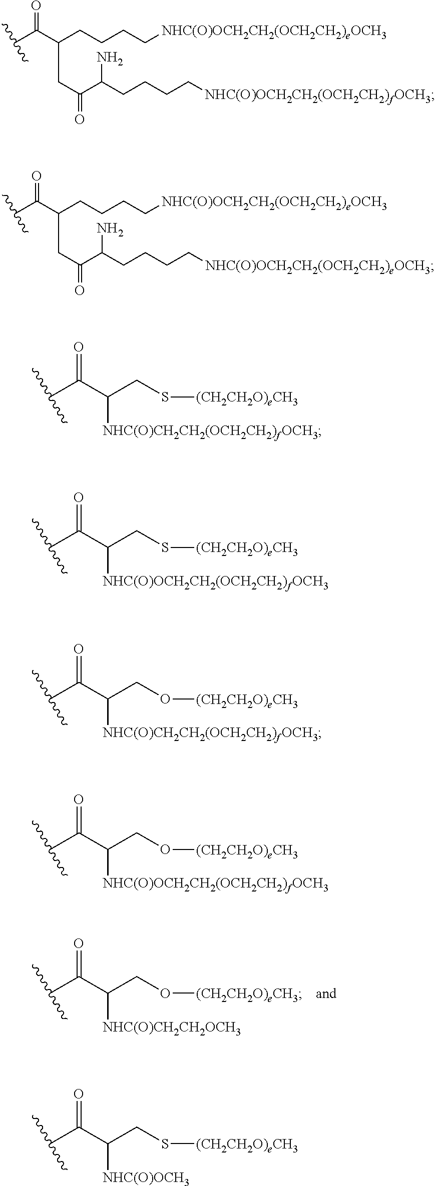

Those of skill will appreciate that the free amine in the di-lysine structures can also be pegylated through an amide or urethane bond with a PEG moiety.

In yet another embodiment, the polymeric modifying moiety is a branched PEG moiety that is based upon a tri-lysine peptide. The tri-lysine can be mono-, di-, tri-, or tetra-PEG-ylated. Exemplary species according to this embodiment have the formulae:

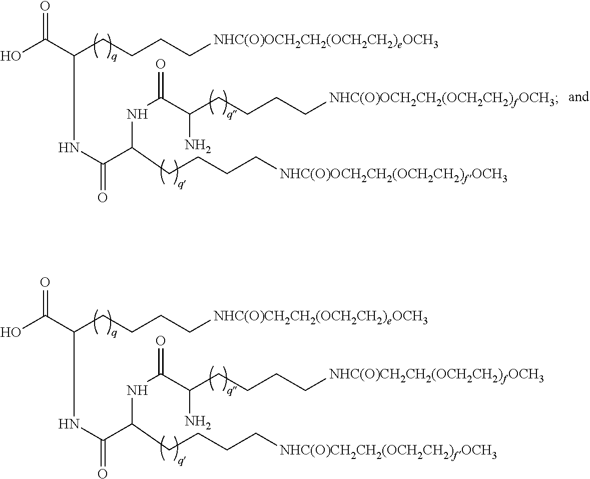

in which the indices e, f and f′ are independently selected integers from 1 to 2500; and the indices q, q′ and q″ are independently selected integers from 1 to 20.

As will be apparent to those of skill, the branched polymers of use in the invention include variations on the themes set forth above. For example the di-lysine-PEG conjugate shown above can include three polymeric subunits, the third bonded to the α-amine shown as unmodified in the structure above. Similarly, the use of a tri-lysine functionalized with three or four polymeric subunits labeled with the polymeric modifying moiety in a desired manner is within the scope of the invention.

An exemplary precursor useful to form a polypeptide conjugate with a branched modifying group that includes one or more polymeric moiety (e.g., PEG) has the formula:

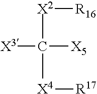

In one embodiment, the branched polymer species according to this formula are essentially pure water-soluble polymers. X^{3′} is a moiety that includes an ionizable (e.g., OH, COOH, H₂PO₄, HSO₃, NH₂, and salts thereof, etc.) or other reactive functional group, e.g., infra. C is carbon. X⁵ is a non-reactive group (e.g., H, CH₃, OH and the like). In one embodiment, X⁵ is preferably not a polymeric moiety. R¹⁶ and R′⁷ are independently selected from non-reactive groups (e.g., H, unsubstituted alkyl, unsubstituted heteroalkyl) and polymeric arms (e.g., PEG). X² and X⁴ are linkage fragments that are preferably essentially non-reactive under physiological conditions. X² and X⁴ are independently selected. An exemplary linker includes neither aromatic nor ester moieties. Alternatively, these linkages can include one or more moiety that is designed to degrade under physiologically relevant conditions, e.g., esters, disulfides, etc. X² and X⁴ join the polymeric arms R¹⁶ and R′⁷ to C. In one embodiment, when X^{3′} is reacted with a reactive functional group of complementary reactivity on a linker, sugar or linker-sugar cassette, X^{3′} is converted to a component of a linkage fragment.

Exemplary linkage fragments including X² and X⁴ are independently selected and include S, SC(O)NH, HNC(O)S, SC(O)O, O, NH, NHC(O), (O)CNH and NHC(O)O, and OC(O)NH, CH₂, CH₂S, CH₂O, CH₂CH₂O, CH₂CH₂S, (CH₂)ₒO, (CH₂)ₒS or (CH₂)ₒY′-PEG wherein, Y′ is S, NH, NHC(O), C(O)NH, NHC(O)O, OC(O)NH, or O and o is an integer from 1 to 50. In an exemplary embodiment, the linkage fragments X² and X⁴ are different linkage fragments.

In an exemplary embodiment, one of the above precursors or an activated derivative thereof, is reacted with, and thereby bound to a sugar, an activated sugar or a sugar nucleotide through a reaction between X^{3′} and a group of complementary reactivity on the sugar moiety, e.g., an amine. Alternatively, X^{3′} reacts with a reactive functional group on a precursor to linker L^{a} according to Scheme 2, below.

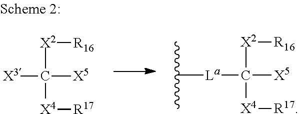

In an exemplary embodiment, the modifying group is derived from a natural or unnatural amino acid, amino acid analogue or amino acid mimetic, or a small peptide formed from one or more such species. For example, certain branched polymers found in the compounds of the invention have the formula:

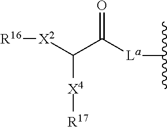

In this example, the linkage fragment C(O)L^{a} is formed by the reaction of a reactive functional group, e.g., X^{3′}, on a precursor of the branched polymeric modifying moiety and a reactive functional group on the sugar moiety, or a precursor to a linker. For example, when X^{3′} is a carboxylic acid, it can be activated and bound directly to an amine group pendent from an amino-saccharide (e.g., Sia, GalNH₂, GlcNH₂, ManNH₂, etc.), forming an amide. Additional exemplary reactive functional groups and activated precursors are described hereinbelow. The symbols have the same identity as those discussed above.

In another exemplary embodiment, L^{a} is a linking moiety having the structure:

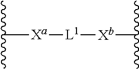

in which X^{a} and X^{b} are independently selected linkage fragments and L¹ is selected from a bond, substituted or unsubstituted alkyl or substituted or unsubstituted heteroalkyl.

Exemplary species for X^{a} and X^{b} include S, SC(O)NH, HNC(O)S, SC(O)O, O, NH, NHC(O), C(O)NH and NHC(O)O, and OC(O)NH.

In another exemplary embodiment, X⁴ is a peptide bond to R¹⁷, which is an amino acid, di-peptide (e.g., Lys-Lys) or tri-peptide (e.g., Lys-Lys-Lys) in which the alpha-amine moiety(ies) and/or side chain heteroatom(s) are modified with a polymeric modifying moiety.

The embodiments of the invention set forth above are further exemplified by reference to species in which the polymer is a water-soluble polymer, particularly poly(ethylene glycol) (“PEG”), e.g., methoxy-poly(ethylene glycol). Those of skill will appreciate that the focus in the sections that follow is for clarity of illustration and the various motifs set forth using PEG as an exemplary polymer are equally applicable to species in which a polymer other than PEG is utilized.

PEG of any molecular weight, e.g., 1 kDa, 2 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa is of use in the present invention.

In other exemplary embodiments, the polypeptide conjugate includes a moiety selected from the group:

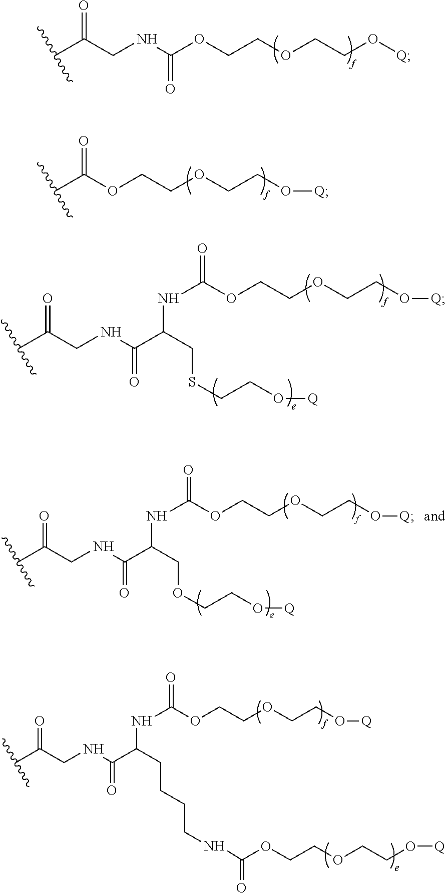

In each of the formulae above, the indices e and f are independently selected from the integers from 1 to 2500. In further exemplary embodiments, e and f are selected to provide a PEG moiety that is about 1 kDa, 2 kDa, 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa and 80 kDa. The symbol Q represents substituted or unsubstituted alkyl (e.g., C₁-C₆ alkyl, e.g., methyl), substituted or unsubstituted heteroalkyl or H.

Other branched polymers have structures based on di-lysine (Lys-Lys) peptides, e.g.:

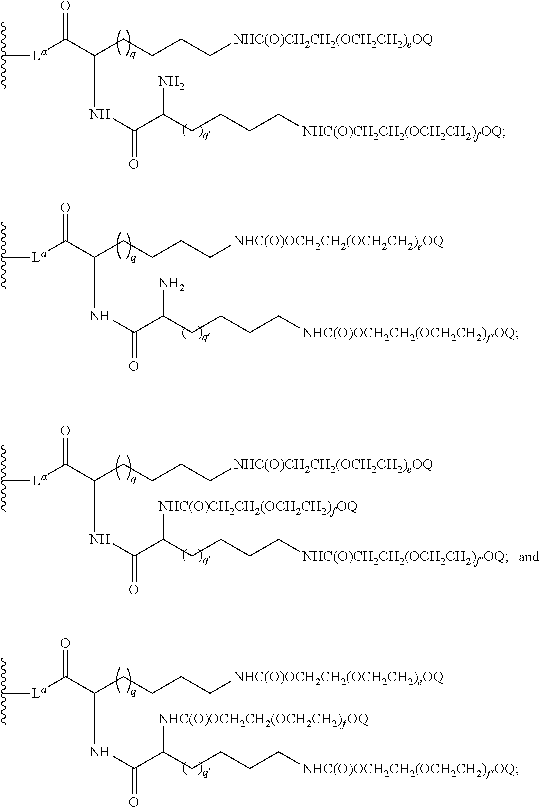

and tri-lysine peptides (Lys-Lys-Lys), e.g.:

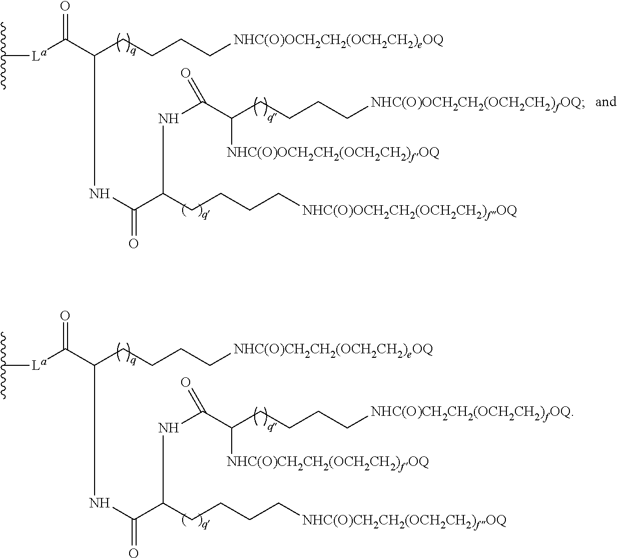

In each of the figures above, the indices e, f, f′ and f″ represent integers independently selected from 1 to 2500. The indices q, q′ and q″ represent integers independently selected from 1 to 20.

In another exemplary embodiment, the conjugates of the invention include a formula which is a member selected from:

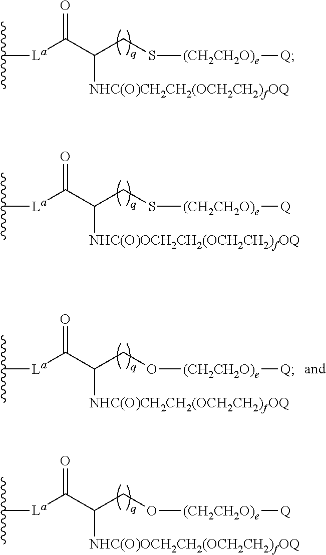

wherein Q is a member selected from H and substituted or unsubstituted C₁-C₆ alkyl. The indices e and f are integers independently selected from 1 to 2500, and the index q is an integer selected from 0 to 20.

In another exemplary embodiment, the conjugates of the invention include a formula which is a member selected from:

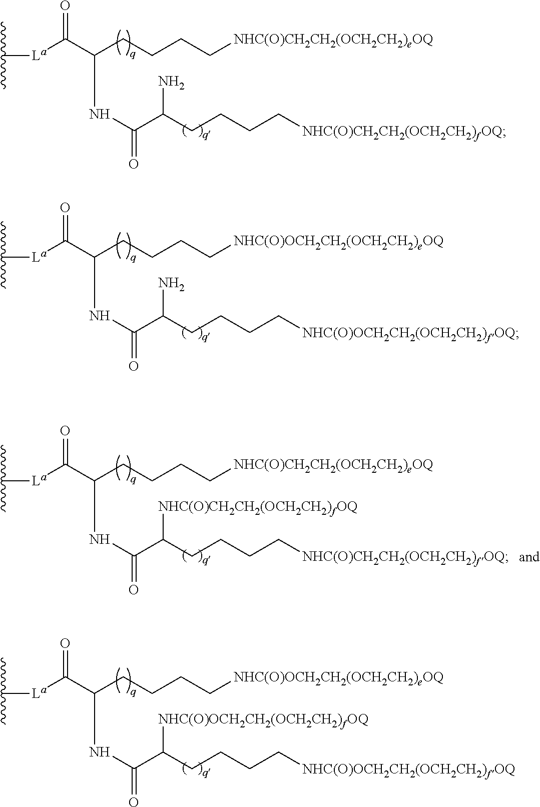

wherein Q is a member selected from H and substituted or unsubstituted C₁-C₆ alkyl, preferably Me. The indices e, f and f′ are integers independently selected from 1 to 2500, and q and q′ are integers independently selected from 1 to 20.

In another exemplary embodiment, the conjugate of the invention includes a structure according to the following formula:

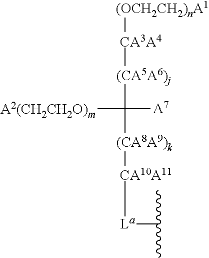

wherein the indices m and n are integers independently selected from 0 to 5000. The indices j and k are integers independently selected from 0 to 20. A¹, A², A³, A⁴, A⁵, A⁶, A⁷, A⁸, A⁹, A¹⁰ and A¹¹ are members independently selected from H, substituted or unsubstituted alkyl, substituted or unsubstituted heteroalkyl, substituted or unsubstituted aryl, substituted or unsubstituted cycloalkyl, substituted or unsubstituted heterocycloalkyl, substituted or unsubstituted heteroaryl, —NA¹²A¹³, —OA¹² and —SiA¹²A¹³. A¹² and A¹³ are members independently selected from substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

In one embodiment according to the formula above, the branched polymer has a structure according to the following formula:

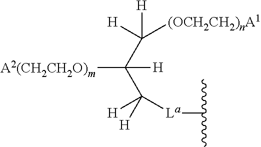

In an exemplary embodiment, A¹ and A² are members independently selected from —OCH₃ and OH.

In another exemplary embodiment, the linker L^{a} is a member selected from aminoglycine derivatives. Exemplary polymeric modifying groups according to this embodiment have a structure according to the following formulae:

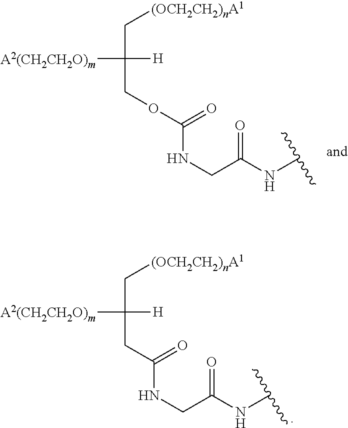

In one example, A¹ and A² are members independently selected from OCH₃ and OH. Exemplary polymeric modifying groups according to this example include:

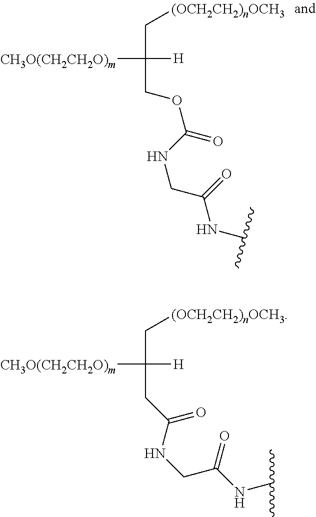

In each of the above embodiment, wherein the modifying group includes a stereocenter, for example those including an amino acid linker or a glycerol-based linker, the stereocenter can be either racemic or defined. In one embodiment, in which such stereocenter is defined, it has (S) configuration. In another embodiment, the stereocenter has (R) configuration.

Those of skill in the art will appreciate that one or more of the m-PEG arms of the branched polymer can be replaced by a PEG moiety with a different terminus, e.g., OH, COOH, NH₂, C₂-C₁₀-alkyl, etc. Moreover, the structures above are readily modified by inserting alkyl linkers (or removing carbon atoms) between the α-carbon atom and the functional group of the side chain. Thus, “homo” derivatives and higher homologues, as well as lower homologues are within the scope of cores for branched PEGs of use in the present invention.

The branched PEG species set forth herein are readily prepared by methods such as that set forth in the Scheme 3, below:

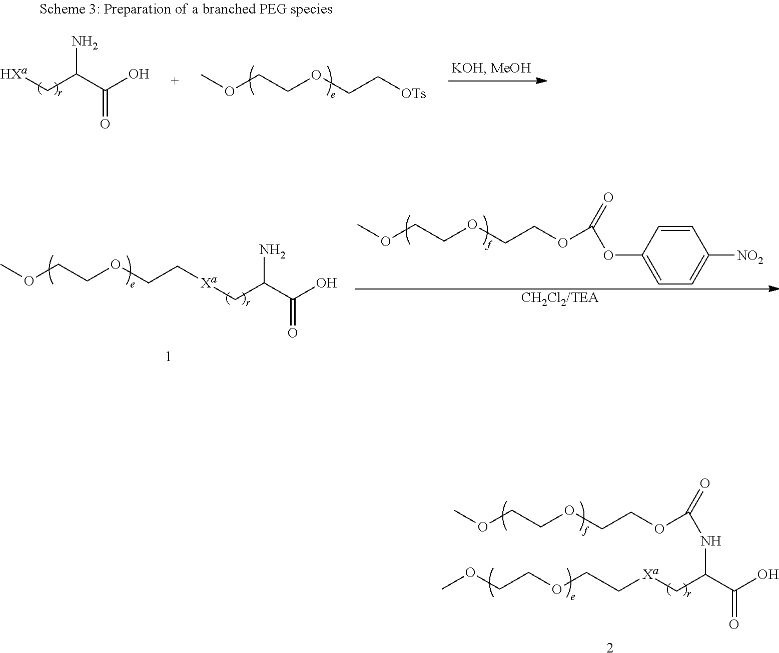

in which X^{a} is O or S and r is an integer from 1 to 5. The indices e and f are independently selected integers from 1 to 2500.

Thus, according to Scheme 3, a natural or unnatural amino acid is contacted with an activated m-PEG derivative, in this case the tosylate, forming 1 by alkylating the side-chain heteroatom X^{a}. The mono-functionalized m-PEG amino acid is submitted to N-acylation conditions with a reactive m-PEG derivative, thereby assembling branched m-PEG 2. As one of skill will appreciate, the tosylate leaving group can be replaced with any suitable leaving group, e.g., halogen, mesylate, triflate, etc. Similarly, the reactive carbonate utilized to acylate the amine can be replaced with an active ester, e.g., N-hydroxysuccinimide, etc., or the acid can be activated in situ using a dehydrating agent such as dicyclohexylcarbodiimide, carbonyldiimidazole, etc.

In an exemplary embodiment, the modifying group is a PEG moiety, however, any modifying group, e.g., water-soluble polymer, water-insoluble polymer, therapeutic moiety, etc., can be incorporated in a glycosyl moiety through an appropriate linkage. The modified sugar is formed by enzymatic means, chemical means or a combination thereof, thereby producing a modified sugar. In an exemplary embodiment, the sugars are substituted with an active amine at any position that allows for the attachment of the modifying moiety, yet still allows the sugar to function as a substrate for an enzyme capable of coupling the modified sugar to the G-CSF polypeptide. In an exemplary embodiment, when galactosamine is the modified sugar, the amine moiety is attached to the carbon atom at the 6-position.

### Water-Insoluble Polymers

In another embodiment, analogous to those discussed above, the modified sugars include a water-insoluble polymer, rather than a water-soluble polymer. The conjugates of the invention may also include one or more water-insoluble polymers. This embodiment of the invention is illustrated by the use of the conjugate as a vehicle with which to deliver a therapeutic polypeptide in a controlled manner. Polymeric drug delivery systems are known in the art. See, for example, Dunn et al., Eds. POLYMERIC DRUGS AND DRUG DELIVERY SYSTEMS, ACS Symposium Series Vol. 469, American Chemical Society, Washington, D.C. 1991. Those of skill in the art will appreciate that substantially any known drug delivery system is applicable to the conjugates of the present invention.

Representative water-insoluble polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics and polyvinylphenol and copolymers thereof.

Synthetically modified natural polymers of use in conjugates of the invention include, but are not limited to, alkyl celluloses, hydroxyalkyl celluloses, cellulose ethers, cellulose esters, and nitrocelluloses. Particularly preferred members of the broad classes of synthetically modified natural polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and polymers of acrylic and methacrylic esters and alginic acid.

These and the other polymers discussed herein can be readily obtained from commercial sources such as Sigma Chemical Co. (St. Louis, Mo.), Polysciences (Warrenton, Pa.), Aldrich (Milwaukee, Wis.), Fluka (Ronkonkoma, N.Y.), and BioRad (Richmond, Calif.), or else synthesized from monomers obtained from these suppliers using standard techniques.

Representative biodegradable polymers of use in the conjugates of the invention include, but are not limited to, polylactides, polyglycolides and copolymers thereof, poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, pluronics and the like.

The polymers of use in the invention include “hybrid’ polymers that include water-insoluble materials having within at least a portion of their structure, a bioresorbable molecule. An example of such a polymer is one that includes a water-insoluble copolymer, which has a bioresorbable region, a hydrophilic region and a plurality of crosslinkable functional groups per polymer chain.

For purposes of the present invention, “water-insoluble materials” includes materials that are substantially insoluble in water or water-containing environments. Thus, although certain regions or segments of the copolymer may be hydrophilic or even water-soluble, the polymer molecule, as a whole, does not to any substantial measure dissolve in water.

For purposes of the present invention, the term “bioresorbable molecule” includes a region that is capable of being metabolized or broken down and resorbed and/or eliminated through normal excretory routes by the body. Such metabolites or break down products are preferably substantially non-toxic to the body.

The bioresorbable region may be either hydrophobic or hydrophilic, so long as the copolymer composition as a whole is not rendered water-soluble. Thus, the bioresorbable region is selected based on the preference that the polymer, as a whole, remains water-insoluble. Accordingly, the relative properties, i.e., the kinds of functional groups contained by, and the relative proportions of the bioresorbable region, and the hydrophilic region are selected to ensure that useful bioresorbable compositions remain water-insoluble.

Exemplary resorbable polymers include, for example, synthetically produced resorbable block copolymers of poly(α-hydroxy-carboxylic acid)/poly(oxyalkylene, (see, Cohn et al., U.S. Pat. No. 4,826,945). These copolymers are not crosslinked and are water-soluble so that the body can excrete the degraded block copolymer compositions. See, Younes et al., J. Biomed. Mater. Res. 21: 1301-1316 (1987); and Cohn et al., J. Biomed. Mater. Res. 22: 993-1009 (1988).

Presently preferred bioresorbable polymers include one or more components selected from poly(esters), poly(hydroxy acids), poly(lactones), poly(amides), poly(ester-amides), poly (amino acids), poly(anhydrides), poly(orthoesters), poly(carbonates), poly(phosphazines), poly(phosphoesters), poly(thioesters), polysaccharides and mixtures thereof. More preferably still, the bioresorbable polymer includes a poly(hydroxy) acid component. Of the poly(hydroxy) acids, polylactic acid, polyglycolic acid, polycaproic acid, polybutyric acid, polyvaleric acid and copolymers and mixtures thereof are preferred.

In addition to forming fragments that are absorbed in vivo (“bioresorbed”), preferred polymeric coatings for use in the methods of the invention can also form an excretable and/or metabolizable fragment.

Higher order copolymers can also be used in the present invention. For example, Casey et al., U.S. Pat. No. 4,438,253, which issued on Mar. 20, 1984, discloses tri-block copolymers produced from the transesterification of poly(glycolic acid) and an hydroxyl-ended poly(alkylene glycol). Such compositions are disclosed for use as resorbable monofilament sutures. The flexibility of such compositions is controlled by the incorporation of an aromatic orthocarbonate, such as tetra-p-tolyl orthocarbonate into the copolymer structure.

Other polymers based on lactic and/or glycolic acids can also be utilized. For example, Spinu, U.S. Pat. No. 5,202,413, which issued on Apr. 13, 1993, discloses biodegradable multi-block copolymers having sequentially ordered blocks of polylactide and/or polyglycolide produced by ring-opening polymerization of lactide and/or glycolide onto either an oligomeric diol or a diamine residue followed by chain extension with a di-functional compound, such as, a diisocyanate, diacylchloride or dichlorosilane.

Bioresorbable regions of coatings useful in the present invention can be designed to be hydrolytically and/or enzymatically cleavable. For purposes of the present invention, “hydrolytically cleavable” refers to the susceptibility of the copolymer, especially the bioresorbable region, to hydrolysis in water or a water-containing environment. Similarly, “enzymatically cleavable” as used herein refers to the susceptibility of the copolymer, especially the bioresorbable region, to cleavage by endogenous or exogenous enzymes.

When placed within the body, the hydrophilic region can be processed into excretable and/or metabolizable fragments. Thus, the hydrophilic region can include, for example, polyethers, polyalkylene oxides, polyols, poly(vinyl pyrrolidine), poly(vinyl alcohol), poly(alkyl oxazolines), polysaccharides, carbohydrates, peptides, proteins and copolymers and mixtures thereof. Furthermore, the hydrophilic region can also be, for example, a poly(alkylene) oxide. Such poly(alkylene) oxides can include, for example, poly(ethylene) oxide, poly(propylene) oxide and mixtures and copolymers thereof.

Polymers that are components of hydrogels are also useful in the present invention. Hydrogels are polymeric materials that are capable of absorbing relatively large quantities of water. Examples of hydrogel forming compounds include, but are not limited to, polyacrylic acids, sodium carboxymethylcellulose, polyvinyl alcohol, polyvinyl pyrrolidine, gelatin, carrageenan and other polysaccharides, hydroxyethylenemethacrylic acid (HEMA), as well as derivatives thereof, and the like. Hydrogels can be produced that are stable, biodegradable and bioresorbable. Moreover, hydrogel compositions can include subunits that exhibit one or more of these properties.

Bio-compatible hydrogel compositions whose integrity can be controlled through crosslinking are known and are presently preferred for use in the methods of the invention. For example, Hubbell et al., U.S. Pat. Nos. 5,410,016, which issued on Apr. 25, 1995 and 5,529,914, which issued on Jun. 25, 1996, disclose water-soluble systems, which are crosslinked block copolymers having a water-soluble central block segment sandwiched between two hydrolytically labile extensions. Such copolymers are further end-capped with photopolymerizable acrylate functionalities. When cross-linked, these systems become hydrogels. The water soluble central block of such copolymers can include poly(ethylene glycol); whereas, the hydrolytically labile extensions can be a poly(α-hydroxy acid), such as polyglycolic acid or polylactic acid. See, Sawhney et al., Macromolecules 26: 581-587 (1993).

In another embodiment, the gel is a thermoreversible gel. Thermoreversible gels including components, such as pluronics, collagen, gelatin, hyalouronic acid, polysaccharides, polyurethane hydrogel, polyurethane-urea hydrogel and combinations thereof are presently preferred.

In yet another exemplary embodiment, the conjugate of the invention includes a component of a liposome. Liposomes can be prepared according to methods known to those skilled in the art, for example, as described in Eppstein et al., U.S. Pat. No. 4,522,811, which issued on Jun. 11, 1985. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its pharmaceutically acceptable salt is then introduced into the container. The container is then swirled by hand to free lipid material from the sides of the container and to disperse lipid aggregates, thereby forming the liposomal suspension.

The above-recited microparticles and methods of preparing the microparticles are offered by way of example and they are not intended to define the scope of microparticles of use in the present invention. It will be apparent to those of skill in the art that an array of microparticles, fabricated by different methods, are of use in the present invention.

The structural formats discussed above in the context of the water-soluble polymers, both straight-chain and branched are generally applicable with respect to the water-insoluble polymers as well. Thus, for example, the cysteine, serine, dilysine, and trilysine branching cores can be functionalized with two water-insoluble polymer moieties. The methods used to produce these species are generally closely analogous to those used to produce the water-soluble polymers.

### Other Modifying Groups

The present invention also provides conjugates analogous to those described above in which the polypeptide is conjugated to a therapeutic moiety, diagnostic moiety, targeting moiety, toxin moiety or the like via a glycosyl linking group. Each of the above-recited moieties can be a small molecule, natural polymer (e.g., polypeptide) or a synthetic polymer.

In a still further embodiment, the invention provides conjugates that localize selectively in a particular tissue due to the presence of a targeting agent as a component of the conjugate. In an exemplary embodiment, the targeting agent is a protein. Exemplary proteins include transferrin (brain, blood pool), HS-glycoprotein (bone, brain, blood pool), antibodies (brain, tissue with antibody-specific antigen, blood pool), coagulation factors V-XII (damaged tissue, clots, cancer, blood pool), serum proteins, e.g., α-acid glycoprotein, fetuin, α-fetal protein (brain, blood pool), β2-glycoprotein (liver, atherosclerosis plaques, brain, blood pool), G-CSF, GM-CSF, M-CSF, and EPO (immune stimulation, cancers, blood pool, red blood cell overproduction, neuroprotection), albumin (increase in half-life), IL-2 and IFN-α.

In an exemplary targeted conjugate, interferon alpha 2β (IFN-α 2β) is conjugated to transferrin via a bifunctional linker that includes a glycosyl linking group at each terminus of the PEG moiety (Scheme 1). For example, one terminus of the PEG linker is functionalized with an intact sialic acid linker that is attached to transferrin and the other is functionalized with an intact C-linked Man linker that is attached to IFN-α2β.

### Biomolecules

In another embodiment, the modified sugar bears a biomolecule. In still further embodiments, the biomolecule is a functional protein, enzyme, antigen, antibody, peptide, nucleic acid (e.g., single nucleotides or nucleosides, oligonucleotides, polynucleotides and single- and higher-stranded nucleic acids), lectin, receptor or a combination thereof.

Preferred biomolecules are essentially non-fluorescent, or emit such a minimal amount of fluorescence that they are inappropriate for use as a fluorescent marker in an assay. Moreover, it is generally preferred to use biomolecules that are not sugars. An exception to this preference is the use of an otherwise naturally occurring sugar that is modified by covalent attachment of another entity (e.g., PEG, biomolecule, therapeutic moiety, diagnostic moiety, etc.). In an exemplary embodiment, a sugar moiety, which is a biomolecule, is conjugated to a linker arm and the sugar-linker arm cassette is subsequently conjugated to a polypeptide via a method of the invention.

Biomolecules useful in practicing the present invention can be derived from any source. The biomolecules can be isolated from natural sources or they can be produced by synthetic methods. Polypeptides can be natural polypeptides or mutated polypeptides. Mutations can be effected by chemical mutagenesis, site-directed mutagenesis or other means of inducing mutations known to those of skill in the art. polypeptides useful in practicing the instant invention include, for example, enzymes, antigens, antibodies and receptors. Antibodies can be either polyclonal or monoclonal; either intact or fragments. The polypeptides are optionally the products of a program of directed evolution

Both naturally derived and synthetic polypeptides and nucleic acids are of use in conjunction with the present invention; these molecules can be attached to a sugar residue component or a crosslinking agent by any available reactive group. For example, polypeptides can be attached through a reactive amine, carboxyl, sulfhydryl, or hydroxyl group. The reactive group can reside at a polypeptide terminus or at a site internal to the polypeptide chain. Nucleic acids can be attached through a reactive group on a base (e.g., exocyclic amine) or an available hydroxyl group on a sugar moiety (e.g., 3′- or 5′-hydroxyl). The peptide and nucleic acid chains can be further derivatized at one or more sites to allow for the attachment of appropriate reactive groups onto the chain. See, Chrisey et al. Nucleic Acids Res. 24: 3031-3039 (1996).

In a further embodiment, the biomolecule is selected to direct the polypeptide modified by the methods of the invention to a specific tissue, thereby enhancing the delivery of the polypeptide to that tissue relative to the amount of underivatized polypeptide that is delivered to the tissue. In a still further embodiment, the amount of derivatized polypeptide delivered to a specific tissue within a selected time period is enhanced by derivatization by at least about 20%, more preferably, at least about 40%, and more preferably still, at least about 100%. Presently, preferred biomolecules for targeting applications include antibodies, hormones and ligands for cell-surface receptors.

In still a further exemplary embodiment, there is provided as conjugate with biotin. Thus, for example, a selectively biotinylated polypeptide is elaborated by the attachment of an avidin or streptavidin moiety bearing one or more modifying groups.

### Therapeutic Moieties

In another embodiment, the modified sugar includes a therapeutic moiety. Those of skill in the art will appreciate that there is overlap between the category of therapeutic moieties and biomolecules; many biomolecules have therapeutic properties or potential.

The therapeutic moieties can be agents already accepted for clinical use or they can be drugs whose use is experimental, or whose activity or mechanism of action is under investigation. The therapeutic moieties can have a proven action in a given disease state or can be only hypothesized to show desirable action in a given disease state. In another embodiment, the therapeutic moieties are compounds, which are being screened for their ability to interact with a tissue of choice. Therapeutic moieties, which are useful in practicing the instant invention include drugs from a broad range of drug classes having a variety of pharmacological activities. Preferred therapeutic moieties are essentially non-fluorescent, or emit such a minimal amount of fluorescence that they are inappropriate for use as a fluorescent marker in an assay. Moreover, it is generally preferred to use therapeutic moieties that are not sugars. An exception to this preference is the use of a sugar that is modified by covalent attachment of another entity, such as a PEG, biomolecule, therapeutic moiety, diagnostic moiety and the like. In another exemplary embodiment, a therapeutic sugar moiety is conjugated to a linker arm and the sugar-linker arm cassette is subsequently conjugated to a polypeptide via a method of the invention.

Methods of conjugating therapeutic and diagnostic agents to various other species are well known to those of skill in the art. See, for example Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991.

In an exemplary embodiment, the therapeutic moiety is attached to the modified sugar via a linkage that is cleaved under selected conditions. Exemplary conditions include, but are not limited to, a selected pH (e.g., stomach, intestine, endocytotic vacuole), the presence of an active enzyme (e.g, esterase, reductase, oxidase), light, heat and the like. Many cleavable groups are known in the art. See, for example, Jung et al., Biochem. Biophys. Acta, 761: 152-162 (1983); Joshi et al., J. Biol. Chem., 265: 14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989).

Classes of useful therapeutic moieties include, for example, non-steroidal anti-inflammatory drugs (NSAIDS). The NSAIDS can, for example, be selected from the following categories: (e.g., propionic acid derivatives, acetic acid derivatives, fenamic acid derivatives, biphenylcarboxylic acid derivatives and oxicams); steroidal anti-inflammatory drugs including hydrocortisone and the like; antihistaminic drugs (e.g., chlorpheniramine, triprolidine); antitussive drugs (e.g., dextromethorphan, codeine, caramiphen and carbetapentane); antipruritic drugs (e.g., methdilazine and trimeprazine); anticholinergic drugs (e.g., scopolamine, atropine, homatropine, levodopa); anti-emetic and antinauseant drugs (e.g., cyclizine, meclizine, chlorpromazine, buclizine); anorexic drugs (e.g., benzphetamine, phentermine, chlorphentermine, fenfluramine); central stimulant drugs (e.g., amphetamine, methamphetamine, dextroamphetamine and methylphenidate); antiarrhythmic drugs (e.g., propanolol, procainamide, disopyramide, quinidine, encamide); β-adrenergic blocker drugs (e.g., metoprolol, acebutolol, betaxolol, labetalol and timolol); cardiotonic drugs (e.g., milrinone, aminone and dobutamine); antihypertensive drugs (e.g., enalapril, clonidine, hydralazine, minoxidil, guanadrel, guanethidine); diuretic drugs (e.g., amiloride and hydrochlorothiazide); vasodilator drugs (e.g., diltiazem, amiodarone, isoxsuprine, nylidrin, tolazoline and verapamil); vasoconstrictor drugs (e.g., dihydroergotamine, ergotamine and methylsergide); antiulcer drugs (e.g., ranitidine and cimetidine); anesthetic drugs (e.g., lidocaine, bupivacaine, chloroprocaine, dibucaine); antidepressant drugs (e.g., imipramine, desipramine, amitryptiline, nortryptiline); tranquilizer and sedative drugs (e.g., chlordiazepoxide, benacytyzine, benzquinamide, flurazepam, hydroxyzine, loxapine and promazine); antipsychotic drugs (e.g., chlorprothixene, fluphenazine, haloperidol, molindone, thioridazine and trifluoperazine); antimicrobial drugs (antibacterial, antifungal, antiprotozoal and antiviral drugs).

Antimicrobial drugs which are preferred for incorporation into the present composition include, for example, pharmaceutically acceptable salts of β-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isothionate, metronidazole, pentamidine, gentamycin, kanamycin, lineomycin, methacycline, methenamine, minocycline, neomycin, netilmycin, paromomycin, streptomycin, tobramycin, miconazole and amantadine.

Other drug moieties of use in practicing the present invention include antineoplastic drugs (e.g., antiandrogens (e.g., leuprolide or flutamide), cytocidal agents (e.g., adriamycin, doxorubicin, taxol, cyclophosphamide, busulfan, cisplatin, β-2-interferon) anti-estrogens (e.g., tamoxifen), antimetabolites (e.g., fluorouracil, methotrexate, mercaptopurine, thioguanine). Also included within this class are radioisotope-based agents for both diagnosis and therapy, and conjugated toxins, such as ricin, geldanamycin, mytansin, CC-1065, the duocarmycins, Chlicheamycin and related structures and analogues thereof.

The therapeutic moiety can also be a hormone (e.g., medroxyprogesterone, estradiol, leuprolide, megestrol, octreotide or somatostatin); muscle relaxant drugs (e.g., cinnamedrine, cyclobenzaprine, flavoxate, orphenadrine, papaverine, mebeverine, idaverine, ritodrine, diphenoxylate, dantrolene and azumolen); antispasmodic drugs; bone-active drugs (e.g., diphosphonate and phosphonoalkylphosphinate drug compounds); endocrine modulating drugs (e.g., contraceptives (e.g., ethinodiol, ethinyl estradiol, norethindrone, mestranol, desogestrel, medroxyprogesterone), modulators of diabetes (e.g., glyburide or chlorpropamide), anabolics, such as testolactone or stanozolol, androgens (e.g., methyltestosterone, testosterone or fluoxymesterone), antidiuretics (e.g., desmopressin) and calcitonins).

Also of use in the present invention are estrogens (e.g., diethylstilbesterol), glucocorticoids (e.g., triamcinolone, betamethasone, etc.) and progestogens, such as norethindrone, ethynodiol, norethindrone, levonorgestrel; thyroid agents (e.g., liothyronine or levothyroxine) or anti-thyroid agents (e.g., methimazole); antihyperprolactinemic drugs (e.g., cabergoline); hormone suppressors (e.g., danazol or goserelin), oxytocics (e.g., methylergonovine or oxytocin) and prostaglandins, such as mioprostol, alprostadil or dinoprostone, can also be employed.

Other useful modifying groups include immunomodulating drugs (e.g., antihistamines, mast cell stabilizers, such as lodoxamide and/or cromolyn, steroids (e.g., triamcinolone, beclomethazone, cortisone, dexamethasone, prednisolone, methylprednisolone, beclomethasone, or clobetasol), histamine H2 antagonists (e.g., famotidine, cimetidine, ranitidine), immunosuppressants (e.g., azathioprine, cyclosporin), etc. Groups with anti-inflammatory activity, such as sulindac, etodolac, ketoprofen and ketorolac, are also of use. Other drugs of use in conjunction with the present invention will be apparent to those of skill in the art.

### Modified Sugar Nucleotides

In certain embodiments of the present invention, a modified sugar nucleotide is utilized to add the modified sugar to the peptide. Exemplary sugar nucleotides that are used in the present invention in their modified form include nucleotide mono-, di- or triphosphates or analogs thereof. In a preferred embodiment, the modified sugar nucleotide is selected from a UDP-glycoside, CMP-glycoside, and a GDP-glycoside. Even more preferably, the modified sugar nucleotide is selected from an UDP-galactose, UDP-galactosamine, UDP-glucose, UDP-glucosamine, GDP-mannose, GDP-fucose, CMP-sialic acid, and CMP-NeuAc. N-acetylamine derivatives of the sugar nucleotides are also of use in the methods of the invention.

In a particularly preferred embodiment, the modified sugar nucleotide useful in the methods of the invention, is a UDP-sugar, in which the sugar moiety is a member selected from a glucosamine moiety and glucosamine-mimetic moiety. Thus, in a third aspect, the invention provides a compound having a structure according to Formula (XI):

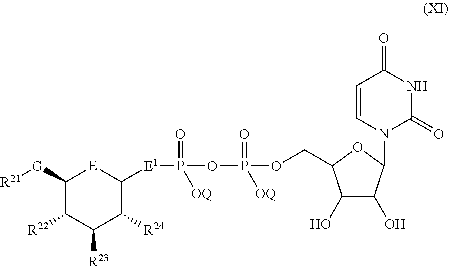

wherein each Q is a member independently selected from H, a negative charge and a salt counter-ion (e.g., Na, K, Li, Mg, Mn, Fe). E is a member selected from O, S, and CH₂. G is a member selected from —CH₂— and C=A, wherein A is a member selected from O, S and NR²⁷, wherein R²⁷ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl. E¹ is a member selected from O and S. R²¹, R²², R²³ and R²⁴ are members independently selected from H, OR²⁵, SR²⁵, NR²⁵R²⁶, NR²⁵S(O)₂R²⁶, S(O)₂NR²⁵R²⁶, NR²⁵C(O)R²⁶, C(O)NR²⁵R²⁶, C(O)OR²⁵, acyl, substituted or unsubstituted alkyl, substituted or unsubstituted heteroalkyl, substituted or unsubstituted aryl, substituted or unsubstituted heteroaryl and substituted or unsubstituted heterocycloalkyl, wherein R²⁵ and R²⁶ are members independently selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl. In an exemplary embodiment, the modified sugar nucleotide has a structure according to Formula (XIa) or (XIb):

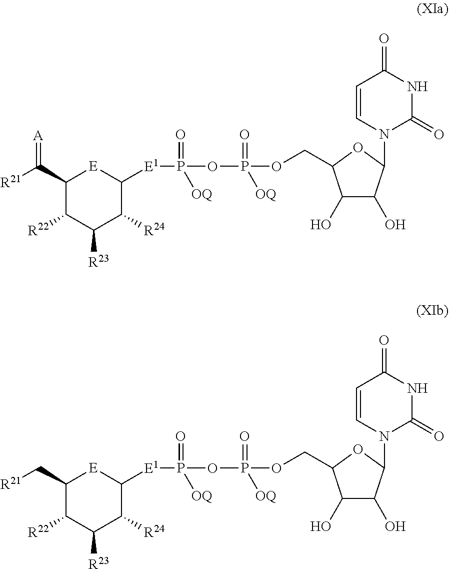

In one example according to any of the above embodiments, at least one of R²¹, R²², R²³ and R²⁴ includes a polymeric modifying group. In another example according to any of the above embodiments, e.g., Formulae (XI), (XIa) and (XIb), E and E¹ are both oxygen (O). In yet another example, the modified sugar nucleotide is modified UDP-GlcNAc or modified GlcNH. In a further example, the modified UDP-GlcNAc or modified GlcNH is modified with a polymeric modifying group at the 2- or 6-position.

In one example, the sugar moiety of the modified sugar nucleotide is modified with a polymeric modifying group that includes a water-soluble polymer, such as a poly(alkylene oxide) moiety (e.g., PEG or a PPG) or a derivative thereof. An exemplary modified sugar nucleotide bears a glycosyl moiety or a glycosyl-mimetic moiety that is modified through an amine moiety on the sugar. For example, a saccharyl amine (without the modifying group) can be enzymatically conjugated to a peptide (or other species) and the free amine moiety subsequently be conjugated to a desired modifying group. Alternatively, the modified sugar nucleotide can function as a substrate for an enzyme that transfers the modified sugar to a saccharyl acceptor on the polypeptide.

In the discussion that follows, a number of specific examples of modified sugar nucleotides that are useful in practicing the present invention are set forth. In the exemplary embodiments, a glucose, a glucose-mimetic moiety, a glucosamine moiety, a glucosamine-mimetic moiety or any derivative thereof is utilized as the sugar moiety to which the modifying group is attached. The focus of the discussion on glucosamine derivatives is for clarity of illustration only and should not be construed to limit the scope of the invention. Those of skill in the art will appreciate that a variety of other sugar moieties can be activated and derivatized in a manner analogous to the examples set forth herein. For example, numerous methods are available for modifying galactose, sialic acid, glucose, N-acetylgalactosamine and fucose to name a few sugar substrates, which are readily modified by art recognized methods. See, for example, Elhalabi et al., Curr. Med. Chem. 6: 93 (1999) and Schafer et al., J. Org. Chem. 65: 24 (2000).

In an exemplary embodiment, the modified sugar nucleotide is based upon a glucosamine moiety. As shown in Scheme 3 and Scheme 4, glucosamine or N-acetylglucosamine can be modified at the 2- or 6-position using standard methods.

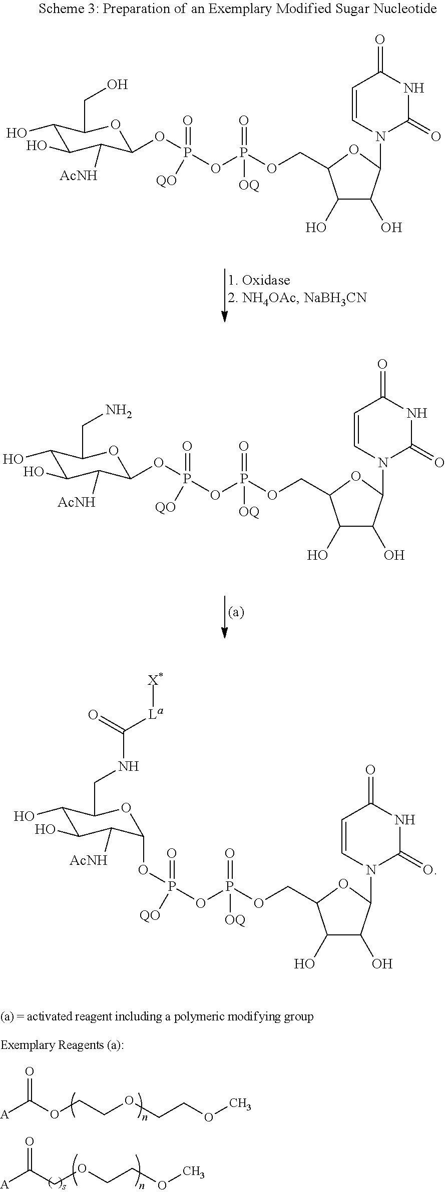

In Scheme 3, above, the index n represents an integer from 0 to 5000, preferably from 10 to 2500, and more preferably from 10 to 1200. L^{a} is a bond or a linker group and X* is a polymeric modifying group selected from linear and branched. The symbol “A” represents an activating group, e.g., a halo, a component of an activated ester (e.g., a N-hydroxysuccinimide ester), a component of a carbonate (e.g., p-nitrophenyl carbonate) and the like. Q is H, a negative charge or a salt counterion (e.g., Na⁺). In Scheme 3, the primary hydroxyl group of the GlcNAc moiety is first oxidized to an aldehyde group (e.g., using an oxidase, such as glucose oxidase), which is further converted to the amine via reductive amination. Those of skill in the art will appreciate that other PEG-amide nucleotide sugars are readily prepared by this and analogous methods.

In other exemplary embodiments, the amide moiety is replaced by a group such as a urethane or a urea.

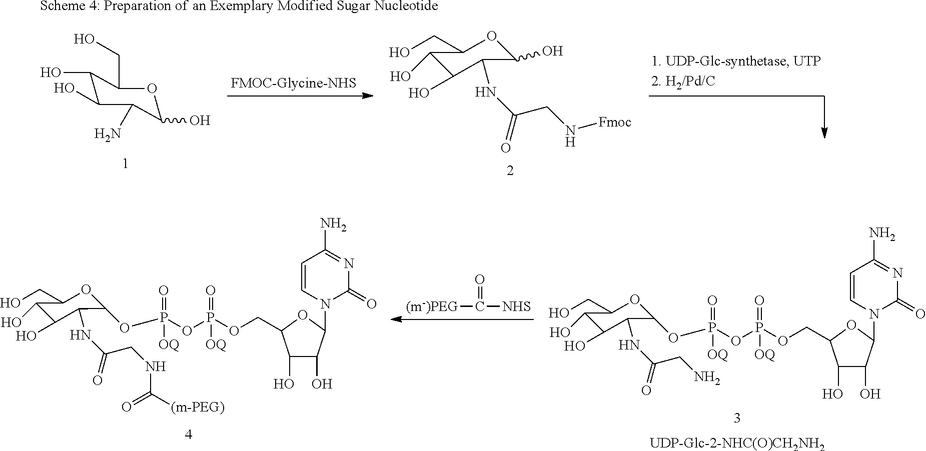

In Scheme 4, glucosamine 1, is treated with an activated ester of a protected amino acid (e.g., glycine) derivative, forming a protected amino acid amide adduct 2. Compound 2 is converted to the corresponding UDP derivative, for example through the action of an enzyme, such as UDP-Glc-synthetase, followed by catalytic hydrogenation of the UDP derivative to produce compound 3. The amino group of the glycine side chain is utilized for the attachment of the polymeric modifying group, such as PEG or PPG, by reacting compound 3 with an activated (m-)PEG derivative (e.g., PEG-C(O)NHS, producing compound 4. Alternatively, compound 3 may be reacted with a (m-)PPG derivative (e.g., PPG-C(O)NHS) to afford the corresponding PPG analog. Amine reactive PEG and PPG analogues are commercially available, or they can be prepared by methods readily accessible to those of skill in the art.

The sugar moiety of the modified sugar nucleotides of use in practicing the present invention can be modified with the polymeric modifying group at any position as illustrated in Figures (XIIa) and (XIIb), below:

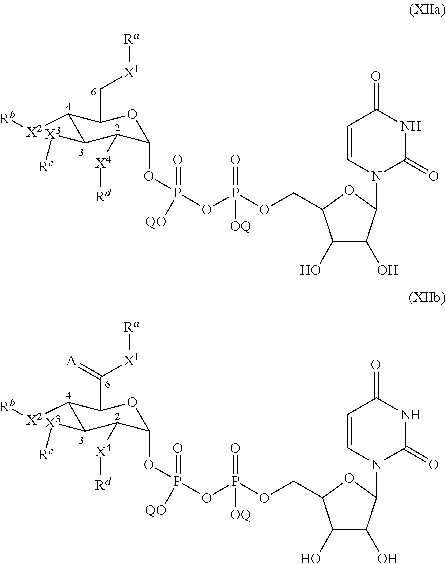

wherein A and Q are defined as herein above.

In Figures (XIIa) and (XIIb), X¹, X², X³ and X⁴ are independently selected linking groups, preferably selected from a single bond, —O—, —NR^{e}—, —S—, and —CH₂—, wherein each R^{e} is a member independently selected from R^{a}, R^{b}, R^{c} and R^{d}. The symbols R^{a}, R^{b}, R^{c} and R^{d} are independently selected from H, acyl (e.g., acetyl), a modifying group (e.g., polymeric modifying group, a therapeutic moiety, a biomolecule and the like) and a linker that is bound to a modifying group.

In the above structures, at least one of R^{a}, R^{b}, R^{c} and R^{d} includes a modifying group, such as a polymeric modifying group. Particularly preferred for the modification of the sugar moiety with a polymeric modifying group are positions 2 and 6. In Figure (XIIb), A is O, S, NR^{f}, wherein R^{f} is a member selected from H, R^{a}, R^{b}, R^{c} and R^{d}, substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl.

In one example, at least one of R^{a}, R^{b}, R^{c} and R^{d} includes a polymeric modifying group that incorporates at least one poly(alkylene oxide) moiety (e.g., PEG or PPG moiety). In another example, at least one of R^{a}, R^{b}, R^{c} and R^{d} includes a moiety selected from PEG, PPG, acyl-PEG, acyl-PPG, alkyl-PEG, acyl-alkyl-PEG, carbamoyl-PEG, carbamoyl-PPG, aryl-PEG, acyl-aryl-PEG, aryl-PPG, acyl-aryl-PPG, mannose-6-phosphate, heparin, heparan, SLex, mannose, chondroitin, keratan, dermatan, albumin, a polypeptide (such as any of those disclosed herein), peptides and the like (e.g., FGF, VFGF, integrins).

Table 12, below sets forth representative examples of modified sugar nucleotides that are derivatized with a modifying group, such as a polymeric modifying group (e.g., water-soluble modifying groups, such as PEG or PPG moieties). Certain of the compounds of Table 12 are prepared by the method of Scheme 3. Other derivatives are prepared by art-recognized methods. See, for example, Keppler et al., Glycobiology 11: 11R (2001); and Charter et al., Glycobiology 10: 1049 (2000)).

TABLE 12
Examples of sugar nucleotides derivatized with a polymeric modifying group

| Modification | |
|---|---|
| of Position | Exemplary Structures |
| 6 | 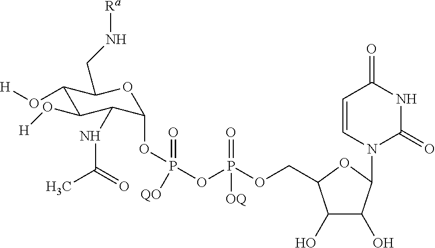 |
| 4 | 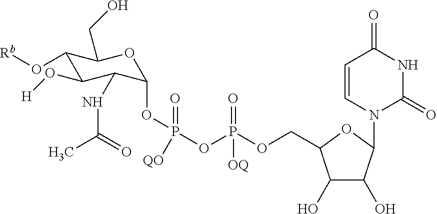 |
| 3 | 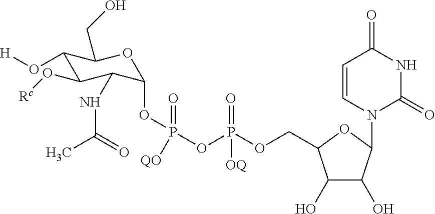 |
| 2 | 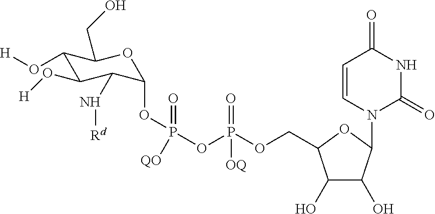 |
| 2 | 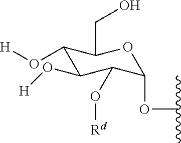 |

In still further embodiments, the polymeric modifying group is a branched PEG, for example, one of those species set forth herein. Illustrative modified sugar nucleotides or polypeptide conjugates according to this embodiment include a moiety selected from:

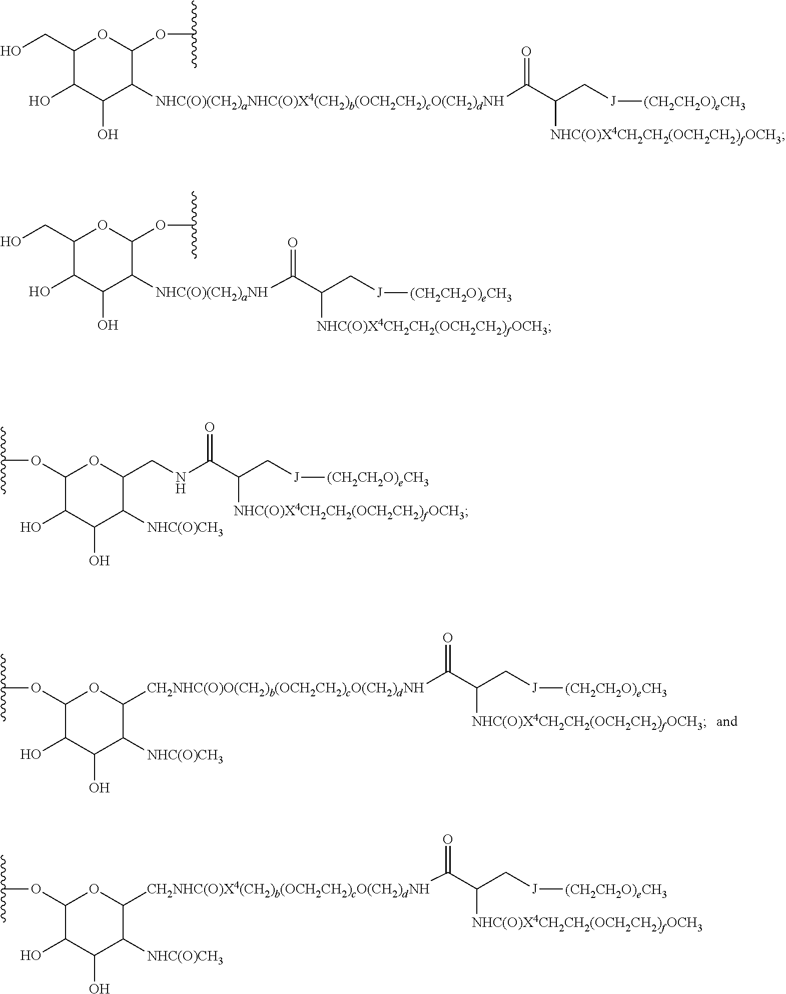

in which X⁴ is a bond or O, and J is S or O.

Exemplary modified sugar nucleotides have a structure selected from:

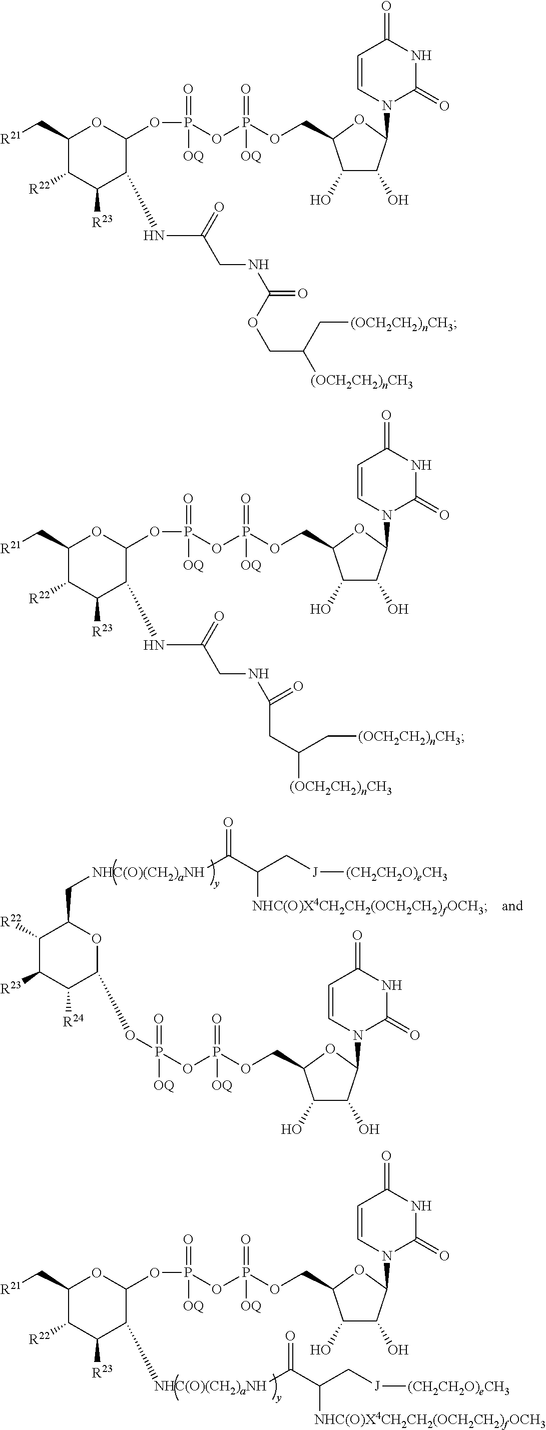

in which X⁴ is a bond or O, J is S or O, and y is 0 or 1.

Other exemplary modified sugar nucleotides have a structure selected from:

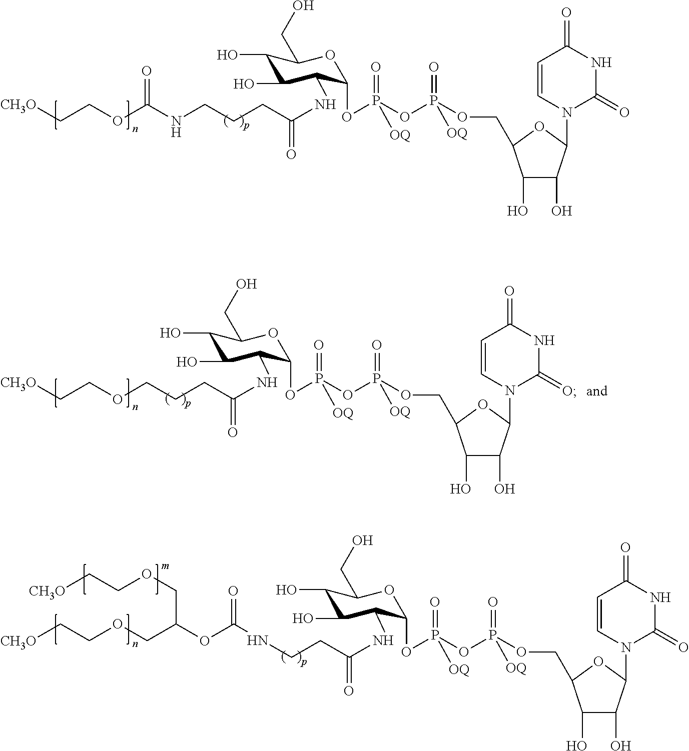

wherein Q is defined as herein above and p is an integer selected from 0 to 50.

Other exemplary modified sugar nucleotides have a structure selected from:

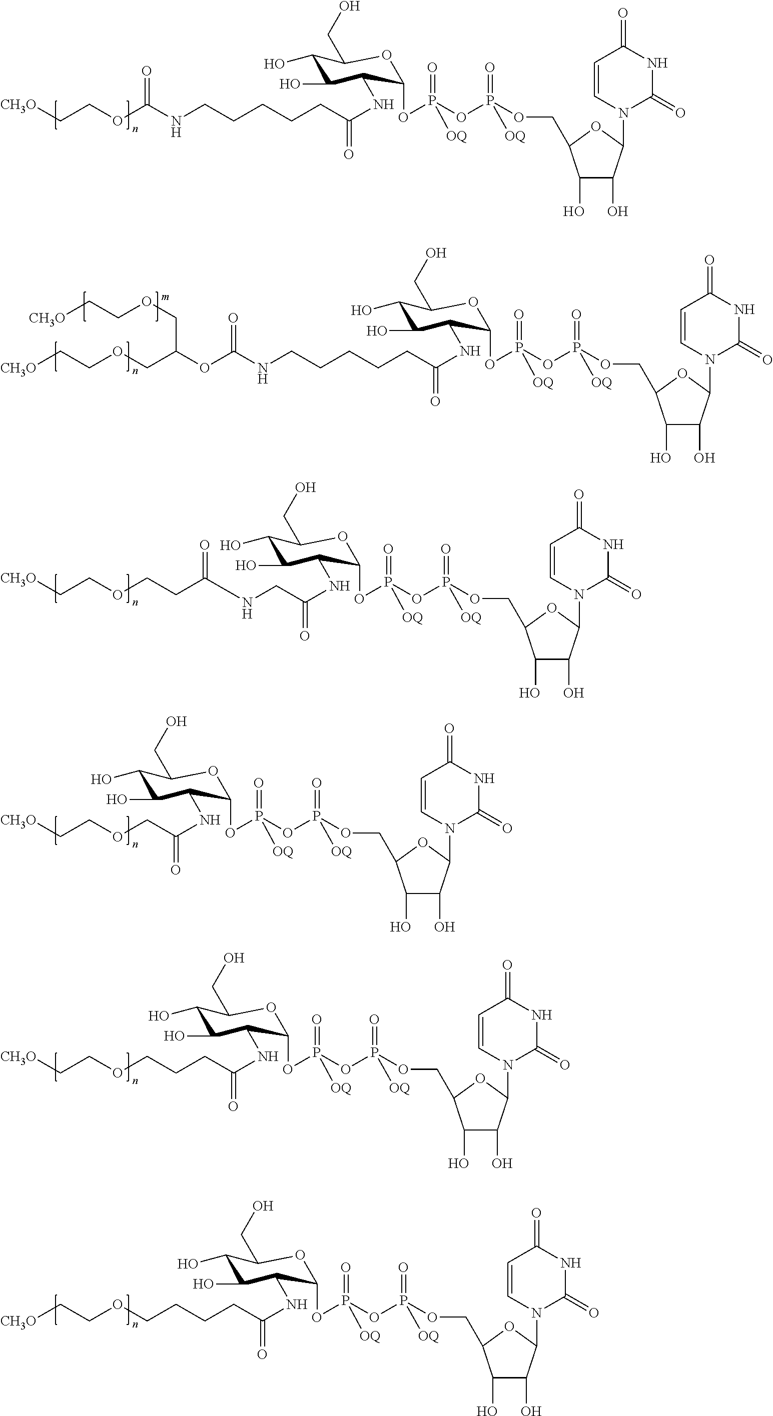

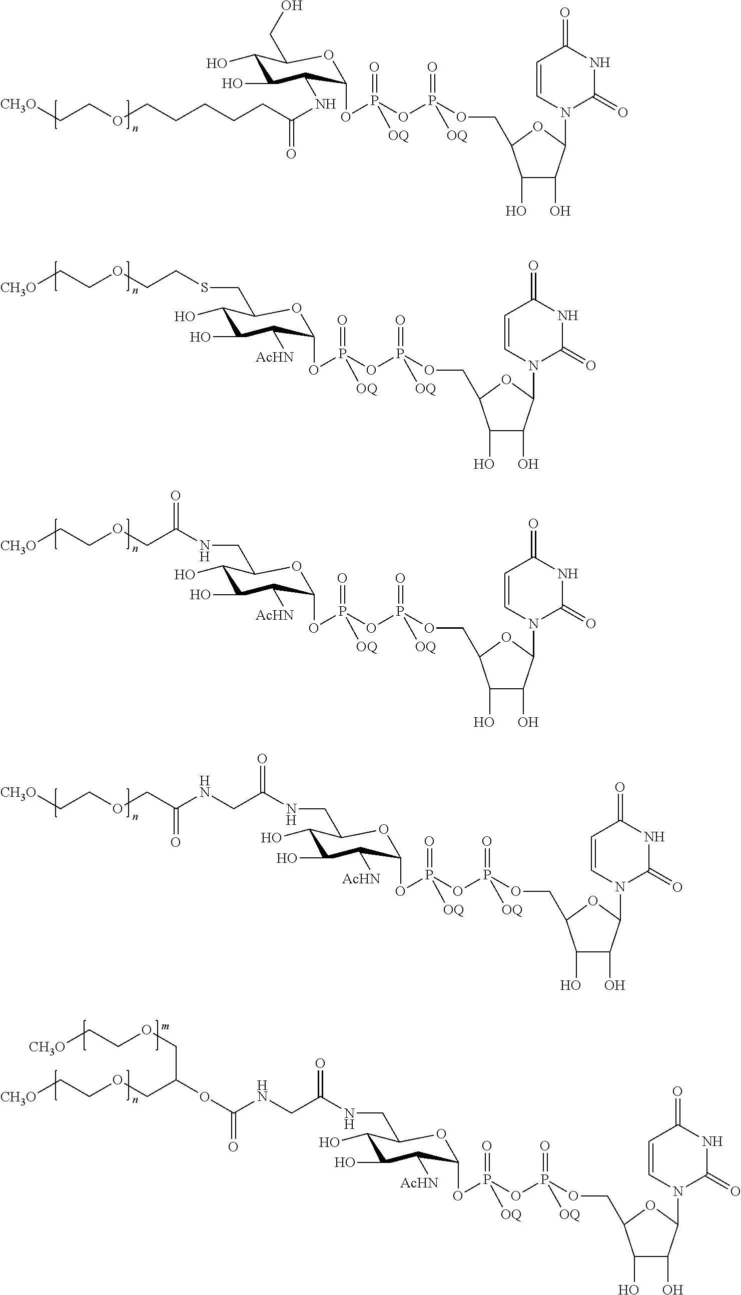

### Activated Sugars

In other embodiments, the modified sugar is an activated sugar. Activated, modified sugars, which are useful in the present invention, are typically glycosides which have been synthetically altered to include a leaving group. In one example, the activated sugar is used in an enzymatic reaction to transfer the activated sugar onto an acceptor on the peptide or glycopeptide. In another example, the activated sugar is added to the peptide or glycopeptide by chemical means. “Leaving group” (or activating group) refers to those moieties, which are easily displaced in enzyme-regulated nucleophilic substitution reactions or alternatively, are replaced in a chemical reaction utilizing a nucleophilic reaction partner (e.g., a glycosyl moiety carrying a sufhydryl group). It is within the abilities of a skilled person to select a suitable leaving group for each type of reaction. Many activated sugars are known in the art. See, for example, Vocadlo et al., In CARBOHYDRATE CHEMISTRY AND BIOLOGY, Vol. 2, Ernst et al. Ed., Wiley-VCH Verlag: Weinheim, Germany, 2000; Kodama et al., Tetrahedron Lett. 34: 6419 (1993); Lougheed, et al., J. Biol. Chem. 274: 37717 (1999)).

Examples of leaving groups include halogen (e.g, fluoro, chloro, bromo), tosylate ester, mesylate ester, triflate ester and the like. Preferred leaving groups, for use in enzyme mediated reactions, are those that do not significantly sterically encumber the enzymatic transfer of the glycoside to the acceptor. Accordingly, preferred embodiments of activated glycoside derivatives include glycosyl fluorides and glycosyl mesylates, with glycosyl fluorides being particularly preferred. Among the glycosyl fluorides, α-galactosyl fluoride, α-mannosyl fluoride, α-glucosyl fluoride, α-fucosyl fluoride, α-xylosyl fluoride, α-sialyl fluoride, α-N-acetylglucosaminyl fluoride, α-N-acetylgalactosaminyl fluoride, β-galactosyl fluoride, β-mannosyl fluoride, β-glucosyl fluoride, β-fucosyl fluoride, β-xylosyl fluoride, β-sialyl fluoride, β-N-acetylglucosaminyl fluoride and β-N-acetylgalactosaminyl fluoride are most preferred. For non-enzymatic, nucleophilic substitutions, these and other leaving groups may be useful. For instance, the activated donor glycoside can be a dinitrophenyl (DNP), or bromo-glycoside.

By way of illustration, glycosyl fluorides can be prepared from the free sugar by first acetylating and then treating the sugar moiety with HF/pyridine. This generates the thermodynamically most stable anomer of the protected (acetylated) glycosyl fluoride (i.e., the α-glycosyl fluoride). If the less stable anomer (i.e., the β-glycosyl fluoride) is desired, it can be prepared by converting the peracetylated sugar with HBr/HOAc or with HCl to generate the anomeric bromide or chloride. This intermediate is reacted with a fluoride salt such as silver fluoride to generate the glycosyl fluoride. Acetylated glycosyl fluorides may be deprotected by reaction with mild (catalytic) base in methanol (e.g. NaOMe/MeOH). In addition, many glycosyl fluorides are commercially available.

Other activated glycosyl derivatives can be prepared using conventional methods known to those of skill in the art. For example, glycosyl mesylates can be prepared by treatment of the fully benzylated hemiacetal form of the sugar with mesyl chloride, followed by catalytic hydrogenation to remove the benzyl groups.

In a further exemplary embodiment, the modified sugar is an oligosaccharide having an antennary structure. In another embodiment, one or more of the termini of the antennae bear the modifying moiety. When more than one modifying moiety is attached to an oligosaccharide having an antennary structure, the oligosaccharide is useful to “amplify” the modifying moiety; each oligosaccharide unit conjugated to the peptide attaches multiple copies of the modifying group to the peptide. The general structure of a typical conjugate of the invention as set forth in the drawing above encompasses multivalent species resulting from preparing a conjugate of the invention utilizing an antennary structure. Many antennary saccharide structures are known in the art, and the present method can be practiced with them without limitation.

### Preparation of Modified Sugars

In general, a covalent bond between the sugar moiety and the modifying group is formed through the use of reactive functional groups, which are typically transformed by the linking process into a new organic functional group or unreactive species. In order to form the bond, the modifying group and the sugar moiety carry complimentary reactive functional groups. The reactive functional group(s), can be located at any position on the sugar moiety.

Reactive groups and classes of reactions useful in practicing the present invention are generally those that are well known in the art of bioconjugate chemistry. Currently favored classes of reactions available with reactive sugar moieties are those, which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

### Reactive Functional Groups

Useful reactive functional groups pendent from a sugar nucleus or modifying group include, but are not limited to:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups, which can be converted to, e.g., esters, ethers, aldehydes, etc.
(c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the functional group of the halogen atom;
(d) dienophile groups, which are capable of participating in Diels-Alder reactions such as, for example, maleimido groups;
(e) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be, for example, converted to disulfides or reacted with acyl halides;
(h) amine or sulfhydryl groups, which can be, for example, acylated, alkylated or oxidized;
(i) alkenes, which can undergo, for example, cycloadditions, acylation, Michael addition, etc; and
(j) epoxides, which can react with, for example, amines and hydroxyl compounds.

The reactive functional groups can be chosen such that they do not participate in, or interfere with, the reactions necessary to assemble the reactive sugar nucleus or modifying group. Alternatively, a reactive functional group can be protected from participating in the reaction by the presence of a protecting group. Those of skill in the art understand how to protect a particular functional group such that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### Cross-Linking Groups

Preparation of the modified sugar for use in the methods of the present invention includes attachment of a modifying group to a sugar residue and forming a stable adduct, which is a substrate for a glycosyltransferase. The sugar and modifying group can be coupled by a zero- or higher-order cross-linking agent. Exemplary bifunctional compounds which can be used for attaching modifying groups to carbohydrate moieties include, but are not limited to, bifunctional poly(ethyleneglycols), polyamides, polyethers, polyesters and the like. General approaches for linking carbohydrates to other molecules are known in the literature. See, for example, Lee et al., Biochemistry 28: 1856 (1989); Bhatia et al., Anal. Biochem. 178: 408 (1989); Janda et al., J. Am. Chem. Soc. 112: 8886 (1990) and Bednarski et al., WO 92/18135. In the discussion that follows, the reactive groups are treated as benign on the sugar moiety of the nascent modified sugar. The focus of the discussion is for clarity of illustration. Those of skill in the art will appreciate that the discussion is relevant to reactive groups on the modifying group as well.

A variety of reagents are used to modify the components of the modified sugar with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

In addition to the use of site-specific reactive moieties, the present invention contemplates the use of non-specific reactive groups to link the sugar to the modifying group.

Exemplary non-specific cross-linkers include photoactivatable groups, completely inert in the dark, which are converted to reactive species upon absorption of a photon of appropriate energy. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In yet a further embodiment, the linker group is provided with a group that can be cleaved to release the modifying group from the sugar residue. Many cleaveable groups are known in the art. See, for example, Jung et al., Biochem. Biophys. Acta 761: 152-162 (1983); Joshi et al., J. Biol. Chem. 265: 14518-14525 (1990); Zarling et al., J. Immunol. 124: 913-920 (1980); Bouizar et al., Eur. J. Biochem. 155: 141-147 (1986); Park et al., J. Biol. Chem. 261: 205-210 (1986); Browning et al., J. Immunol. 143: 1859-1867 (1989). Moreover a broad range of cleavable, bifunctional (both homo- and hetero-bifunctional) linker groups is commercially available from suppliers such as Pierce.

Exemplary cleaveable moieties can be cleaved using light, heat or reagents such as thiols, hydroxylamine, bases, periodate and the like. Moreover, certain preferred groups are cleaved in vivo in response to being endocytized (e.g., cis-aconityl; see, Shen et al., Biochem. Biophys. Res. Commun. 102: 1048 (1991)). Preferred cleaveable groups comprise a cleaveable moiety which is a member selected from the group consisting of disulfide, ester, imide, carbonate, nitrobenzyl, phenacyl and benzoin groups.

Exemplary moieties attached to the conjugates disclosed herein include, but are not limited to, PEG derivatives (e.g., alkyl-PEG, acyl-PEG, acyl-alkyl-PEG, alkyl-acyl-PEG carbamoyl-PEG, aryl-PEG), PPG derivatives (e.g., alkyl-PPG, acyl-PPG, acyl-alkyl-PPG, alkyl-acyl-PPG carbamoyl-PPG, aryl-PPG), therapeutic moieties, diagnostic moieties, mannose-6-phosphate, heparin, heparan, SLeₓ, mannose, mannose-6-phosphate, Sialyl Lewis X, FGF, VFGF, proteins, chondroitin, keratan, dermatan, albumin, integrins, antennary oligosaccharides, peptides and the like. Methods of conjugating the various modifying groups to a saccharide moiety are readily accessible to those of skill in the art (POLY(ETHYLENE GLYCOL CHEMISTRY: BIOTECHNICAL AND BIOMEDICAL APPLICATIONS, J. Milton Harris, Ed., Plenum Pub. Corp., 1992; POLY (ETHYLENE GLYCOL) CHEMICAL AND BIOLOGICAL APPLICATIONS, J. Milton Harris, Ed., ACS Symposium Series No. 680, American Chemical Society, 1997; Hermanson, BIOCONJUGATE TECHNIQUES, Academic Press, San Diego, 1996; and Dunn et al., Eds. POLYMERIC DRUGS AND DRUG DELIVERY SYSTEMS, ACS Symposium Series Vol. 469, American Chemical Society, Washington, D.C. 1991).

An exemplary strategy involves incorporation of a protected sulfhydryl onto the sugar using the heterobifunctional crosslinker SPDP (n-succinimidyl-3-(2-pyridyldithio)propionate and then deprotecting the sulfhydryl for formation of a disulfide bond with another sulfhydryl on the modifying group.

If SPDP detrimentally affects the ability of the modified sugar to act as a glycosyltransferase substrate, one of an array of other crosslinkers such as 2-iminothiolane or N-succinimidyl S-acetylthioacetate (SATA) is used to form a disulfide bond. 2-iminothiolane reacts with primary amines, instantly incorporating an unprotected sulfhydryl onto the amine-containing molecule. SATA also reacts with primary amines, but incorporates a protected sulfhydryl, which is later deacetaylated using hydroxylamine to produce a free sulfhydryl. In each case, the incorporated sulfhydryl is free to react with other sulfhydryls or protected sulfhydryl, like SPDP, forming the required disulfide bond.

The above-described strategy is exemplary, and not limiting, of linkers of use in the invention. Other crosslinkers are available that can be used in different strategies for crosslinking the modifying group to the peptide. For example, TPCH(S-(2-thiopyridyl)-L-cysteine hydrazide and TPMPH ((S-(2-thiopyridyl) mercapto-propionohydrazide) react with carbohydrate moieties that have been previously oxidized by mild periodate treatment, thus forming a hydrazone bond between the hydrazide portion of the crosslinker and the periodate generated aldehydes. TPCH and TPMPH introduce a 2-pyridylthione protected sulfhydryl group onto the sugar, which can be deprotected with DTT and then subsequently used for conjugation, such as forming disulfide bonds between components.

If disulfide bonding is found unsuitable for producing stable modified sugars, other crosslinkers may be used that incorporate more stable bonds between components. The heterobifunctional crosslinkers GMBS (N-gama-malimidobutyryloxy)succinimide) and SMCC (succinimidyl 4-(N-maleimido-methyl)cyclohexane) react with primary amines, thus introducing a maleimide group onto the component. The maleimide group can subsequently react with sulfhydryls on the other component, which can be introduced by previously mentioned crosslinkers, thus forming a stable thioether bond between the components. If steric hindrance between components interferes with either component's activity or the ability of the modified sugar to act as a glycosyltransferase substrate, crosslinkers can be used which introduce long spacer arms between components and include derivatives of some of the previously mentioned crosslinkers (i.e., SPDP). Thus, there is an abundance of suitable crosslinkers, which are useful; each of which is selected depending on the effects it has on optimal peptide conjugate and modified sugar production.

A variety of reagents are used to modify the components of the modified sugar with intramolecular chemical crosslinks (for reviews of crosslinking reagents and crosslinking procedures see: Wold, F., Meth. Enzymol. 25: 623-651, 1972; Weetall, H. H., and Cooney, D. A., In: ENZYMES AS DRUGS. (Holcenberg, and Roberts, eds.) pp. 395-442, Wiley, New York, 1981; Ji, T. H., Meth. Enzymol. 91: 580-609, 1983; Mattson et al., Mol. Biol. Rep. 17: 167-183, 1993, all of which are incorporated herein by reference). Preferred crosslinking reagents are derived from various zero-length, homo-bifunctional, and hetero-bifunctional crosslinking reagents. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. In addition to these chemical reagents, the enzyme transglutaminase (glutamyl-peptide γ-glutamyltransferase; EC 2.3.2.13) may be used as zero-length crosslinking reagent. This enzyme catalyzes acyl transfer reactions at carboxamide groups of protein-bound glutaminyl residues, usually with a primary amino group as substrate. Preferred homo- and hetero-bifunctional reagents contain two identical or two dissimilar sites, respectively, which may be reactive for amino, sulfhydryl, guanidino, indole, or nonspecific groups.

### Preferred Specific Sites in Crosslinking Reagents

### 1. Amino-Reactive Groups

In one embodiment, the sites on the cross-linker are amino-reactive groups. Useful non-limiting examples of amino-reactive groups include N-hydroxysuccinimide (NHS) esters, imidoesters, isocyanates, acylhalides, arylazides, p-nitrophenyl esters, aldehydes, and sulfonyl chlorides.

NHS esters react preferentially with the primary (including aromatic) amino groups of a modified sugar component. The imidazole groups of histidines are known to compete with primary amines for reaction, but the reaction products are unstable and readily hydrolyzed. The reaction involves the nucleophilic attack of an amine on the acid carboxyl of an NHS ester to form an amide, releasing the N-hydroxysuccinimide. Thus, the positive charge of the original amino group is lost.

Imidoesters are the most specific acylating reagents for reaction with the amine groups of the modified sugar components. At a pH between 7 and 10, imidoesters react only with primary amines. Primary amines attack imidates nucleophilically to produce an intermediate that breaks down to amidine at high pH or to a new imidate at low pH. The new imidate can react with another primary amine, thus crosslinking two amino groups, a case of a putatively monofunctional imidate reacting bifunctionally. The principal product of reaction with primary amines is an amidine that is a stronger base than the original amine. The positive charge of the original amino group is therefore retained.

Isocyanates (and isothiocyanates) react with the primary amines of the modified sugar components to form stable bonds. Their reactions with sulfhydryl, imidazole, and tyrosyl groups give relatively unstable products.

Acylazides are also used as amino-specific reagents in which nucleophilic amines of the affinity component attack acidic carboxyl groups under slightly alkaline conditions, e.g. pH 8.5.

Arylhalides such as 1,5-difluoro-2,4-dinitrobenzene react preferentially with the amino groups and tyrosine phenolic groups of modified sugar components, but also with sulfhydryl and imidazole groups.

p-Nitrophenyl esters of mono- and dicarboxylic acids are also useful amino-reactive groups. Although the reagent specificity is not very high, α- and ε-amino groups appear to react most rapidly.

Aldehydes such as glutaraldehyde react with primary amines of modified sugar. Although unstable Schiff bases are formed upon reaction of the amino groups with the aldehydes of the aldehydes, glutaraldehyde is capable of modifying the modified sugar with stable crosslinks. At pH 6-8, the pH of typical crosslinking conditions, the cyclic polymers undergo a dehydration to form α-β unsaturated aldehyde polymers. Schiff bases, however, are stable, when conjugated to another double bond. The resonant interaction of both double bonds prevents hydrolysis of the Schiff linkage. Furthermore, amines at high local concentrations can attack the ethylenic double bond to form a stable Michael addition product.

Aromatic sulfonyl chlorides react with a variety of sites of the modified sugar components, but reaction with the amino groups is the most important, resulting in a stable sulfonamide linkage.

### 2. Sulfhydryl-Reactive Groups

In another embodiment, the sites are sulfhydryl-reactive groups. Useful, non-limiting examples of sulfhydryl-reactive groups include maleimides, alkyl halides, pyridyl disulfides, and thiophthalimides.

Maleimides react preferentially with the sulfhydryl group of the modified sugar components to form stable thioether bonds. They also react at a much slower rate with primary amino groups and the imidazole groups of histidines. However, at pH 7 the maleimide group can be considered a sulfhydryl-specific group, since at this pH the reaction rate of simple thiols is 1000-fold greater than that of the corresponding amine.

Alkyl halides react with sulfhydryl groups, sulfides, imidazoles, and amino groups. At neutral to slightly alkaline pH, however, alkyl halides react primarily with sulfhydryl groups to form stable thioether bonds. At higher pH, reaction with amino groups is favored.

Pyridyl disulfides react with free sulfhydryls via disulfide exchange to give mixed disulfides. As a result, pyridyl disulfides are the most specific sulfhydryl-reactive groups.

Thiophthalimides react with free sulfhydryl groups to form disulfides.

### 3. Carboxyl-Reactive Residue

In another embodiment, carbodiimides soluble in both water and organic solvent, are used as carboxyl-reactive reagents. These compounds react with free carboxyl groups forming a pseudourea that can then couple to available amines yielding an amide linkage teach how to modify a carboxyl group with carbodiimde (Yamada et al., Biochemistry 20: 4836-4842, 1981).

### Preferred Nonspecific Sites in Crosslinking Reagents

In addition to the use of site-specific reactive moieties, the present invention contemplates the use of non-specific reactive groups to link the sugar to the modifying group.

Exemplary non-specific cross-linkers include photoactivatable groups, completely inert in the dark, which are converted to reactive species upon absorption of a photon of appropriate energy. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In another preferred embodiment, photoactivatable groups are selected from fluorinated arylazides. The photolysis products of fluorinated arylazides are arylnitrenes, all of which undergo the characteristic reactions of this group, including C—H bond insertion, with high efficiency (Keana et al., J. Org. Chem. 55: 3640-3647, 1990).

In another embodiment, photoactivatable groups are selected from benzophenone residues. Benzophenone reagents generally give higher crosslinking yields than arylazide reagents.

In another embodiment, photoactivatable groups are selected from diazo compounds, which form an electron-deficient carbene upon photolysis. These carbenes undergo a variety of reactions including insertion into C—H bonds, addition to double bonds (including aromatic systems), hydrogen attraction and coordination to nucleophilic centers to give carbon ions.

In still another embodiment, photoactivatable groups are selected from diazopyruvates. For example, the p-nitrophenyl ester of p-nitrophenyl diazopyruvate reacts with aliphatic amines to give diazopyruvic acid amides that undergo ultraviolet photolysis to form aldehydes. The photolyzed diazopyruvate-modified affinity component will react like formaldehyde or glutaraldehyde forming crosslinks.

### Homobifunctional Reagents

### 1. Homobifunctional Crosslinkers Reactive With Primary Amines

Synthesis, properties, and applications of amine-reactive cross-linkers are commercially described in the literature (for reviews of crosslinking procedures and reagents, see above). Many reagents are available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional NHS esters include disuccinimidyl glutarate (DSG), disuccinimidyl suberate (DSS), bis(sulfosuccinimidyl) suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxy-carbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidyl-propionate (DSP), and dithiobis(sulfosuccinimidylpropionate (sulfo-DSP). Preferred, non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DMDP), dimethyl-,3′-(dimethylenedioxy)dipropionimidate (DDDP), dimethyl-3,3′-(tetramethylenedioxy)-dipropionimidate (DTDP), and dimethyl-3,3′-dithiobispropionimidate (DTBP).

Preferred, non-limiting examples of homobifunctional isothiocyanates include: p-phenylenediisothiocyanate (DITC), and 4,4′-diisothiocyano-2,2′-disulfonic acid stilbene (DIDS).

Preferred, non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate.

Preferred, non-limiting examples of homobifunctional arylhalides include 1,5-difluoro-2,4-dinitrobenzene (DFDNB), and 4,4′-difluoro-3,3′-dinitrophenyl-sulfone.

Preferred, non-limiting examples of homobifunctional aliphatic aldehyde reagents include glyoxal, malondialdehyde, and glutaraldehyde.

Preferred, non-limiting examples of homobifunctional acylating reagents include nitrophenyl esters of dicarboxylic acids.

Preferred, non-limiting examples of homobifunctional aromatic sulfonyl chlorides include phenol-2,4-disulfonyl chloride, and α-naphthol-2,4-disulfonyl chloride.

Preferred, non-limiting examples of additional amino-reactive homobifunctional reagents include erythritolbiscarbonate which reacts with amines to give biscarbamates.

2. Homobifunctional Crosslinkers Reactive with Free Sulfhydryl Groups

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional maleimides include bismaleimidohexane (BMH), N,N′-(1,3-phenylene) bismaleimide, N,N′-(1,2-phenylene)bismaleimide, azophenyldimaleimide, and bis(N-maleimidomethyl)ether.

Preferred, non-limiting examples of homobifunctional pyridyl disulfides include 1,4-di-3′-(2′-pyridyldithio)propionamidobutane (DPDPB).

Preferred, non-limiting examples of homobifunctional alkyl halides include 2,2′-dicarboxy-4,4′-diiodoacetamidoazobenzene, α,α′-diiodo-p-xylenesulfonic acid, α,α′-dibromo-p-xylenesulfonic acid, N,N′-bis(b-bromoethyl)benzylamine, N,N′-di(bromoacetyl)phenylthydrazine, and 1,2-di(bromoacetyl)amino-3-phenylpropane.

### 3. Homobifunctional Photoactivatable Crosslinkers

Synthesis, properties, and applications of such reagents are described in the literature (for reviews of crosslinking procedures and reagents, see above). Some of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of homobifunctional photoactivatable crosslinker include bis-β-(4-azidosalicylamido)ethyldisulfide (BASED), di-N-(2-nitro-4-azidophenyl)-cystamine-S,S-dioxide (DNCO), and 4,4′-dithiobisphenylazide.

### HeteroBifunctional Reagents

1. Amino-Reactive HeteroBifunctional Reagents with a Pyridyl Disulfide Moiety

Synthesis, properties, and applications of such reagents are described in the literature (for reviews of crosslinking procedures and reagents, see above). Many of the reagents are commercially available (e.g., Pierce Chemical Company, Rockford, Ill.; Sigma Chemical Company, St. Louis, Mo.; Molecular Probes, Inc., Eugene, Oreg.).

Preferred, non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-α-methyl-α-(2-pyridyldithio)toluene (SMPT), and sulfosuccinimidyl 6-α-methyl-α-(2-pyridyldithio)toluamidohexanoate (sulfo-LC-SMPT).

2. Amino-Reactive HeteroBifunctional Reagents with a Maleimide Moiety

Synthesis, properties, and applications of such reagents are described in the literature. Preferred, non-limiting examples of hetero-bifunctional reagents with a maleimide moiety and an amino-reactive NHS ester include succinimidyl maleimidylacetate (AMAS), succinimidyl 3-maleimidylpropionate (BMPS), N-γ-maleimidobutyryloxysuccinimide ester (GMBS)N-γ-maleimidobutyryloxysulfo succinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethyl)-cyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

3. Amino-Reactive HeteroBifunctional Reagents with an Alkyl Halide Moiety

Synthesis, properties, and applications of such reagents are described in the literature Preferred, non-limiting examples of hetero-bifunctional reagents with an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)-methyl)-cyclohexane-1-carbonyl)aminohexanoate (SIACX), and succinimidyl-4((iodoacetyl)-amino)methylcyclohexane-1-carboxylate (SIAC).

An example of a hetero-bifunctional reagent with an amino-reactive NHS ester and an alkyl dihalide moiety is N-hydroxysuccinimidyl 2,3-dibromopropionate (SDBP). SDBP introduces intramolecular crosslinks to the affinity component by conjugating its amino groups. The reactivity of the dibromopropionyl moiety towards primary amine groups is controlled by the reaction temperature (McKenzie et al., Protein Chem. 7: 581-592 (1988)).

Preferred, non-limiting examples of hetero-bifunctional reagents with an alkyl halide moiety and an amino-reactive p-nitrophenyl ester moiety include p-nitrophenyl iodoacetate (NPIA).

Other cross-linking agents are known to those of skill in the art. See, for example, Pomato et al., U.S. Pat. No. 5,965,106. It is within the abilities of one of skill in the art to choose an appropriate cross-linking agent for a particular application.

### Cleavable Linker Groups

In yet a further embodiment, the linker group is provided with a group that can be cleaved to release the modifying group from the sugar residue. Many cleaveable groups are known in the art. See, for example, Jung et al., Biochem. Biophys. Acta 761: 152-162 (1983); Joshi et al., J. Biol. Chem. 265: 14518-14525 (1990); Zarling et al., J. Immunol. 124: 913-920 (1980); Bouizar et al., Eur. J. Biochem. 155: 141-147 (1986); Park et al., J. Biol. Chem. 261: 205-210 (1986); Browning et al., J. Immunol. 143: 1859-1867 (1989). Moreover a broad range of cleavable, bifunctional (both homo- and hetero-bifunctional) linker groups is commercially available from suppliers such as Pierce.

Exemplary cleaveable moieties can be cleaved using light, heat or reagents such as thiols, hydroxylamine, bases, periodate and the like. Moreover, certain preferred groups are cleaved in vivo in response to being endocytized (e.g., cis-aconityl; see, Shen et al., Biochem. Biophys. Res. Commun. 102: 1048 (1991)). Preferred cleaveable groups comprise a cleaveable moiety which is a member selected from the group consisting of disulfide, ester, imide, carbonate, nitrobenzyl, phenacyl and benzoin groups.

Specific embodiments according to the invention include:

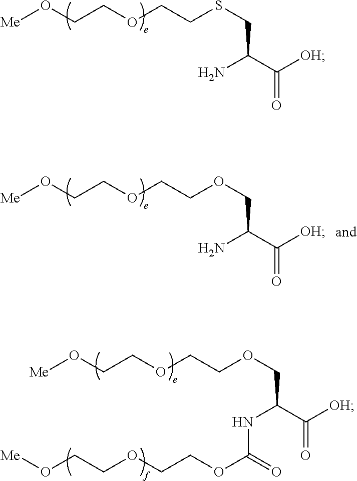

and carbonates and active esters of these species, such as:

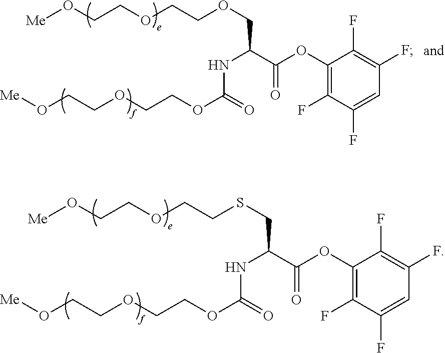

### Exemplary Conjugates of the Invention

In an exemplary embodiment, the polypeptide is an interferon. The interferons are antiviral glycoproteins that, in humans, are secreted by human primary fibroblasts after induction with virus or double-stranded RNA. Interferons are of interest as therapeutics, e.g, antiviral agents (e.g., hepatitis B and C), antitumor agents (e.g., hepatocellular carcinoma) and in the treatment of multiple sclerosis. For references relevant to interferon-α, see, Asano, et al., Eur. J. Cancer, 27(Suppl 4):S21-S25 (1991); Nagy, et al., Anticancer Research, 8(3):467-470 (1988); Dron, et al., J. Biol. Regul. Homeost. Agents, 3(1):13-19 (1989); Habib, et al., Am. Surg., 67(3):257-260 (3/2001); and Sugyiama, et al., Eur. J. Biochem., 217:921-927 (1993). For references discussing intefereon-β, see, e.g., Yu, et al., J. Neuroimmunol., 64(1):91-100 (1996); Schmidt, J., J. Neurosci. Res., 65(1):59-67 (2001); Wender, et al., Folia Neuropathol., 39(2):91-93 (2001); Martin, et al., Springer Semin. Immunopathol., 18(1):1-24 (1996); Takane, et al., J. Pharmacol. Exp. Ther., 294(2):746-752 (2000); Sburlati, et al., Biotechnol. Prog., 14:189-192 (1998); Dodd, et al., Biochimica et Biophysica Acta, 787:183-187 (1984); Edelbaum, et al., J. Interferon Res., 12:449-453 (1992); Conradt, et al., J. Biol. Chem., 262(30):14600-14605 (1987); Civas, et al., Eur. J. Biochem., 173:311-316 (1988); Demolder, et al., J. Biotechnol., 32:179-189 (1994); Sedmak, et al., J. Interferon Res., 9(Suppl 1):561-565 (1989); Kagawa, et al., J. Biol. Chem., 263(33):17508-17515 (1988); Hershenson, et al., U.S. Pat. No. 4,894,330; Jayaram, et al., J. Interferon Res., 3(2):177-180 (1983); Menge, et al., Develop. Biol. Standard., 66:391-401 (1987); Vonk, et al., J. Interferon Res., 3(2):169-175 (1983); and Adolf, et al., J. Interferon Res., 10:255-267 (1990).

In an exemplary interferon conjugate, interferon alpha, e.g., interferon alpha 2b and 2a, is conjugated to a water soluble polymer through an intact glycosyl linker.

In a further exemplary embodiment, the invention provides a conjugate of human granulocyte colony stimulating factor (G-CSF). G-CSF is a glycoprotein that stimulates proliferation, differentiation and activation of neutropoietic progenitor cells into functionally mature neutrophils. Injected G-CSF is rapidly cleared from the body. See, for example, Nohynek, et al., Cancer Chemother. Pharmacol., 39:259-266 (1997); Lord, et al., Clinical Cancer Research, 7(7):2085-2090 (07/2001); Rotondaro, et al., Molecular Biotechnology, 11(2):117-128 (1999); and Bonig, et al., Bone Marrow Transplantation, 28: 259-264 (2001).

The present invention encompasses a method for the modification of GM-CSF. GM-CSF is well known in the art as a cytokine produced by activated T-cells, macrophages, endothelial cells, and stromal fibroblasts. GM-CSF primarily acts on the bone marrow to increase the production of inflammatory leukocytes, and further functions as an endocrine hormone to initiate the replenishment of neutrophils consumed during inflammatory functions. Further GM-CSF is a macrophage-activating factor and promotes the differentiation of Lagerhans cells into dendritic cells. Like G-CSF, GM-CSF also has clinical applications in bone marrow replacement following chemotherapy

### Nucleic Acids

In another aspect, the invention provides an isolated nucleic acid encoding a non-naturally occurring polypeptide of the invention. In one embodiment, the nucleic acid of the invention is part of an expression vector. In another related embodiment, the present invention provides a cell including the nucleic acid of the present invention. Exemplary cells include host cells such as various strains of E. coli, insect cells and mammalian cells, such as CHO cells.

### Pharmaceutical Compositions

In another aspect, the invention provides pharmaceutical compositions including at least one polypeptide or polypeptide conjugate of the invention and a pharmaceutically acceptable carrier. In an exemplary embodiment, the pharmaceutical composition includes a covalent conjugate between a water-soluble polymer (e.g., a non-naturally-occurring water-soluble polymer), and a glycosylated or non-glycosylated polypeptide of the invention as well as a pharmaceutically acceptable carrier. Exemplary water-soluble polymers include poly(ethylene glycol) and methoxy-poly(ethylene glycol). Alternatively, the polypeptide is conjugated to a modifying group other than a poly(ethylene glycol) derivative, such as a therapeutic moiety or a biomolecule.

Polypeptide conjugates of the invention have a broad range of pharmaceutical applications. For example, glycoconjugated erythropoietin (EPO) may be used for treating general anemia, aplastic anemia, chemo-induced injury (such as injury to bone marrow), chronic renal failure, nephritis, and thalassemia. Modified EPO may be further used for treating neurological disorders such as brain/spine injury, multiple sclerosis, and Alzheimer's disease.

A second example is interferon-α (IFN-α), which may be used for treating AIDS and hepatitis B or C, viral infections caused by a variety of viruses such as human papilloma virus (HBV), coronavirus, human immunodeficiency virus (HIV), herpes simplex virus (HSV), and varicella-zoster virus (VZV), cancers such as hairy cell leukemia, AIDS-related Kaposi's sarcoma, malignant melanoma, follicular non-Hodgkins lymphoma, Philladephia chromosome (Ph)-positive, chronic phase myelogenous leukemia (CML), renal cancer, myeloma, chronic myelogenous leukemia, cancers of the head and neck, bone cancers, as well as cervical dysplasia and disorders of the central nervous system (CNS) such as multiple sclerosis. In addition, IFN-α modified according to the methods of the present invention is useful for treating an assortment of other diseases and conditions such as Sjogren's syndrome (an autoimmune disease), Behcet's disease (an autoimmune inflammatory disease), fibromyalgia (a musculoskeletal pain/fatigue disorder), aphthous ulcer (canker sores), chronic fatigue syndrome, and pulmonary fibrosis.

Another example is interferon-β, which is useful for treating CNS disorders such as multiple sclerosis (either relapsing/remitting or chronic progressive), AIDS and hepatitis B or C, viral infections caused by a variety of viruses such as human papilloma virus (HBV), human immunodeficiency virus (HIV), herpes simplex virus (HSV), and varicella-zoster virus (VZV), otological infections, musculoskeletal infections, as well as cancers including breast cancer, brain cancer, colorectal cancer, non-small cell lung cancer, head and neck cancer, basal cell cancer, cervical dysplasia, melanoma, skin cancer, and liver cancer. IFN-β modified according to the methods of the present invention is also used in treating other diseases and conditions such as transplant rejection (e.g., bone marrow transplant), Huntington's chorea, colitis, brain inflammation, pulmonary fibrosis, macular degeneration, hepatic cirrhosis, and keratoconjunctivitis.

Granulocyte colony stimulating factor (G-CSF) is a further example. G-CSF modified according to the methods of the present invention may be used as an adjunct in chemotherapy for treating cancers, and to prevent or alleviate conditions or complications associated with certain medical procedures, e.g., chemo-induced bone marrow injury; leucopenia (general); chemo-induced febrile neutropenia; neutropenia associated with bone marrow transplants; and severe, chronic neutropenia. Modified G-CSF may also be used for transplantation; peripheral blood cell mobilization; mobilization of peripheral blood progenitor cells for collection in patients who will receive myeloablative or myelosuppressive chemotherapy; and reduction in duration of neutropenia, fever, antibiotic use, hospitalization following induction/consolidation treatment for acute myeloid leukemia (AML). Other condictions or disorders may be treated with modified G-CSF include asthma and allergic rhinitis.

As one additional example, human growth hormone (hGH) modified according to the methods of the present invention may be used to treat growth-related conditions such as dwarfism, short-stature in children and adults, cachexia/muscle wasting, general muscular atrophy, and sex chromosome abnormality (e.g., Turner's Syndrome). Other conditions may be treated using modified hGH include: short-bowel syndrome, lipodystrophy, osteoporosis, uraemaia, burns, female infertility, bone regeneration, general diabetes, type II diabetes, osteo-arthritis, chronic obstructive pulmonary disease (COPD), and insomia. Moreover, modified hGH may also be used to promote various processes, e.g., general tissue regeneration, bone regeneration, and wound healing, or as a vaccine adjunct.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable matrices, such as microspheres (e.g., polylactate polyglycolate), may also be employed as carriers for the pharmaceutical compositions of this invention. Suitable biodegradable microspheres are disclosed, for example, in U.S. Pat. Nos. 4,897,268 and 5,075,109.

Commonly, the pharmaceutical compositions are administered subcutaneously or parenterally, e.g., intravenously. Thus, the invention provides compositions for parenteral administration, which include the compound dissolved or suspended in an acceptable carrier, preferably an aqueous carrier, e.g., water, buffered water, saline, PBS and the like. The compositions may also contain detergents such as Tween 20 and Tween 80; stabilizers such as mannitol, sorbitol, sucrose, and trehalose; and preservatives such as EDTA and meta-cresol. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the glycopeptides of the invention can be incorporated into liposomes formed from standard vesicle-forming lipids. A variety of methods are available for preparing liposomes, as described in, e.g., Szoka et al., Ann. Rev. Biophys. Bioeng. 9: 467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

Standard methods for coupling targeting agents to liposomes can be used. These methods generally involve incorporation into liposomes of lipid components, such as phosphatidylethanolamine, which can be activated for attachment of targeting agents, or derivatized lipophilic compounds, such as lipid-derivatized glycopeptides of the invention.

Targeting mechanisms generally require that the targeting agents be positioned on the surface of the liposome in such a manner that the target moieties are available for interaction with the target, for example, a cell surface receptor. The carbohydrates of the invention may be attached to a lipid molecule before the liposome is formed using methods known to those of skill in the art (e.g., alkylation or acylation of a hydroxyl group present on the carbohydrate with a long chain alkyl halide or with a fatty acid, respectively). Alternatively, the liposome may be fashioned in such a way that a connector portion is first incorporated into the membrane at the time of forming the membrane. The connector portion must have a lipophilic portion, which is firmly embedded and anchored in the membrane. It must also have a reactive portion, which is chemically available on the aqueous surface of the liposome. The reactive portion is selected so that it will be chemically suitable to form a stable chemical bond with the targeting agent or carbohydrate, which is added later. In some cases it is possible to attach the target agent to the connector molecule directly, but in most instances it is more suitable to use a third molecule to act as a chemical bridge, thus linking the connector molecule which is in the membrane with the target agent or carbohydrate which is extended, three dimensionally, off of the vesicle surface.

The compounds prepared by the methods of the invention may also find use as diagnostic reagents. For example, labeled compounds can be used to locate areas of inflammation or tumor metastasis in a patient suspected of having an inflammation. For this use, the compounds can be labeled with ¹²⁵I, ¹⁴C, or tritium

### V. Methods

### Identification of Mutant Polypeptides as Substrates for Glycosyltransferases

One strategy for the identification of mutant polypeptides, which are glycosylated with a satisfactory yield when subjected to a glycosylation reaction, is to prepare a library of non-naturally occurring (i.e., mutant) polypeptides, wherein each mutant polypeptide includes at least one O-linked glycosylation sequence of the invention, and to test each mutant polypeptide for its ability to function as an efficient substrate for a glycosyltransferase (e.g., a GlcNAc-transferase). A library of mutant polypeptides can be generated by creating a selected O-linked glycosylation sequence of the invention at different positions within the amino acid sequence of a parent polypeptide by mutation.

### Library of Mutant Polypeptides

In one aspect, the invention provides methods of generating a library of mutant polypeptides, wherein the mutant polypeptides are derived from a wild-type or parent polypeptide. In one embodiment, the parent polypeptide has an amino acid sequence including m amino acids. Each amino acid position within the amino acid sequence is represented by (AA)ₙ, wherein n is a member selected from 1 to m. An exemplary method of generating a library of mutant polypeptides includes the steps of: (i) generating a mutant polypeptide by introducing a mutant O-linked glycosylation sequence of the invention at a first amino acid position (AA)ₙ within the parent polypeptide; (ii) generating at least one additional mutant polypeptide by repeating step (i) a desired number of times, wherein the same mutant O-linked glycosylation sequence is introduced at a second amino acid position, which is a member selected from (AA)ₙ₊ₓ and (AA)ₙ₋ₓ, wherein x is a member selected from 1 to (m−n). Embodiments of this method are described herein above. In an exemplary embodiment, the library of mutant polypeptides is generated by “Sequon Scanning”.

Identification of Lead polypeptides

After generating a library of mutant polypeptides it may be desirable to select among the members of the library those mutants that are effectively glycosylated and/or glycoPEGylated when subjected to an enzymatic glycosylation and/or glycoPEGylation reaction. Mutant polypeptides, which are found to be effectively glycosylated and/or glycoPEGylated are termed “lead polypeptides”. In an exemplary embodiment, the yield of the enzymatic glycosylation or glycoPEGylation reaction is used to select one or more lead polypeptides. In another exemplary embodiment, the yield of the enzymatic glycosylation or glycoPEGylation for a lead polypeptide is between about 10% and about 100%, preferably between about 30% and about 100%, more preferably between about 50% and about 100% and most preferably between about 70% and about 100%. Lead polypeptides that can be efficiently glycosylated are optionally further evaluated by subjecting the glycosylated lead polypeptide to another enzymatic glycosylation or glycoPEGylation reaction.

Thus, the invention provides methods for identifying a lead polypeptide. An exemplary method includes the steps of: (i) generating a library of mutant polypeptides of the invention (e.g., according to the methods of the invention); (ii) subjecting at least one member of the library to an enzymatic glycosylation reaction (or optionally an enzymatic glycoPEGylation reaction), transferring a glycosyl moiety from a glycosyl donor molecule onto at least one of the mutant O-linked glycosylation sequence, wherein the glycosyl moiety is optionally derivatized with a modifying group; and (iii) measuring the yield of the enzymatic glycosylation or glycoPEGylation reaction for at least one member of the library.

The transferred glycosyl moiety can be any glycosyl moiety including mono- and oligosaccharides as well as glycosyl-mimetic groups. In an exemplary embodiment, the glycosyl moiety, which is added to the mutant polypeptide in an initial glycosylation reaction, is a GalNAc moiety. Subsequent glycosylation reactions can be employed to add additional glycosyl residues (e.g, Gal) to the resulting GalNAc-polypeptide. The modifying group can be any modifying group of the invention, including water soluble polymers such as mPEG.

Methods of generating mutant polypeptides (including any lead polypeptide) are known in the art. Exemplary methods are described herein. The method may include one or more of the following steps: (iv) generating an expression vector including a nucleic acid sequence corresponding to the mutant polypeptide; (v) transfecting a host cell with the expression vector; (vi) expressing the mutant polypeptide in the host cell; and (vii) isolating the mutant polypeptide. A mutant polypeptide of interest (e.g., a selected lead polypeptide) can be expressed on an industrial scale (e.g., leading to the isolation of more than 250 mg, preferably more than 500 mg of protein).

In an exemplary embodiment, each member of a library of mutant polypeptides is subjected to an enzymatic glycosylation reaction. For example, each mutant polypeptide is separately subjected to a glycosylation reaction and the yield of the glycosylation reaction is determined for one or more selected reaction condition.

In an exemplary embodiment, one or more mutant polypeptide of the library is purified prior to further processing, such as glycosylation and/or glycoPEGylation.

In another example, groups of mutant polypeptides can be combined and the resulting mixture of mutant polypeptides can be subjected to a glycosylation or glycoPEGylation reaction. In one exemplary embodiment, a mixture containing all members of the library is subjected to a glycosylation reaction. In one example, according to this embodiment, the glycosyl donor reagent can be added to the glycosylation reaction mixture in a less than stoichiometric amount (with respect to glycosylation sites present) creating an environment in which the mutant polypeptides compete as substrates for the enzyme. Those mutant polypeptides, which are substrates for the enzyme, can then be identified, for instance by virtue of mass spectral analysis with or without prior separation or purification of the glycosylated mixture. This same approach may be used for a group of mutant polypeptides which each contain a different O-linked glycosylation sequences of the invention.

An exemplary assay, which is useful for the screening of polypeptides for their ability to function as a substrate for a GlcNAc transferase is described in T. M. Leavy and C. R. Bertozzi, Bioorg. Med. Chem. Lett. 2007, 17: 3851-3854, incorporated herein by reference in its entirety for all purposes. Enzymatic glycosylation reaction yields can also be determined using any suitable method known in the art. In one embodiment, mass spectroscopy (e.g., MALDI-TOF) or gel electrophoresis is used to distinguish between a glycosylated polypeptide and an unreacted (e.g., non-glycosylated) polypeptide. In another preferred embodiment, HPLC is used to determine the extent of glycosylation. Nuclear magnetic resonance techniques may also be used for this purpose. In one embodiment a multi-well plate (e.g., a 96-well plate) is used to carry out a number of glycosylation reactions in parallel. The plate may optionally be equipped with a separation or filtration medium (e.g., gel-filtration membrane) in the bottom of each well. Spinning may be used to pre-condition each sample prior to analysis by mass spectroscopy or other means.

Glycosylation within a Host Cell

Initial glycosylation of a mutant O-linked glycosylation sequence, which is part of a mutant polypeptide of the invention, can also occur within a host cell, in which the polypeptide is expressed. This technology is, for instance, described in U.S. Provisional Patent Application No. 60/842,926 filed on Sep. 6, 2006, which is incorporated herein by reference in its entirety. The host cell may be a prokaryotic microorganism, such as E. coli or Pseudomonas strains). In an exemplary embodiment, the host cell is a trxB gor supp mutant E. coli cell.

In another exemplary embodiment, intracellular glycosylation is accomplished by co-expressing the polypeptide and an “active nucleotide sugar:polypeptide glycosyltransferase protein” (e.g., a soluble active eukaryotic N-acetylgalactosaminyl transferase) in the host cell and growing the host cell under conditions that allow intracellular transfer of a sugar moiety to the glycosylation sequence. In another exemplary embodiment, the microorganism in which the mutant polypeptide is expressed has an intracelluar oxidizing environment. The microorganism may be genetically modified to have the intracellular oxidizing environment. Intracellualr glycosylation is not limited to the transfer of a single glycosyl residue. Several glycosyl residues can be added sequentially by co-expression of required enzymes and the presence of respective glycosyl donors. This approach can also be used to produce mutant polypeptides on a commercial scale.

Methods are available to determine whether or not a mutant polypeptide is efficiently glycosylated within the mutant O-linked glycosylation sequence inside the host cell. For example the cell lysate (after one or more purification steps) is analyzed by mass spectroscopy to measure the ratio between glycosylated and non-glycosylated mutant polypeptide. In another example, the cell lysate is analyzed by gel electrophoresis separating glycosylated from non-glycosylated peptide.

Further Evaluation of Lead polypeptides

In one embodiment, in which the initial screening procedure involves enzymatic glycosylation using an unmodified glycosyl moiety (e.g., transfer of a GalNAc moiety by GalNAc-T2), selected lead polypeptides may be further evaluated for their capability of being an efficient substrate for further modification, e.g., through another enzymatic reaction or a chemical modification. In an exemplary embodiment, subsequent “screening” involves subjecting a glycosylated lead polypeptide to another glycosylation—(e.g., addition of Gal) and/or PEGylation reaction.

A PEGylation reaction can, for instance, be a chemical PEGylation reaction or an enzymatic glycoPEGylation reaction. In order to identify a lead polypeptide, which is efficiently glycoPEGylated, at least one lead polypeptide (optionally previously glycosylated) is subjected to a PEGylation reaction and the yield for this reaction is determined. In one example, PEGylation yields for each lead polypeptide are determined. In an exemplary embodiment, the yield for the PEGylation reaction is between about 10% and about 100%, preferably between about 30% and about 100%, more preferably between about 50% and about 100% and most preferably between about 70% and about 100%. The PEGylation yield can be determined using any analytical method known in the art, which is suitable for polypeptide analysis, such as mass spectroscopy (e.g., MALDI-TOF, Q-TOF), gel electrophoresis (e.g., in combination with means for quantification, such as densitometry), NMR techniques as well as chromatographic methods, such as HPLC using appropriate column materials useful for the separation of PEGylated and non-PEGylated species of the analyzed polypeptide. As described above for glycosylation, a multi-well plate (e.g., a 96-well plate) can be used to carry out a number of PEGylation reactions in parallel. The plate may optionally be equipped with a separation or filtration medium (e.g., gel-filtration membrane) in the bottom of each well. Spinning and reconstitution may be used to pre-condition each sample prior to analysis by mass spectroscopy or other means.

In another exemplary embodiment, glycosylation and glycoPEGylation of a mutant polypeptide occur in a “one pot reaction” as described below. In one example, the mutant polypeptide is contacted with a first enzyme (e.g., GalNAc-T2) and an appropriate donor molecule (e.g., UDP-GalNAc). The mixture is incubated for a suitable amount of time before a second enzyme (e.g., Core-1-GalT1) and a second glycosyl donor (e.g., UDP-Gal) are added. Any number of additional glycosylation/glycoPEGylation reactions can be performed in this manner. Alternatively, more than one enzyme and more than one glycosyl donor can be contacted with the mutant polypeptide to add more than one glycosyl residue in one reaction step. For example, the mutant polypeptide is contacted with 3 different enzymes (e.g., GalNAc-T2, Core-1-GalT1 and ST3Gal1) and three different glycosyl donor moieties (e.g, UDP-GalNAc, UDP-Gal and CMP-SA-PEG) in a suitable buffer system to generate a glycoPEGylated mutant polypeptide, such as polypeptide-GalNAc-Gal-SA-PEG (see, Example 4.6). Overall yields can be determined using the methods described above.

### Formation of Polypeptide Conjugates

In another aspect, the invention provides methods of forming a covalent conjugate between a modifying group and a polypeptide. The polypeptide conjugates of the invention are formed between glycosylated or non-glycosylated polypeptides and diverse species such as water-soluble polymers, therapeutic moieties, biomolecules, diagnostic moieties, targeting moieties and the like. The polymer, therapeutic moiety or biomolecule is conjugated to the peptide via a glycosyl linking group, which is interposed between, and covalently linked to both the polypeptide and the modifying group (e.g. water-soluble polymer). The sugar moiety of the modified sugar is preferably selected from nucleotide sugars, activated sugars and sugars, which are neither nucleotides nor activated.

In an exemplary embodiment, the polypeptide conjugate is formed through enzymatic attachment of a modified sugar to the polypeptide. In contrast to known chemical and enzymatic peptide elaboration strategies, the methods of the invention, make it possible to assemble peptides and glycopeptides that have a substantially homogeneous derivatization pattern. The enzymes used in the invention are generally selective for a particular amino acid residue or combination of amino acid residues of the peptide. The methods of the invention also provide practical means for large-scale production of modified peptides and glycopeptides.

Using the exquisite selectivity of enzymes, such as glycosyltransferases, the present method provides polypeptides that bear modifying groups at one or more specific locations. Thus, according to the present invention, a modified sugar is attached directly to an O-linked glycosylation sequence within the polypeptide chain or, alternatively, the modified sugar is appended onto a carbohydrate moiety of a glycopeptide. Peptides in which modified sugars are bound to both a glycosylated site and directly to an amino acid residue of the polypeptide backbone are also within the scope of the present invention. In a preferred embodiment, a modified glucosamine moiety is added directly to an amino acid side chain of an O-linked glycosylation sequence of the invention, preferably through the action of a GlcNAc transferase.

Thus, in one aspect, the invention provides a method of forming a covalent conjugate between a polypeptide and a modifying group (e.g., a polymeric modifying group, which is optionally water-soluble) wherein said polypeptide comprises an O-linked glycosylation sequence that includes an amino acid residue having a hydroxyl group. The O-linked glycosylation sequence as part of the polypeptide is a substrate for a glucosamine transferase (e.g., GlcNAc-transferase). The polymeric modifying group is covalently linked to the polypeptide via a glucosamine-linking group interposed between and covalently linked to both the polypeptide and the modifying group. An exemplary method comprises: (i) contacting the polypeptide and a glucosamine-donor, which includes a glucosamine-moiety or a glucosamine-mimetic moiety covalently linked to the polymeric modifying group, in the presence of a glycosyltransferase (e.g., human GlcNAc-transferase) for which the glucosamine-donor is a substrate. The reaction is conducted under conditions sufficient for the glycosyltransferase to transfer the glucosamine moiety or glucosamine-mimetic moiety from the glucosamine donor onto said hydroxyl group of the O-linked glycosylation sequence.

Another exemplary method of forming a polypeptide conjugate of the invention includes the steps of: (i) recombinantly producing a polypeptide that includes an O-linked glycosylation sequence of the invention, and (ii) enzymatically transferring a glucosamine moiety or a glucosamine-mimetic moiety from a glucosamine-donor (e.g., a modified sugar nucleotide incorporating a GlcNAc or GlcNAc-mimetic moiety) onto a hydroxyl group of an amino acid side chain, wherein the amino acid is part of the O-linked glycosylation sequence.

In the methods above, the glucosamine-moiety can also be a glucosamine-mimetic moiety. In a preferred embodiment, the glucosamine transferase is a GlcNAc transferase. The glucosamine transferase is preferably a recombinant enzyme. In a particularly preferred embodiment, the GlcNAc transferase used in the methods of the invention is expressed in a bacterial host cell, such as E. coli.

In one embodiment, the polypeptide used in the methods of the invention is a wild-type polypeptide that naturally includes an O-linked glycosylation sequence. In another embodiment, the polypeptide is a non-naturally occurring polypeptide of the invention, derived from a parent-polypeptide, into which at least one O-linked glycosylation sequence has been introduced by mutation.

In one embodiment, the glucosamine-donor used in the methods of the invention has a structure according to Formula (XI), which is described herein, above with the difference that the donor is not required to incorporate a modifying group. In one embodiment, in Formula (XI), E and E¹ are both oxygen. In a particularly preferred embodiment, the glucosamine-donor is selected from modified or non-modified UDP-GlcNAc and modified or non-modified UDP-GlcNH.

Glycosylation or glycomodification steps may be performed separately, or combined in a “single pot” reaction using multiple enzymes and saccharyl donors. For example, a glycosidase, which is used to trim-off unwanted glycosyl residues from the expressed polypeptide and one or more glycosyltransferase as well as the respective glycosyl donor molecules may be combined in a single vessel. Another example involves adding each enzyme and an appropriate glycosyl donor sequentially conducting the reaction in a “single pot” motif. In one embodiment, time points of addition are interrupted by reaction time necessary for each enzyme to perform the desired enzymatic reaction. Combinations of the methods set forth above are also useful in preparing the compounds of the invention.

The present invention also provides means of adding (or removing) one or more selected glycosyl residues to a peptide, after which a modified sugar is conjugated to at least one of the selected glycosyl residues of the peptide. The present embodiment is useful, for example, when it is desired to conjugate the modified sugar to a selected glycosyl residue that is either not present on a peptide or is not present in a desired amount. Thus, prior to coupling a modified sugar to a peptide, the selected glycosyl residue is conjugated to the peptide by enzymatic or chemical coupling. In another embodiment, the glycosylation pattern of a glycopeptide is altered prior to the conjugation of the modified sugar by the removal of a carbohydrate residue from the glycopeptide. See, for example WO 98/31826.

Addition or removal of any carbohydrate moieties present on the glycopeptide is accomplished either chemically or enzymatically. Chemical deglycosylation is preferably brought about by exposure of the polypeptide to trifluoromethanesulfonic acid, or an equivalent compound. This treatment results in the cleavage of most or all sugars except the linking sugar (N-acetylglucosamine or N-acetylgalactosamine), while leaving the peptide intact. Chemical deglycosylation is described by Hakimuddin et al., Arch. Biochem. Biophys. 259: 52 (1987) and by Edge et al., Anal. Biochem. 118: 131 (1981). Enzymatic cleavage of carbohydrate moieties on polypeptide variants can be achieved by the use of a variety of endo- and exo-glycosidases as described by Thotakura et al., Meth. Enzymol. 138: 350 (1987).

Chemical addition of glycosyl moieties is carried out by any art-recognized method. Enzymatic addition of sugar moieties is preferably achieved using a modification of the methods set forth herein, substituting native glycosyl units for the modified sugars used in the invention. Other methods of adding sugar moieties are disclosed in U.S. Pat. Nos. 5,876,980; 6,030,815; 5,728,554 and 5,922,577. Exemplary methods of use in the present invention are described in WO 87/05330 published Sep. 11, 1987, and in Aplin and Wriston, CRC CRIT. REV. BIOCHEM., pp. 259-306 (1981).

### Polypeptide Conjugates Including Two or More Polypeptides

Also provided are conjugates that include two or more polypeptides linked together through a linker arm, i.e., multifunctional conjugates; at least one peptide being O-glycosylated or including a mutant O-linked glycosylation sequence. The multi-functional conjugates of the invention can include two or more copies of the same peptide or a collection of diverse peptides with different structures, and/or properties. In exemplary conjugates according to this embodiment, the linker between the two peptides is attached to at least one of the peptides through an O-linked glycosyl residue, such as an O-linked glycosyl intact glycosyl linking group.

In one embodiment, the invention provides a method for linking two or more peptides through a linking group. The linking group is of any useful structure and may be selected from straight- and branched-chain structures. Preferably, each terminus of the linker, which is attached to a peptide, includes a modified sugar (i.e., a nascent intact glycosyl linking group).

In an exemplary method of the invention, two peptides are linked together via a linker moiety that includes a PEG linker. The construct conforms to the general structure set forth in the cartoon above. As described herein, the construct of the invention includes two intact glycosyl linking groups (i.e., s+t=1). The focus on a PEG linker that includes two glycosyl groups is for purposes of clarity and should not be interpreted as limiting the identity of linker arms of use in this embodiment of the invention.

Thus, a PEG moiety is functionalized at a first terminus with a first glycosyl unit and at a second terminus with a second glycosyl unit. The first and second glycosyl units are preferably substrates for different transferases, allowing orthogonal attachment of the first and second peptides to the first and second glycosylunits, respectively. In practice, the (glycosyl)¹-PEG-(glycosyl)² linker is contacted with the first peptide and a first transferase for which the first glycosyl unit is a substrate, thereby forming (peptide)¹-(glycosyl)¹-PEG-(glycosyl). Transferase and/or unreacted peptide is then optionally removed from the reaction mixture. The second peptide and a second transferase for which the second glycosyl unit is a substrate are added to the (peptide)¹-(glycosyl)¹-PEG-(glycosyl)² conjugate, forming (peptide)¹-(glycosyl)¹-PEG-(glycosyl)²-(peptide)²; at least one of the glycosyl residues is either directly or indirectly O-linked. Those of skill in the art will appreciate that the method outlined above is also applicable to forming conjugates between more than two peptides by, for example, the use of a branched PEG, dendrimer, poly(amino acid), polsaccharide or the like

The processes described above can be carried through as many cycles as desired, and is not limited to forming a conjugate between two peptides with a single linker Moreover, those of skill in the art will appreciate that the reactions functionalizing the intact glycosyl linking groups at the termini of the PEG (or other) linker with the peptide can occur simultaneously in the same reaction vessel, or they can be carried out in a step-wise fashion. When the reactions are carried out in a step-wise manner, the conjugate produced at each step is optionally purified from one or more reaction components (e.g., enzymes, peptides).

### Enzymatic Conjugation of Modified Sugars to Peptides

The modified sugars are conjugated to a glycosylated or non-glycosylated peptide using an appropriate enzyme to mediate the conjugation. Preferably, the concentrations of the modified donor sugar(s), enzyme(s) and acceptor peptide(s) are selected such that glycosylation proceeds until the acceptor is consumed.

A number of methods of using glycosyltransferases to synthesize desired oligosaccharide structures are known and are generally applicable to the instant invention. Exemplary methods are described, for instance, in WO 96/32491 and Ito et al., Pure Appl. Chem. 65: 753 (1993), as well as U.S. Pat. Nos. 5,352,670; 5,374,541 and 5,545,553.

The present invention is practiced using a single enzyme (e.g., a glycosyltransferase) or a combination of glycosyltransferases and optionally one or more glycosidases. For example, one can use a combination of a glucosamine transferase and a galactosyltransferase. In those embodiments using more than one enzyme, the enzymes and substrates are preferably combined in an initial reaction mixture, or the enzymes and reagents for a second enzymatic reaction are added to the reaction medium once the first enzymatic reaction is complete or nearly complete. By conducting two enzymatic reactions in sequence in a single vessel, overall yields are improved over procedures in which an intermediate species is isolated. Moreover, cleanup and disposal of extra solvents and by-products is reduced.

The O-linked glycosyl moieties of the conjugates of the invention are generally originate with a glucosamine moiety that is attached to the peptide. Any member of the family of glucosamine transferases (e.g., GlcNAc transferases described herein, e.g., SEQ ID NOs: 1-9 and 228 to 230) can be used to bind a glucosamine moiety to the peptide (see e.g., Hassan H, Bennett E P, Mandel U, Hollingsworth M A, and Clausen H (2000); and Control of Mucin-Type O-Glycosylation: O-Glycan Occupancy is Directed by Substrate Specificities of Polypeptide GalNAc-Transferases; Eds. Ernst, Hart, and Sinay; Wiley-VCH chapter “Carbohydrates in Chemistry and Biology—a Comprehension Handbook”, 273-292). The GlcNAc moiety itself can be the glycosyl linking group and derivatized with a modifying group. Alternatively, the saccharyl residue is built out using one or more enzyme and one or more appropriate glycosyl donor substrate. The modified sugar may then be added to the extended glycosyl moiety.

The enzyme catalyzes the reaction, usually by a synthesis step that is analogous to the reverse reaction of the endoglycanase hydrolysis step. In these embodiments, the glycosyl donor molecule (e.g., a desired oligo- or mono-saccharide structure) contains a leaving group and the reaction proceeds with the addition of the donor molecule to a GlcNAc residue on the protein. For example, the leaving group can be a halogen, such as fluoride. In other embodiments, the leaving group is a Asn, or a Asn-peptide moiety. In yet further embodiments, the GlcNAc residue on the glycosyl donor molecule is modified. For example, the GlcNAc residue may comprise a 1,2 oxazoline moiety.

In another embodiment, each of the enzymes utilized to produce a conjugate of the invention are present in a catalytic amount. The catalytic amount of a particular enzyme varies according to the concentration of that enzyme's substrate as well as to reaction conditions such as temperature, time and pH value. Means for determining the catalytic amount for a given enzyme under preselected substrate concentrations and reaction conditions are well known to those of skill in the art.

The temperature at which an above process is carried out can range from just above freezing to the temperature at which the most sensitive enzyme denatures. Preferred temperature ranges are about 0° C. to about 55° C., and more preferably about 20° C. to about 32° C. In another exemplary embodiment, one or more components of the present method are conducted at an elevated temperature using a thermophilic enzyme.

The reaction mixture is maintained for a period of time sufficient for the acceptor to be glycosylated, thereby forming the desired conjugate. Some of the conjugate can often be detected after a few hours, with recoverable amounts usually being obtained within 24 hours or less. Those of skill in the art understand that the rate of reaction is dependent on a number of variable factors (e.g, enzyme concentration, donor concentration, acceptor concentration, temperature, solvent volume), which are optimized for a selected system.

The present invention also provides for the industrial-scale production of modified peptides. As used herein, an industrial scale generally produces at least about 250 mg, preferably at least about 500 mg, and more preferably at least about 1 gram of finished, purified conjugate, preferably after a single reaction cycle, i.e., the conjugate is not a combination the reaction products from identical, consecutively iterated synthesis cycles.

In the discussion that follows, the invention is exemplified by the conjugation of modified sialic acid moieties to a glycosylated peptide. The exemplary modified sialic acid is labeled with (m-) PEG. The focus of the following discussion on the use of PEG-modified sialic acid and glycosylated peptides is for clarity of illustration and is not intended to imply that the invention is limited to the conjugation of these two partners. One of skill understands that the discussion is generally applicable to the additions of modified glycosyl moieties other than sialic acid. Moreover, the discussion is equally applicable to the modification of a glycosyl unit with agents other than PEG including other water-soluble polymers, therapeutic moieties, and biomolecules.

An enzymatic approach can be used for the selective introduction of a modifying group (e.g., mPEG or mPPG) onto a peptide or glycopeptide. In one embodiment, the method utilizes modified sugars, which include the modifying group in combination with an appropriate glycosyltransferase or glycosynthase. By selecting the glycosyltransferase that will make the desired carbohydrate linkage and utilizing the modified sugar as the donor substrate, the modifying group can be introduced directly onto the peptide backbone, onto existing sugar residues of a glycopeptide or onto sugar residues that have been added to a peptide. In another embodiment, the method utilizes modified sugars, which carry a masked reactive functional group, which can be used for attachment of the modifying group after transfer of the modified sugar onto the peptide or glycopeptide.

In an exemplary embodiment, a GalNAc residue is added to an O-linked glycosylation sequence by the action of a GalNAc transferase. Hassan H, Bennett E P, Mandel U, Hollingsworth M A, and Clausen H (2000), Control of Mucin-Type O-Glycosylation: O-Glycan Occupancy is Directed by Substrate Specificities of Polypeptide GalNAc-Transferases (Eds. Ernst, Hart, and Sinay), Wiley-VCH chapter “Carbohydrates in Chemistry and Biology—a Comprehension Handbook”, pages 273-292. The method includes incubating the peptide to be modified with a reaction mixture that contains a suitable amount of a galactosyltransferase and a suitable galactosyl donor. The reaction is allowed to proceed substantially to completion or, alternatively, the reaction is terminated when a preselected amount of the galactose residue is added. Other methods of assembling a selected saccharide acceptor will be apparent to those of skill in the art.

In the discussion that follows, the method of the invention is exemplified by the use of modified sugars having a water-soluble polymer attached thereto. The focus of the discussion is for clarity of illustration. Those of skill will appreciate that the discussion is equally relevant to those embodiments in which the modified sugar bears a therapeutic moiety, a biomolecule or the like.

In another exemplary embodiment, a water-soluble polymer is added to a GlcNAc residue via a modified GlcNAc or GlcNH residue, galactosyl (Gal) residue, fucosyl residue (Fuc), sialyl residue (Sia) or mannosyl (Man) residue. Alternatively, an unmodified glycosyl residue can be added to the terminal GlcNAc residue.

In yet a further example, a water-soluble polymer (e.g., PEG) is added onto a terminal GlcNAc residue using a modified GlcNAc, Gal, Sia, Fuc or Man moiety and an appropriate transferase.

In yet a further approach, a masked reactive functionality is present on the transferred glycosyl residue. The masked reactive group is preferably unaffected by the conditions used to attach the modified sugar to the peptide. After the covalent attachment of the modified sugar to the peptide, the mask is removed and the peptide is conjugated to the modifying group, such as a water soluble polymer (e.g., PEG or PPG) by reaction of the unmasked reactive group on the modified sugar residue with a reactive modifying group.

In an alternative embodiment, the modified sugar is added directly to the peptide backbone using a glycosyltransferase known to transfer sugar residues to the O-linked glycosylation sequence on the peptide backbone. Exemplary glycosyltransferases useful in practicing the present invention include, but are not limited to, GlcNAc transferasese, and the like. Use of this approach allows for the direct addition of modified sugars onto peptides that lack any carbohydrates. In a preferred embodiment, the modified sugar nucleotide is modified UDP-glucosamine and the glycosyltransferase is a GlcNAc transferase. This exemplary embodiment is set forth in Scheme 5, below.

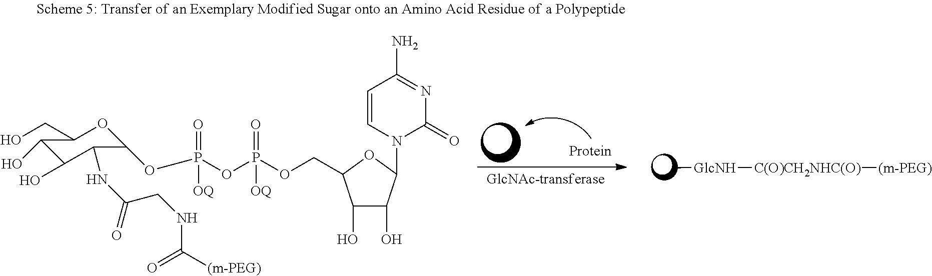

In another exemplary embodiment, the glycopeptide is conjugated to a targeting agent, e.g., transferrin (to deliver the peptide across the blood-brain barrier, and to endosomes), carnitine (to deliver the peptide to muscle cells; see, for example, LeBorgne et al., Biochem. Pharmacol. 59: 1357-63 (2000), and phosphonates, e.g., bisphosphonate (to target the peptide to bone and other calciferous tissues; see, for example, Modern Drug Discovery, August 2002, page 10). Other agents useful for targeting are apparent to those of skill in the art. For example, glucose, glutamine and IGF are also useful to target muscle.

The targeting moiety and therapeutic peptide are conjugated by any method discussed herein or otherwise known in the art. Those of skill will appreciate that peptides in addition to those set forth above can also be derivatized as set forth herein. Exemplary peptides are set forth in the Appendix attached to copending, commonly owned U.S. Provisional Patent Application No. 60/328,523 filed Oct. 10, 2001.

In an exemplary embodiment, the targeting agent and the therapeutic peptide are coupled via a linker moiety. In this embodiment, at least one of the therapeutic peptide or the targeting agent is coupled to the linker moiety via an intact glycosyl linking group according to a method of the invention. In an exemplary embodiment, the linker moiety includes a poly(ether) such as poly(ethylene glycol). In another exemplary embodiment, the linker moiety includes at least one bond that is degraded in vivo, releasing the therapeutic peptide from the targeting agent, following delivery of the conjugate to the targeted tissue or region of the body.

In yet another exemplary embodiment, the in vivo distribution of the therapeutic moiety is altered via altering a glycoform on the therapeutic moiety without conjugating the therapeutic peptide to a targeting moiety. For example, the therapeutic peptide can be shunted away from uptake by the reticuloendothelial system by capping a terminal galactose moiety of a glycosyl group with sialic acid (or a derivative thereof).

### Enzymes

### Glycosyltransferases

The glycosyltransferase to be used in the present invention may be any as long as it can utilize the modified sugar as a sugar donor. Examples of such enzymes include Leloir pathway glycosyltransferase, such as galactosyltransferase, N-acetylglucosaminyltransferase, N-acetylgalactosaminyltransferase, fucosyltransferase, sialyltransferase, mannosyltransferase, xylosyltransferase, glucurononyltransferase and the like.

For enzymatic saccharide syntheses that involve glycosyltransferase reactions, glycosyltransferase can be cloned, or isolated from any source. Many cloned glycosyltransferases are known, as are their polynucleotide sequences. Glycosyltransferase amino acid sequences and nucleotide sequences encoding glycosyltransferases from which the amino acid sequences can be deduced are found in various publicly available databases, including GenBank, Swiss-Prot, EMBL, and others.

Glycosyltransferases that can be employed in the methods of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, and oligosaccharyltransferases. Suitable glycosyltransferases include those obtained from eukaryotes, as well as from prokaryotes.

DNA encoding glycosyltransferases may be obtained by chemical synthesis, by screening reverse transcripts of mRNA from appropriate cells or cell line cultures, by screening genomic libraries from appropriate cells, or by combinations of these procedures. Screening of mRNA or genomic DNA may be carried out with oligonucleotide probes generated from the glycosyltransferases gene sequence. Probes may be labeled with a detectable group such as a fluorescent group, a radioactive atom or a chemiluminescent group in accordance with known procedures and used in conventional hybridization assays. In the alternative, glycosyltransferases gene sequences may be obtained by use of the polymerase chain reaction (PCR) procedure, with the PCR oligonucleotide primers being produced from the glycosyltransferases gene sequence (See, for example, U.S. Pat. No. 4,683,195 to Mullis et al. and U.S. Pat. No. 4,683,202 to Mullis).

The glycosyltransferase may be synthesized in host cells transformed with vectors containing DNA encoding the glycosyltransferases enzyme. Vectors are used either to amplify DNA encoding the glycosyltransferases enzyme and/or to express DNA which encodes the glycosyltransferases enzyme. An expression vector is a replicable DNA construct in which a DNA sequence encoding the glycosyltransferases enzyme is operably linked to suitable control sequences capable of effecting the expression of the glycosyltransferases enzyme in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences which control the termination of transcription and translation. Amplification vectors do not require expression control domains. All that is needed is the ability to replicate in a host, usually conferred by an origin of replication, and a selection gene to facilitate recognition of transformants.

In an exemplary embodiment, the invention utilizes a prokaryotic enzyme. Such glycosyltransferases include enzymes involved in synthesis of lipooligosaccharides (LOS), which are produced by many gram negative bacteria (Preston et al., Critical Reviews in Microbiology 23(3): 139-180 (1996)). Such enzymes include, but are not limited to, the proteins of the rfa operons of species such as E. coli and Salmonella typhimurium, which include a β1,6 galactosyltransferase and a β1,3 galactosyltransferase (see, e.g., EMBL Accession Nos. M80599 and M86935 (E. coli); EMBL Accession No. S56361 (S. typhimurium)), a glucosyltransferase (Swiss-Prot Accession No. P25740 (E. coli), an β1,2-glucosyltransferase (rfaJ) (Swiss-Prot Accession No. P27129 (E. coli) and Swiss-Prot Accession No. P19817 (S. typhimurium)), and an β1,2-N-acetylglucosaminyltransferase (rfaK) (EMBL Accession No. U00039 (E. coli). Other glycosyltransferases for which amino acid sequences are known include those that are encoded by operons such as rfaB, which have been characterized in organisms such as Klebsiella pneumoniae, E. coli, Salmonella typhimurium, Salmonella enterica, Yersinia enterocolitica, Mycobacterium leprosum, and the rhl operon of Pseudomonas aeruginosa.

Also suitable for use in the present invention are glycosyltransferases that are involved in producing structures containing lacto-N-neotetraose, D-galactosyl-β-1,4-N-acetyl-D-glucosaminyl-β-1,3-D-galactosyl-β-1,4-D-glucose, and the P^{k} blood group trisaccharide sequence, D-galactosyl-α-1,4-D-galactosyl-β-1,4-D-glucose, which have been identified in the LOS of the mucosal pathogens Neisseria gonnorhoeae and N. meningitidis (Scholten et al., J. Med. Microbiol. 41: 236-243 (1994)). The genes from N. meningitidis and N. gonorrhoeae that encode the glycosyltransferases involved in the biosynthesis of these structures have been identified from N. meningitidis immunotypes L3 and L1 (Jennings et al., Mol. Microbiol. 18: 729-740 (1995)) and the N. gonorrhoeae mutant F62 (Gotshlich, J. Exp. Med. 180: 2181-2190 (1994)). In N. meningitidis, a locus consisting of three genes, lgtA, lgtB and lg E, encodes the glycosyltransferase enzymes required for addition of the last three of the sugars in the lacto-N-neotetraose chain (Wakarchuk et al., J. Biol. Chem. 271: 19166-73 (1996)). Recently the enzymatic activity of the lgtB and lgtA gene product was demonstrated, providing the first direct evidence for their proposed glycosyltransferase function (Wakarchuk et al., J. Biol. Chem. 271(45): 28271-276 (1996)). In N. gonorrhoeae, there are two additional genes, lgtD which adds β-D-GalNAc to the 3 position of the terminal galactose of the lacto-N-neotetraose structure and lgtC which adds a terminal α-D-Gal to the lactose element of a truncated LOS, thus creating the P^{k} blood group antigen structure (Gotshlich (1994), supra.). In N. meningitidis, a separate immunotype L1 also expresses the P^{k} blood group antigen and has been shown to carry an lgtC gene (Jennings et al., (1995), supra.). Neisseria glycosyltransferases and associated genes are also described in U.S. Pat. No. 5,545,553 (Gotschlich). Genes for α1,2-fucosyltransferase and α1,3-fucosyltransferase from Helicobacter pylori has also been characterized (Martin et al., J. Biol. Chem. 272: 21349-21356 (1997)). Also of use in the present invention are the glycosyltransferases of Campylobacter jejuni (see, for example, http://afmb.cnrs-mrs.fr/˜pedro/CAZY/gtf 42.html).

### (a) N-Acetylglucosamine Transferases

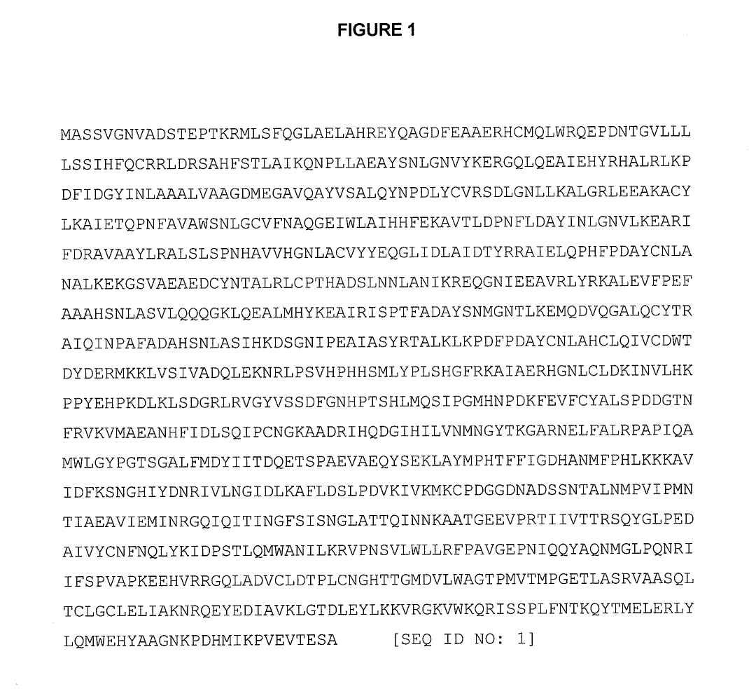

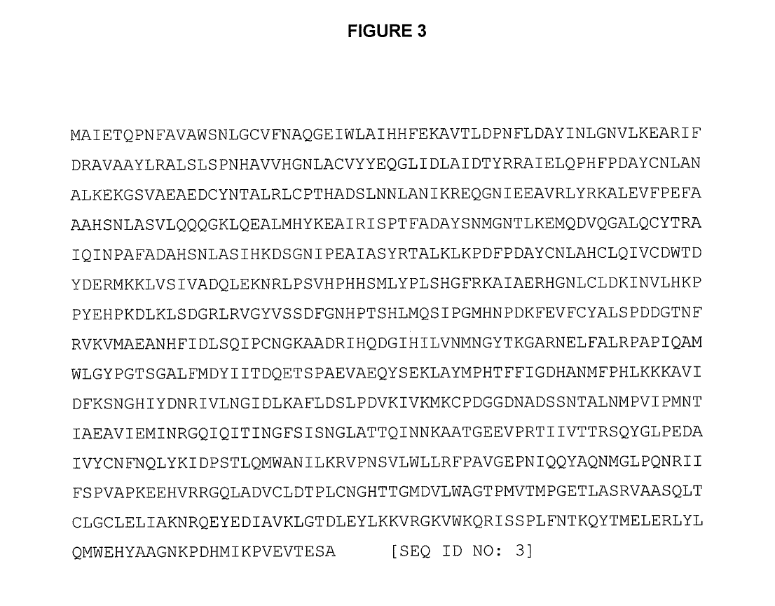

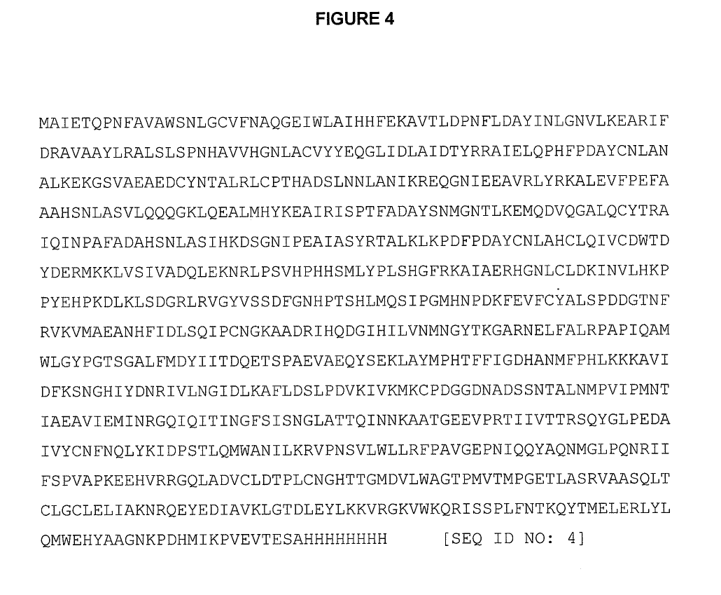

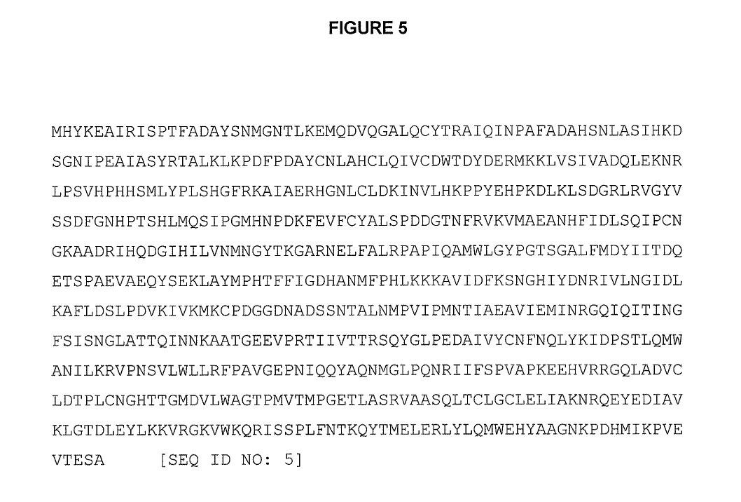

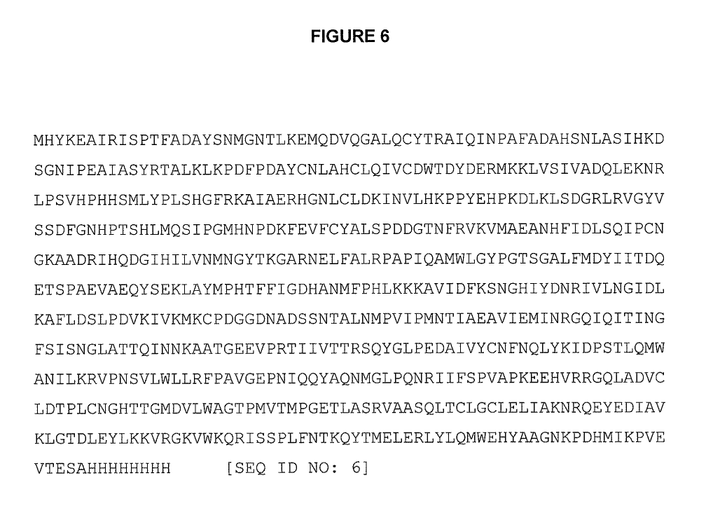

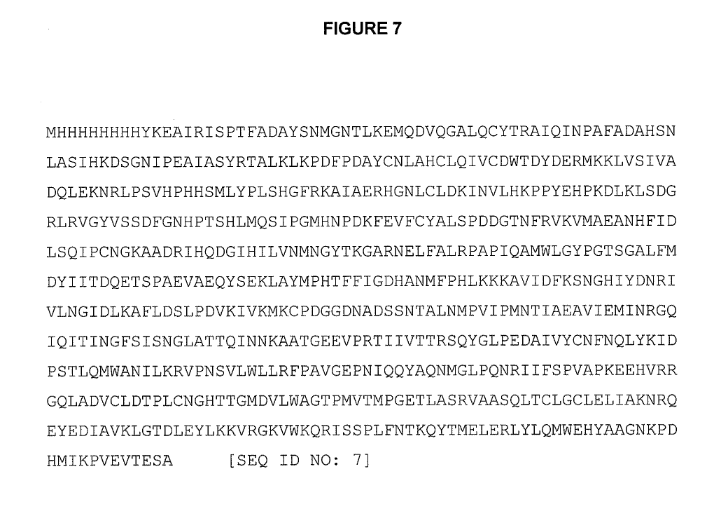

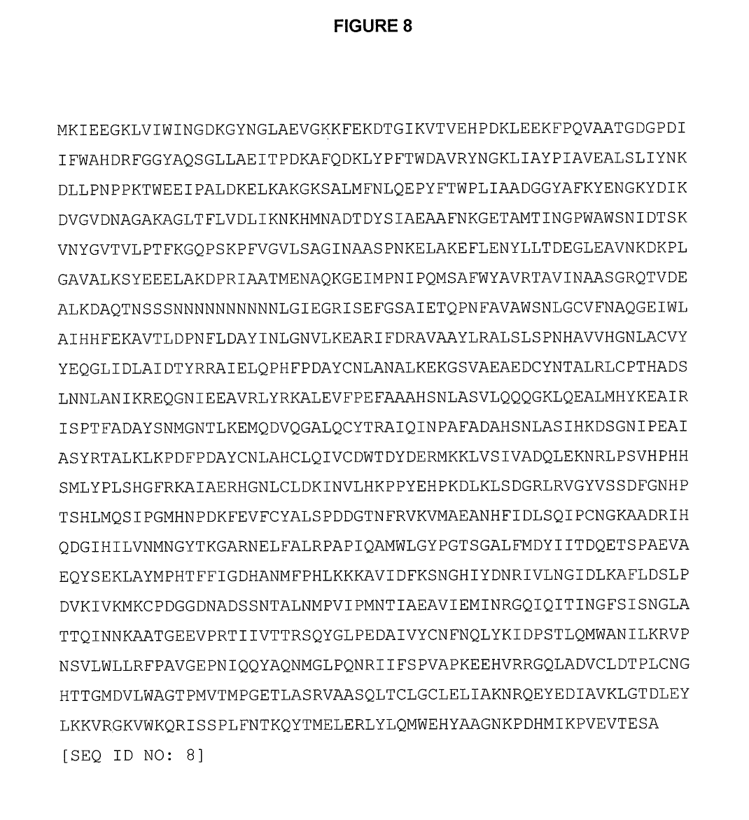

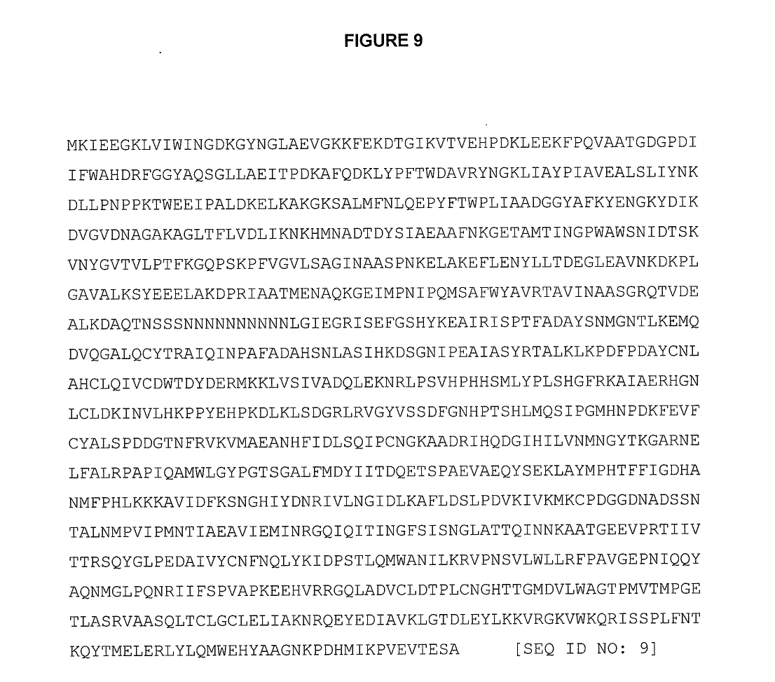

In some embodiments, the glycosyltransferase is an N-acetylglucosamine transferase, such as uridine diphospho-N-acetylglucosamine:polypeptide β-N-acetylglucosaminyltransferase described, for example in Kreppel et al., J. Biol. Chem. 1997, 272: 9308-9315 and Lubas et al., J. Biol. Chem. 1997, 272: 9316-9324. Other exemplary GlcNAc transferases are disclosed in Kreppel, L. and G. Hart, J. Biol. Chem. 1999, 274: 32015-32022; Lubas, W. and J. Hanover, J. Biol. Chem. 2000, 275: 10983-10988; Hanover, J. et al., Arch. Biochem. Biophys. 2003, 409: 287-297; Gross, B., Kraybill, B., and S. Walker, J. Am. Chem. Soc. 2005, 127: 14588-14589 and Gross, B., Swoboda, J., and S. Walker, J. Am. Chem. Soc. 2008, 130: 440-441, the disclosures of which are incorporated herein by reference in their entirety. Exemplary glucosamine transferases include GnT-I to GnT-VI. Exemplary amino acid sequences for GlcNAc transferases useful in the methods of the invention are shown, e.g., in FIGS. 1 to 9 (SEQ ID NOs: 1 to 9) and herein below (SEQ ID NOs: 228 to 230):

Sequence of Human GlcNAc transferase isoform 1 (NP858058)
(SEQ ID NO: 228)
MASSVGNVADSTEPTKRMLSFQGLAELAHREYQAGDFEAAERHCMQLWRQEPDNTGVLLLLSSIHFQ
CRRLDRSAHFSTLAIKQNPLLAEAYSNLGNVYKERGQLQEAIEHYRHALRLKPDFIDGYINLAAALVAA
GDMEGAVQAYVSALQYNPDLYCVRSDLGNLLKALGRLEEAKACYLKAIETQPNFAVAWSNLGCVFN
AQGEIWLAIHHFEKAVTLDPNFLDAYINLGNVLKEARIFDRAVAAYLRALSLSPNHAVVHGNLACVYY
EQGLIDLAIDTYRRAIELQPHFPDAYCNLANALKEKGSVAEAEDCYNTALRLCPTHADSLNNLANIKRE
QGNIEEAVRLYRKALEVFPEFAAAHSNLASVLQQQGKLQEALMHYKEAIRISPTFADAYSNMGNTLKE
MQDVQGALQCYTRAIQINPAFADAHSNLASIHKDSGNIPEAIASYRTALKLKPDFPDAYCNLAHCLQIV
CDWTDYDERMKKLVSIVADQLEKNRLPSVHPHHSMLYPLSHGFRKAIAERHGNLCLDKINVLHKPPYE
HPKDLKLSDGRLRVGYVSSDFGNHPTSHLMQSIPGMHNPDKFEVFCYALSPDDGTNFRVKVMAEANH
FIDLSQIPCNGKAADRIHQDGIHILVNMNGYTKGARNELFALRPAPIQAMWLGYPGTSGALFMDYHTD
QETSPAEVAEQYSEKLAYMPHTFFIGDHANMFPHLKKKAVIDFKSNGHIYDNRIVLNGIDLKAFLDSLP
DVKIVKMKCPDGGDNADSSNTALNMPVIPMNTIAEAVIEMINRGQIQITINGFSISNGLATTQINNKAAT
GEEVPRTIIVTTRSQYGLPEDAIVYCNFNQLYKIDPSTLQMWANILKRVPNSVLWLLRFPAVGEPNIQQY
AQNMGLPQNRIIFSPVAPKEEHVRRGQLADVCLDTPLCNGHTTGMDVLWAGTPMVTMPGETLASRVA
ASQLTCLGCLELIAKNRQEYEDIAVKLGTDLEYLKKVRGKVWKQRISSPLFNTKQYTMELERLYLQM
WEHYAAGNKPDHMIKPVEVTESA
Sequence of Human GlcNAc transferase isoform 2 (NP858059)
(SEQ ID NO: 229)
MASSVGNVADSTGLAELAHREYQAGDFEAAERHCMQLWRQEPDNTGVLLLLSSIHFQCRRLDRSAHF
STLAIKQNPLLAEAYSNLGNVYKERGQLQEAIEHYRHALRLKPDFIDGYINLAAALVAAGDMEGAVQA
YVSALQYNPDLYCVRSDLGNLLKALGRLEEAKACYLKAIETQPNFAVAWSNLGCVFNAQGEIWLAIH
HFEKAVTLDPNFLDAYINLGNVLKEARIFDRAVAAYLRALSLSPNHAVVHGNLACVYYEQGLIDLAID
TYRRAIELQPHFPDAYCNLANALKEKGSVAEAEDCYNTALRLCPTHADSLNNLANIKREQGNIEEAVR
LYRKALEVFPEFAAAHSNLASVLQQQGKLQEALMHYKEAIRISPTFADAYSNMGNTLKEMQDVQGAL
QCYTRAIQINPAFADAHSNLASIHKDSGNIPEAIASYRTALKLKPDFPDAYCNLAHCLQIVCDWTDYDE
RMKKLVSIVADQLEKNRLPSVHPHHSMLYPLSHGFRKAIAERHGNLCLDKINVLHKPPYEHPKDLKLS
DGRLRVGYVSSDFGNHPTSHLMQSIPGMHNPDKFEVFCYALSPDDGTNFRVKVMAEANHFIDLSQIPC
NGKAADRIHQDGIHILVNMNGYTKGARNELFALRPAPIQAMWLGYPGTSGALFMDYIITDQETSPAEV
AEQYSEKLAYMPHTFFIGDHANMFPHLKKKAVIDFKSNGHIYDNRIVLNGIDLKAFLDSLPDVKIVKMK
CPDGGDNADSSNTALNMPVIPMNTIAEAVIEMINRGQIQITINGFSISNGLATTQINNKAATGEEVPRTIIV
TTRSQYGLPEDAIVYCNFNQLYKIDPSTLQMWANILKRVPNSVLWLLRFPAVGEPNIQQYAQNMGLPQ
NRIIFSPVAPKEEHVRRGQLADVCLDTPLCNGHTTGMDVLWAGTPMVTMPGETLASRVAASQLTCLG
CLELIAKNRQEYEDIAVKLGTDLEYLKKVRGKVWKQRISSPLFNTKQYTMELERLYLQMWEHYAAGN
KPDHMIKPVEVTESA
Sequence of Human GlcNAc transferase isoform CRA_a (EAX05285
CH471132.2)
(SEQ ID NO: 230)
MLQGHFWLVREGIMISPSSPPPPNLFFFPLQIFPFPFTSFPSHLLSLTPPKACYLKAIETQPNFAVAWSNLG
CVFNAQGEIWLAIHHFEKAVTLDPNFLDAYINLGNVLKEARIFDRAVAAYLRALSLSPNHAVVHGNLA
CVYYEQGLIDLAIDTYRRAIELQPHFPDAYCNLANALKEKGSVAEAEDCYNTALRLCPTHADSLNNLA
NIKREQGNIEEAVRLYRKALEVFPEFAAAHSNLASVLQQQGKLQEALMHYKEAIRISPTFADAYSNMG
NTLKEMQDVQGALQCYTRAIQINPAFADAHSNLASIHKDSGNIPEAIASYRTALKLKPDFPDAYCNLAH
CLQIVCDWTDYDERMKKLVSIVADQLEKNRLPSVHPHHSMLYPLSHGFRKAIAERHGNLCLDKINVLH
KPPYEHPKDLKLSDGRLRVGYVSSDFGNHPTSHLMQSIPGMHNPDKFEVFCYALSPDDGTNFRVKVM
AEANHFIDLSQIPCNGKAADRIHQDGIHILVNMNGYTKGARNELFALRPAPIQAMWLGYPGTSGALFM
DYIITDQETSPAEVAEQYSEKLAYMPHTFFIGDHANMFPHLKKKAVIDFKSNGHIYDNRIVLNGIDLKAF
LDSLPDVKIVKMKCPDGGDNADSSNTALNMPVIPMNTIAEAVIEMINRGQIQITINGFSISNGLATTQINN
KAATGEEVPRTIIVTTRSQYGLPEDAIVYCNFNQLYKIDPSTLQMWANILKRVPNSVLWLLRFPAVGEP
NIQQYAQNMGLPQNRIIFSPVAPKEEHVRRGQLADVCLDTPLCNGHTTGMDVLWAGTPMVTMPGETL
ASRVAASQLTCLGCLELIAKNRQEYEDIAVKLGTDLEYLKKVRGKVWKQRISSPLFNTKQYTMELERL
YLQMWEHYAAGNKPDHMIKPVEVTESA

Other glucosamine transferases, for example those originating from other organisms, such as other mammals (e.g., murine, bovine, porcine, rat), insects (drosophila sp.), yeast (e.g., candida sp.), bacteria (e.g., E. coli) and C. elegans are also useful within the methods of the invention. In addition, any mutated or truncated form of the above glucosamine transferases (SEQ ID NOs: 228 to 230) or of any other glucosamine transferase are also useful within the methods of the current invention. In one embodiment, the GlcNAc transferase lacks one or more tetratricopeptide repeat (TPR) domain. Particularly preferred are those enzymes, which are capable of adding only one glucosamine moiety per O-linked glycosylation sequence and those, which are essentially specific for a particular O-linked glycosylation sequence of the invention.

### (b) GalNAc Transferases

The first step in O-linked glycosylation can be catalyzed by one or more members of a large family of UDP-GalNAc: polypeptide N-acetylgalactosaminyltransferases (GalNAc-transferases), which normally transfer GalNAc to serine and threonine acceptor sites (Hassan et al., J. Biol. Chem. 275: 38197-38205 (2000)). To date twelve members of the mammalian GalNAc-transferase family have been identified and characterized (Schwientek et al., J. Biol. Chem. 277: 22623-22638 (2002)), and several additional putative members of this gene family have been predicted from analysis of genome databases. The GalNAc-transferase isoforms have different kinetic properties and show differential expression patterns temporally and spatially, suggesting that they have distinct biological functions (Hassan et al., J. Biol. Chem. 275: 38197-38205 (2000)). Sequence analysis of GalNAc-transferases have led to the hypothesis that these enzymes contain two distinct subunits: a central catalytic unit, and a C-terminal unit with sequence similarity to the plant lectin ricin, designated the “lectin domain” (Hagen et al., J. Biol. Chem. 274: 6797-6803 (1999); Hazes, Protein Eng. 10: 1353-1356 (1997); Breton et al., Curr. Opin. Struct. Biol. 9: 563-571 (1999)). Previous experiments involving site-specific mutagenesis of selected conserved residues confirmed that mutations in the catalytic domain eliminated catalytic activity. In contrast, mutations in the “lectin domain” had no significant effects on catalytic activity of the GalNAc-transferase isoform, GalNAc-T1 (Tenno et al., J. Biol. Chem. 277(49): 47088-96 (2002)). Thus, the C-terminal “lectin domain” was believed not to be functional and not to play roles for the enzymatic functions of GalNAc-transferases (Hagen et al., J. Biol. Chem. 274: 6797-6803 (1999)).

Polypeptide GalNAc-transferases, which have not displayed apparent GalNAc-glycopeptide specificities, also appear to be modulated by their putative lectin domains (PCT WO 01/85215 A2). Recently, it was found that mutations in the GalNAc-T1 putative lectin domain, similarly to those previously analysed in GalNAc-T4 (Hassan et al., J. Biol. Chem. 275: 38197-38205 (2000)), modified the activity of the enzyme in a similar fashion as GalNAc-T4. Thus, while wild type GalNAc-T1 added multiple consecutive GalNAc residues to a peptide substrate with multiple acceptor sites, mutated GalNAc-T1 failed to add more than one GalNAc residue to the same substrate (Tenno et al., J. Biol. Chem. 277(49): 47088-96 (2002)). More recently, the x-ray crystal structures of murine GalNAc-T1 (Fritz et al., PNAS 2004, 101(43): 15307-15312) as well as human GalNAc-T2 (Fritz et al., J. Biol. Chem. 2006, 281(13):8613-8619) have been determined. The human GalNAc-T2 structure revealed an unexpected flexibility between the catalytic and lectin domains and suggested a new mechanism used by GalNAc-T2 to capture glycosylated substrates. Kinetic analysis of GalNAc-T2 lacking the lectin domain confirmed the importance of this domain in acting on glycopeptide substrates. However, the enzymes activity with respect to non-glycosylated substrates was not significantly affected by the removal of the lectin domain. Thus, truncated human GalNAc-T2 enzymes lacking the lectin domain can be useful for the glycosylation of peptide substrates where further glycosylation of the resulting mono-glycosylated peptide is not desired.

Recent evidence demonstrates that some GalNAc-transferases exhibit unique activities with partially GalNAc-glycosylated glycopeptides. The catalytic actions of at least three GalNAc-transferase isoforms, GalNAc-T4, -T7, and -T10, selectively act on glycopeptides corresponding to mucin tandem repeat domains where only some of the clustered potential glycosylation sequences have been GalNAc glycosylated by other GalNAc-transferases (Bennett et al., FEBS Letters 460: 226-230 (1999); Ten Hagen et al., J. Biol. Chem. 276: 17395-17404 (2001); Bennett et al., J. Biol. Chem. 273: 30472-30481 (1998); Ten Hagen et al., J. Biol. Chem. 274: 27867-27874 (1999)). GalNAc-T4 and -T7 recognize different GalNAc-glycosylated peptides and catalyse transfer of GalNAc to acceptor substrate sites in addition to those that were previously utilized. One of the functions of such GalNAc-transferase activities is predicted to represent a control step of the density of O-glycan occupancy in glycoproteins with high density of O-linked glycosylation.

One example of this is the glycosylation of the cancer-associated mucin MUC1. MUC1 contains a tandem repeat O-linked glycosylated region of 20 residues (HGVTSAPDTRPAPGSTAPPA) with five potential O-linked glycosylation sequences. GalNAc-T1, -T2, and -T3 can initiate glycosylation of the MUC1 tandem repeat and incorporate at only three sites (HGVTSAPDTRPAPGSTAPPA (SEQ ID NO: 231), GalNAc attachment sites underlined). GalNAc-T4 is unique in that it is the only GalNAc-transferase isoform identified so far that can complete the O-linked glycan attachment to all five acceptor sites in the 20 amino acid tandem repeat sequence of the breast cancer associated mucin, MUC1. GalNAc-T4 transfers GalNAc to at least two sites not used by other GalNAc-transferase isoforms on the GalNAc₄TAP24 glycopeptide (TAPPAHGVTSAPDTRPAPGSTAPP (SEQ ID NO: 232), unique GalNAc-T4 attachment sites are in bold) (Bennett et al., J. Biol. Chem. 273: 30472-30481 (1998). An activity such as that exhibited by GalNAc-T4 appears to be required for production of the glycoform of MUC1 expressed by cancer cells where all potential sites are glycosylated (Muller et al., J. Biol. Chem. 274: 18165-18172 (1999)). Normal MUC1 from lactating mammary glands has approximately 2.6 O-linked glycans per repeat (Muller et al., J. Biol. Chem. 272: 24780-24793 (1997) and MUC1 derived from the cancer cell line T47D has 4.8 O-linked glycans per repeat (Muller et al., J. Biol. Chem. 274: 18165-18172 (1999)). The cancer-associated form of MUC1 is therefore associated with higher density of O-linked glycan occupancy and this is accomplished by a GalNAc-transferase activity identical to or similar to that of GalNAc-T4. Another enzyme, GalNAc-T11 is described, for example, in T. Schwientek et al., J. Biol. Chem. 2002, 277 (25):22623-22638.

Production of proteins such as the enzyme GalNAc T_{I-XX} from cloned genes by genetic engineering is well known. See, e.g., U.S. Pat. No. 4,761,371. One method involves collection of sufficient samples, then the amino acid sequence of the enzyme is determined by N-terminal sequencing. This information is then used to isolate a cDNA clone encoding a full-length (membrane bound) transferase which upon expression in the insect cell line Sf9 resulted in the synthesis of a fully active enzyme. The acceptor specificity of the enzyme is then determined using a semiquantitative analysis of the amino acids surrounding known glycosylation sequences in 16 different proteins followed by in vitro glycosylation studies of synthetic peptides. This work has demonstrated that certain amino acid residues are overrepresented in glycosylated peptide segments and the residues in specific positions surrounding glycosylated serine and threonine residues may have a more marked influence on acceptor efficiency than other amino acid moieties.

Since it has been demonstrated that mutations of GalNAc transferases can be utilized to produce glycosylation patterns that are distinct from those produced by the wild-type enzymes, it is within the scope of the present invention to utilize one or more mutant or truncated GalNAc transferase in preparing the O-linked glycosylated polypeptides of the invention. Catalytic domains and truncation mutants of GalNAc-T2 proteins are described, for example, in U.S. Provisional Patent Application 60/576,530 filed Jun. 3, 2004; and U.S. Provisional Patent Application 60/598,584, filed Aug. 3, 2004; both of which are herein incorporated by reference for all purposes. Catalytic domains can also be identified by alignment with known glycosyltransferases. Truncated GalNAc-T2 enzymes, such as human GalNAc-T2 (Δ51), human GalNAc-T2 (Δ51 Δ445) and methods of obtaining those enzymes are also described in WO 06/102652 (PCT/US06/011065, filed Mar. 24, 2006) and PCT/US05/00302, filed Jan. 6, 2005, which are herein incorporated by reference for all purposes.

### (c) Fucosyltransferases

In some embodiments, a glycosyltransferase used in the method of the invention is a fucosyltransferase. Fucosyltransferases are known to those of skill in the art. Exemplary fucosyltransferases include enzymes, which transfer L-fucose from GDP-fucose to a hydroxy position of an acceptor sugar. Fucosyltransferases that transfer non-nucleotide sugars to an acceptor are also of use in the present invention.

In some embodiments, the acceptor sugar is, for example, the GlcNAc in a Galβ(1→3,4)GlcNAcβ-group in an oligosaccharide glycoside. Suitable fucosyltransferases for this reaction include the Galβ(1→3,4)GlcNAcβ1-α(1→3,4)fucosyltransferase (FTIII E.C. No. 2.4.1.65), which was first characterized from human milk (see, Palcic, et al., Carbohydrate Res. 190:1-11 (1989); Prieels, et al., J. Biol. Chem. 256: 10456-10463 (1981); and Nunez, et al., Can. J. Chem. 59: 2086-2095 (1981)) and the Galβ(1→4)GlcNAcβ-αfucosyltransferases (FTIV, FTV, FTVI) which are found in human serum. FTVII (E.C. No. 2.4.1.65), a sialyl α(2→3)Galβ((1→3)GlcNAcβ fucosyltransferase, has also been characterized. A recombinant form of the Galβ(1→3,4) GlcNAcβ-α(1→3,4)fucosyltransferase has also been characterized (see, Dumas, et al., Bioorg. Med. Letters 1: 425-428 (1991) and Kukowska-Latallo, et al., Genes and Development 4: 1288-1303 (1990)). Other exemplary fucosyltransferases include, for example, α1,2 fucosyltransferase (E.C. No. 2.4.1.69). Enzymatic fucosylation can be carried out by the methods described in Mollicone, et al., Eur. J. Biochem. 191: 169-176 (1990) or U.S. Pat. No. 5,374,655. Cells that are used to produce a fucosyltransferase will also include an enzymatic system for synthesizing GDP-fucose.

### (d) Galactosyltransferases

In another group of embodiments, the glycosyltransferase is a galactosyltransferase. Exemplary galactosyltransferases include α(1,3) galactosyltransferases (E.C. No. 2.4.1.151, see, e.g., Dabkowski et al., Transplant Proc. 25:2921 (1993) and Yamamoto et al. Nature 345: 229-233 (1990), bovine (GenBank j04989, Joziasse et al., J. Biol. Chem. 264: 14290-14297 (1989)), murine (GenBank m26925; Larsen et al., Proc. Nat'l. Acad. Sci. USA 86: 8227-8231 (1989)), porcine (GenBank L36152; Strahan et al., Immunogenetics 41: 101-105 (1995)). Another suitable α1,3 galactosyltransferase is that which is involved in synthesis of the blood group B antigen (EC 2.4.1.37, Yamamoto et al., J. Biol. Chem. 265: 1146-1151 (1990) (human)). Also suitable in the practice of the invention are soluble forms of α1,3-galactosyltransferase such as that reported by Cho, S. K. and Cummings, R. D. (1997) J. Biol. Chem., 272, 13622-13628.

In another embodiment, the galactosyltransferase is a β(1,3)-galactosyltransferases, such as Core-1-GalT1. Human Core-1-β1,3-galactosyltransferase has been described (see, e.g., Ju et al., J. Biol. Chem. 2002, 277(1): 178-186). Drosophila melanogaster enzymes are described in Correia et al., PNAS 2003, 100(11): 6404-6409 and Muller et al., FEBS J. 2005, 272(17): 4295-4305. Additional Core-1-β3 galactosyltransferases, including truncated versions thereof, are disclosed in WO/0144478 and U.S. Provisional Patent Application No. 60/842,926 filed Sep. 6, 2006. In an exemplary embodiment, the β(1,3)-galactosyltransferase is a member selected from enzymes described by PubMed Accession Number AAF52724 (transcript of CG9520-PC) and modified versions thereof, such as those variations, which are codon optimized for expression in bacteria. The sequence of an exemplary, soluble Core-1-GalT1 (Core-1-GalT1 Δ31) enzyme is shown below:

Sequence of Core-1-GalT1 Δ31

(SEQ ID NO: 233)

GFCLAELFVYSTPERSEFMPYDGHRHGDVNDAHHSHDMMEMSGPEQDVG
GHEHVHENSTIAERLYSEVRVLCWIMTNPSNHQKKARHVKRTWGKRCNK
LIFMSSAKDDELDAVALPVGEGRNNLWGKTKEAYKYIYEHHINDADWFL
KADDDTYTIVENMRYMLYPYSPETPVYFGCKFKPYVKQGYMSGGAGYVL
SREAVRRFVVEALPNPKLCKSDNSGAEDVEIGKCLQNVNVLAGDSRDSN
GRGRFFPFVPEHHLIPSHTDKKFWYWQYIFYKTDEGLDCCSDNAISFHY
VSPNQMYVLDYLIYHLRPYGIINTPDALPNKLAVGELMPEIKEQATEST
SDGVSKRSAETKTQ

Also suitable for use in the methods of the invention are β(1,4) galactosyltransferases, which include, for example, EC 2.4.1.90 (LacNAc synthetase) and EC 2.4.1.22 (lactose synthetase) (bovine (D'Agostaro et al., Eur. J. Biochem. 183: 211-217 (1989)), human (Masri et al., Biochem. Biophys. Res. Commun. 157: 657-663 (1988)), murine (Nakazawa et al., J. Biochem. 104: 165-168 (1988)), as well as E.C. 2.4.1.38 and the ceramide galactosyltransferase (EC 2.4.1.45, Stahl et al., J. Neurosci. Res. 38: 234-242 (1994)). Other suitable galactosyltransferases include, for example, α1,2 galactosyltransferases (from e.g., Schizosaccharomyces pombe, Chapell et al., Mol. Biol. Cell 5: 519-528 (1994)).

### (e) Sialyltransferases

Sialyltransferases are another type of glycosyltransferase that is useful in the recombinant cells and reaction mixtures of the invention. Cells that produce recombinant sialyltransferases will also produce CMP-sialic acid, which is a sialic acid donor for sialyltransferases. Examples of sialyltransferases that are suitable for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al., Glycobiology 6: v-xiv (1996)). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem. 256: 3159 (1981), Weinstein et al., J. Biol. Chem. 257: 13845 (1982) and Wen et al., J. Biol. Chem. 267: 21011 (1992). Another exemplary α2,3-sialyltransferase (EC 2.4.99.4) transfers sialic acid to the non-reducing terminal Gal of the disaccharide or glycoside. see, Rearick et al., J. Biol. Chem. 254: 4444 (1979) and Gillespie et al., J. Biol. Chem. 267: 21004 (1992). Further exemplary enzymes include Gal-β-1,4-GlcNAc α-2,6 sialyltransferase (See, Kurosawa et al. Eur. J. Biochem. 219: 375-381 (1994)).

Preferably, for glycosylation of carbohydrates of glycopeptides the sialyltransferase will be able to transfer sialic acid to the sequence Galβ1,4GlcNAc-, the most common penultimate sequence underlying the terminal sialic acid on fully sialylated carbohydrate structures (see, Table 13, below).

TABLE 13
Sialyltransferases which use the Galβ1,4GlcNAc sequence as an acceptor
substrate

| Sialyltransferase | Source | Sequence(s) formed | Ref. |
|---|---|---|---|
| ST6Gal I | Mammalian | NeuAcα2,6Galβ1,4GlcNAc- | 1 |
| ST3Gal III | Mammalian | NeuAcα2,3Galβ1,4GlcNAc- | 1 |
| | | NeuAcα2,3Galβ1,3GlcNAc- | |
| ST3Gal IV | Mammalian | NeuAcα2,3Galβ1,4GlcNAc- | 1 |
| | | NeuAcα2,3Galβ1,3GlcNAc- | |
| ST6Gal II | Mammalian | NeuAcα2,6Galβ1,4GlcNAc | |
| ST6Gal II | photobacterium | NeuAcα2,6Galβ1,4GlcNAc- | 2 |
| ST3Gal V | N. | NeuAcα2,3Galβ1,4GlcNAc- | 3 |
| | meningitides | | |
| | N. | | |
| | gonorrhoeae | | |

1 Goochee et al., Bio/Technology 9: 1347-1355 (1991)
2 Yamamoto et al., J. Biochem. 120: 104-110 (1996)
3 Gilbert et al., J. Biol. Chem. 271: 28271-28276 (1996)

An example of a sialyltransferase that is useful in the claimed methods is ST3Gal III, which is also referred to as α(2,3)sialyltransferase (EC 2.4.99.6). This enzyme catalyzes the transfer of sialic acid to the Gal of a Galβ1,3GlcNAc or Galβ1,4GlcNAc glycoside (see, e.g., Wen et al., J. Biol. Chem. 267: 21011 (1992); Van den Eijnden et al., J. Biol. Chem. 256: 3159 (1991)) and is responsible for sialylation of asparagine-linked oligosaccharides in glycopeptides. The sialic acid is linked to a Gal with the formation of an α-linkage between the two saccharides. Bonding (linkage) between the saccharides is between the 2-position of NeuAc and the 3-position of Gal. This particular enzyme can be isolated from rat liver (Weinstein et al., J. Biol. Chem. 257: 13845 (1982)); the human cDNA (Sasaki et al. (1993) J. Biol. Chem. 268: 22782-22787; Kitagawa & Paulson (1994) J. Biol. Chem. 269: 1394-1401) and genomic (Kitagawa et al. (1996) J. Biol. Chem. 271: 931-938) DNA sequences are known, facilitating production of this enzyme by recombinant expression. In another embodiment, the claimed sialylation methods use a rat ST3Gal III.

Other exemplary sialyltransferases of use in the present invention include those isolated from Campylobacter jejuni, including the α(2,3). See, e.g, WO99/49051.

Sialyltransferases other those listed in Table 13, are also useful in an economic and efficient large-scale process for sialylation of commercially important glycopeptides. As a simple test to find out the utility of these other enzymes, various amounts of each enzyme (1-100 mU/mg protein) are reacted with asialo-α₁ AGP (at 1-10 mg/ml) to compare the ability of the sialyltransferase of interest to sialylate glycopeptides relative to either bovine ST6Gal I, ST3Gal III or both sialyltransferases. Alternatively, other glycopeptides or glycopeptides, or N-linked oligosaccharides enzymatically released from the peptide backbone can be used in place of asialo-α₁ AGP for this evaluation. Sialyltransferases with the ability to sialylate N-linked oligosaccharides of glycopeptides more efficiently than ST6Gal I are useful in a practical large-scale process for peptide sialylation (as illustrated for ST3Gal III in this disclosure). Other exemplary sialyltransferases are shown in FIG. 10.

### Fusion Proteins

In other exemplary embodiments, the methods of the invention utilize fusion proteins that have more than one enzymatic activity that is involved in synthesis of a desired glycopeptide conjugate. The fusion polypeptides can be composed of, for example, a catalytically active domain of a glycosyltransferase that is joined to a catalytically active domain of an accessory enzyme. The accessory enzyme catalytic domain can, for example, catalyze a step in the formation of a nucleotide sugar that is a donor for the glycosyltransferase, or catalyze a reaction involved in a glycosyltransferase cycle. For example, a polynucleotide that encodes a glycosyltransferase can be joined, in-frame, to a polynucleotide that encodes an enzyme involved in nucleotide sugar synthesis. The resulting fusion protein can then catalyze not only the synthesis of the nucleotide sugar, but also the transfer of the sugar moiety to the acceptor molecule. The fusion protein can be two or more cycle enzymes linked into one expressible nucleotide sequence. In other embodiments the fusion protein includes the catalytically active domains of two or more glycosyltransferases. See, for example, U.S. Pat. No. 5,641,668. The modified glycopeptides of the present invention can be readily designed and manufactured utilizing various suitable fusion proteins (see, for example, PCT Patent Application PCT/CA98/01180, which was published as WO 99/31224 on Jun. 24, 1999.)

### Immobilized Enzymes

In addition to cell-bound enzymes, the present invention also provides for the use of enzymes that are immobilized on a solid and/or soluble support. In an exemplary embodiment, there is provided a glycosyltransferase that is conjugated to a PEG via an intact glycosyl linker according to the methods of the invention. The PEG-linker-enzyme conjugate is optionally attached to solid support. The use of solid supported enzymes in the methods of the invention simplifies the work up of the reaction mixture and purification of the reaction product, and also enables the facile recovery of the enzyme. The glycosyltransferase conjugate is utilized in the methods of the invention. Other combinations of enzymes and supports will be apparent to those of skill in the art.

### Purification of Peptide Conjugates

The polypeptide conjugates produced by the processes described herein above can be used without purification. However, it is usually preferred to recover such products. Standard, well-known techniques for the purification of glycosylated saccharides, such as thin or thick layer chromatography, column chromatography, ion exchange chromatography, or membrane filtration. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have a molecular weight cutoff of about 3000 to about 10,000 can be used to remove proteins such as glycosyl transferases. Nanofiltration or reverse osmosis can then be used to remove salts and/or purify the product saccharides (see, e.g., WO 98/15581). Nanofilter membranes are a class of reverse osmosis membranes that pass monovalent salts but retain polyvalent salts and uncharged solutes larger than about 100 to about 2,000 Daltons, depending upon the membrane used. Thus, in a typical application, saccharides prepared by the methods of the present invention will be retained in the membrane and contaminating salts will pass through.

If the modified glycoprotein is produced intracellularly, as a first step, the particulate debris, including cells and cell debris, is removed, for example, by centrifugation or ultrafiltration. Optionally, the protein may be concentrated with a commercially available protein concentration filter, followed by separating the polypeptide variant from other impurities by one or more chromatographic steps, such as immunoaffinity chromatography, ion-exchange chromatography (e.g., on diethylaminoethyl (DEAE) or matrices containing carboxymethyl or sulfopropyl groups), hydroxy apatite chromatography and hydrophobic interaction chromatography (HIC). Exemplary stationary phases include Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, SP-Sepharose, or protein A Sepharose.

Other chromatographic techniques include SDS-PAGE chromatography, silica chromatography, chromatofocusing, reverse phase HPLC (e.g., silica gel with appended aliphatic groups), gel filtration using, e.g., Sephadex molecular sieve or size-exclusion chromatography, chromatography on columns that selectively bind the polypeptide, and ethanol or ammonium sulfate precipitation.

Modified glycopeptides produced in culture are usually isolated by initial extraction from cells, enzymes, etc., followed by one or more concentration, salting-out, aqueous ion-exchange, or size-exclusion chromatography steps, e.g., SP Sepharose. Additionally, the modified glycoprotein may be purified by affinity chromatography. HPLC may also be employed for one or more purification steps.

A protease inhibitor, e.g., methylsulfonylfluoride (PMSF) may be included in any of the foregoing steps to inhibit proteolysis and antibiotics may be included to prevent the growth of adventitious contaminants.

Within another embodiment, supernatants from systems which produce the modified glycopeptide of the invention are first concentrated using a commercially available protein concentration filter, for example, an Amicon or Millipore Pellicon ultrafiltration unit. Following the concentration step, the concentrate may be applied to a suitable purification matrix. For example, a suitable affinity matrix may comprise a ligand for the peptide, a lectin or antibody molecule bound to a suitable support. Alternatively, an anion-exchange resin may be employed, for example, a matrix or substrate having pendant DEAE groups. Suitable matrices include acrylamide, agarose, dextran, cellulose, or other types commonly employed in protein purification. Alternatively, a cation-exchange step may be employed. Suitable cation exchangers include various insoluble matrices comprising sulfopropyl or carboxymethyl groups. Sulfopropyl groups are particularly preferred.

Finally, one or more RP-HPLC steps employing hydrophobic RP-HPLC media, e.g., silica gel having pendant methyl or other aliphatic groups, may be employed to further purify a polypeptide variant composition. Some or all of the foregoing purification steps, in various combinations, can also be employed to provide a homogeneous modified glycoprotein.

The modified glycopeptide of the invention resulting from a large-scale fermentation may be purified by methods analogous to those disclosed by Urdal et al., J. Chromatog. 296:171 (1984). This reference describes two sequential, RP-HPLC steps for purification of recombinant human IL-2 on a preparative HPLC column. Alternatively, techniques such as affinity chromatography may be utilized to purify the modified glycoprotein.

### Acquisition of Peptide Coding Sequences

### General Recombinant Technology

The creation of mutant polypeptides, which incorporate an O-linked glycosylation sequence of the invention can be accomplished by altering the amino acid sequence of a correponding parent polypeptide, by either mutation or by full chemical synthesis of the polypeptide. The peptide amino acid sequence is preferably altered through changes at the DNA level, particularly by mutating the DNA sequence encoding the peptide at preselected bases to generate codons that will translate into the desired amino acids. The DNA mutation(s) are preferably made using methods known in the art.

This invention relies on routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

Nucleic acid sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Entire genes can also be chemically synthesized. Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of the cloned wild-type peptide genes, polynucleotide encoding mutant peptides, and synthetic oligonucleotides can be verified after cloning using, e.g., the chain termination method for sequencing double-stranded templates of Wallace et al., Gene 16: 21-26 (1981).

In an exemplary embodiment, the glycosylation sequence is added by shuffling polynucleotides. Polynucleotides encoding a candidate peptide can be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer, Proc. Natl. Acad. Sci. USA 91:10747-10751 (1994); Stemmer, Nature 370:389-391 (1994); and U.S. Pat. Nos. 5,605,793, 5,837,458, 5,830,721 and 5,811,238.

### Cloning and Subcloning of a Wild-Type Peptide Coding Sequence

Numerous polynucleotide sequences encoding wild-type peptides have been determined and are available from a commercial supplier, e.g., human growth hormone, e.g., GenBank Accession Nos. NM 000515, NM 002059, NM 022556, NM 022557, NM 022558, NM 022559, NM 022560, NM 022561, and NM 022562.

The rapid progress in the studies of human genome has made possible a cloning approach where a human DNA sequence database can be searched for any gene segment that has a certain percentage of sequence homology to a known nucleotide sequence, such as one encoding a previously identified peptide. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence encoding a peptide can be isolated from a human cDNA or genomic DNA library using standard cloning techniques such as polymerase chain reaction (PCR), where homology-based primers can often be derived from a known nucleic acid sequence encoding a peptide. Most commonly used techniques for this purpose are described in standard texts, e.g., Sambrook and Russell, supra.

cDNA libraries suitable for obtaining a coding sequence for a wild-type peptide may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full-length polynucleotide sequence encoding the wild-type peptide from the cDNA library. A general description of appropriate procedures can be found in Sambrook and Russell, supra.

A similar procedure can be followed to obtain a full length sequence encoding a wild-type peptide, e.g., any one of the GenBank Accession Nos mentioned above, from a human genomic library. Human genomic libraries are commercially available or can be constructed according to various art-recognized methods. In general, to construct a genomic library, the DNA is first extracted from a tissue where a peptide is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full-length nucleic acid encoding a wild-type peptide is obtained.

Upon acquiring a nucleic acid sequence encoding a wild-type peptide, the coding sequence can be subcloned into a vector, for instance, an expression vector, so that a recombinant wild-type peptide can be produced from the resulting construct. Further modifications to the wild-type peptide coding sequence, e.g., nucleotide substitutions, may be subsequently made to alter the characteristics of the molecule.

Introducing Mutations into a Peptide Sequence

From an encoding polynucleotide sequence, the amino acid sequence of a wild-type peptide can be determined. Subsequently, this amino acid sequence may be modified to alter the protein's glycosylation pattern, by introducing additional glycosylation sequence(s) at various locations in the amino acid sequence.

Several types of protein glycosylation sequences are well known in the art. For instance, in eukaryotes, N-linked glycosylation occurs on the asparagine of the consensus sequence Asn-Xₐₐ-Ser/Thr, in which Xₐₐ is any amino acid except proline (Kornfeld et al., Ann Rev Biochem 54:631-664 (1985); Kukuruzinska et al., Proc. Natl. Acad. Sci. USA 84:2145-2149 (1987); Herscovics et al., FASEB J 7:540-550 (1993); and Orlean, Saccharomyces Vol. 3 (1996)). O-linked glycosylation takes place at serine or threonine residues (Tanner et al., Biochim. Biophys. Acta. 906:81-91 (1987); and Hounsell et al., Glycoconj. J. 13:19-26 (1996)). Other glycosylation patterns are formed by linking glycosylphosphatidylinositol to the carboxyl-terminal carboxyl group of the protein (Takeda et al., Trends Biochem. Sci. 20:367-371 (1995); and Udenfriend et al., Ann. Rev. Biochem. 64:593-591 (1995). Based on this knowledge, suitable mutations can thus be introduced into a wild-type peptide sequence to form new glycosylation sequences.

Although direct modification of an amino acid residue within a peptide polypeptide sequence may be suitable to introduce a new N-linked or O-linked glycosylation sequence, more frequently, introduction of a new glycosylation sequence is accomplished by mutating the polynucleotide sequence encoding a peptide. This can be achieved by using any of known mutagenesis methods, some of which are discussed below.

A variety of mutation-generating protocols are established and described in the art. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortle, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

### Modification of Nucleic Acids for Preferred Codon Usage in a Host Organism

The polynucleotide sequence encoding a mutant peptide can be further altered to coincide with the preferred codon usage of a particular host. For example, the preferred codon usage of one strain of bacterial cells can be used to derive a polynucleotide that encodes a mutant peptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell. U.S. Pat. No. 5,824,864, for example, provides the frequency of codon usage by highly expressed genes exhibited by dicotyledonous plants and monocotyledonous plants.

At the completion of modification, the mutant peptide coding sequences are verified by sequencing and are then subcloned into an appropriate expression vector for recombinant production in the same manner as the wild-type peptides.

### Expression of Mutant Polypeptide

In an exemplary embodiment, the polypeptide that is modified by a method of the invention is produced in prokaryotic cells (e.g., E. coli), eukaryotic cells including yeast and mammalian cells (e.g., CHO cells), or in a transgenic animal.

Following sequence verification, the mutant peptide of the present invention can be produced using routine techniques in the field of recombinant genetics, relying on the polynucleotide sequences encoding the polypeptide disclosed herein.

### Expression Systems

To obtain high-level expression of a nucleic acid encoding a mutant peptide of the present invention, one typically subclones a polynucleotide encoding the mutant peptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the wild-type or mutant peptide are available in, e.g., E. coli, Bacillus sp., Salmonella, Caulobacter, and the like. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the mutant peptide in host cells. A typical expression cassette thus contains a promoter operably linked to the nucleic acid sequence encoding the mutant peptide and signals required for efficient polyadenylation of the transcript, ribosome binding sites, and translation termination. The nucleic acid sequence encoding the peptide is typically linked to a cleavable signal peptide sequence to promote secretion of the peptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322-based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

In some exemplary embodiments the expression vector is chosen from pCWin1, pCWin2, pCWin2/MBP, pCWin2-MBP-SBD (pMS₃₉), and pCWin2-MBP-MCS-SBD (pMXS₃₉) as disclosed in co-owned U.S. Patent application filed Apr. 9, 2004 which is incorporated herein by reference.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the mutant peptide under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

When periplasmic expression of a recombinant protein (e.g., a hgh mutant of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

As discussed above, a person skilled in the art will recognize that various conservative substitutions can be made to any wild-type or mutant peptide or its coding sequence while still retaining the biological activity of the peptide. Moreover, modifications of a polynucleotide coding sequence may also be made to accommodate preferred codon usage in a particular expression host without altering the resulting amino acid sequence.

### Transfection Methods

Standard transfection methods are used to produce bacterial, mammalian, yeast or insect cell lines that express large quantities of the mutant peptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the mutant peptide.

### Detection of Expression of Mutant Peptide in Host Cells

After the expression vector is introduced into appropriate host cells, the transfected cells are cultured under conditions favoring expression of the mutant peptide. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding a mutant peptide in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a mutant peptide of the present invention (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the mutant peptide or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6: 511-519 (1976). More detailed descriptions of preparing antibody against the mutant peptide of the present invention and conducting immunological assays detecting the mutant peptide are provided in a later section.

### Purification of Recombinantly Produced Mutant Polypeptides

Once the expression of a recombinant mutant peptide in transfected host cells is confirmed, the host cells are then cultured in an appropriate scale for the purpose of purifying the recombinant polypeptide.

1. Purification from Bacteria

When the mutant peptides of the present invention are produced recombinantly by transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant peptide from bacterial inclusion body, see, e.g., Patra et al., Protein Expression and Purification 18: 182-190 (2000).

Alternatively, it is possible to purify recombinant polypeptides, e.g., a mutant peptide, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### 2. Standard Protein Separation Techniques for Purification

When a recombinant polypeptide, e.g., the mutant peptide of the present invention, is expressed in host cells in a soluble form, its purification can follow standard protein purification procedures, for instance those described herein, below or purification can be accomplished using methods disclosed elsewhere, e.g., in PCT Publication No. WO2006/105426, which is incorporated by reference herein.

### Solubility Fractionation

Often as an initial step, and if the protein mixture is complex, an initial salt fractionation can separate many of the unwanted host cell proteins (or proteins derived from the cell culture media) from the recombinant protein of interest, e.g., a mutant peptide of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

### Ultrafiltration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a mutant peptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

### Column Chromatography

The proteins of interest (such as the mutant peptide of the present invention) can also be separated from other proteins on the basis of their size, net surface charge, hydrophobicity, or affinity for ligands. In addition, antibodies raised against peptide can be conjugated to column matrices and the peptide immunopurified. All of these methods are well known in the art.

It will be apparent to one of skill that chromatographic techniques can be performed at any scale and using equipment from many different manufacturers (e.g., Pharmacia Biotech).

### Immunoassays for Detection of Mutant Peptide Expression

To confirm the production of a recombinant mutant peptide, immunological assays may be useful to detect in a sample the expression of the polypeptide. Immunological assays are also useful for quantifying the expression level of the recombinant hormone. Antibodies against a mutant peptide are necessary for carrying out these immunological assays.

Production of Antibodies against Mutant Peptide

Methods for producing polyclonal and monoclonal antibodies that react specifically with an immunogen of interest are known to those of skill in the art (see, e.g., Coligan, Current Protocols in Immunology Wiley/Greene, N.Y., 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256: 495-497, 1975). Such techniques include antibody preparation by selection of antibodies from libraries of recombinant antibodies in phage or similar vectors (see, Huse et al., Science 246: 1275-1281, 1989; and Ward et al., Nature 341: 544-546, 1989).

In order to produce antisera containing antibodies with desired specificity, the polypeptide of interest (e.g., a mutant peptide of the present invention) or an antigenic fragment thereof can be used to immunize suitable animals, e.g., mice, rabbits, or primates. A standard adjuvant, such as Freund's adjuvant, can be used in accordance with a standard immunization protocol. Alternatively, a synthetic antigenic peptide derived from that particular polypeptide can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. Further fractionation of the antisera to enrich antibodies specifically reactive to the antigen and purification of the antibodies can be performed subsequently, see, Harlow and Lane, supra, and the general descriptions of protein purification provided above.

Monoclonal antibodies are obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. The general principles and methods of recombinant polypeptide production discussed above are applicable for antibody production by recombinant methods.

When desired, antibodies capable of specifically recognizing a mutant peptide of the present invention can be tested for their cross-reactivity against the wild-type peptide and thus distinguished from the antibodies against the wild-type protein. For instance, antisera obtained from an animal immunized with a mutant peptide can be run through a column on which a wild-type peptide is immobilized. The portion of the antisera that passes through the column recognizes only the mutant peptide and not the wild-type peptide. Similarly, monoclonal antibodies against a mutant peptide can also be screened for their exclusivity in recognizing only the mutant but not the wild-type peptide.

Polyclonal or monoclonal antibodies that specifically recognize only the mutant peptide of the present invention but not the wild-type peptide are useful for isolating the mutant protein from the wild-type protein, for example, by incubating a sample with a mutant peptide-specific polyclonal or monoclonal antibody immobilized on a solid support.

### Immunoassays for Detecting Recombinant Peptide Expression

Once antibodies specific for a mutant peptide of the present invention are available, the amount of the polypeptide in a sample, e.g., a cell lysate, can be measured by a variety of immunoassay methods providing qualitative and quantitative results to a skilled artisan. For a review of immunological and immunoassay procedures in general see, e.g., Stites, supra; U.S. Pat. Nos. 4,366,241; 4,376,110; 4,517,288; and 4,837,168.

### Labeling in Immunoassays

Immunoassays often utilize a labeling agent to specifically bind to and label the binding complex formed by the antibody and the target protein. The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In some cases, the labeling agent is a second antibody bearing a detectable label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111: 1401-1406 (1973); and Akerstrom, et al., J. Immunol., 135: 2589-2542 (1985)).

### Immunoassay Formats

Immunoassays for detecting a target protein of interest (e.g., a mutant human growth hormone) from samples may be either competitive or noncompetitive. Noncompetitive immunoassays are assays in which the amount of captured target protein is directly measured. In one preferred “sandwich” assay, for example, the antibody specific for the target protein can be bound directly to a solid substrate where the antibody is immobilized. It then captures the target protein in test samples. The antibody/target protein complex thus immobilized is then bound by a labeling agent, such as a second or third antibody bearing a label, as described above.

In competitive assays, the amount of target protein in a sample is measured indirectly by measuring the amount of an added (exogenous) target protein displaced (or competed away) from an antibody specific for the target protein by the target protein present in the sample. In a typical example of such an assay, the antibody is immobilized and the exogenous target protein is labeled. Since the amount of the exogenous target protein bound to the antibody is inversely proportional to the concentration of the target protein present in the sample, the target protein level in the sample can thus be determined based on the amount of exogenous target protein bound to the antibody and thus immobilized.

In some cases, western blot (immunoblot) analysis is used to detect and quantify the presence of a mutant peptide in the samples. The technique generally comprises separating sample proteins by gel electrophoresis on the basis of molecular weight, transferring the separated proteins to a suitable solid support (such as a nitrocellulose filter, a nylon filter, or a derivatized nylon filter) and incubating the samples with the antibodies that specifically bind the target protein. These antibodies may be directly labeled or alternatively may be subsequently detected using labeled antibodies (e.g., labeled sheep anti-mouse antibodies) that specifically bind to the antibodies against a mutant peptide.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5: 34-41 (1986)).

### Methods of Treatment

In addition to the conjugates discussed above, the present invention provides methods of preventing, curing or ameliorating a disease state by administering a polypeptide conjugate of the invention to a subject at risk of developing the disease or a subject that has the disease. Additionally, the invention provides methods for targeting conjugates of the invention to a particular tissue or region of the body.

The following examples are provided to illustrate the compositions and methods of the present invention, but not to limit the claimed invention.

### EXAMPLES

### Example 1

### Preparation of Mutant Interferon-alpha-2b-GlcNH-Glycine-PEG-30 kDa

The mutant IFN-alpha-2b (30 mg, 1.55 micromoles) was buffer exchanged into reaction buffer (50 mM Tris, MgCl₂, pH 7.8) using a Centricon Plus-20 centrifugal filter, 5 kDa MWCO, to a final protein concentration of 10 mg/mL. The UDP-GlcNH-glycine-PEG-30 kDa (2 mole eq) and MBP-GlcNAc Transferase (20 mU/mg protein) were then added. The reaction mixture was incubated at 32° C. until the reaction was complete. The extent of reaction was determined by SDS-PAGE gel. The product, IFN-alpha-2b-GlcNH-glycine-PEG-30 kDa, was purified as described in the literature (SP-sepharose and Superdex 200 chromatography) prior to formulation.

IFNalpha mutant:

(SEQ ID NO: 234)

MCDLPQTHSLGSRRTLMLLAQMRRISLFSCLKDRHDFGFPQEEFGNQFQK
AETIPVLHEMIQQIFNLFSTKDSSAAWDETLLDKFYTELYQQLNDLEACV
IQGVPVS¹⁰⁶RAPLMKEDSILAVRKYFQRITLYLKEKKYSPCAWEVVRAE
IMRSFSLSTNLQESLRSKE

### UDP-GlcNH-glycine-PEG-30 kDa:

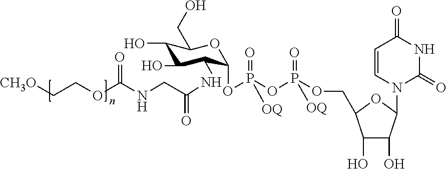

### Example 2

### Preparation of Mutant Interferon-alpha-2b-GlcNH-caproylamido-PEG-40 kDa

The mutant IFN-alpha-2b (1 mg) was buffer exchanged into reaction buffer (50 mM HEPES, MgCl₂, pH 7.4, 100 mM NaCl) using a Centricon Plus-20 centrifugal filter, 5 kDa MWCO, to a final protein concentration of 1 mg/mL. The UDP-GlcNH-caproylamido-PEG-40 kDa (2 mole eq) and MBP-GlcNAc Transferase (100 mU/mg protein) were then added. The reaction mixture was incubated at 32° C. until the reaction was complete. The extent of reaction was determined by SDS-PAGE gel. The product, IFN-alpha-2b-GlcNH-caproylamido-PEG-40 kDa, was purified as described in the literature (SP-sepharose and Superdex 200 chromatography) prior to formulation.

IFNalpha mutant:

(SEQ ID NO: 235)

MCDLPQTHSLGSRRTLMLLAQMRRISLFSCLKDRHDFGFPQEEFGNQFQK
AETIPVLHEMIQQIFNLFSTKDSSAAWDETLLDKFYTELYQQLNDLEACV
IQGVGPV¹⁰⁶SRPLMKEDSILAVRKYFQRITLYLKEKKYSPCAWEVVRAE
IMRSFSLSTNLQESLRSKE

### UDP-GlcNH-caproylamido-PEG-40 kDa:

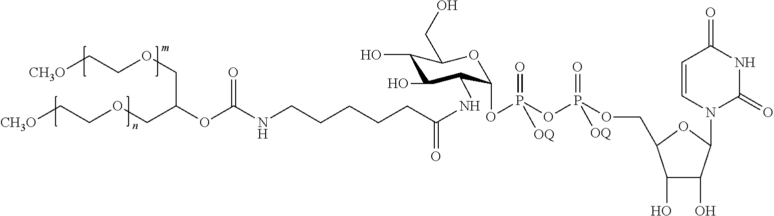

### Example 3

### Preparation of Mutant BMP7-GlcNH-Glycine-PEG-30 kDa

The mutant BMP7 (1 mg) was buffer exchanged into reaction buffer (50 mM MES, MgCl₂, pH 6.2) using a Centricon Plus-20 centrifugal filter, 5 kDa MWCO, to a final protein concentration of 1 mg/mL. The UDP-GlcNH-glycine-PEG-30 kDa (1.5 mole eq) and MBP-GlcNAc Transferase (100 mU/mg protein) were then added. The reaction mixture was incubated at 32° C. until the reaction was complete. The extent of reaction was determined by SDS-PAGE gel. The product, BMP7-GlcNH-glycine-PEG-30 kDa, was purified as described in the literature (SP-sepharose and Superdex 200 chromatography) prior to formulation.

### Mutant BMP7:

(SEQ ID NO: 236)

MVPVSGSTGSKQRSQNRSKTPKNQEALRMANVAENSSSDQRQACKKHELY
VSFRDLGWQDWIIAPEGYAAYYCEGECAFPLNSYMNATNHAIVQTLVHFI
NPETVPKPCCAPTQLNAISVLYFDDSSNVILKKYRNMVVRACGCH

### UDP-GlcNH-glycine-PEG-30 kDa:

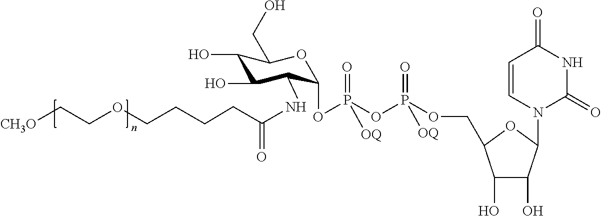

### Example 4

### Preparation of Mutant Human Growth Hormone-GlcNH-Glycine-PEG-40 kDa

The mutant growth hormone (1 mg) was buffer exchanged into reaction buffer (50 mM HEPES, CaCl₂, 50 mM NaCl, pH 7.4) using a Centricon Plus-20 centrifugal filter, 5 kDa MWCO, to a final protein concentration of 1 mg/mL. The UDP-GlcNH-glycine-PEG-40 kDa (1.5 mole eq) and MBP-GlcNAc Transferase (50 mU/mg protein) were then added. The reaction mixture was incubated at room temperature until the reaction was complete. The extent of reaction was determined by SDS-PAGE gel. The product, growth hormone-GlcNH-glycine-PEG-40 kDa, was purified as described in the literature (DEAE Sepharose and Superdex 200 chromatography) prior to formulation.

### Mutant Growth Hormone:

(SEQ ID NO: 237)

MFPTIPLSRLFDNAMLRAHRLHQLAFDTYQEFEEAYIPKEQKYSFLQNPQ
TSLCFSESIPTPSNREETQQKSNLELLRISLLLIQSWLEPVQFLRSVFAN
SLVYGASDSNVYDLLKDLEEGIQTLMGRLEDGSPVSGSIFKQTYSKFDTN
SHNDDALLKNYGLLYCFRKDMDKVETFLRIVQCRSVEGSCG

UDP-GlcNH-6′-glycine-PEG-40 kDa:

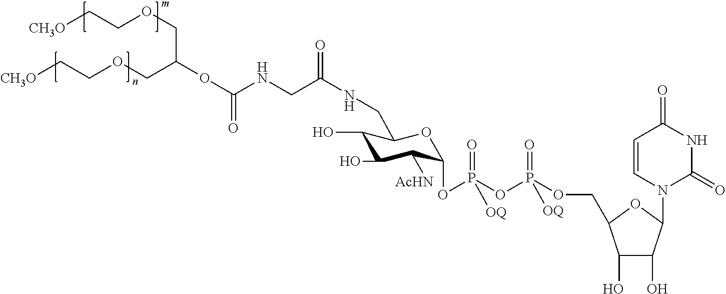

### Example 5

### Preparation of Mutant GC SF-GlcNH-Glycine-PEG-20 kDa

The mutant GCSF (1 mg) was buffer exchanged into reaction buffer (50 mM MES, MgCl₂, pH 6.2) using a Centricon Plus-20 centrifugal filter, 5 kDa MWCO, to a final protein concentration of 1 mg/mL. The UDP-GlcNH-glycine-PEG-20 kDa (2.0 mole eq) and MBP-GlcNAc Transferase (100 mU/mg protein) were then added. The reaction mixture was incubated at 32° C. until the reaction was complete. The extent of reaction was determined by SDS-PAGE gel. The product, GSCF-GlcNH-glycine-PEG-20 kDa, was purified as described in the literature (SP-sepharose and Superdex 200 chromatography) prior to formulation.

### Mutant GCSF:

(SEQ ID NO: 238)

MPVSGTPLGPASSLPQSFLLKCLEQVRKIQGDGAALQEKLCATYKLCHPE
ELVLLGHSLGIPWAPLSSCPSQALQLAGCLSQLHSGLFLYQGLLQALEGI
SPELGPTLDTLQLDVADFATTIWQQMEELGMAPALQPTQGAMPAFASAFQ
RRAGGVLVASHLQSFLEVSYRVLRHLAQP

### UDP-GlcNH-caproylamide-PEG-20 kDa:

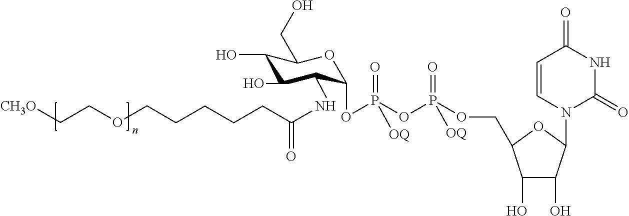

### Example 6

### Preparation of Mutant Enbrel-[GlcNH-caproylamido-PEG-80 kDa]₂

Mutant Enbrel containing an O-linked glycosylation sequence of the invention (100 mg) was buffer exchanged into reaction buffer (50 mM Tris, MgCl₂, pH 7.8) using a Centricon Plus-20 centrifugal filter, 5 kDa MWCO, to a final protein concentration of 10 mg/mL. The UDP-GlcNH-caproylamido-PEG-80 kDa (2.2 mole eq) and MBP-GlcNAc Transferase (75 mU/mg protein) were then added. The reaction mixture was incubated at 32° C. until the reaction was complete. The extent of reaction was determined by SDS-PAGE gel. The product, Enbrel-[GlcNH-caproylamido-PEG-80 kDa]₂, was purified as described in the literature (Q-sepharose and Superdex 200 chromatography) prior to formulation.

### UDP-GlcNH-caproylamido-PEG-80 kDa:

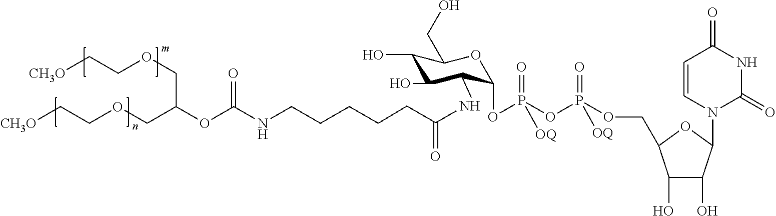

### Example 7

### Expression of GlcNAc transferases in E. coli

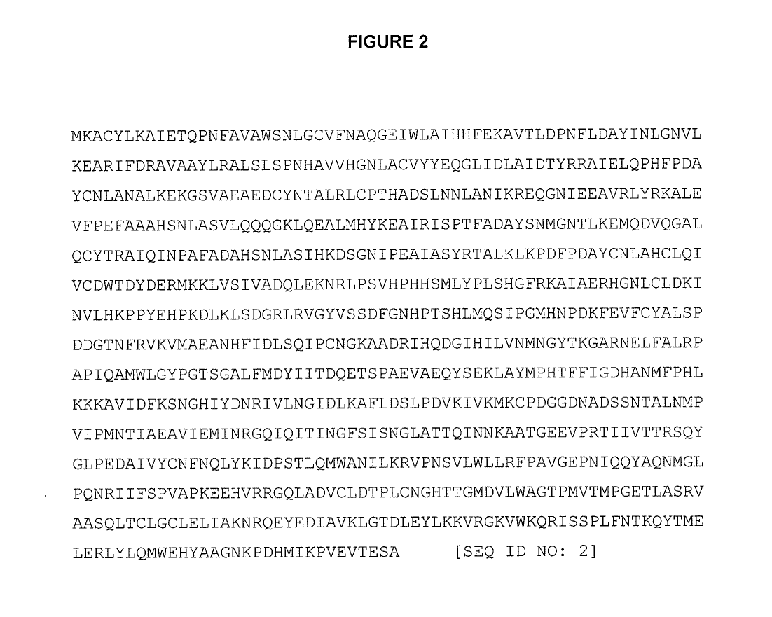

DNA encoding human OGT with accession number 015294 (SEQ ID NO: 1, FIG. 1), lacking the first 176 amino acids (Δ176, SEQ ID NO: 2, FIG. 2) was synthesized using codons selected for high expression in E. coli. Using common methods known in the art, various truncated and/or tagged forms of human OGT were generated (see Table 14, below) in Plasmid7 expression vector. See, e.g., U.S. Provisional Patent Application 60/956,332, filed Aug. 16, 2007 (e.g., sequence id number 8, therein), which is incorporated herein in its entirety for all purposes. Constructs generated by PCR were confirmed by sequence analysis.

TABLE 14
Human OGT Expression Constructs

| human OGT N-terminal truncation | tag |
|---|---|
| Δ176 (SEQ ID NO: 239) | none |
| Δ182 (SEQ ID NO: 240) | C-terminal His₈ |
| Δ382 (SEQ ID NO: 241) | C-terminal His₈ |
| Δ382 (SEQ ID NO: 242) | N-terminal His₇ |

For expression, overnight cultures E. coli cells bearing each OGT construct were used to inoculate a 200 mL culture of prewarmed animal-free LB (1% martone B-1, 0.5% yeast extract, 1% NaCl) containing 50 μg/ml kanamycin. The culture was incubated at 37° C. with shaking, and monitored at OD₆₀₀. When the OD₆₀₀ reached 0.5-1, the cultures transferred to a 20° C. shaking incubator for 20-40 minutes. IPTG was then added to 0.2 mM final concentration, and shaking incubation was continued overnight. At harvest, the OD₆₀₀ was again measured, and the cells were collected by centrifugation at 4° C., 7,000×g for 15 minutes. Unless otherwise specified, all OGT constructs were expressed in trxB gor supp mutant E. coli. Additional methods and procedures as well as sequences can be found, e.g., in Ausubel, F., et al., eds. 2007 Current Protocols in Molecular Biology (John Wiley & Sons, Inc. Hoboken, N.J.); Coligan, J., et al., eds. 2007 Current Protocols in Protein Science (John Wiley & Sons, Inc. Hoboken, N.J.); Kreppel, L. and G. Hart, J. Biol. Chem. 1999, 274: 32015-32022; Lubas, W. and J. Hanover, J. Biol. Chem. 2000, 275: 10983-10988; Hanover, J. et al., Arch. Biochem. Biophys. 2003, 409: 287-297; Gross, B., Kraybill, B., and S. Walker, J. Am. Chem. Soc. 2005, 127: 14588-14589; Gross, B., Swoboda, J., and S. Walker, J. Am. Chem. Soc. 2008, 130: 440-441, the disclosures of which are incorporated herein by reference in their entirety.

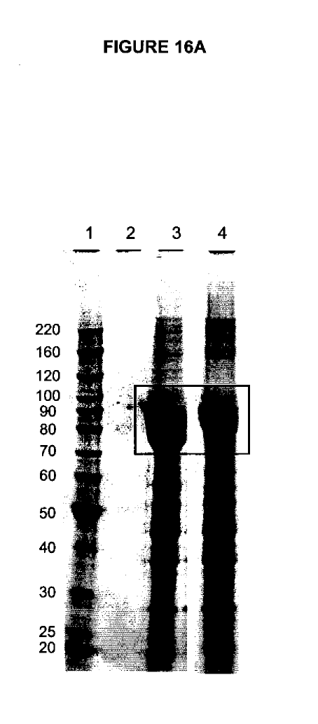

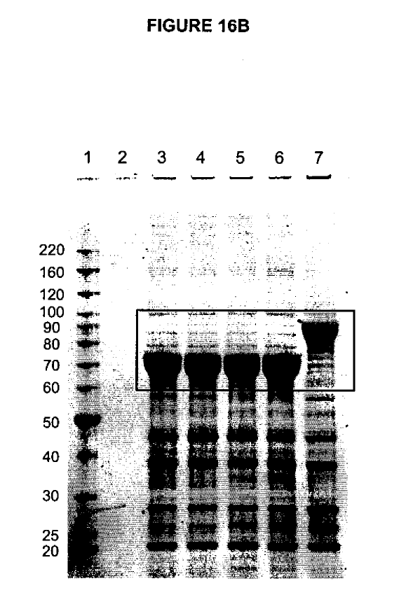

To monitor protein expression, total cell lysates were analyzed by SDS-PAGE. Equal samples of cells, based on OD₆₀₀ at harvest, were solubilized with detergents, and released bacterial DNA degraded with DNAse. Following reduction and heat denaturation, samples were resolved by electrophoresis, and stained with Coomassie Fluor Orange. As shown in FIG. 16, expression of all OGT constructs was observed. Bacterially-expressed untagged or His-tagged OGT can be purified and assayed using methods known in the art.

1. A covalent conjugate between a non-naturally occurring polypeptide and a polymeric modifying group, said non-naturally occurring polypeptide corresponding to a parent-polypeptide and comprising an exogenous O-linked glycosylation sequence that is not present, or not present at the same position, in said parent polypeptide, said O-linked glycosylation sequence being a substrate for a GlcNAc-transferase and comprising an amino acid residue having a hydroxyl group, wherein said polymeric modifying group is covalently linked to said polypeptide at said hydroxyl group of said O-linked glycosylation sequence via a glycosyl linking group.

2. The covalent conjugate according to claim 1, wherein said O-linked glycosylation sequence comprises an amino acid sequence, which is a member selected from Formulae (I)-(VI):

| | |
|---|---|
| (B¹)ₐ P (B²)_{b} U S (B³)_{c} | (I) (SEQ ID NO: 247) |
| (B¹)ₐ P (B²)_{b} U T (B³)_{c} | (II) (SEQ ID NO: 248) |
| (B⁴)_{d} P S Z (B⁵)ₑ | (III) (SEQ ID NO: 249) |
| (B⁴)_{d} P T Z (B⁵)ₑ | (IV) (SEQ ID NO: 250) |
| (B⁶)_{f} S (B⁷)_{g} P (B⁸)ₕ | (V) (SEQ ID NO: 251) |
| (B⁶)_{f} T (B⁷)_{g} P (B⁸)ₕ | (VI) (SEQ ID NO: 252) |

wherein
b and g are integers selected from 0 to 2;
a, c, d, e, f and h are integers selected from 0 to 5;
T is threonine;
S is serine;
P is proline;
U is a member selected from V, S, T, E, Q and uncharged amino acids;
Z is a member selected from P, E, Q, S, T and uncharged amino acids; and
each B¹, B², B³, B⁴, B⁵, B⁶, B⁷ and B⁸ is a member independently selected from an amino acid.

3. The covalent conjugate according to claim 1, wherein said O-linked glycosylation sequence comprises an amino acid sequence, which is a member selected from:

| | |
|---|---|
| (B¹)ₐ P V S (B³)_{c}; | (SEQ ID NO: 16) |
| (B¹)ₐ P V T (B³)_{c}; | (SEQ ID NO: 17) |
| (B¹)ₐ P S S (B³)_{c}; | (SEQ ID NO: 18) |
| (B¹)ₐ P S T (B³)_{c}; | (SEQ ID NO: 19) |
| (B¹)ₐ P T S (B³)_{c}; | (SEQ ID NO: 20) |
| (B¹)ₐ P B² V T (B³)_{c}; | (SEQ ID NO: 21) |
| (B¹)ₐ P B² V S (B³)_{c}; | (SEQ ID NO: 22) |
| (B¹)ₐ P K U T (B³)_{c}; | (SEQ ID NO: 23) |
| (B¹)ₐ P K U S (B³)_{c}; | (SEQ ID NO: 24) |
| (B¹)ₐ P Q U T (B³)_{c}; | (SEQ ID NO: 25) |
| (B¹)ₐ P Q U S (B³)_{c}; | (SEQ ID NO: 26) |
| (B¹)ₐ P (B²)₂ V S (B³)_{c}; | (SEQ ID NO: 27) |
| (B¹)ₐ P (B²)₂ V T (B³)_{c}; | (SEQ ID NO: 28) |
| (B¹)ₐ P (B²)₂ T S (B³)_{c}; | (SEQ ID NO: 29) |
| (B¹)ₐ P (B²)₂ T T (B³)_{c}; | (SEQ ID NO: 30) |
| (B⁴)_{d} P T P (B⁵)ₑ; | (SEQ ID NO: 31) |
| (B⁴)_{d} P T E (B⁵)ₑ; | (SEQ ID NO: 32) |
| (B⁴)_{d} P S A (B⁵)ₑ; | (SEQ ID NO: 33) |
| (B⁶)_{f} S B⁷ T P (B⁸)ₕ; | (SEQ ID NO: 34) |
| and | |
| (B⁶)_{f} S B⁷ S P (B⁸)ₕ. | (SEQ ID NO: 35) |

4. The covalent conjugate according to claim 1, wherein said O-linked glycosylation sequence comprises an amino acid sequence, which is a member selected from:

| | |
|---|---|
| PVS, | (SEQ ID NO: 36) |
| PVSG, | (SEQ ID NO: 37) |
| PVSGS, | (SEQ ID NO: 38) |
| VPVS, | (SEQ ID NO: 39 |
| VPVSG, | (SEQ ID NO: 40) |
| VPVSGS, | (SEQ ID NO: 41 |
| PVSR, | (SEQ ID NO: 42 |
| PVSRE, | (SEQ ID NO: 43) |
| PVSRA, PVSRP, PVSA, | (SEQ ID NO: 44) |
| PVSAS, | (SEQ ID NO: 45) |
| APVS, | (SEQ ID NO: 46) |
| APVSA, | (SEQ ID NO: 47) |
| APVSAS, | (SEQ ID NO: 48) |
| APVSS, | (SEQ ID NO: 49) |
| APVSSS, | (SEQ ID NO: 50) |
| PVSS, | (SEQ ID NO: 51) |
| PVSSA, | (SEQ ID NO: 52) |
| PVSSAP, | (SEQ ID NO: 53) |
| IPVS, | (SEQ ID NO: 54) |
| IPVSR, | (SEQ ID NO: 57) |
| VPVS, | (SEQ ID NO: 58) |
| VPVSS, | (SEQ ID NO: 59) |
| VPVSSA, | (SEQ ID NO: 60) |
| RPVS, | (SEQ ID NO: 61) |
| RPVSS, | (SEQ ID NO: 62) |
| RPVSSA, | (SEQ ID NO: 63) |
| PVT, | (SEQ ID NO: 64) |
| PSS, | (SEQ ID NO: 65) |
| PSST, | (SEQ ID NO: 66) |
| PSSTA, | (SEQ ID NO: 67) |
| PPSS, | (SEQ ID NO: 68) |
| PPSST, | (SEQ ID NO: 69) |
| PSSG, | (SEQ ID NO: 70) |
| PSSGF, | (SEQ ID NO: 71) |
| SPST, | (SEQ ID NO: 72 |
| SPSTS, | (SEQ ID NO: 73) |
| SPSTSP, | (SEQ ID NO: 74) |
| SPSS, | (SEQ ID NO: 75) |
| SPSSG, | (SEQ ID NO: 76) |
| SPSSGF, | (SEQ ID NO: 77) |
| PST, | (SEQ ID NO: 78) |
| PSTS, | (SEQ ID NO: 79) |
| PSTST, | (SEQ ID NO: 80) |
| PSTV, | (SEQ ID NO: 81) |
| PSTVS, | (SEQ ID NO: 82) |
| PSVT, | (SEQ ID NO: 83) |
| PSVTI, | (SEQ ID NO: 84) |
| PSVS, | (SEQ ID NO: 85) |
| PAVT, | (SEQ ID NO: 86) |
| PAVTA, | (SEQ ID NO: 87) |
| PAVTAA, | (SEQ ID NO: 88) |
| KPAVT, | (SEQ ID NO: 89) |
| KPAVTA, | (SEQ ID NO: 90) |
| PAVS, | (SEQ ID NO: 91) |
| PQQS, | (SEQ ID NO: 92) |
| PQQSA, | (SEQ ID NO: 93) |
| PQQSAS, | (SEQ ID NO: 94) |
| PQQT, | (SEQ ID NO: 95) |
| PKGS, | (SEQ ID NO: 96) |
| PKGSR, | (SEQ ID NO: 97) |
| PKGT, | (SEQ ID NO: 98) |
| PKSS, | (SEQ ID NO: 99) |
| PKSSA, | (SEQ ID NO: 100) |
| PKSSAP, | (SEQ ID NO: 101) |
| PKST, | (SEQ ID NO: 102) |
| PADTS, | (SEQ ID NO: 103) |
| PADTSD, | (SEQ ID NO: 104) |
| PADTT, | (SEQ ID NO: 105) |
| PIKVT, | (SEQ ID NO: 106) |
| PIKVTE, | (SEQ ID NO: 107) |
| PIKVS, | (SEQ ID NO: 108) |
| SPST, | (SEQ ID NO: 109) |
| SPSTS, | (SEQ ID NO: 110) |
| SPTS, | (SEQ ID NO: 111) |
| SPTSP, | (SEQ ID NO: 112) |
| PTSPX, | (SEQ ID NO: 113) |
| SPTSPX, | (SEQ ID NO: 114) |
| SPSA, | (SEQ ID NO: 115) |
| SPSAK, | (SEQ ID NO: 116) |
| TSPS, | (SEQ ID NO: 117) |
| TSPSA, | (SEQ ID NO: 118) |
| LPTP, | (SEQ ID NO: 119) |
| LPTPP, | (SEQ ID NO: 120) |
| PTPP, | (SEQ ID NO: 121) |
| PTPPL, | (SEQ ID NO: 122) |
| VPTE, | (SEQ ID NO: 123) |
| VPTET, | (SEQ ID NO: 124) |
| PTE, | (SEQ ID NO: 125) |
| PTET, | (SEQ ID NO: 126) |
| TSETP, | (SEQ ID NO: 127) |
| ITSETP, | (SEQ ID NO: 128) |
| ASVSP, | (SEQ ID NO: 129) |
| SASVSP, | (SEQ ID NO: 130) |
| VETP, | (SEQ ID NO: 131) |
| VETPR, | (SEQ ID NO: 132) |
| ETPR, | (SEQ ID NO: 133) |
| ACTQ, | (SEQ ID NO: 134) |
| ACTQG | (SEQ ID NO: 135) |
| and | |
| CTQG, | (SEQ ID NO: 136) |

wherein each threonine (T) independently can optionally be replaced with serine (S) and each serine independently can optionally be replaced with threonine.

5. The covalent conjugate according to claim 1, wherein said polymeric modifying group is a water-soluble polymer.

6. The covalent conjugate according to claim 5, wherein said water-soluble polymer is a member selected from poly(alkylene oxide), dextran and polysialic acid.

7. The covalent conjugate according to claim 6, wherein said poly(alkylene oxide) is a member selected from poly(ethylene glycol) and poly(propylene glycol) and derivatives thereof.

8. The covalent conjugate according to claim 7, wherein said poly(ethylene glycol) is monomethoxy-poly(ethylene glycol) (mPEG).

9. The covalent conjugate according to claim 7, wherein said poly(ethylene glycol) has a molecular weight that is essentially homodisperse.

10. The covalent conjugate according to claim 1, wherein said parent-polypeptide is a therapeutic polypeptide.

11. The covalent conjugate according to claim 1, wherein said parent-polypeptide is a member selected from bone morphogenetic protein 2 (BMP-2), bone morphogenetic protein 7 (BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (α-1 protease inhibitor), glucocerebrosidase, tissue-type plasminogen activator (TPA), interleukin-2 (IL-2), leptin, hirudin, urokinase, human DNase, insulin, hepatitis B surface protein (HbsAg), chimeric diphtheria toxin-IL-2, human growth hormone (hGH), human chorionic gonadotropin (hCG), alpha-galactosidase, alpha-L-iduronidase, beta-glucosidase, alpha-galactosidase A, acid α-glucosidase (acid maltase), anti-thrombin III (AT III), follicle stimulating hormone, glucagon-like peptide-1 (GLP-1), glucagon-like peptide-2 (GLP-2), fibroblast growth factor 7 (FGF-7), fibroblast growth factor 21 (FGF-21), fibroblast growth factor 23 (FGF-23), Factor VII, Factor VIII, B-domain deleted Factor VIII, Factor IX, Factor XIII, prokinetisin, extendin-4, CD4, tumor necrosis factor receptor (TNF-R), α-CD20, P-selectin glycoprotein ligand-1 (PSGL-1), complement, transferrin, glycosylation-dependent cell adhesion molecule (GlyCAM), neural-cell adhesion molecule (N-CAM), TNF receptor-IgG Fc region fusion protein, anti-HER2 monoclonal antibody, monoclonal antibody to respiratory syncytial virus, monoclonal antibody to protein F of respiratory syncytial virus, monoclonal antibody to TNF-α, monoclonal antibody to glycoprotein IIb/IIIa, monoclonal antibody to CD20, monoclonal antibody to VEGF-A, monoclonal antibody to PSGL-1, monoclonal antibody to CD4, monoclonal antibody to a-CD3, monoclonal antibody to EGF, monoclonal antibody to carcinoembryonic antigen (CEA) and monoclonal antibody to IL-2 receptor.

12. The covalent conjugate according to claim 1, wherein said GlcNAc-transferase is a recombinant enzyme.

13. The covalent conjugate according to claim 12, wherein said GlcNAc-transferase is expressed in a bacterial cell.

14. The covalent conjugate according to claim 1, wherein said glycosyl linking group is an intact glycosyl linking group.

15. The covalent conjugate according to claim 1, wherein said covalent conjugate comprises a moiety according to Formula (VII): 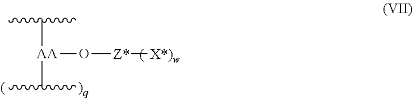
wherein
q is an integer selected from 0 and 1;
w is an integer selected from 0 and 1;
AA-0 is a moiety derived from said amino acid residue comprising a hydroxyl group, wherein said amino acid is located within said O-linked glycosylation sequence;
Z* is a member selected from a glucosamine moiety, a glucosamine-mimetic moiety, an oligosaccharide comprising a glucosamine-moiety and an oligosaccharide comprising a glucosamine-mimetic moiety; and
X* is a member selected from a polymeric modifying group and a glycosyl linking group comprising a polymeric modifying group.

16. The covalent conjugate according to claim 15, wherein Z* is a member selected from GlcNAc, GlcNH, Glc, GlcNAc-Fuc, GlcNAc-GlcNAc, GlcNH-GlcNH, GlcNAc-GlcNH, GlcNH-GlcNAc, GlcNAc-Gal, GlcNH-Gal, GlcNAc-Sia, GlcNH-Sia, GlcNAc-Gal-Sia, GlcNH-Gal-Sia, GlcNAc-GlcNAc-Gal-Sia, GlcNH-GlcNH-Gal-Sia, GlcNAc-GlcNH-Gal-Sia, GlcNH-GlcNAc-Gal-Sia, GlcNAc-GlcNAc-Man, GlcNAc-GlcNAc-Man(Man)₂ and GlcNAc-Gal-Gal-Sia.

17. The covalent conjugate according to claim 15, wherein Z* is a member selected from GlcNAc and GlcNH and X* is a polymeric modifying group.

18. The covalent conjugate according to claim 1, wherein said polymeric modifying group includes a moiety, which is a member selected from: 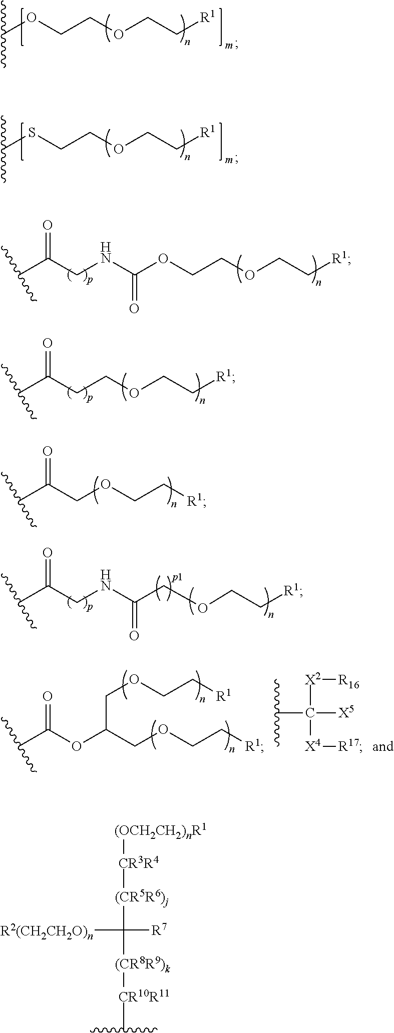
wherein
p and p1 are integers independently selected from 1 to 20;
j and k are integers independently selected from 0 to 20;
each n is an integer independently selected from 1 to 5000;
m is an integer from 1-5;
R¹⁶ and R¹⁷ are independently selected polymeric moieties;
X² and X⁴ are independently selected linkage fragments joining polymeric moieties R¹⁶ and R¹⁷ to C;
X⁵ is a member selected from H, substituted or unsubstituted alkyl, substituted or unsubstituted heteroalkyl, substituted or unsubstituted heterocycloalkyl, substituted or unsubstituted aryl, substituted or unsubstituted heteroaryl, —NR¹²R¹³ and —OR¹²;
R¹, R², R³, R⁴, R⁵, R⁶, R⁷, R⁸, R⁹, R¹⁰ and R¹¹ are members independently selected from H, substituted or unsubstituted alkyl, substituted or unsubstituted heteroalkyl, substituted or unsubstituted heterocycloalkyl, substituted or unsubstituted aryl, substituted or unsubstituted heteroaryl, —NR¹²R¹³, —OR¹² and —SiR¹²R¹³
wherein
R¹² and R¹³ are members independently selected from substituted or unsubstituted alkyl, substituted or unsubstituted heteroalkyl, substituted or unsubstituted heterocycloalkyl, substituted or unsubstituted aryl, and substituted or unsubstituted heteroaryl.

19. The covalent conjugate according to claim 1, wherein said covalent conjugate comprises a moiety according to Formula (VIII): 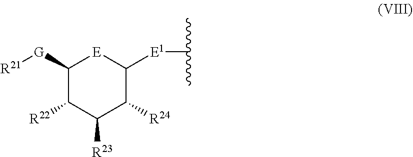
wherein
G is a member selected from —CH₂— and C=A, wherein A is a member selected from O, S and NR²⁷, wherein R²⁷ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl;
E is a member selected from O, S and CH₂;
E¹ is a member selected from O and S;
R²¹, R²², R²³ and R²⁴ are members independently selected from H, OR²⁵, SR²⁵, NR²⁵R²⁶, NR²⁵S(O)₂R²⁶, S(O)₂NR²⁵R²⁶, NR²⁵C(O)R²⁶, C(O)NR²⁵R²⁶, C(O)OR²⁵, acyl, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R²⁵ and R²⁶ are members independently selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl and a polymeric modifying group; and
wherein at least one of R²¹, R²², R²³, R²⁴ and R²⁷ comprises a polymeric modifying group.

20. The covalent conjugate according to claim 19, comprising a moiety according to Formula (IX): 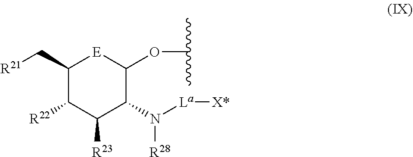
wherein
R²⁸ is a member selected from H, substituted or unsubstituted alkyl, substituted or unsubstituted heteroalkyl, substituted or unsubstituted aryl, substituted or unsubstituted heteroaryl and substituted or unsubstituted heterocycloalkyl;
X* is a polymeric modifying group selected from linear and branched; and
L^{a} is a member selected from a bond and a linker group selected from substituted or unsubstituted alkyl, substituted or unsubstituted heteroalkyl, substituted or unsubstituted aryl, substituted or unsubstituted heteroaryl and substituted or unsubstituted heterocycloalkyl.

21. The covalent conjugate according to claim 20, wherein said covalent conjugate comprises a moiety according to Formula (X):

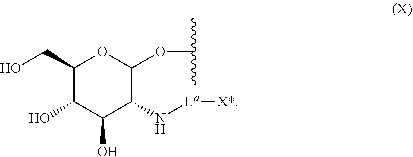

22. The covalent conjugate according to claim 21, wherein said covalent conjugate comprises a structure, which is a member selected from: 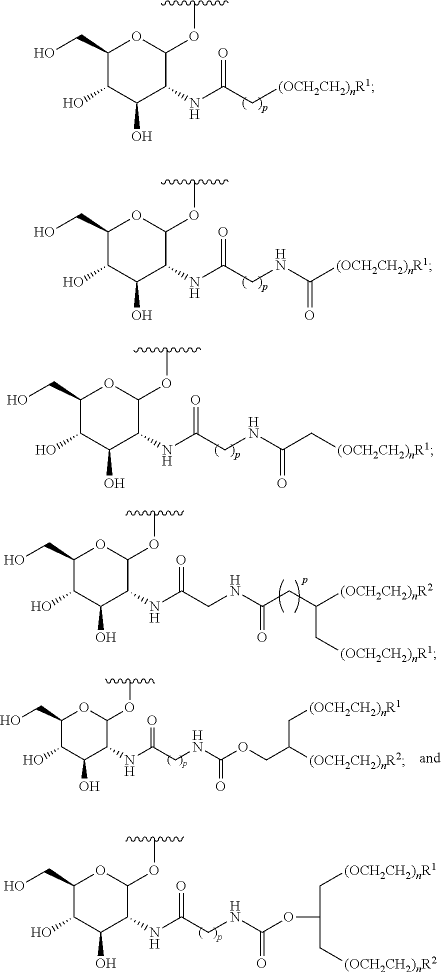
wherein
p is an integer selected from 1 to 20; and
R¹ and R² are members independently selected from OH and OMe.

23. A pharmaceutical composition comprising a covalent conjugate according to claim 1 and a pharmaceutically acceptable carrier.

24. A non-naturally occurring polypeptide corresponding to a parent polypeptide and comprising an exogenous O-linked glycosylation sequence that is not present, or not present at the same position, in said parent polypeptide, said O-linked glycosylation sequence being a substrate for a GlcNAc-transferase and comprising an amino acid sequence, which is a member selected from Formulae (I) to (VI):

| | |
|---|---|
| (B¹)ₐ P (B²)_{b} U S (B³)_{c} | (I) (SEQ ID NO: 247) |
| (B¹)ₐ P (B²)_{b} U T (B³)_{c} | (II) (SEQ ID NO: 248) |
| (B⁴)_{d} P S Z (B⁵)ₑ | (III) (SEQ ID NO: 249) |
| (B⁴)_{d} P T Z (B⁵)ₑ | (IV) (SEQ ID NO: 250) |
| (B⁶)_{f} S (B⁷)_{g} P (B⁸)ₕ | (V) (SEQ ID NO: 251) |
| (B⁶)_{f} T (B⁷)_{g} P (B⁸)ₕ | (VI) (SEQ ID NO: 252) |

wherein
b and g are integers selected from 0 to 2;
a, c, d, e, f and h are integers selected from 0 to 5;
T is threonine;
S is serine;
U is a member selected from V, S, T, E, Q and uncharged amino acids;
Z is a member selected from P, E, Q, S, T and uncharged amino acids; and
each B¹, B², B³, B⁴, B⁵, B⁶, B⁷ and B⁸ is a member independently selected from an amino acid.

25. The non-naturally occurring polypeptide according to claim 24, wherein said O-linked glycosylation sequence comprises an amino acid sequence, which is a member selected from:

| | |
|---|---|
| (B¹)ₐ P V S (B³)_{c}; | (SEQ ID NO: 16) |
| (B¹)ₐ P V T (B³)_{c}; | (SEQ ID NO: 17) |
| (B¹)ₐ P S S (B³)_{c}; | (SEQ ID NO: 18) |
| (B¹)ₐ P S T (B³)_{c}; | (SEQ ID NO: 19) |
| (B¹)ₐ P T S (B³)_{c}; | (SEQ ID NO: 20) |
| (B¹)ₐ P B² V T (B³)_{c}; | (SEQ ID NO: 21) |
| (B¹)ₐ P B² V S (B³)_{c}; | (SEQ ID NO: 22) |
| (B¹)ₐ P K U T (B³)_{c}; | (SEQ ID NO: 23) |
| (B¹)ₐ P K U S (B³)_{c}; | (SEQ ID NO: 24) |
| (B¹)ₐ P Q U T (B³)_{c}; | (SEQ ID NO: 25) |
| (B¹)ₐ P Q U S (B³)_{c}; | (SEQ ID NO: 26) |
| (B¹)ₐ P (B²)₂ V S (B³)_{c}; | (SEQ ID NO: 27) |
| (B¹)ₐ P (B²)₂ V T (B³)_{c}; | (SEQ ID NO: 28) |
| (B¹)ₐ P (B²)₂ T S (B³)_{c}; | (SEQ ID NO: 29) |
| (B¹)ₐ P (B²)₂ T T (B³)_{c}; | (SEQ ID NO: 30) |
| (B⁴)_{d} P T P (B⁵)ₑ; | (SEQ ID NO: 31) |
| (B⁴)_{d} P T E (B⁵)ₑ; | (SEQ ID NO: 32) |
| (B⁴)_{d} P S A (B⁵)ₑ; | (SEQ ID NO: 33) |
| (B⁶)_{f} S B⁷ T P (B⁸)ₕ; | (SEQ ID NO: 34) |
| and | |
| (B⁶)_{f} S B⁷ S P (B⁸)ₕ. | (SEQ ID NO: 35) |

26. An isolated nucleic acid encoding said non-naturally occurring polypeptide of claim 24.

27. An expression vector comprising said nucleic acid of claim 26.

28. A cell comprising said nucleic acid of claim 26.

29. A compound having a structure according to Formula (XI): 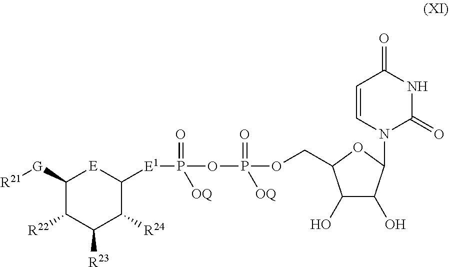
wherein
G is a member selected from —CH₂— and C=A, wherein A is a member selected from O, S and NR²⁷, wherein R²⁷ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl;
Q is a member selected from H, a negative charge and a salt counter ion;
E is a member selected from O, S, and CH₂;
E¹ is a member selected from O and S;
R²¹, R²², R²³ and R²⁴ are members independently selected from H, OR²⁵, SR²⁵, NR²⁵R²⁶, NR²⁵S(O)₂R²⁶, S(O)₂NR²⁵R²⁶, NR²⁵C(O)R²⁶, C(O)NR²⁵R²⁶, C(O)OR²⁵, acyl, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R²⁵ and R²⁶ are members independently selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl and a modifying group; and
wherein at least one of R²¹, R²², R²³, R²⁴ and R²⁷ comprises a polymeric modifying group.

30. The compound according to claim 29, wherein said polymeric modifying group is a water-soluble polymer.

31. The compound according to claim 30, wherein said water-soluble polymer is a member selected from poly(alkylene glycol), dextran and polysialic acid.

32. The compound according to claim 31, wherein said poly(alkylene glycol) is a member selected from poly(ethylene glycol) and poly(propylene glycol) and derivatives thereof.

33. The compound according to claim 32, wherein said poly(ethylene glycol) is monomethoxy-poly(ethylene glycol) (mPEG).

34. The compound according to claim 32, wherein said poly(ethylene glycol) has a molecular weight that is essentially homodisperse.

35. A method of forming a covalent conjugate between a polypeptide and a polymeric modifying group, wherein said polypeptide comprises an exogenous O-linked glycosylation sequence, said O-linked glycosylation sequence including an amino acid residue having a hydroxyl group, wherein said O-linked glycosylation sequence is a substrate for a GlcNAc-transferase and wherein said polymeric modifying group is covalently linked to said polypeptide via a glucosamine-linking group interposed between and covalently linked to both said polypeptide and said modifying group, said method comprising:
(i) contacting said polypeptide and a glucosamine-donor comprising a glucosamine-moiety covalently linked to said polymeric modifying group, in the presence of a GlcNAc-transferase under conditions sufficient for said GlcNAc-transferase to transfer said glucosamine-moiety from said glucosamine-donor onto said hydroxyl group of said O-linked glycosylation sequence,
thereby forming said covalent conjugate.

36. The method according to claim 35, further comprising:
(ii) recombinantly producing said polypeptide comprising said O-linked glycosylation sequence.

37. The method according to claim 35, further comprising:
(iii) isolating said covalent conjugate.

38. The method according to claim 35, wherein said polymeric modifying group is a water-soluble polymer.

39. The method according to claim 38, wherein said water-soluble polymer is a member selected from poly(alkylene glycol), dextran and polysialic acid.

40. The method according to claim 39, wherein said poly(alkylene glycol) is a member selected from poly(ethylene glycol) and poly(propylene glycol) and derivatives thereof.

41. The method according to claim 40, wherein said poly(ethylene glycol) is monomethoxy-poly(ethylene glycol) (mPEG).

42. The method according to claim 40, wherein said poly(ethylene glycol) has a molecular weight that is essentially homodisperse.

43. The method according to claim 35, wherein said polypeptide is a non-naturally occurring polypeptide.

44. The method according to claim 35, wherein said polypeptide is a therapeutic polypeptide.

45. The method according to claim 35, wherein said polypeptide is a member selected from bone morphogenetic protein 2 (BMP-2), bone morphogenetic protein 7 (BMP-7), neurotrophin-3 (NT-3), erythropoietin (EPO), granulocyte colony stimulating factor (G-CSF), granulocyte-macrophage colony stimulating factor (GM-CSF), interferon alpha, interferon beta, interferon gamma, α₁-antitrypsin (α-1 protease inhibitor), glucocerebrosidase, tissue-type plasminogen activator (TPA), interleukin-2 (IL-2), leptin, hirudin, urokinase, human DNase, insulin, hepatitis B surface protein (HbsAg), chimeric diphtheria toxin-IL-2, human growth hormone (hGH), human chorionic gonadotropin (hCG), alpha-galactosidase, alpha-L-iduronidase, beta-glucosidase, alpha-galactosidase A, acid α-glucosidase (acid maltase), anti-thrombin III (AT III), follicle stimulating hormone, glucagon-like peptide-1 (GLP-1), glucagon-like peptide-2 (GLP-2), fibroblast growth factor 7 (FGF-7), fibroblast growth factor 21 (FGF-21), fibroblast growth factor 23 (FGF-23), Factor VII, Factor VIII, B-domain deleted Factor VIII, Factor IX, Factor XIII, prokinetisin, extendin-4, CD4, tumor necrosis factor receptor (TNF-R), α-CD20, P-selectin glycoprotein ligand-1 (PSGL-1), complement, transferrin, glycosylation-dependent cell adhesion molecule (GlyCAM), neural-cell adhesion molecule (N-CAM), anti-TNF-alpha monoclonal antibody, TNF receptor-IgG Fc region fusion protein, anti-HER² monoclonal antibody, monoclonal antibody to respiratory syncytial virus, monoclonal antibody to protein F of respiratory syncytial virus, monoclonal antibody to TNF-α, monoclonal antibody to glycoprotein IIb/IIIa, monoclonal antibody to CD20, monoclonal antibody to VEGF-A, monoclonal antibody to PSGL-1, monoclonal antibody to CD4, monoclonal antibody to a-CD3, monoclonal antibody to EGF, monoclonal antibody to carcinoembryonic antigen (CEA) and monoclonal antibody to IL-2 receptor.

46. The method according to claim 35, wherein said glucosamine-moiety is a member selected from GlcNAc and GlcNH.

47. The method according to claim 35, wherein said GlcNAc-transferase is a recombinant enzyme.

48. The method according to claim 47, wherein said GlcNAc-transferase is expressed in a bacterial cell.

49. The method according to claim 35, wherein said glucosamine-donor has a structure according to Formula (XI): 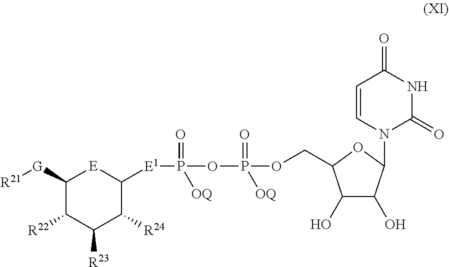
wherein
G is a member selected from CH₂ and C=A, wherein A is a member selected from O, S and NR²⁷, wherein R²⁷ is a member selected from substituted or unsubstituted alkyl, substituted or unsubstituted heteroalkyl, substituted or unsubstituted aryl, substituted or unsubstituted heteroaryl and substituted or unsubstituted heterocycloalkyl;
Q is a member selected from H, a negative charge and a salt counter ion;
E is a member selected from O, S, and CH₂;
E¹ is a member selected from O and S;
R²¹, R²², R²³ and R²⁴ are members independently selected from H, OR²⁵, SR²⁵, NR²⁵R²⁶, NR²⁵S(O)₂R²⁶, S(O)₂NR²⁵R²⁶, NR²⁵C(O)R²⁶, C(O)NR²⁵R²⁶, C(O)OR²⁵, acyl, substituted or unsubstituted alkyl, substituted or unsubstituted heteroalkyl, substituted or unsubstituted aryl, substituted or unsubstituted heteroaryl and substituted or unsubstituted heterocycloalkyl,
wherein
R²⁵ and R²⁶ are members independently selected from H, acyl, substituted or unsubstituted alkyl, substituted or unsubstituted heteroalkyl, substituted or unsubstituted aryl, substituted or unsubstituted heteroaryl, substituted or unsubstituted heterocycloalkyl and a modifying group; and
wherein at least one of R²¹, R²², R²³, R²⁴ and R²⁷ comprises a polymeric modifying group.


SEQUENCE LISTING (252 sequences)

SEQ ID NO: 1 - protein, 1046 aa, Homo sapiens
MASSVGNVADSTEPTKRMLSFQGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 2 - protein, 871 aa, Artificial Sequence
  recombinant human OGT delta176
MKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 3 - protein, 865 aa, Artificial Sequence
  recombinant human OGT delta182
MAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 4 - protein, 873 aa, Artificial Sequence
  recombinant human OGT delta182His8
MAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESAHHHHHHHH

SEQ ID NO: 5 - protein, 665 aa, Artificial Sequence
  recombinant human OGT delta382
MHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 6 - protein, 673 aa, Artificial Sequence
  recombinant human OGT delta382His8
MHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESAHHHHHHHH

SEQ ID NO: 7 - protein, 672 aa, Artificial Sequence
  recombinant human OGT His7delta382
MHHHHHHHHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 8 - protein, 1257 aa, Artificial Sequence
  recombinant human OGT delta182 MBP tagged
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFGSAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 9 - protein, 1057 aa, Artificial Sequence
  recombinant human OGT delta382 MBP tagged
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRISEFGSHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 10 - protein, 2351 aa, Homo sapiens [sequence omitted]

SEQ ID NO: 11 - protein, 2332 aa, Homo sapiens
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYEGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 12 - protein, 1424 aa, Artificial Sequence
  B-domain deleted factor VIII
ATRRYYLGAVELSWDYMQSDLGELPVDARFPPRVPKSFPFNTSVVYKKTLFVEFTVHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - protein, 1438 aa, Artificial Sequence
  B-domain deleted factor VIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 14 - protein, 1465 aa, Artificial Sequence
  B-domain deleted factor VIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRRRAQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 15 - protein, 1445 aa, Artificial Sequence
  B-domain deleted factor VIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 16 - protein, 13 aa, Artificial Sequence
  Synthetic O-linked glycosylation sequence motif
XXXXXPVSXXXXX

SEQ ID NO: 17 - protein, 13 aa, Artificial Sequence
  Synthetic O-linked glycosylation sequence motif
XXXXXPVTXXXXX

SEQ ID NO: 18 - protein, 13 aa, Artificial Sequence
  Synthetic O-linked glycosylation sequence motif
XXXXXPSSXXXXX

SEQ ID NO: 19 - protein, 13 aa, Artificial Sequence
  Synthetic O-linked glycosylation sequence motif
XXXXXPSTXXXXX

SEQ ID NO: 20 - protein, 13 aa, Artificial Sequence
  Synthetic O-linked glycosylation sequence motif
XXXXXPTSXXXXX

SEQ ID NO: 21 - protein, 14 aa, Artificial Sequence
  Synthetic O-linked glycosylation sequence motif
XXXXXPXVTXXXXX

SEQ ID NO: 22 - protein, 14 aa, Artificial Sequence
  Synthetic O-linked glycosylation sequence motif
XXXXXPXVSXXXXX

SEQ ID NO: 23 - protein, 14 aa, Artificial Sequence
  Synthetic O-linked glycosylation sequence motif
XXXXXPKXTXXXXX

SEQ ID NO: 24 - protein, 14 aa, Artificial Sequence
  Synthetic O-linked glycosylation sequence motif
XXXXXPKXSXXXXX

SEQ ID NO: 25 - protein, 14 aa, Artificial Sequence
  Synthetic O-linked glycosylation sequence motif
XXXXXPQXTXXXXX

SEQ ID NO: 26 - protein, 14 aa, Artificial Sequence
  Synthetic O-linked glycosylation sequence motif
XXXXXPQXSXXXXX

SEQ ID NO: 27 - protein, 15 aa, Artificial Sequence
  Synthetic O-linked glycosylation sequence motif
XXXXXPXXVSXXXXX

SEQ ID NO: 28 - protein, 15 aa, Artificial Sequence
  Synthetic O-linked glycosylation sequence motif
XXXXXPXXVTXXXXX

SEQ ID NO: 29 - protein, 15 aa, Artificial Sequence
  Synthetic O-linked glycosylation sequence motif
XXXXXPXXTSXXXXX

SEQ ID NO: 30 - protein, 15 aa, Artificial Sequence
  Synthetic O-linked glycosylation sequence motif
XXXXXPXXTTXXXXX

SEQ ID NO: 31 - protein, 13 aa, Artificial Sequence
  Synthetic O-linked glycosylation sequence motif
XXXXXPTPXXXXX

SEQ ID NO: 32 - protein, 13 aa, Artificial Sequence
  Synthetic O-linked glycosylation sequence motif
XXXXXPTEXXXXX

SEQ ID NO: 33 - protein, 13 aa, Artificial Sequence
  Synthetic O-linked glycosylation sequence motif
XXXXXPSAXXXXX

SEQ ID NO: 34 - protein, 14 aa, Artificial Sequence
  Synthetic O-linked glycosylation sequence motif
XXXXXSXTPXXXXX

SEQ ID NO: 35 - protein, 14 aa, Artificial Sequence
  Synthetic O-linked glycosylation sequence motif
XXXXXSXSPXXXXX

SEQ ID NO: 36 - protein, 3 aa, Artificial Sequence
  O-linked glycosylation sequence
PVS

SEQ ID NO: 37 - protein, 4 aa, Artificial Sequence
  O-linked glycosylation sequence
PVSG

SEQ ID NO: 38 - protein, 5 aa, Artificial Sequence
  O-linked glycosylation sequence
PVSGS

SEQ ID NO: 39 - protein, 4 aa, Artificial Sequence
  O-linked glycosylation sequence
VPVS

SEQ ID NO: 40 - protein, 5 aa, Artificial Sequence
  O-linked glycosylation sequence
VPVSG

SEQ ID NO: 41 - protein, 6 aa, Artificial Sequence
  O-linked glycosylation sequence
VPVSGS

SEQ ID NO: 42 - protein, 4 aa, Artificial Sequence
  O-linked glycosylation sequence
PVSR

SEQ ID NO: 43 - protein, 5 aa, Artificial Sequence
  O-linked glycosylation sequence
PVSRE

SEQ ID NO: 44 - protein, 4 aa, Artificial Sequence
  O-linked glycosylation sequence
PVSA

SEQ ID NO: 45 - protein, 5 aa, Artificial Sequence
  O-linked glycosylation sequence
PVSAS

SEQ ID NO: 46 - protein, 4 aa, Artificial Sequence
  O-linked glycosylation sequence
APVS

SEQ ID NO: 47 - protein, 5 aa, Artificial Sequence
  O-linked glycosylation sequence
APVSA

SEQ ID NO: 48 - protein, 6 aa, Artificial Sequence
  O-linked glycosylation sequence
APVSAS

SEQ ID NO: 49 - protein, 5 aa, Artificial Sequence
  O-linked glycosylation sequence
APVSS

SEQ ID NO: 50 - protein, 6 aa, Artificial Sequence
  O-linked glycosylation sequence
APVSSS

SEQ ID NO: 51 - protein, 4 aa, Artificial Sequence
  O-linked glycosylation sequence
PVSS

SEQ ID NO: 52 - protein, 5 aa, Artificial Sequence
  O-linked glycosylation sequence
PVSSA

SEQ ID NO: 53 - protein, 6 aa, Artificial Sequence
  O-linked glycosylation sequence
PVSSAP

SEQ ID NO: 54 - protein, 4 aa, Artificial Sequence
  O-linked glycosylation sequence
IPVS

SEQ ID NO: 55 - protein, 4 aa, Artificial Sequence
  O-linked glycosylation sequence
PVSR

SEQ ID NO: 56 - protein, 5 aa, Artificial Sequence
  O-linked glycosylation sequence
PVSRE

SEQ ID NO: 57 - protein, 5 aa, Artificial Sequence
  O-linked glycosylation sequence
IPVSR

SEQ ID NO: 58 - protein, 4 aa, Artificial Sequence
  O-linked glycosylation sequence
VPVS

SEQ ID NO: 59 - protein, 5 aa, Artificial Sequence
  O-linked glycosylation sequence
VPVSS

SEQ ID NO: 60 - protein, 6 aa, Artificial Sequence
  O-linked glycosylation sequence
VPVSSA

SEQ ID NO: 61 - protein, 4 aa, Artificial Sequence
  O-linked glycosylation sequence
RPVS

SEQ ID NO: 62 - protein, 5 aa, Artificial Sequence
  O-linked glycosylation sequence
RPVSS

SEQ ID NO: 63 - protein, 6 aa, Artificial Sequence
  O-linked glycosylation sequence
RPVSSA

SEQ ID NO: 64 - protein, 3 aa, Artificial Sequence
  O-linked glycosylation sequence
PVT

SEQ ID NO: 65 - protein, 3 aa, Artificial Sequence
  O-linked glycosylation sequence
PSS

SEQ ID NO: 66 - protein, 4 aa, Artificial Sequence
  O-linked glycosylation sequence
PSST

SEQ ID NO: 67 - protein, 5 aa, Artificial Sequence
  O-linked glycosylation sequence
PSSTA

SEQ ID NO: 68 - protein, 4 aa, Artificial Sequence
  O-linked glycosylation sequence
PPSS

SEQ ID NO: 69 - protein, 5 aa, Artificial Sequence
  O-linked glycosylation sequence
PPSST

SEQ ID NO: 70 - protein, 4 aa, Artificial Sequence
  O-linked glycosylation sequence
PSSG

SEQ ID NO: 71 - protein, 5 aa, Artificial Sequence
  O-linked glycosylation sequence
PSSGF

SEQ ID NO: 72 - protein, 4 aa, Artificial Sequence
  O-linked glycosylation sequence
SPST

SEQ ID NO: 73 - protein, 5 aa, Artificial Sequence
  O-linked glycosylation sequence
SPSTS

SEQ ID NO: 74 - protein, 6 aa, Artificial Sequence
  O-linked glycosylation sequence
SPSTSP

SEQ ID NO: 75 - protein, 4 aa, Artificial Sequence
  O-linked glycosylation sequence
SPSS

SEQ ID NO: 76 - protein, 5 aa, Artificial Sequence
  O-linked glycosylation sequence
SPSSG

SEQ ID NO: 77 - protein, 6 aa, Artificial Sequence
  O-linked glycosylation sequence
SPSSGF

SEQ ID NO: 78 - protein, 3 aa, Artificial Sequence
  O-linked glycosylation sequence
PST

SEQ ID NO: 79 - protein, 4 aa, Artificial Sequence
  O-linked glycosylation sequence
PSTS

SEQ ID NO: 80 - protein, 5 aa, Artificial Sequence
  O-linked glycosylation sequence
PSTST

SEQ ID NO: 81 - protein, 4 aa, Artificial Sequence
  O-linked glycosylation sequence
PSTV

SEQ ID NO: 82 - protein, 5 aa, Artificial Sequence
  O-linked glycosylation sequence
PSTVS

SEQ ID NO: 83 - protein, 4 aa, Artificial Sequence
  O-linked glycosylation sequence
PSVT

SEQ ID NO: 84 - protein, 5 aa, Artificial Sequence
  O-linked glycosylation sequence
PSVTI

SEQ ID NO: 85 - protein, 4 aa, Artificial Sequence
  O-linked glycosylation sequence
PSVS

SEQ ID NO: 86 - protein, 4 aa, Artificial Sequence
  O-linked glycosylation sequence
PAVT

SEQ ID NO: 87 - protein, 5 aa, Artificial Sequence
  O-linked glycosylation sequence
PAVTA

SEQ ID NO: 88 - protein, 6 aa, Artificial Sequence
  O-linked glycosylation sequence
PAVTAA

SEQ ID NO: 89 - protein, 5 aa, Artificial Sequence
  O-linked glycosylation sequence
KPAVT

SEQ ID NO: 90 - protein, 6 aa, Artificial Sequence
  O-linked glycosylation sequence
KPAVTA

SEQ ID NO: 91 - protein, 4 aa, Artificial Sequence
  O-linked glycosylation sequence
PAVS

SEQ ID NO: 92 - protein, 4 aa, Artificial Sequence
  O-linked glycosylation sequence
PQQS

SEQ ID NO: 93 - protein, 5 aa, Artificial Sequence
  O-linked glycosylation sequence
PQQSA

SEQ ID NO: 94 - protein, 6 aa, Artificial Sequence
  O-linked glycosylation sequence
PQQSAS

SEQ ID NO: 95 - protein, 4 aa, Artificial Sequence
  O-linked glycosylation sequence
PQQT

SEQ ID NO: 96 - protein, 4 aa, Artificial Sequence
  O-linked glycosylation sequence
PKGS

SEQ ID NO: 97 - protein, 5 aa, Artificial Sequence
  O-linked glycosylation sequence
PKGSR

SEQ ID NO: 98 - protein, 4 aa, Artificial Sequence
  O-linked glycosylation sequence
PKGT

SEQ ID NO: 99 - protein, 4 aa, Artificial Sequence
  O-linked glycosylation sequence
PKSS

SEQ ID NO: 100 - protein, 5 aa, Artificial Sequence
  O-linked glycosylation sequence
PKSSA

SEQ ID NO: 101 - protein, 6 aa, Artificial Sequence
  O-linked glycosylation sequence
PKSSAP

SEQ ID NO: 102 - protein, 4 aa, Artificial Sequence
  O-linked glycosylation sequence
PKST

SEQ ID NO: 103 - protein, 5 aa, Artificial Sequence
  O-linked glycosylation sequence
PADTS

SEQ ID NO: 104 - protein, 6 aa, Artificial Sequence
  O-linked glycosylation sequence
PADTSD

SEQ ID NO: 105 - protein, 5 aa, Artificial Sequence
  O-linked glycosylation sequence
PADTT

SEQ ID NO: 106 - protein, 5 aa, Artificial Sequence
  O-linked glycosylation sequence
PIKVT

SEQ ID NO: 107 - protein, 6 aa, Artificial Sequence
  O-linked glycosylation sequence
PIKVTE

SEQ ID NO: 108 - protein, 5 aa, Artificial Sequence
  O-linked glycosylation sequence
PIKVS

SEQ ID NO: 109 - protein, 4 aa, Artificial Sequence
  O-linked glycosylation sequence
SPST

SEQ ID NO: 110 - protein, 5 aa, Artificial Sequence
  O-linked glycosylation sequence
SPSTS

SEQ ID NO: 111 - protein, 4 aa, Artificial Sequence
  O-linked glycosylation sequence
SPTS

SEQ ID NO: 112 - protein, 5 aa, Artificial Sequence
  O-linked glycosylation sequence
SPTSP

SEQ ID NO: 113 - protein, 4 aa, Artificial Sequence
  O-linked glycosylation sequence
PTSP

SEQ ID NO: 114 - protein, 5 aa, Artificial Sequence
  O-linked glycosylation sequence
SPTSP

SEQ ID NO: 115 - protein, 4 aa, Artificial Sequence
  O-linked glycosylation sequence
SPSA

SEQ ID NO: 116 - protein, 5 aa, Artificial Sequence
  O-linked glycosylation sequence
SPSAK

SEQ ID NO: 117 - protein, 4 aa, Artificial Sequence
  O-linked glycosylation sequence
TSPS

SEQ ID NO: 118 - protein, 5 aa, Artificial Sequence
  O-linked glycosylation sequence
TSPSA

SEQ ID NO: 119 - protein, 4 aa, Artificial Sequence
  O-linked glycosylation sequence
LPTP

SEQ ID NO: 120 - protein, 5 aa, Artificial Sequence
  O-linked glycosylation sequence
LPTPP

SEQ ID NO: 121 - protein, 4 aa, Artificial Sequence
  O-linked glycosylation sequence
PTPP

SEQ ID NO: 122 - protein, 5 aa, Artificial Sequence
  O-linked glycosylation sequence
PTPPL

SEQ ID NO: 123 - protein, 4 aa, Artificial Sequence
  O-linked glycosylation sequence
VPTE

SEQ ID NO: 124 - protein, 5 aa, Artificial Sequence
  O-linked glycosylation sequence
VPTET

SEQ ID NO: 125 - protein, 3 aa, Artificial Sequence
  O-linked glycosylation sequence
PTE

SEQ ID NO: 126 - protein, 4 aa, Artificial Sequence
  O-linked glycosylation sequence
PTET

SEQ ID NO: 127 - protein, 5 aa, Artificial Sequence
  O-linked glycosylation sequence
TSETP

SEQ ID NO: 128 - protein, 6 aa, Artificial Sequence
  O-linked glycosylation sequence
ITSETP

SEQ ID NO: 129 - protein, 5 aa, Artificial Sequence
  O-linked glycosylation sequence
ASVSP

SEQ ID NO: 130 - protein, 6 aa, Artificial Sequence
  O-linked glycosylation sequence
SASVSP

SEQ ID NO: 131 - protein, 4 aa, Artificial Sequence
  O-linked glycosylation sequence
VETP

SEQ ID NO: 132 - protein, 5 aa, Artificial Sequence
  O-linked glycosylation sequence
VETPR

SEQ ID NO: 133 - protein, 4 aa, Artificial Sequence
  O-linked glycosylation sequence
ETPR

SEQ ID NO: 134 - protein, 4 aa, Artificial Sequence
  O-linked glycosylation sequence
ACTQ

SEQ ID NO: 135 - protein, 5 aa, Artificial Sequence
  O-linked glycosylation sequence
ACTQG

SEQ ID NO: 136 - protein, 4 aa, Artificial Sequence
  O-linked glycosylation sequence
CTQG

SEQ ID NO: 137 - protein, 140 aa, Homo sapiens
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 138 - protein, 140 aa, Artificial Sequence
  human BMP-7 variant
MPVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 139 - protein, 140 aa, Artificial Sequence
  Human BMP-7 variant
MSPVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 140 - protein, 140 aa, Artificial Sequence
  Human BMP-7 variant
MSTPVSQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 141 - protein, 140 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACPVS

SEQ ID NO: 142 - protein, 143 aa, Artificial Sequence
  Human BMP-7 variant
MPVSSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 143 - protein, 142 aa, Artificial Sequence
  Human BMP-7 variant
MPVSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 144 - protein, 141 aa, Artificial Sequence
  Human BMP-7 variant
MPVSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 145 - protein, 141 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGPVS

SEQ ID NO: 146 - protein, 142 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCPVS

SEQ ID NO: 147 - protein, 143 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHPVS

SEQ ID NO: 148 - protein, 141 aa, Artificial Sequence; identical to SEQ ID NO: 144
  Human BMP-7 variant

SEQ ID NO: 149 - protein, 141 aa, Artificial Sequence
  Human BMP-7 variant
MSPVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 150 - protein, 141 aa, Artificial Sequence
  Human BMP-7 variant
MSTPVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 151 - protein, 141 aa, Artificial Sequence
  Human BMP-7 variant
MSTGPVSQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 152 - protein, 141 aa, Artificial Sequence
  Human BMP-7 variant
MSTGSPVSRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 153 - protein, 142 aa, Artificial Sequence; identical to SEQ ID NO: 143
  Human BMP-7 variant

SEQ ID NO: 154 - protein, 142 aa, Artificial Sequence
  Human BMP-7 variant
MSPVSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 155 - protein, 142 aa, Artificial Sequence
  Human BMP-7 variant
MSTPVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 156 - protein, 142 aa, Artificial Sequence
  Human BMP-7 variant
MSTGPVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 157 - protein, 142 aa, Artificial Sequence
  Human BMP-7 variant
MSTGSPVSQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 158 - protein, 143 aa, Artificial Sequence
  Human BMP-7 variant
MSPVSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 159 - protein, 143 aa, Artificial Sequence
  Human BMP-7 variant
MSTPVSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 160 - protein, 143 aa, Artificial Sequence
  Human BMP-7 variant
MSTGPVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 161 - protein, 140 aa, Artificial Sequence
  Human BMP-7 variant
MPIKVSQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 162 - protein, 140 aa, Artificial Sequence
  Human BMP-7 variant
MSPIKVSRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 163 - protein, 140 aa, Artificial Sequence
  Human BMP-7 variant
MSTPIKVSSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 164 - protein, 140 aa, Artificial Sequence
  Human BMP-7 variant
MSTGPIKVSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 165 - protein, 145 aa, Artificial Sequence
  Human BMP-7 variant
MSTGSPIKVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 166 - protein, 145 aa, Artificial Sequence
  Human BMP-7 variant
MPIKVSSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 167 - protein, 144 aa, Artificial Sequence
  Human BMP-7 variant
MPIKVSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 168 - protein, 143 aa, Artificial Sequence
  Human BMP-7 variant
MPIKVSGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 169 - protein, 142 aa, Artificial Sequence
  Human BMP-7 variant
MPIKVSSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 170 - protein, 141 aa, Artificial Sequence
  Human BMP-7 variant
MPIKVSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 171 - protein, 145 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCHPIKVS

SEQ ID NO: 172 - protein, 144 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCPIKVS

SEQ ID NO: 173 - protein, 143 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGPIKVS

SEQ ID NO: 174 - protein, 142 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACPIKVS

SEQ ID NO: 175 - protein, 141 aa, Artificial Sequence
  Human BMP-7 variant
MSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRAPIKVS

SEQ ID NO: 176 - protein, 141 aa, Artificial Sequence
  Human BMP-7 variant
MTSETPKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 177 - protein, 141 aa, Artificial Sequence
  Human BMP-7 variant
MSTSETPQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 178 - protein, 141 aa, Artificial Sequence
  Human BMP-7 variant
MSTTSETPRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 179 - protein, 141 aa, Artificial Sequence
  Human BMP-7 variant
MSTGTSETPSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 180 - protein, 142 aa, Artificial Sequence
  Human BMP-7 variant
MTSETPSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 181 - protein, 142 aa, Artificial Sequence
  Human BMP-7 variant
MSTSETPKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 182 - protein, 142 aa, Artificial Sequence
  Human BMP-7 variant
MSTTSETPQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 183 - protein, 143 aa, Artificial Sequence
  Human BMP-7 variant
MTSETPGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 184 - protein, 143 aa, Artificial Sequence
  Human BMP-7 variant
MSTSETPSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 185 - protein, 143 aa, Artificial Sequence
  Human BMP-7 variant
MSTTSETPKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 186 - protein, 143 aa, Artificial Sequence
  Human BMP-7 variant
MSTGTSETPQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 187 - protein, 144 aa, Artificial Sequence
  Human BMP-7 variant
MTSETPTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 188 - protein, 144 aa, Artificial Sequence
  Human BMP-7 variant
MSTSETPGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 189 - protein, 144 aa, Artificial Sequence
  Human BMP-7 variant
MSTTSETPSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 190 - protein, 144 aa, Artificial Sequence
  Human BMP-7 variant
MSTGTSETPKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 191 - protein, 145 aa, Artificial Sequence
  Human BMP-7 variant
MTSETPSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 192 - protein, 145 aa, Artificial Sequence
  Human BMP-7 variant
MSTSETPTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 193 - protein, 145 aa, Artificial Sequence
  Human BMP-7 variant
MSTTSETPGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 194 - protein, 145 aa, Artificial Sequence
  Human BMP-7 variant
MSTGTSETPSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 195 - protein, 31 aa, Artificial Sequence
  Human BMP-7 partial sequence
AFPLNSYMNATNHAIVQTLVHFINPETVPKP

SEQ ID NO: 196 - protein, 31 aa, Artificial Sequence
  Human BMP-7 variant partial sequence
PVSLNSYMNATNHAIVQTLVHFINPETVPKP

SEQ ID NO: 197 - protein, 31 aa, Artificial Sequence
  Human BMP-7 variant partial sequence
APVSNSYMNATNHAIVQTLVHFINPETVPKP

SEQ ID NO: 198 - protein, 31 aa, Artificial Sequence
  Human BMP-7 variant partial sequence
AFPVSSYMNATNHAIVQTLVHFINPETVPKP

SEQ ID NO: 199 - protein, 31 aa, Artificial Sequence
  Human BMP-7 variant partial sequence
AFPPVSYMNATNHAIVQTLVHFINPETVPKP

SEQ ID NO: 200 - protein, 31 aa, Artificial Sequence
  Human BMP-7 variant partial sequence
AFPLPVSMNATNHAIVQTLVHFINPETVPKP

SEQ ID NO: 201 - protein, 31 aa, Artificial Sequence
  Human BMP-7 variant partial sequence
AFPLNPVSNATNHAIVQTLVHFINPETVPKP

SEQ ID NO: 202 - protein, 31 aa, Artificial Sequence
  Human BMP-7 variant partial sequence
AFPLNSPVSATNHAIVQTLVHFINPETVPKP

SEQ ID NO: 203 - protein, 31 aa, Artificial Sequence
  Human BMP-7 variant partial sequence
AFPLNSYPVSTNHAIVQTLVHFINPETVPKP

SEQ ID NO: 204 - protein, 31 aa, Artificial Sequence
  Human BMP-7 variant partial sequence
AFPLNSYMNATNHAIVQTLVHFPVSETVPKP

SEQ ID NO: 205 - protein, 31 aa, Artificial Sequence
  Human BMP-7 variant partial sequence
AFPLNSYMNATNHAIVQTLVHFIPVSTVPKP

SEQ ID NO: 206 - protein, 31 aa, Artificial Sequence
  Human BMP-7 variant partial sequence
AFPLNSYMNATNHAIVQTLVHFINPVSVPKP

SEQ ID NO: 207 - protein, 31 aa, Artificial Sequence
  Human BMP-7 variant partial sequence
AFPLNSYMNATNHAIVQTLVHFINPPVSPKP

SEQ ID NO: 208 - protein, 31 aa, Artificial Sequence
  Human BMP-7 variant partial sequence
AFPLNSYMNATNHAIVQTLVHFINPEPVSKP

SEQ ID NO: 209 - protein, 31 aa, Artificial Sequence
  Human BMP-7 variant partial sequence
AFPLNSYMNATNHAIVQTLVHFINPETPVSP

SEQ ID NO: 210 - protein, 31 aa, Artificial Sequence
  Human BMP-7 variant partial sequence
AFPLNSYMNATNHAIVQTLVHFINPETVPVS

SEQ ID NO: 211 - protein, 32 aa, Artificial Sequence
  Human BMP-7 variant partial sequence
PVSPLNSYMNATNHAIVQTLVHFINPETVPKP

SEQ ID NO: 212 - protein, 32 aa, Artificial Sequence
  Human BMP-7 variant partial sequence
APVSLNSYMNATNHAIVQTLVHFINPETVPKP

SEQ ID NO: 213 - protein, 32 aa, Artificial Sequence
  Human BMP-7 variant partial sequence
AFPVSNSYMNATNHAIVQTLVHFINPETVPKP

SEQ ID NO: 214 - protein, 32 aa, Artificial Sequence
  Human BMP-7 variant partial sequence
AFPPVSSYMNATNHAIVQTLVHFINPETVPKP

SEQ ID NO: 215 - protein, 32 aa, Artificial Sequence
  Human BMP-7 variant partial sequence
AFPLPVSYMNATNHAIVQTLVHFINPETVPKP

SEQ ID NO: 216 - protein, 32 aa, Artificial Sequence
  Human BMP-7 variant partial sequence
AFPLNPVSMNATNHAIVQTLVHFINPETVPKP

SEQ ID NO: 217 - protein, 32 aa, Artificial Sequence
  Human BMP-7 variant partial sequence
AFPLNSPVSNATNHAIVQTLVHFINPETVPKP

SEQ ID NO: 218 - protein, 32 aa, Artificial Sequence
  Human BMP-7 variant partial sequence
AFPLNSYPVSATNHAIVQTLVHFINPETVPKP

SEQ ID NO: 219 - protein, 32 aa, Artificial Sequence
  Human BMP-7 variant partial sequence
AFPLNSYMPVSTNHAIVQTLVHFINPETVPKP

SEQ ID NO: 220 - protein, 32 aa, Artificial Sequence
  Human BMP-7 variant partial sequence
AFPLNSYMNATNHAIVQTLVHFPVSPETVPKP

SEQ ID NO: 221 - protein, 32 aa, Artificial Sequence
  Human BMP-7 variant partial sequence
AFPLNSYMNATNHAIVQTLVHFIPVSETVPKP

SEQ ID NO: 222 - protein, 32 aa, Artificial Sequence
  Human BMP-7 variant partial sequence
AFPLNSYMNATNHAIVQTLVHFINPVSTVPKP

SEQ ID NO: 223 - protein, 32 aa, Artificial Sequence
  Human BMP-7 variant partial sequence
AFPLNSYMNATNHAIVQTLVHFINPPVSVPKP

SEQ ID NO: 224 - protein, 32 aa, Artificial Sequence
  Human BMP-7 variant partial sequence
AFPLNSYMNATNHAIVQTLVHFINPEPVSPKP

SEQ ID NO: 225 - protein, 32 aa, Artificial Sequence
  Human BMP-7 variant partial sequence
AFPLNSYMNATNHAIVQTLVHFINPETPVSKP

SEQ ID NO: 226 - protein, 32 aa, Artificial Sequence
  Human BMP-7 variant partial sequence
AFPLNSYMNATNHAIVQTLVHFINPETVPVSP

SEQ ID NO: 227 - protein, 32 aa, Artificial Sequence
  Human BMP-7 variant partial sequence
AFPLNSYMNATNHAIVQTLVHFINPETVPPVS

SEQ ID NO: 228 - protein, 1046 aa, Homo sapiens; identical to SEQ ID NO: 1

SEQ ID NO: 229 - protein, 1036 aa, Homo sapiens
MASSVGNVADSTGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 230 - protein, 920 aa, Homo sapiens
MLQGHFWLVREGIMISPSSPPPPNLFFFPLQIFPFPFTSFPSHLLSLTPPKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 231 - protein, 20 aa, Artificial Sequence
  Muc 1 region
HGVTSAPDTRPAPGSTAPPA

SEQ ID NO: 232 - protein, 24 aa, Artificial Sequence
  Muc 1 region
TAPPAHGVTSAPDTRPAPGSTAPP

SEQ ID NO: 233 - protein, 357 aa, Artificial Sequence
  Core-1-GalT1 delta31
GFCLAELFVYSTPERSEFMPYDGHRHGDVNDAHHSHDMMEMSGPEQDVGGHEHVHENSTIAERLYSEVRVLCWIMTNPSNHQKKARHVKRTWGKRCNKLIFMSSAKDDELDAVALPVGEGRNNLWGKTKEAYKYIYEHHINDADWFLKADDDTYTIVENMRYMLYPYSPETPVYFGCKFKPYVKQGYMSGGAGYVLSREAVRRFVVEALPNPKLCKSDNSGAEDVEIGKCLQNVNVLAGDSRDSNGRGRFFPFVPEHHLIPSHTDKKFWYWQYIFYKTDEGLDCCSDNAISFHYVSPNQMYVLDYLIYHLRPYGIINTPDALPNKLAVGELMPEIKEQATESTSDGVSKRSAETKTQ

SEQ ID NO: 234 - protein, 166 aa, Artificial Sequence
  IFN alpha variant
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVPVSRAPLMKEDSILAVRKYFQRITLYLKEKKYSPCAWEVVRAEIMRSFSLSTNLQESLRSKE

SEQ ID NO: 235 - protein, 166 aa, Artificial Sequence
  IFN alpha variant
MCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGPVSRPLMKEDSILAVRKYFQRITLYLKEKKYSPCAWEVVRAEIMRSFSLSTNLQESLRSKE

SEQ ID NO: 236 - protein, 145 aa, Artificial Sequence
  BMP-7 variant
MVPVSGSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 237 - protein, 191 aa, Artificial Sequence
  human growth hormone variant
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPVSGSIFKQTYSKFDTNSHNDDALLKNYGLLYCFRKDMDKVETFLRIVQCRSVEGSCG

SEQ ID NO: 238 - protein, 179 aa, Artificial Sequence
  GCSF variant
MPVSGTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 239 - protein, 7 aa, Artificial Sequence
  O-linked glycosylation sequence
PIPVSRE

SEQ ID NO: 240 - protein, 7 aa, Artificial Sequence
  O-linked glycosylation sequence
RIPVSRE

SEQ ID NO: 241 - protein, 7 aa, Artificial Sequence
  O-linked glycosylation sequence
RIPVSRA

SEQ ID NO: 242 - protein, 7 aa, Artificial Sequence
  O-linked glycosylation sequence
PIPVSRA

SEQ ID NO: 243 - protein, 7 aa, Artificial Sequence
  O-linked glycosylation sequence
RIPVSRP

SEQ ID NO: 244 - protein, 7 aa, Artificial Sequence
  O-linked glycosylation sequence
PIPVSRP

SEQ ID NO: 245 - protein, 7 aa, Artificial Sequence
  O-linked glycosylation sequence
AIPVSRA

SEQ ID NO: 246 - protein, 7 aa, Artificial Sequence
  O-linked glycosylation sequence
AIPVSRP

SEQ ID NO: 247 - protein, 15 aa, Artificial Sequence
  Synthetic O-linked glycosylation sequence motif
XXXXXPXXXSXXXXX

SEQ ID NO: 248 - protein, 15 aa, Artificial Sequence
  Synthetic O-linked glycosylation sequence motif
XXXXXPXXXTXXXXX

SEQ ID NO: 249 - protein, 13 aa, Artificial Sequence
  Synthetic O-linked glycosylation sequence motif
XXXXXPSXXXXXX

SEQ ID NO: 250 - protein, 13 aa, Artificial Sequence
  Synthetic O-linked glycosylation sequence motif
XXXXXPTXXXXXX

SEQ ID NO: 251 - protein, 14 aa, Artificial Sequence
  Synthetic O-linked glycosylation sequence motif
XXXXXSXXPXXXXX

SEQ ID NO: 252 - protein, 14 aa, Artificial Sequence
  Synthetic O-linked glycosylation sequence motif
XXXXXTXXPXXXXX